(19) United States
(12) Patent Application Publication
(10) Pub. No.: US 2024/0122865 A1
(43) Pub. Date: Apr. 18, 2024

(54) Methods of Protecting RNA

(71) Applicant: Pfizer Inc., New York, NY (US)

(72) Inventors: Justin Leigh Bellenger, Cheshire, CT (US); Ye Che, Niantic, CT (US); Miguel Angel Garcia, Midland Park, NJ (US); Lei Hu, New City, NY (US); Mark Carl Noe, East Lyme, CT (US); Shuai Shi, Whippany, NJ (US)

(73) Assignee: Pfizer Inc., New York, NY (US)

(21) Appl. No.: 18/275,634

(22) PCT Filed: Feb. 15, 2022
(86) PCT No.: PCT/IB2022/051339

§ 371 Date: Aug. 3, 2023

Related U.S. Application Data
(60) Provisional application No. 63/151,165, filed on Feb. 19, 2021.
(60) Provisional application No. 63/261,437, filed on Sep. 21, 2021.

(51) Int. Cl. A61K 9/51; A61K 9/48; A61K 39/12

(52) U.S. Cl. CPC A61K 9/5123; A61K 9/4858; A61K 39/12

(57) ABSTRACT

The present disclosure provides methods of protecting ribonucleic acid (RNA) from degradation under certain conditions by contacting the RNA with an amino acid. The present disclosure also provides compositions comprising such amino acid(s) and RNA.

This disclosure relates to the field of oligonucleotides, specifically to RNA, methods and compositions for protecting oligonucleotides from degradation, and to methods of increasing immunogenicity of and/or in vitro expression of oligonucleotide compositions.

### BACKGROUND

Over the last 30 years, RNA has been shown to be capable of producing therapeutic effects in cells and complex organisms, and recently, RNA-based drugs have been approved for use in subjects. RNA's potential as a therapeutic agent is an active area of biomedical research, and this area is expected to mature rapidly in the coming years. To date, RNA has been used to target nucleic acids and proteins, and to encode proteins. Single-strand antisense oligonucleotides and double-strand interfering RNAs can prevent protein synthesis by preventing transcription and/or translation. RNA can also alter protein function directly by forming aptamers to control protein activity, thereby realizing a therapeutic goal related to altering protein function. Further, messenger RNAs (mRNAs) can be used to produce desired proteins, for example, two important therapeutic uses of mRNAs are as vaccines or for protein replacement (Sarah Deweerdt, Nature, Vol. 574, 17 Oct. 2019).

While it is clear that RNA can have powerful and beneficial therapeutic effects, a major challenge to its use involves manufacture, dosing, and delivery. (Kenneth Lundstrom, Gene Therapy, Vol. 27, 183-185, 2020). RNA is highly susceptible to the ever-ubiquitous RNAse, an enzyme that biological organisms produce and secrete in high abundance to degrade foreign RNA. In addition to biochemical attack, RNA is prone to acid or base-mediated chemical degradation, especially at the 2′ hydroxyl group in the phosphate backbone. The rate of this chemical degradation follows first order kinetics and is highly influenced by temperature and pH. With regard to dosing and delivery, RNA is a macromolecule and is therefore unable to cross cell membranes readily via simple diffusion. RNA likely cannot be administered alone, either orally or via injection, as the body will rapidly clear the RNA before any therapeutic effect can be produced at the intended site of action. Effective delivery involves allowing the RNA to reach the desired site of action and allowing it to enter the cell by processes beyond simple diffusion.

Typically a delivery vehicle, such as a lipid nanoparticle that effectively encapsulates the RNA, must be used to deliver RNA into cells, usually via endocytosis, to achieve the desired effect. Although such lipoidal formulations have shown to be effective for in vivo and in vitro delivery, they can pose problems to the RNA therapeutic itself. Specifically, when RNA is formulated with commonly used lipoidal excipients to create nanoparticles, liposomes, or micelles, the RNA can experience a highly basic local environment resulting in chemical degradation of the RNA. Amelioration of this degradation is typically achieved by short latency between formulation and dosing, or by maintaining the RNA at ultra-low temperatures (such as −20° C. to −80° C.), and thus a need exists to overcome these degradation issues. Discovering ways to protect RNA from chemically mediated RNA degradation is currently an unmet challenge where solutions would have a large benefit towards the manufacture, dosing, and delivery of RNA therapeutics. Furthermore, the discovery of methods that protect RNA from degradation would enable research groups world-wide to evaluate and develop RNA as a viable therapeutic for treating a variety of conditions/disorders in subjects in need of such treatment.

Previous studies have explored the structural nature of interactions between amino acids and the bases found in DNA and RNA nucleic acids (see, e.g. Lustig et al., Nucleic Acids Res. 25(13); pp. 2562-2565 (1997); Lustig et al., Nucleic Acids Res. 23; pp. 4707-4711 (1995)).

### SUMMARY

The present disclosure provides compositions and methods for protecting a wide variety of RNAs from degradation, increasing immunogenicity of compositions comprising RNAs, and increasing in vitro expression of RNAs by contacting the RNAs with amino acids. Improving the in vitro expression level and/or immunogenicity of a therapeutic RNA by protecting the RNA from degradation is a desirable outcome in the manufacture, dosing and delivery of RNA therapeutics. The present disclosure is based, at least in part, on the discovery that contacting an RNA molecule with a free amino acid provides protection against degradation of the RNA and also increases immunogenicity of the RNA and in vitro expression of the RNA.

Without being bound by theory or mechanism, contacting an isolated RNA molecule with a free amino acid according to the present disclosure may protect against degradation of the RNA, increase immunogenicity of the RNA, and/or increase in vitro expression of the RNA by decreasing the pH of a composition comprising the RNA.

Disclosed herein, in some embodiments, is a method of protecting an isolated RNA from degradation by forming a solution containing the isolated RNA and at least 1 mM of a free amino acid, the method comprising contacting the isolated RNA with the amino acid. Also disclosed herein, in some embodiments, is a method of protecting an isolated RNA that is encapsulated in a capsule from degradation by forming a solution containing the encapsulated isolated RNA and at least 1 mM of a free amino acid, the method comprising contacting the capsule encapsulating the RNA with the free amino acid. Disclosed herein, in further embodiments, is a method of increasing immunogenicity of isolated RNA that is encapsulated and/or increasing in vitro expression of isolated RNA that is encapsulated by forming a composition in solution comprising the isolated RNA and at least 1 mM of a free amino acid, the method comprising contacting a capsule comprising the isolated RNA with the free amino acid.

Also disclosed herein, in some embodiments, is a composition comprising in solution: at least 1 mM of a free amino acid; and an isolated RNA that, in some embodiments, is comprised in a capsule.

In specific embodiments, the RNA is double stranded. In specific embodiments, the RNA is single stranded. In specific embodiments, the RNA is antisense single stranded. In specific embodiments, the RNA is messenger RNA (mRNA). In specific embodiments, the RNA is self-amplifying RNA (saRNA). In some embodiments, the RNA comprises a 5′ Cap represented by Formula I,

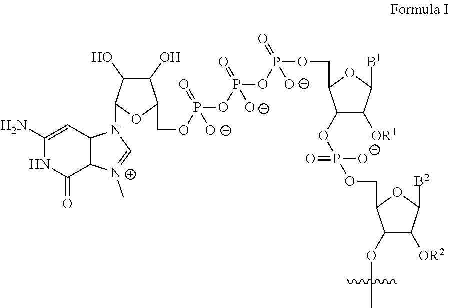

where R¹ and R² are each independently H or Me; and B¹ and B² are each independently guanine, adenine, or uracil. In some embodiments, the RNA comprises an open reading frame encoding a gene of interest. In some embodiments, the RNA comprises a 5′ untranslated region; a coding region for a nonstructural protein derived from an alphavirus; a subgenomic promoter derived from an alphavirus; an open reading frame encoding a gene of interest; a 3′ untranslated region; and a 3′ poly A sequence. In some embodiments, the RNA is modified RNA (modRNA). In some embodiments, the RNA is viral RNA. In some embodiments, the RNA encodes an antigen. In some embodiments, the RNA encodes a viral antigen. In some embodiments, the RNA is part of a vaccine.

In some embodiments, the RNA is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

In some embodiments, the solution further comprises a sugar and/or a buffer. In specific cases, the sugar is at a concentration of about 10% w/v. In specific cases, the buffer is at a concentration of about 10 mM. In specific cases, the solution is buffered at a pH of about 7.4. In some embodiments, the sugar comprises sucrose and/or trehalose. In specific cases, the sugar comprises sucrose. In some embodiments, the buffer comprises Tris buffer, PBS, or HEPES buffer. In specific cases, the buffer comprises Tris buffer. In specific embodiments, the sugar comprises sucrose and the buffer comprises Tris buffer.

In some embodiments disclosed herein, the RNA is encapsulated in, bound to, or adsorbed on a material or capsule encapsulating the RNA. In some embodiments, the material or a capsule encapsulating the RNA comprises a lipid. In certain embodiments, the material or capsule encapsulating the RNA comprises a cationic lipid. In certain embodiments, the material or capsule encapsulating the RNA comprises a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

In some embodiments in which the RNA is encapsulated, the material or capsule encapsulating the RNA further encapsulates the at least 1 mM of a free amino acid. In some embodiments in which the RNA is encapsulated, the isolated RNA is contacted with the free amino acid before the isolated RNA is encapsulated. In some embodiments in which the RNA is encapsulated, the isolated RNA is contacted with the at least 1 mM of a free amino acid during encapsulation of the RNA. In some embodiments in which the RNA is encapsulated, the material or capsule encapsulating the RNA the material does not encapsulate the at least 1 mM of a free amino acid. In some embodiments in which the RNA is encapsulated, the capsule comprising the isolated RNA is contacted with the at least 1 mM of a free amino acid after the isolated RNA is encapsulated in the capsule.

In some embodiments, the solution comprises the free amino acid at a concentration equal to any one of, at least any one of, at most any one of, or between any two of 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, or 40 mM. In specific embodiments, the solution comprises at least 5 mM of the free amino acid.

In specific embodiments, the solution comprises at least 10 mM of the free amino acid. In specific embodiments, the solution comprises at least 15 mM of the free amino acid. In specific embodiments, the solution comprises at least 20 mM of the free amino acid. In some embodiments, the free amino acid is selected from the group consisting of glutamic acid, glutamate, aspartic acid, aspartate, arginine, lysine, tyrosine, cysteine, glutamine, asparagine, threonine, and serine, or a pharmaceutically acceptable salt thereof, or combinations thereof. In specific embodiments, the free amino acid is glutamic acid, glutamate, or a pharmaceutically acceptable salt thereof. In specific embodiments, the free amino acid is aspartic acid, aspartate, or a pharmaceutically acceptable salt thereof. In specific embodiments, the free amino acid is arginine, or a pharmaceutically acceptable salt thereof. In specific embodiments, the free amino acid is lysine, or a pharmaceutically acceptable salt thereof.

In some embodiments of the methods disclosed herein, the method further comprises the step of of storing the solution comprising the RNA and at least 1 mM of the free amino acid. In some embodiments, the composition disclosed herein is stored. In certain embodiments, the storage is for a time equal to any one of, at least any one of, at most any one of, or between any two of about 2 hours, 4 hours, 6 hours, 12 hours, 1 day, 2 days, 3 days, 4 days, 5 days, 6 days, 7 days, 2 weeks, 3 weeks, 4 weeks, 5 weeks, 6 weeks, 7 weeks, 8 weeks, 2 months, 3 months, 4 months, 5 months, 6 months, 7 months, 8 months, 9 months, 10 months, 11 months, 1 year, 2 years, 3 years, 4 years, 5 years, etc. In certain embodiments, the storage is at a temperature equal to any one of, at least any one of, at most any one of, or between any two of about 20° C., 15° C., 10° C., 5° C., 0° C., −10° C., −20° C., −30° C., −40° C., −50° C., −60° C., −70° C., or −80° C.

In some embodiments, the wherein the at least 1 mM of a free amino acid provides a protective effect to the RNA in a Droplet Digital Polymerase Chain Reaction Assay. In some embodiments, the Droplet Digital Polymerase Chain Reaction Assay conditions accelerate degradation of the RNA. In some embodiments, the at least 1 mM of a free amino acid provides a protective effect to the RNA in a TapeStation Assay. In some embodiments, the TapeStation Assay conditions accelerate degradation of the RNA. In some embodiments, the at least 1 mM of a free amino acid provides a protective effect to the RNA in a Size Exclusion Base Hydrolysis Assay. In some embodiments, the Size Exclusion Base Hydrolysis Assay conditions accelerate degradation of the RNA.

In some embodiments disclosed herein, protection of the isolated RNA from degradation is assessed by measuring in vitro expression of the RNA. In some embodiments, contacting the capsule comprising the isolated RNA with the at least 1 mM of a free amino acid increases in vitro expression of the isolated RNA compared to in vitro expression of encapsulated isolated RNA that has not been contacted with the at least 1 mM of a free amino acid. In some embodiments, in vitro expression of the isolated RNA is increased equal to any one of, at least any one of, at most any one of, or between any two of about 20%, 21%, 22%, 23%, 24%, 25%, 26%, 27%, 28%, 29%, 30%, 32%, 33%, 34%, 35%, 36%, 37%, 38%, 39%, or 40%. In specific embodiments, in vitro expression of the isolated RNA is increased at least about 33%.

In some embodiments disclosed herein, protection of the isolated RNA from degradation is assessed by measuring immunogenicity of the isolated RNA. In some embodiments, contacting the capsule comprising the isolated RNA with the at least 1 mM of a free amino acid increases immunogenicity of the isolated RNA compared to immunogenicity of encapsulated isolated RNA that has not been contacted with the at least 1 mM of a free amino acid. In some embodiments, immunogenicity of the isolated RNA is increased equal to any one of, at least any one of, at most any one of, or between any two of about 15%, 16%, 17%, 18%, 19%, 20%, 21%, 22%, 23%, 24%, 25%, 26%, 27%, 28%, 29%, 30%, 32%, 33%, 34%, or 35%. In specific embodiments, immunogenicity of the isolated RNA is increased at least about 25%.

In some embodiments of the methods, the isolated RNA is comprised in any one of the compositions disclosed herein. In some embodiments of the methods, the capsule comprising the isolated RNA is comprised in any one of the compositions disclosed herein.

Disclosed herein, in some embodiments, are methods of producing the compositions described herein. In some embodiments, the methods comprise: (a) combining the isolated RNA and the at least 1 mM of a free amino acid, optionally, before the isolated RNA is encapsulated in the capsule; (b) combining the isolated RNA and the at least 1 mM of a free amino acid during RNA encapsulation in the capsule; or (c) combining the capsule comprising the isolated RNA and the at least 1 mM of a free amino acid after the isolated RNA is encapsulated in the capsule.

Also disclosed herein, in some embodiments, are methods of: (a) vaccinating a subject, comprising administering to the subject in need thereof an effective amount of a composition described herein; and (b) treating or preventing an infectious disease, comprising administering to a subject in need thereof an effective amount of a composition described herein. In some embodiments, the composition elicits an immune response comprising an antibody response. In some embodiments, the composition elicits an immune response comprising a T cell response. In some embodiments, immunogenicity of the composition is increased compared to immunogenicity of a composition in which the capsule comprising the isolated RNA has not been contacted with the at least 1 mM of a free amino acid. In some embodiments, immunogenicity of the composition is increased equal to any one of, at least any one of, at most any one of, or between any two of about 15%, 16%, 17%, 18%, 19%, 20%, 21%, 22%, 23%, 24%, 25%, 26%, 27%, 28%, 29%, 30%, 32%, 33%, 34%, or 35%. In specific embodiments, immunogenicity of the composition is increased at least about 25%.

Also disclosed are the following aspects or embodiments (E) 1 to 466 of the present disclosure.

E1. According to a first aspect of the present disclosure, there is provided a method of protecting an RNA from degradation by contacting the RNA with at least one amino acid.

E2. The method according to embodiment E1 wherein the RNA is double stranded RNA.

E3. The method according to embodiment E1 wherein the RNA is a single strand RNA.

E4. The method according to embodiment E1 wherein the RNA is an antisense single strand RNA.

E5. The method according to embodiment E1 wherein the RNA is a messenger RNA (mRNA).

E6. The method according to embodiment E1 wherein the RNA is a self-amplifying RNA (saRNA).

E7. The method according to embodiment E1 wherein the RNA is replicon RNA.

E8. The method according to embodiment E1 wherein the RNA is modified RNA (modRNA).

E9. The method according to any one of embodiments E1 to E8 wherein the RNA is viral RNA.

E10. The method according to any one of embodiments E1 to E9 wherein the RNA encodes an antigen.

E11. The method according to any one of embodiments E1 to E10 wherein the RNA is part of a vaccine.

E12. The method according to embodiment E11 wherein the RNA is part of a vaccine against an arenavirus.

E13. The method according to embodiment E12 wherein the arenavirus is Lassa virus.

E14. The method according to embodiment E12 wherein the arenavirus is lymphocytic choriomeningitis virus (LCMV).

E15. The method according to embodiment E11 wherein the RNA is part of a vaccine against an astrovirus.

E16. The method according to embodiment E15 wherein the vaccine is a vaccine against a bunyavirus.

E17. The method according to embodiment E16 wherein the bunyavirus is a Hantavirus.

E18. The method according to embodiment E11 wherein the RNA is part of a vaccine against a calicivirus.

E19. The method according to embodiment E11 wherein the RNA is part of a vaccine against a coronavirus.

E20. The method according to embodiment E19 wherein the coronavirus is a middle east respiratory syndrome (MERS) virus.

E21. The method according to embodiment E19 wherein the coronavirus is a severe acute respiratory syndrome (SARS) virus.

E22. The method according to embodiment E11 wherein the RNA is part of a vaccine against a filovirus.

E23. The method according to embodiment E22 wherein the filovirus is Ebola virus.

E24. The method according to embodiment E22 wherein the filovirus is Marburg virus.

E25. The method according to embodiment E11 wherein the RNA is part of a vaccine against a flavivirus.

E26. The method according to embodiment E25 wherein the flavivirus is Yellow Fever virus.

E27. The method according to embodiment E25 wherein the flavivirus is West Nile virus. E28. The method according to embodiment E25 wherein the flavivirus is Hepatitis C virus (HCV).

E29. The method according to embodiment E11 wherein the RNA is part of a vaccine against a hepadnavirus.

E30. The method according to embodiment E11 wherein the RNA is part of a vaccine against a hepevirus.

E31. The method according to embodiment E11 wherein the RNA is part of a vaccine against an orthomyxovirus.

E32. The method according to embodiment E31 wherein the orthomyxovirus is Influenza A virus.

E33. The method according to embodiment E31 wherein the orthomyxovirus is Influenza B virus.

E34. The method according to embodiment E31 wherein the orthomyxovirus is Influenza C virus.

E35. The method according to embodiment E11 wherein the RNA is part of a vaccine against a paramyxovirus.

E36. The method according to embodiment E35 wherein the paramyxovirus is Rubeola virus.

E37. The method according to embodiment E35 wherein the paramyxovirus is Rubulavirus.

E38. The method according to embodiment E11 wherein the RNA is part of a vaccine against a picornavirus.

E39. The method according to embodiment E38 wherein the picornvirus is Poliovirus.

E40. The method according to embodiment E38 wherein the picornvirus is Hepatitis A virus (HAV).

E41. The method according to embodiment E38 wherein the picornvirus is a Rhinovirus.

E42. The method according to embodiment E11 wherein the RNA is part of a vaccine against a reovirus.

E43. The method according to embodiment E42 wherein the reovirus is a Rotavirus.

E44. The method according to embodiment E11 wherein the RNA is part of a vaccine against a retrovirus.

E45. The method according to embodiment E44 wherein the retrovirus is Human Immunodeficiency Virus (HIV).

E46. The method according to embodiment E44 wherein the retrovirus is Human T-lymphotropic virus (HTLV).

E47. The method according to embodiments E11 wherein the RNA is part of a vaccine against a rhabdovirus.

E48. The method according to embodiment E47 wherein the rhabdovirus is Rabies virus or Rabies lyssavirus.

E49. The method according to any one of embodiments E11 wherein the RNA is part of a vaccine against a togavirus.

E50. The method according to embodiment E49 wherein the togavirus is Sindbis virus (SINV).

E51. The method according to embodiment E49 wherein the togavirus is Eastern Equine Encephalitis virus (EEEV).

E52. The method according to embodiment E49 wherein the togavirus is Western Equine Encephalitis virus (WEEV).

E53. The method according to embodiment E49 wherein the togavirus is Rubella virus.

E54. The method according to any one of embodiments E1 to E8, wherein the RNA has 80% to 100% sequence homology with one of the RNAs selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, and SEQ ID NO: 8.

E55. The method according to embodiment E1 wherein the RNA has 80% to 100% sequence homology with one of the RNAs selected from the group consisting of SEQ ID NO: 1.

E56. The method according to embodiment E1 wherein the RNA has at least 80% sequence homology with the RNA of SEQ ID NO: 1.

E57. The method according to embodiment E1 wherein the RNA has at least 90% sequence homology with the RNA of SEQ ID NO: 1.

E58. The method according to embodiment E1 wherein the RNA has at least 95% sequence homology with the RNA of SEQ ID NO: 1.

E59. The method according to embodiment E1 wherein the RNA has 100% sequence homology with the RNA of SEQ ID NO: 1.

E60. The method according to embodiment E1 wherein the RNA has 80% to 100% sequence homology with one of the RNAs selected from the group consisting of SEQ ID NO: 2.

E61. The method according to embodiment E1 wherein the RNA has at least 80% sequence homology with the RNA of SEQ ID NO: 2.

E62. The method according to embodiment E1 wherein the RNA has at least 90% sequence homology with the RNA of SEQ ID NO: 2.

E63. The method according to embodiment E1 wherein the RNA has at least 95% sequence homology with the RNA of SEQ ID NO: 2.

E64. The method according to embodiment E1 wherein the RNA has 100% sequence homology with the RNA of SEQ ID NO: 2.

E65. The method according to embodiment E1 wherein the RNA has 80% to 100% sequence homology with one of the RNAs selected from the group consisting of SEQ ID NO: 3.

E66. The method according to embodiment E1 wherein the RNA has at least 80% sequence homology with the RNA of SEQ ID NO: 3.

E67. The method according to embodiment E1 wherein the RNA has at least 90% sequence homology with the RNA of SEQ ID NO: 3.

E68. The method according to embodiment E1 wherein the RNA has at least 95% sequence homology with the RNA of SEQ ID NO: 3.

E69. The method according to embodiment E1 wherein the RNA has 100% sequence homology with the RNA of SEQ ID NO: 3.

E70. The method according to embodiment E1 wherein the RNA has 80% to 100% sequence homology with one of the RNAs selected from the group consisting of SEQ ID NO: 4.

E71. The method according to embodiment E1 wherein the RNA has at least 80% sequence homology with the RNA of SEQ ID NO: 4.

E72. The method according to embodiment E1 wherein the RNA has at least 90% sequence homology with the RNA of SEQ ID NO: 4.

E73. The method according to embodiment E1 wherein the RNA has at least 95% sequence homology with the RNA of SEQ ID NO: 4.

E74. The method according to embodiment E1 wherein the RNA has at least 100% sequence homology with the RNA of SEQ ID NO: 4.

E75. The method according to embodiment E1 wherein the RNA has 80% to 100% sequence homology with one of the RNAs selected from the group consisting of SEQ ID NO: 5.

E76. The method according to embodiment E1 wherein the RNA has at least 80% sequence homology with the RNA of SEQ ID NO: 5.

E77. The method according to embodiment E1 wherein the RNA has at least 90% sequence homology with the RNA of SEQ ID NO: 5.

E78. The method according to embodiment E1 wherein the RNA has at least 95% sequence homology with the RNA of SEQ ID NO: 5.

E79. The method according to embodiment E1 wherein the RNA has 100% sequence homology with the RNA of SEQ ID NO: 5.

E80. The method according to embodiment E1 wherein the RNA has 80% to 100% sequence homology with one of the RNAs selected from the group consisting of SEQ ID NO: 6.

E81. The method according to embodiment E1 wherein the RNA has at least 80% sequence homology with the RNA of SEQ ID NO: 6.

E82. The method according to embodiment E1 wherein the RNA has at least 90% sequence homology with the RNA of SEQ ID NO: 6.

E83. The method according to embodiment E1 wherein the RNA has at least 95% sequence homology with the RNA of SEQ ID NO: 6.

E84. The method according to embodiment E1 wherein the RNA has 100% sequence homology with the RNA of SEQ ID NO: 6.

E85. The method according to embodiment E1 wherein the RNA has 80% to 100% sequence homology with one of the RNAs selected from the group consisting of SEQ ID NO: 7.

E86. The method according to embodiment E1 wherein the RNA has at least 80% sequence homology with the RNA of SEQ ID NO: 7.

E87. The method according to embodiment E1 wherein the RNA has at least 90% sequence homology with the RNA of SEQ ID NO: 7.

E88. The method according to embodiment E1 wherein the RNA has at least 95% sequence homology with the RNA of SEQ ID NO: 7.

E89. The method according to embodiment E1 wherein the RNA has 100% sequence homology with the RNA of SEQ ID NO: 7.

E90. The method according to embodiment E1 wherein the RNA has 80% to 100% sequence homology with one of the RNAs selected from the group consisting of SEQ ID NO: 8.

E91. The method according to embodiment E1 wherein the RNA has at least 80% sequence homology with the RNA of SEQ ID NO: 8.

E92. The method according to embodiment E1 wherein the RNA has at least 90% sequence homology with the RNA of SEQ ID NO: 8.

E93. The method according to embodiment E1 wherein the RNA has at least 95% sequence homology with the RNA of SEQ ID NO: 8.

E94. The method according to embodiment E1 wherein the RNA has 100% sequence homology with the RNA of SEQ ID NO: 8.

E95. The method according to any one of embodiments E1 to E94 wherein the RNA is contained within a non-viral vector.

E96. The method according to any one of embodiments E1 to E94 wherein the RNA is contained within a nanoparticle.

E97. The method according to any one of embodiments E1 to E94 wherein the RNA is contained within a liposome.

E98. The method according to any one of embodiments E1 to E94 wherein the RNA is contained within a micelle.

E99. The method according to any one of embodiments E1 to E94 wherein the RNA is contained within a viral vector.

E100. The method according to any one of embodiments E1 to E94 wherein the RNA is contained within an Adenovirus (Ad).

E101. The method according to any one of embodiments E1 to E94 wherein the RNA is contained within an adeno-associated virus (AAV).

E102. The method according to any one of embodiments E1 to E94 wherein the RNA is contained within a retrovirus.

E103. The method according to any one of embodiments E1 to E94 wherein the RNA is contained within a lentivirus.

E104. The method according to any one of embodiments E1 to E103 wherein the amino acid is glutamic acid, glutamate, or a pharmaceutically acceptable salt thereof.

E105. The method according to any one of embodiments E1 to E103 wherein the amino acid is aspartic acid, aspartate, or a pharmaceutically acceptable salt thereof.

E106. The method according to any one of embodiments E1 to E103 wherein the amino acid is tyrosine or a pharmaceutically acceptable salt thereof.

E107. The method according to any one of embodiments E1 to E103 wherein the amino acid is glutamine or a pharmaceutically acceptable salt thereof.

E108. The method according to any one of embodiments E1 to E103 wherein the amino acid is asparagine or a pharmaceutically acceptable salt thereof.

E109. The method according to any one of embodiments E1 to E103 wherein the amino acid is threonine or a pharmaceutically acceptable salt thereof.

E110. The method according to any one of embodiments E1 to E103 wherein the amino acid is serine or a pharmaceutically acceptable salt thereof.

E111. The method according to any one of embodiments E1 to E110 wherein the amino acid has a protective effect in the Droplet Digital Polymerase Chain Reaction (ddPCR) Assay.

E112. The method according to any one of embodiments E1 to E111 wherein the amino acid produces a measurable percent protection in the Droplet Digital Polymerase Chain Reaction (ddPCR) Assay.

E113. The method according to embodiment E112 wherein the amino acid produces a percent protection of at least 10% in the ddPCR assay.

E114. The method according to embodiment E112 wherein the amino acid produces a percent protection of at least 20% in the ddPCR assay.

E115. The method according to embodiment E112 wherein the amino acid produces a percent protection of at least 30% in the ddPCR assay.

E116. The method according to embodiment E112 wherein the amino acid produces a percent protection of at least 40% in the ddPCR assay.

E117. The method according to embodiment E112 wherein the amino acid produces a percent protection of at least 50% in the ddPCR assay.

E118. The method according to embodiment E112 wherein the amino acid produces a percent protection of at least 60% in the ddPCR assay.

E119. The method according to embodiment E112 wherein the amino acid produces a percent protection of at least 70% in the ddPCR assay.

E120. The method according to embodiment E112 wherein the amino acid produces a percent protection of at least 80% in the ddPCR assay.

E121. The method according to embodiment E112 wherein the amino acid produces a percent protection of at least 90% in the ddPCR assay.

E122. The method according to any one of embodiments E1 to E121 wherein the amino acid has an EC50 equal to or less than 700 QM in the ddPCR assay.

E123. The method according to any one of embodiments E1 to E121 wherein the amino acid has an EC50 equal to or less than 600 QM in the ddPCR assay.

E124. The method according to any one of embodiments E1 to E121 wherein the amino acid has an EC50 equal to or less than 500 QM in the ddPCR assay.

E125. The method according to any one of embodiments E1 to E121 wherein the amino acid has an EC50 equal to or less than 400 QM in the ddPCR assay.

E126. The method according to any one of embodiments E1 to E121 wherein the amino acid has an EC50 equal to or less than 300 QM in the ddPCR assay.

E127. The method according to any one of embodiments E1 to E121 wherein the amino acid has an EC50 equal to or less than 200 QM in the ddPCR assay.

E128. The method according to any one of embodiments E1 to E121 wherein the amino acid has an EC50 equal to or less than 100 DM in the ddPCR assay.

E129. The method according to any one of embodiments E1 to E121 wherein the amino acid has an EC50 equal to or less than 50 QM in the ddPCR assay.

E130. The method according to any one of embodiments E112 to E129 wherein the percent protection is measured in the ddPCR assay under conditions that accelerate RNA degradation.

E131. The method according to any one of embodiments E112 to E129 wherein the percent protection is measured in the ddPCR assay under conditions pH 11.0, 30° C., and 16 hours.

E132. The method according to any one of embodiments E112 to E129 wherein the percent protection is measured in the ddPCR assay under conditions pH 11.0, 44° C., and 16 hours.

E133. The method according to any one of embodiments E112 to E129 wherein the amino acid's EC50 is measured in the ddPCR assay under conditions that accelerate RNA degradation.

E134. The method according to any one of embodiments E112 to E129 wherein the amino acid's EC50 is measured under conditions pH 11.0, 30° C., and 16 hours in the ddPCR assay.

E135. The method according to any one of embodiments E112 to E129 wherein the amino acid's EC50 is measured under conditions pH 11.0, 44° C., and 16 hours in the ddPCR assay.

E136. The method according to any one of embodiments E1 to E135 wherein the amino acid has a protective effect in the TapeStation Assay.

E137. The method according to embodiment 136 wherein the amino acid protects at least 10% of the RNA in the TapeStation Assay.

E138. The method according to embodiment 136 wherein the amino acid protects at least 20% of the RNA in the TapeStation Assay.

E139. The method according to embodiment 136 wherein the amino acid protects at least 30% of the RNA in the TapeStation Assay.

E140. The method according to embodiment 136 wherein the amino acid protects at least 40% of the RNA in the TapeStation Assay.

E141. The method according to embodiment 136 wherein the amino acid protects at least 50% of the RNA in the TapeStation Assay.

E142. The method according to embodiment 136 wherein the amino acid protects at least 60% of the RNA in the TapeStation Assay.

E143. The method according to embodiment 136 wherein the amino acid protects at least 70% of the RNA in the TapeStation Assay.

E144. The method according to embodiment 136 wherein the amino acid protects at least 80% of the RNA in the TapeStation Assay.

E145. The method according to embodiment 136 wherein the amino acid protects at least 90% of the RNA in the TapeStation Assay.

E146. The method according to embodiment 136 wherein the amino acid protects 100% of the RNA in the TapeStation Assay.

E147. The method according to any one of embodiments E136 to E146 wherein the percentage of RNA protected is measured in the TapeStation Assay under conditions that accelerate RNA degradation.

E148. The method according to any one of embodiments E136 to E146 wherein the percentage of RNA protected is measured in the TapeStation Assay under conditions pH 11.0, 50° C., and 15 minutes.

E149. A composition comprising an RNA and at least one amino acid.

E150. The composition according to embodiment E149 wherein the RNA is a double stranded RNA.

E151. The composition according to embodiment E149 wherein the RNA is a single strand RNA.

E152. The composition according to embodiment E149 wherein the RNA is an antisense single strand RNA.

E153. The composition according to embodiment E149 wherein the RNA is a messenger RNA (mRNA).

E154. The composition according to embodiment E149 wherein the RNA is a modified RNA (modRNA).

E155. The composition according to embodiment E149 wherein the RNA is a self-amplifying RNA (saRNA).

E156. The composition according to embodiment E149 wherein the RNA is replicon RNA.

E157. The composition according to any one of embodiments E149 to E156 wherein the RNA is viral RNA.

E158. The composition according to any one of embodiments E149 to E157 wherein the RNA encodes an antigen.

E159. The composition according to any one of embodiments E149 to E158 wherein the RNA is part of a vaccine.

E160. The composition according to any one of embodiments E149 to E159 wherein the RNA is part of a vaccine against an arenavirus.

E161. The composition according to embodiment E160 wherein the arenavirus is Lassa virus.

E162. The composition according to embodiment E160 wherein the arenavirus is lymphocytic choriomeningitis virus (LCMV).

E163. The composition according to any one of embodiments E149 to E159 wherein the RNA is part of a vaccine against an astrovirus.

E164. The composition according to any one of embodiments E149 to E159 wherein the RNA is part of a vaccine against a bunyavirus.

E165. The composition according to embodiment E164 wherein the bunyavirus is a Hantavirus.

E166. The composition according to any one of embodiments E149 to E159 wherein the RNA is part of a vaccine against a calicivirus.

E167. The composition according to any one of embodiments E149 to E159 wherein the RNA is part of a vaccine against a coronavirus.

E168. The composition according to embodiment E167 wherein the coronavirus is a middle east respiratory syndrome (MERS) virus.

E169. The composition according to embodiment E167 wherein the coronavirus is a severe acute respiratory syndrome (SARS) virus.

E170. The composition according to any one of embodiments E149 to E159 wherein the RNA is part of a vaccine against a filovirus.

E171. The composition according to embodiment E170 wherein the filovirus is Ebola virus.

E172. The composition according to embodiment E170 wherein the filovirus is Marburg virus.

E173. The composition according to any one of embodiments E149 to E159 wherein the RNA is part of a vaccine against a flavivirus.

E174. The composition according to embodiment E173 wherein the flavivirus is Yellow Fever virus.

E175. The composition according to embodiment E173 wherein the flavivirus is West Nile virus.

E176. The composition according to embodiment E173 wherein the flavivirus is Hepatitis C virus (HCV).

E177. The composition according to any one of embodiments E149 to E159 wherein the RNA is part of a vaccine against a hepadnavirus.

E178. The composition according to any one of embodiments E149 to E159 wherein the RNA is part of a vaccine against a hepevirus.

E179. The composition according to any one of embodiments E149 to E159 wherein the RNA is part of a vaccine against an orthomyxovirus.

E180. The composition according to embodiment E179 wherein the orthomyxovirus is Influenza A virus.

E181. The composition according to embodiment E179 wherein the orthomyxovirus is Influenza B virus.

E182. The composition according to embodiment E179 wherein the orthomyxovirus is Influenza C virus.

E183. The composition according to any one of embodiments E149 to E159 wherein the RNA is part of a vaccine against a paramyxovirus.

E184. The composition according to embodiment E183 wherein the paramyxovirus is Rubeola virus.

E185. The composition according to embodiment E183 wherein the paramyxovirus is Rubulavirus.

E186. The composition according to any one of embodiments E149 to E159 wherein the RNA is part of a vaccine against a picornavirus.

E187. The composition according to embodiment E186 wherein the picornvirus is Poliovirus.

E188. The composition according to embodiment E186 wherein the picornvirus is Hepatitis A virus (HAV).

E189. The composition according to embodiment E186 wherein the picornvirus is a Rhinovirus.

E190. The composition according to any one of embodiments E149 to E159 wherein the RNA is a vaccine against a reovirus.

E191. The composition according to embodiment E190 wherein the reovirus is a Rotavirus.

E192. The composition according to any one of embodiments E149 to E159 wherein the RNA is part of a vaccine against a retrovirus.

E193. The composition according to embodiment E192 wherein the retrovirus is Human Immunodeficiency Virus (HIV).

E194. The composition according to embodiment E192 wherein the retrovirus is Human T-lymphotropic virus (HTLV).

E195. The composition according to any one of embodiments E149 to E159 wherein the RNA is part of a vaccine against a rhabdovirus.

E196. The composition according to embodiment E195 wherein the rhabdovirus is Rabies virus or Rabies lyssavirus.

E197. The composition according to any one of embodiments E149 to E159 wherein the RNA is part of a vaccine against a togavirus.

E198. The composition according to embodiment E197 wherein the togavirus is Sindbis virus (SINV).

E199. The composition according to embodiment E197 wherein the togavirus is Eastern Equine Encephalitis virus (EEEV).

E200. The composition according to embodiment E197 wherein the togavirus is Western Equine Encephalitis virus (WEEV).

E201. The composition according to embodiment E197 wherein the togavirus is Rubella virus.

E202. The composition according to embodiment E149 wherein the RNA is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

E203. The composition according to any one of embodiments E149 to E202 wherein the at least one amino acid is glutamic acid, glutamate, or a pharmaceutically acceptable salt thereof.

E204. The composition according to any one of embodiments E149 to E202 wherein the at least one amino acid is aspartic acid, aspartate, or a pharmaceutically acceptable salt thereof.

E205. The composition according to any one of embodiments E149 to E202 wherein the at least one amino acid is tyrosine or a pharmaceutically acceptable salt thereof.

E206. The composition according to any one of embodiments E149 to E202 wherein the at least one amino acid is glutamine or a pharmaceutically acceptable salt thereof.

E207. The composition according to any one of embodiments E149 to E202 wherein the at least one amino acid is asparagine or a pharmaceutically acceptable salt thereof.

E208. The composition according to any one of embodiments E149 to E202 wherein the at least one amino acid is threonine or a pharmaceutically acceptable salt thereof.

E209. The composition according to any one of embodiments E149 to E202 wherein the at least one amino acid is serine or a pharmaceutically acceptable salt thereof.

E210. The composition according to any one of embodiments E149 to E209 wherein the RNA is contained within a non-viral vector.

E211. The composition according to any one of embodiments E149 to E210 wherein the RNA and the at least one amino acid are contained within a nanoparticle.

E212. The composition according to any one of embodiments E149 to E210 wherein the RNA and the at least one amino acid are contained within a liposome.

E213. The composition according to any one of embodiments E149 to E210 wherein the RNA and the at least one amino acid are contained within a micelle.

E214. The composition according to any one of embodiments E149 to E210 wherein the RNA is contained within a viral vector.

E215. The composition according to any one of embodiments E149 to E210 wherein the RNA is contained within an Adenovirus.

E216. The composition according to any one of embodiments E149 to E210 wherein the RNA is contained within an adeno-associated virus.

E217. The composition according to any one of embodiments E149 to E210 wherein the RNA is contained within a retrovirus.

E218. The composition according to any one of embodiments E149 to E210 wherein the RNA is contained within a lentivirus.

E219. The method according to any one of embodiments E1 to E148 wherein the amino acid has a protective effect in the Size Exclusion Base Hydrolysis Degradation Assay.

E220. The method according to embodiment E219 wherein the absorbance units at T=2 hours at 250 nm are at least 10% of the peak area at T=0 in the Size Exclusion Base Hydrolysis Assay.

E221. The method according to embodiment E219 wherein the absorbance units at T=10 hours at 250 nm are at least 10% of the peak area at T=0 in the Size Exclusion Base Hydrolysis Assay.

E222. The method according to embodiment E219 wherein the absorbance units at T=24 hours at 250 nm are at least 10% of the peak area at T=0 in the Size Exclusion Base Hydrolysis Assay.

E223. The method according to embodiment E219 wherein the absorbance units at T=2 hours at 250 nm are at least 20% of the peak area at T=0 in the Size Exclusion Base Hydrolysis Assay.

E224. The method according to embodiment E219 wherein the absorbance units at T=10 hours at 250 nm are at least 20% of the peak area at T=0 in the Size Exclusion Base Hydrolysis Assay.

E225. The method according to embodiment E219 wherein the absorbance units at T=24 hours at 250 nm are at least 20% of the peak area at T=0 in the Size Exclusion Base Hydrolysis Assay.

E226. The method according to embodiment E219 wherein the absorbance units at T=2 hours at 250 nm are at least 30% of the peak area at T=0 in the Size Exclusion Base Hydrolysis Assay.

E227. The method according to embodiment E219 wherein the absorbance units at T=10 hours at 250 nm are at least 30% of the peak area at T=0 in the Size Exclusion Base Hydrolysis Assay.

E228. The method according to embodiment E219 wherein the absorbance units at T=24 hours at 250 nm are at least 30% of the peak area at T=0 in the Size Exclusion Base Hydrolysis Assay.

E229. The method according to embodiment E219 wherein the absorbance units at T=2 hours at 250 nm are at least 40% of the peak area at T=0 in the Size Exclusion Base Hydrolysis Assay.

E230. The method according to embodiment E219 wherein the absorbance units at T=10 hours at 250 nm are at least 40% of the peak area at T=0 in the Size Exclusion Base Hydrolysis Assay.

E231. The method according to embodiment E219 wherein the absorbance units at T=24 hours at 250 nm are at least 40% of the peak area at T=0 in the Size Exclusion Base Hydrolysis Assay.

E232. The method according to embodiment E219 wherein the absorbance units at T=2 hours at 250 nm are at least 50% of the peak area at T=0 in the Size Exclusion Base Hydrolysis Assay.

E233. The method according to embodiment E219 wherein the absorbance units at T=10 hours at 250 nm are at least 50% of the peak area at T=0 in the Size Exclusion Base Hydrolysis Assay.

E234. The method according to embodiment E219 wherein the absorbance units at T=24 hours at 250 nm are at least 50% of the peak area at T=0 in the Size Exclusion Base Hydrolysis Assay.

E235. The method according to embodiment E219 wherein the absorbance units at T=2 hours at 250 nm are at least 60% of the peak area at T=0 in the Size Exclusion Base Hydrolysis Assay.

E236. The method according to embodiment E219 wherein the absorbance units at T=10 hours at 250 nm are at least 60% of the peak area at T=0 in the Size Exclusion Base Hydrolysis Assay.

E237. The method according to embodiment E219 wherein the absorbance units at T=24 hours at 250 nm are at least 60% of the peak area at T=0 in the Size Exclusion Base Hydrolysis Assay.

E238. The method according to embodiment E219 wherein the absorbance units at T=2 hours at 250 nm are at least 70% of the peak area at T=0 in the Size Exclusion Base Hydrolysis Assay.

E239. The method according to embodiment E219 wherein the absorbance units at T=10 hours at 250 nm are at least 70% of the peak area at T=0 in the Size Exclusion Base Hydrolysis Assay.

E240. The method according to embodiment E219 wherein the absorbance units at T=24 hours at 250 nm are at least 70% of the peak area at T=0 in the Size Exclusion Base Hydrolysis Assay.

E241. The method according to any one of embodiments E219 to E240 wherein the level of RNA protection is measured in the Size Exclusion Base Hydrolysis Degradation Assay under conditions that accelerate RNA degradation.

E242. According to another aspect of the present disclosure, there is provided a method of protecting an isolated RNA from degradation by by forming a solution containing the isolated RNA and at least 1 mM of a free amino acid, the method comprising contacting the isolated RNA with the amino acid.

E243. The method according to embodiment E242, wherein the RNA is double stranded.

E244. The method according to embodiment E242, wherein the RNA is single stranded.

E245. The method according to embodiment E242, wherein the RNA is antisense single stranded.

E246. The method according to embodiment E242, wherein the RNA is messenger RNA (mRNA).

E247. The method according to embodiment E242, wherein the RNA is self-amplifying RNA (saRNA).

E248. The method according to any one of embodiments E242 to E247, wherein the RNA comprises:
a 5′ Cap represented by Formula I,

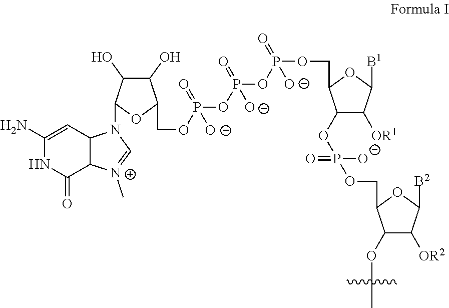

where R¹ and R² are each independently H or Me; and
B¹ and B² are each independently guanine, adenine, or uracil;
a 5′ untranslated region;
a coding region for a nonstructural protein derived from an alphavirus;
a subgenomic promoter derived from an alphavirus;
an open reading frame encoding a gene of interest;
a 3′ untranslated region; and a 3′ poly A sequence.

E249. The method according to any one of embodiments E242 to E248, wherein the RNA is modified RNA (modRNA).

E250. The method according to any one of embodiments E242 to E249, wherein the RNA is viral RNA.

E251. The method according to any one of embodiments E242 to E250, wherein the RNA encodes an antigen.

E252. The method according to any one of embodiments E242 to E251, wherein the RNA encodes a viral antigen.

E253. The method according to any one of embodiments E242 to E252, wherein the RNA is part of a vaccine.

E254. The method according to any one of embodiments E242 to E253, wherein the RNA is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, and SEQ ID NO: 9.

E255. The method according to any one of embodiments E242 to E254, wherein the solution further comprises a sugar and/or a buffer.

E256. The method according to embodiment E255, wherein the sugar is at a concentration of about 10% w/v.

E257. The method according to embodiment E255 or embodiment E256, wherein the buffer is at a concentration of about 10 mM.

E258. The method according to any one of embodiments E255 to E257, wherein the buffer is at a pH of about 7.4.

E259. The method according to any one of embodiments E255 to E258, wherein the sugar comprises sucrose and/or trehalose.

E260. The method according to any one of embodiments E255 to E259, wherein the sugar comprises sucrose.

E261. The method according to any one of embodiments E255 to E260, wherein the buffer comprises Tris buffer, PBS, or HEPES buffer.

E262. The method according to any one of embodiments E255 to E261, wherein the buffer comprises Tris buffer.

E263. The method according to any one of embodiments E255 to E262, wherein the sugar comprises sucrose and the buffer comprises Tris buffer.

E264. The method according to any one of embodiments E242 to E263, wherein the RNA is encapsulated in, bound to, or adsorbed on a material.

E265. The method according to any one of embodiments E242 to E264, wherein the material comprises a lipid.

E266. The method according to any one of embodiments E242 to E265, wherein the material comprises a cationic lipid.

E267. The method according to any one of embodiments E242 to E266, wherein the RNA is encapsulated in, bound to, or adsorbed on a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

E268. The method according to any one of embodiments E242 to E267, wherein the solution comprises at least 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, or 40 mM of the free amino acid.

E269. The method according to any one of embodiments E242 to E268, wherein the solution comprises at least 5 mM of the free amino acid.

E270. The method according to any one of embodiments E242 to E269, wherein the solution comprises at least 10 mM of the free amino acid.

E271. The method according to any one of embodiments E242 to E270, wherein the solution comprises at least 15 mM of the free amino acid.

E272. The method according to any one of embodiments E242 to E271, wherein the solution comprises at least 20 mM of the free amino acid.

E273. The method according to any one of embodiments E242 to E272, wherein free amino acid is selected from the group consisting of glutamic acid, glutamate, aspartic acid, aspartate, arginine, lysine, tyrosine, cysteine, glutamine, asparagine, threonine, and serine, or a pharmaceutically acceptable salt thereof.

E274. The method according to any one of embodiments E242 to E273, wherein the free amino acid is glutamic acid, glutamate, or a pharmaceutically acceptable salt thereof.

E275. The method according to any one of embodiments E242 to E273, wherein the free amino acid is aspartic acid, aspartate, or a pharmaceutically acceptable salt thereof.

E276. The method according to any one of embodiments E242 to E273, wherein the free amino acid is arginine, or a pharmaceutically acceptable salt thereof.

E277. The method according to any one of embodiments E242 to E273, wherein the free amino acid is lysine, or a pharmaceutically acceptable salt thereof.

E278. The method according to any one of embodiments E242 to E277, further comprising the step of storing the solution comprising the RNA and at least 1 mM of the free amino acid.

E279. The method according to embodiment E278, wherein the isolated RNA is stored for at least about 2 hours, 4 hours, 6 hours, 12 hours, 1 day, 2 days, 3 days, 4 days, 5 days, 6 days, 7 days, 2 weeks, 3 weeks, four weeks, five weeks, six weeks, seven weeks, or 8 weeks.

E280. The method according to embodiment E278 or embodiment E279, wherein the isolated RNA is stored at a temperature of about 5° C., 0° C., −10° C., −20° C., −30° C., −40° C., −50° C., −60° C., −70° C., or −80° C.

E281. The method according to any one of embodiments E242 to E280, wherein the at least 1 mM of a free amino acid provides a protective effect to the RNA in a Droplet Digital Polymerase Chain Reaction Assay.

E282. The method according to embodiment E281, wherein the Droplet Digital Polymerase Chain Reaction Assay conditions accelerate degradation of the RNA.

E283. The method according to any one of embodiments E242 to E282, wherein the at least 1 mM of a free amino acid provides a protective effect to the RNA in a TapeStation Assay.

E284. The method according to embodiment E283, wherein the TapeStation Assay conditions accelerate degradation of the RNA.

E285. The method according to any one of embodiments E242 to E284, wherein the at least 1 mM of a free amino acid provides a protective effect to the RNA in a Size Exclusion Base Hydrolysis Assay.

E286. The method according to embodiment E285, wherein the Size Exclusion Base Hydrolysis Assay conditions accelerate degradation of the RNA.

E287. The method according to any one of embodiments E242 to E286, wherein the isolated RNA is comprised in any one of the compositions according to embodiments E149 to E218 or E288 to E324.

E288. According to another aspect of the present disclosure, there is provided a composition comprising in solution: at least 1 mM of a free amino acid; and an isolated RNA.

E289. The composition according to embodiment E288, wherein the RNA is double stranded.

E290. The composition according to embodiment E288, wherein the RNA is single stranded.

E291. The composition according to embodiment E288, wherein the RNA is antisense single stranded.

E292. The composition according to embodiment E288, wherein the RNA is messenger RNA (mRNA).

E293. The composition according to embodiment E288, wherein the RNA is self-amplifying RNA (saRNA).

E294. The composition according to any one of embodiments E288 to E293, wherein the RNA comprises:
a 5′ Cap represented by Formula I,

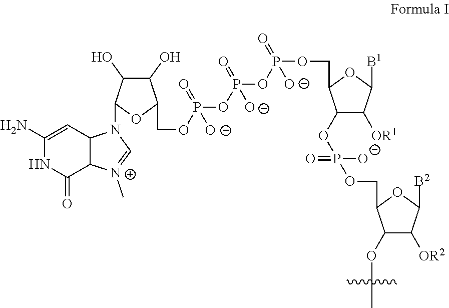

where R¹ and R² are each independently H or Me; and
B¹ and B² are each independently guanine, adenine, or uracil;
a 5′ untranslated region;
a coding region for a nonstructural protein derived from an alphavirus;
a subgenomic promoter derived from an alphavirus;
an open reading frame encoding a gene of interest;
a 3′ untranslated region; and a 3′ poly A sequence.

E295. The composition according to any one of embodiment E288 to E294, wherein the RNA is modified RNA (modRNA).

E296. The composition according to any one of embodiments E288 to E295, wherein the RNA is viral RNA.

E297. The composition according to any one of embodiments E288 to E296, wherein the RNA encodes a viral antigen.

E298. The composition according to any one of embodiments E288 to E297, wherein the RNA is part of a vaccine.

E299. The composition according to any one of embodiments E288 to E298, wherein the solution further comprises a sugar and/or a buffer.

E300. The composition according to embodiment E299, wherein the sugar is at a concentration of about 10% w/v.

E301. The composition according to embodiment E299 or embodiment E300, wherein the buffer is at a concentration of about 10 mM.

E302. The composition according to any one of embodiments E299 to E301, wherein the buffer is at a pH of about 7.4.

E303. The composition according to any one of embodiments E299 to E302, wherein the sugar comprises sucrose and/or trehalose.

E304. The composition according to any one of embodiments E299 to E303, wherein the sugar comprises sucrose.

E305. The composition according to any one of embodiments E299 to E304, wherein the buffer comprises Tris buffer, PBS, or HEPES buffer.

E306. The composition according to any one of embodiments E299 to E305, wherein the buffer comprises Tris buffer.

E307. The composition according to any one of embodiments E299 to E306, wherein the sugar comprises sucrose and the buffer comprises Tris buffer.

E308. The composition according to any one of embodiments E288 to E307, wherein the isolated RNA is encapsulated in, bound to, or adsorbed on a material.

E309. The composition according to embodiment E308, wherein the material comprises a cationic lipid.

E310. The composition according to embodiment E309, wherein the isolated RNA is encapsulated in the cationic lipid.

E311. The composition according to any one of embodiments E308 to E310, wherein the material comprises a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

E312. The composition according to embodiment E311, wherein the isolated RNA is encapsulated in the liposome, lipid nanoparticle, polyplex, cochleate, virosome, immune-stimulating complex, microparticle, microsphere, nanosphere, unilamellar vesicle, multilamellar vesicle, oil-in water emulsion, water-in-oil emulsion, emulsome, polycationic peptide, cationic nanoemulsion, or combination thereof.

E313. The composition according to any one of embodiments E308 to E312, wherein the material further encapsulates the at least 1 mM of a free amino acid.

E314. The composition according to any one of embodiments E308 to E312, wherein the material does not encapsulate the at least 1 mM of a free amino acid.

E315. The composition according to any one of embodiments E288 to E314, wherein the free amino acid is selected from the group consisting of glutamic acid, glutamate, aspartic acid, aspartate, arginine, lysine, tyrosine, cysteine, glutamine, asparagine, threonine, and serine, or a pharmaceutically acceptable salt thereof.

E316. The composition according to any one of embodiments E288 to E315, wherein the free amino acid is glutamic acid, glutamate, or a pharmaceutically acceptable salt thereof.

E317. The composition according to any one of embodiments E288 to E315, wherein the free amino acid is aspartic acid, aspartate, or a pharmaceutically acceptable salt thereof.

E318. The composition according to any one of embodiments E288 to E315, wherein the free amino acid is arginine, or a pharmaceutically acceptable salt thereof.

E319. The composition according to any one of embodiments E288 to E315, wherein the free amino acid is lysine, or a pharmaceutically acceptable salt thereof.

E320. The composition according to any one of embodiments E288 to E319, wherein the solution comprises at least 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, or 40 mM of the free amino acid.

E321. The composition according to any one of embodiments E288 to E320, wherein the solution comprises at least 5 mM of the free amino acid.

E322. The composition according to any one of embodiments E288 to E321, wherein the solution comprises at least 10 mM of the free amino acid.

E323. The composition according to any one of embodiments E288 to E322, wherein the solution comprises at least 15 mM of the free amino acid.

E324. The composition according to any one of embodiments E288 to E323, wherein the solution comprises at least 20 mM of the free amino acid.

E325. According to another aspect of the present disclosure, there is provided a method of protecting an isolated RNA that is encapsulated in a capsule from degradation by forming a solution containing the encapsulated isolated RNA and at least 1 mM of a free amino acid, the method comprising contacting the capsule encapsulating the RNA with the free amino acid.

E326. The method according to embodiment E325, wherein the RNA is double stranded.

E327. The method according to embodiment E325, wherein the RNA is single stranded.

E328. The method according to embodiment E325, wherein the RNA is antisense single stranded.

E329. The method according to embodiment E325, wherein the RNA is messenger RNA (mRNA).

E330. The method according to embodiment E325, wherein the RNA is self-amplifying RNA (saRNA).

E331. The method according to any one of embodiments E325 to E330, wherein the saRNA comprises:
a 5′ Cap represented by Formula I,

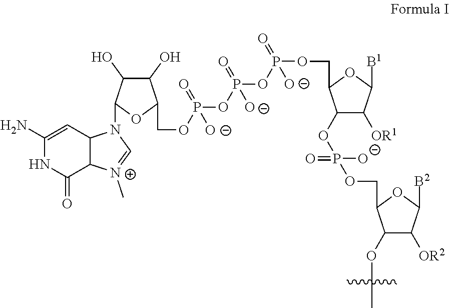

where R¹ and R² are each independently H or Me; and
B¹ and B² are each independently guanine, adenine, or uracil;
a 5′ untranslated region;
a coding region for a nonstructural protein derived from an alphavirus;
a subgenomic promoter derived from an alphavirus;
an open reading frame encoding a gene of interest;
a 3′ untranslated region; and
a 3′ poly A sequence.

E332. The method according to any one of embodiments E325 to E331, wherein the RNA is modified RNA (modRNA).

E333. The method according to any one of embodiments E325 to E332, wherein the RNA is viral RNA.

E334. The method according to any one of embodiments E325 to E333, wherein the RNA encodes a viral antigen.

E335. The method according to any one of embodiments E325 to E334, wherein the RNA is part of a vaccine.

E336. The method according to any one of embodiments E325 to E335, wherein the solution further comprises a sugar and/or a buffer.

E337. The method according to embodiment E336, wherein the sugar is at a concentration of about 10% w/v.

E338. The method according to embodiment E336 or embodiment E337, wherein the buffer is at a concentration of about 10 mM.

E339. The method according to any one of embodiments E336 to E338, wherein the buffer is at a pH of about 7.4.

E340. The method according to any one of embodiments E336 to E339, wherein the sugar comprises sucrose and/or trehalose.

E341. The method according to any one of embodiments E336 to E340, wherein the sugar comprises sucrose.

E342. The method according to any one of embodiments E336 to E341, wherein the buffer comprises Tris buffer, PBS, or HEPES buffer.

E343. The method according to any one of embodiments E336 to E342, wherein the buffer comprises Tris buffer.

E344. The method according to any one of embodiments E336 to E343, wherein the sugar comprises sucrose and the buffer comprises Tris buffer.

E345. The method according to any one of embodiments E325 to E344, wherein the capsule comprises a cationic lipid.

E346. The method according to any one of embodiments E325 to E345, wherein the capsule comprises a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

E347. The method according to any one of embodiments E325 to E346, wherein the free amino acid is selected from the group consisting of glutamic acid, glutamate, aspartic acid, aspartate, arginine, lysine, tyrosine, cysteine, glutamine, asparagine, threonine, and serine, or a pharmaceutically acceptable salt thereof.

E348. The method according to any one of embodiments E325 to E347, wherein the free amino acid is glutamic acid, glutamate, or a pharmaceutically acceptable salt thereof.

E349. The method according to any one of embodiments E325 to E347, wherein the free amino acid is aspartic acid, aspartate, or a pharmaceutically acceptable salt thereof.

E350. The method according to any one of embodiments E325 to E347, wherein the free amino acid is arginine, or a pharmaceutically acceptable salt thereof.

E351. The method according to any one of embodiments E325 to E347, wherein the free amino acid is lysine, or a pharmaceutically acceptable salt thereof.

E352. The method according to any one of embodiments E325 to E351, wherein the solution comprises at least 2 mM, 3 mM, 4 mM, 5 mM, 6 mM, 7 mM, 8 mM, 9 mM, 10 mM, 11 mM, 12 mM, 13 mM, 14 mM, 66 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, or 40 mM of the free amino acid.

E353. The method according to any one of embodiments E325 to E352, wherein the solution comprises at least 5 mM of the free amino acid.

E354. The method according to any one of embodiments E325 to E353, wherein the solution comprises at least 10 mM of the free amino acid.

E355. The method according to any one of embodiments E325 to E354, wherein the solution comprises at least 15 mM of the free amino acid.

E356. The method according to any one of embodiments E325 to E355, wherein the solution comprises at least 20 mM of the free amino acid.

E357. The method according to any one of embodiments E325 to E356, further comprising the step of storing the solution comprising the encapsulated isolated RNA and at least 1 mM of the free amino acid.

E358. The method according to embodiment E357, wherein the encapsulated isolated RNA is stored for at least about 2 hours, 4 hours, 6 hours, 12 hours, 1 day, 2 days, 3 days, 4 days, 5 days, 6 days, 7 days, 2 weeks, 3 weeks, four weeks, five weeks, six weeks, seven weeks, or 8 weeks.

E359. The method according to embodiment E357 or embodiment E358, wherein the encapsulated isolated RNA is stored at a temperature of about 5° C., 0° C., −10° C., −20° C., −30° C., −40° C., −50° C., −60° C., −70° C. or −80° C.

E360. The method according to any one of embodiments E325 to E359, wherein the capsule further encapsulates the at least 1 mM of a free amino acid.

E361. The method according to embodiment E360, wherein the isolated RNA is contacted with the at least 1 mM of a free amino acid before the isolated RNA is encapsulated.

E362. The method according to any one of embodiments E325 to E359, wherein the capsule does not encapsulate the at least 1 mM of a free amino acid.

E363. The method according to embodiment E362, wherein the capsule comprising the isolated RNA is contacted with the at least 1 mM of a free amino acid after the isolated RNA is encapsulated in the capsule.

E364. The method according to any one of embodiments E325 to E363, wherein the at least 1 mM of a free one amino acid provides a protective effect to the RNA in a Droplet Digital Polymerase Chain Reaction Assay.

E365. The method according to embodiment E364, wherein the Droplet Digital Polymerase Chain Reaction Assay conditions accelerate degradation of the RNA.

E366. The method according to any one of embodiments E325 to E365, wherein the at least 1 mM of a free amino acid provides a protective effect to the RNA in a TapeStation Assay.

E367. The method according to embodiment E366, wherein the TapeStation Assay conditions accelerate degradation of the RNA.

E368. The method according to any one of embodiments E325 to E367, wherein the at least 1 mM of a free amino acid provides a protective effect to the RNA in a Size Exclusion Base Hydrolysis Assay.

E369. The method according to embodiment E368, wherein the Size Exclusion Base Hydrolysis Assay conditions accelerate degradation of the RNA.

E370. The method according to any one of embodiments E325 to E369, wherein protection of the isolated RNA from degradation is assessed by measuring in vitro expression of the RNA.

E371. The method according to embodiment E370, wherein contacting the capsule comprising the isolated RNA with the at least 1 mM of a free amino acid increases in vitro expression of the isolated RNA compared to in vitro expression of encapsulated isolated RNA that has not been contacted with the at least 1 mM of a free amino acid.

E372. The method according to embodiment E371, wherein in vitro expression of the isolated RNA is increased at least about 20%, 21%, 22%, 23%, 24%, 25%, 26%, 27%, 28%, 29%, 30%, 32%, 33%, 34%, 35%, 36%, 37%, 38%, 39%, or 40%.

E373. The method according to embodiment E371 or E372, wherein in vitro expression of the isolated RNA is increased at least about 33%.

E374. The method according to any one of embodiments E325 to E373, wherein protection of the isolated RNA from degradation is assessed by measuring immunogenicity of the isolated RNA.

E375. The method according to embodiment E374, wherein contacting the capsule comprising the isolated RNA with the at least 1 mM of a free amino acid increases immunogenicity of the isolated RNA compared to immunogenicity of encapsulated isolated RNA that has not been contacted with the at least 1 mM of a free amino acid.

E376. The method according to embodiment E375, wherein immunogenicity of the isolated RNA is increased at least about 15%, 16%, 17%, 18%, 19%, 20%, 21%, 22%, 23%, 24%, 25%, 26%, 27%, 28%, 29%, 30%, 32%, 33%, 34%, or 35%.

E377. The method according to embodiment E375 or E376, wherein immunogenicity of the isolated RNA is increased at least about 25%.

E378. The method according to any one of embodiments E325 to E377, wherein the capsule comprising the isolated RNA is comprised in any one of the compositions according to embodiments E149 to E218 or E379 to E412.

E379. According to another aspect of the present disclosure, there is provided a composition comprising in solution: at least 1 mM of a free amino acid; and a capsule comprising isolated RNA.

E380. The composition according to embodiment E379, wherein the RNA is double stranded.

E381. The composition according to embodiment E379, wherein the RNA is single stranded.

E382. The composition according to embodiment E379, wherein the RNA is antisense single stranded.

E383. The composition according to embodiment E379, wherein the RNA is messenger RNA (mRNA).

E384. The composition according to embodiment E379, wherein the RNA is self-amplifying RNA (saRNA).

E385. The composition according to any one of embodiments E379 to E384, wherein the RNA comprises:
a 5′ Cap represented by Formula I,

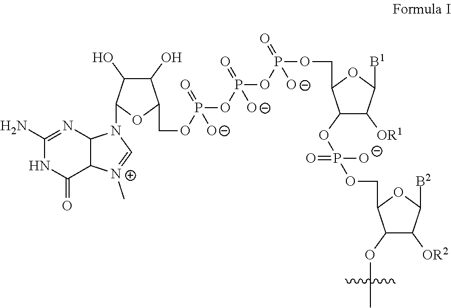

where R¹ and R² are each independently H or Me; and
B¹ and B² are each independently guanine, adenine, or uracil;
a 5′ untranslated region;
a coding region for a nonstructural protein derived from an alphavirus;
a subgenomic promoter derived from an alphavirus;
an open reading frame encoding a gene of interest;
a 3′ untranslated region; and
a 3′ poly A sequence.

E386. The composition according to any one of embodiments E379 to E385, wherein the RNA is modified RNA (modRNA).

E387. The composition according to any one of embodiments E379 to E386 wherein the RNA is viral RNA.

E388. The composition according to any one of embodiments E379 to E387, wherein the RNA encodes a viral antigen.

E389. The composition according to any one of embodiment E379 to E388, wherein the RNA is part of a vaccine.

E390. The composition according to any one of embodiments E379 to E389, wherein the capsule comprises a cationic lipid.

E391. The composition according to any one of embodiments E379 to E390, wherein the capsule comprises a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

E392. The composition according to any one of embodiments E379 to E391, wherein the capsule comprising the isolated RNA further encapsulates the at least 1 mM of a free amino acid.

E393. The composition according to any one of embodiments E379 to E391, wherein the capsule comprising the isolated RNA does not encapsulate the at least 1 mM of a free amino acid.

E394. The composition according to any one of embodiments E379 to E393, wherein the free amino acid is selected from the group consisting of glutamic acid, glutamate, aspartic acid, aspartate, arginine, lysine, tyrosine, cysteine, glutamine, asparagine, threonine, and serine, or a pharmaceutically acceptable salt thereof.

E395. The composition according to any one of embodiments E379 to E394, wherein the free amino acid is glutamic acid, glutamate, or a pharmaceutically acceptable salt thereof.

E396. The composition according to any one of embodiments E379 to E394, wherein the free amino acid is aspartic acid, aspartate, or a pharmaceutically acceptable salt thereof.

E397. The composition according to any one of embodiments E379 to E394, wherein the free amino acid is arginine, or a pharmaceutically acceptable salt thereof.

E398. The composition according to any one of embodiments E379 to E394, wherein the free amino acid is lysine, or a pharmaceutically acceptable salt thereof.

E399. The composition according to any one of embodiments E379 to E398, wherein the solution comprises at least 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, or 40 mM of the free amino acid.

E400. The composition according to any one of embodiments E379 to E399, wherein the solution comprises at least 5 mM of the free amino acid.

E401. The composition according to any one of embodiments E379 to E400, wherein the solution comprises at least 10 mM of the free amino acid.

E402. The composition according to any one of embodiments E379 to E401, wherein the solution comprises at least 15 mM of the free amino acid.

E403. The composition according to any one of embodiments E379 to E402, wherein the solution comprises at least 20 mM of the free amino acid.

E404. The composition according to any one of embodiments E379 to E403, wherein the solution further comprises a sugar and/or buffer.

E405. The composition according to embodiment E404, wherein the sugar is at a concentration of about 10% w/v.

E406. The composition according to embodiment E404 or embodiment E405, wherein the buffer is at a concentration of about 10 mM.

E407. The composition according to any one of embodiments E404 to E406, wherein the buffer is at a pH of about 7.4.

E408. The composition according to any one of embodiments E404 to E407, wherein the sugar comprises sucrose and/or trehalose.

E409. The composition according to any one of embodiments E404 to E408, wherein the sugar comprises sucrose.

E410. The composition according to any one of embodiments E404 to E409, wherein the buffer comprises Tris buffer, PBS, or HEPES buffer.

E411. The composition according to any one of embodiments E404 to E410, wherein the buffer comprises Tris buffer.

E412. The composition according to any one of embodiments E404 to E411, wherein the sugar comprises sucrose and the buffer comprises Tris buffer.

E413. According to another aspect of the present disclosure, there is provided a method of producing the composition of any one of embodiments E379 to E412, comprising combining the isolated RNA and the at least 1 mM of a free amino acid before the isolated RNA is encapsulated in the capsule.

E414. According to another aspect of the present disclosure, there is provided a method of producing the composition of any one of embodiments E379 to E412, comprising combining the isolated RNA and the at least 1 mM of a free amino acid during RNA encapsulation in the capsule.

E415. According to another aspect of the present disclosure, there is provided a method of producing the composition of any one of embodiments E379 to E412, comprising combining the capsule comprising the isolated RNA and the at least 1 mM of a free amino acid after the isolated RNA is encapsulated in the capsule.

E416. According to another aspect of the present disclosure, there is provided a method of vaccinating a subject, comprising administering to the subject in need thereof an effective amount of the composition of any of embodiments E149 to E218 or E379 to E412.

E417. According to another aspect of the present disclosure, there is provided a method for treating or preventing an infectious disease, comprising administering to a subject in need thereof an effective amount of the composition of any of embodiments E149 to E218 or E379 to E412.

E418. The method of embodiment E417, wherein the composition elicits an immune response comprising an antibody response.

E419. The method according of embodiment E417 or E418, wherein the composition elicits an immune response comprising a T cell response.

E420. The method according to any one of embodiments E417 to E419, wherein immunogenicity of the composition is increased compared to immunogenicity of a composition in which the capsule comprising the isolated RNA has not been contacted with the at least 1 mM of a free amino acid.

E421. The method according to embodiment E420, wherein immunogenicity of the composition is increased at least about 15%, 16%, 17%, 18%, 19%, 20%, 21%, 22%, 23%, 24%, 25%, 26%, 27%, 28%, 29%, 30%, 32%, 33%, 34%, or 35%.

E422. The method according to embodiment E420 or E421, wherein immunogenicity of the composition is increased at least about 25%.

E423. According to another aspect of the present disclosure, there is provided a method of increasing immunogenicity of isolated RNA that is encapsulated and/or increasing in vitro expression of isolated RNA that is encapsulated by forming a composition comprising in solution the isolated RNA and at least 1 mM of a free amino acid, the method comprising contacting a capsule comprising the isolated RNA with the free amino acid.

E424. The method according to embodiment E423, wherein the RNA is double stranded.

E425. The method according to embodiment E423, wherein the RNA is single stranded.

E426. The method according to embodiment E423, wherein the RNA is antisense single stranded.

E427. The method according to embodiment E423, wherein the RNA is messenger RNA (mRNA).

E428. The method according to embodiment E423, wherein the RNA is self-amplifying RNA (saRNA).

E429. The method according to any one of embodiments E423 to E428, wherein the RNA comprises:
a 5′ Cap represented by Formula I,

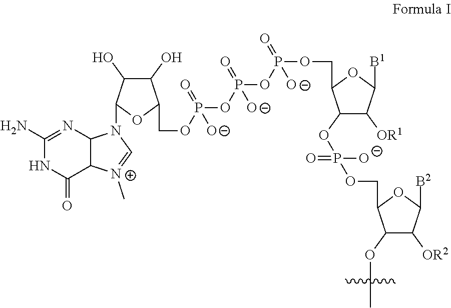

where R¹ and R² are each independently H or Me; and
B¹ and B² are each independently guanine, adenine, or uracil;
a 5′ untranslated region;
a coding region for a nonstructural protein derived from an alphavirus;
a subgenomic promoter derived from an alphavirus;
an open reading frame encoding a gene of interest;
a 3′ untranslated region; and
a 3′ poly A sequence.

E430. The method according to any one of embodiments E423 to E429, wherein the RNA is modified RNA (modRNA).

E431. The method according to any one of embodiments E423 to E430, wherein the RNA is viral RNA.

E432. The method according to any one of embodiment E423 to E431, wherein the RNA encodes a viral antigen.

E433. The method according to any one of embodiment E423 to E432, wherein the RNA is part of a vaccine.

E434. The method according to any one of embodiments E423 to E433, wherein the solution further comprises a sugar and/or a buffer.

E435. The method according to embodiment E434, wherein the sugar is at a concentration of about 10% w/v.

E436. The method according to embodiment E434 or embodiment E435, wherein the buffer is at a concentration of about 10 mM.

E437. The method according to any one of embodiments E434 to E436, wherein the buffer is at a pH of about 7.4.

E438. The method according to any one of embodiments E434 to E437, wherein the sugar comprises sucrose and/or trehalose.

E439. The method according to any one of embodiments E434 to E438, wherein the sugar comprises sucrose.

E440. The method according to any one of embodiments E434 to E439, wherein the buffer comprises Tris buffer, PBS, or HEPES buffer.

E441. The method according to any one of embodiments E434 to E440, wherein the buffer comprises Tris buffer.

E442. The method according to any one of embodiments E434 to E441, wherein the sugar comprises sucrose and the buffer comprises Tris buffer.

E443. The method according to any one of embodiments E423 to E442, wherein the capsule comprises a cationic lipid.

E444. The method according to any one of embodiments E423 to E443, wherein the capsule comprises a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in water emulsion, a water-in-oil emulsion, an emulsome, a polycationic peptide, a cationic nanoemulsion, or a combination thereof.

E445. The method according to any one of embodiments E423 to E444, wherein free amino acid is selected from the group consisting of glutamic acid, glutamate, aspartic acid, aspartate, arginine, lysine, tyrosine, cysteine, glutamine, asparagine, threonine, and serine, or a pharmaceutically acceptable salt thereof.

E446. The method according to any one of embodiments E423 to E445, wherein the free amino acid is glutamic acid, glutamate, or a pharmaceutically acceptable salt thereof.

E447. The method according to any one of embodiments E423 to E445, wherein the free amino acid is aspartic acid, aspartate, or a pharmaceutically acceptable salt thereof.

E448. The method according to any one of embodiments E423 to E445, wherein the free amino acid is arginine, or a pharmaceutically acceptable salt thereof.

E449. The method according to any one of embodiments E423 to E445, wherein the free amino acid is lysine, or a pharmaceutically acceptable salt thereof.

E450. The method according to any one of embodiments E423 to E449, wherein the solution comprises at least 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM 36 mM, 37 mM, 38 mM, 39 mM, or 40 mM of the free amino acid.

E451. The method according to any one of embodiments E423 to E450, wherein the solution comprises at least 5 mM of the free amino acid.

E452. The method according to any one of embodiments E423 to E451, wherein the solution comprises at least 10 mM of the free amino acid.

E453. The method according to any one of embodiments E423 to E452, wherein the solution comprises at least 15 mM of the free amino acid.

E454. The method according to any one of embodiments E423 to E453, wherein the solution comprises at least 20 mM of the free amino acid.

E455. The method according to any one of embodiments E423 to E454, further comprising the step of storing the composition.

E456. The method according to embodiment E455, wherein the encapsulated isolated RNA composition is stored for at least about 2 hours, 4 hours, 6 hours, 12 hours, 1 day, 2 days, 3 days, 4 days, 5 days, 6 days, 7 days, 2 weeks, 3 weeks, four weeks, five weeks, six weeks, seven weeks, or 8 weeks.

E457. The method according to embodiment E455 or embodiment E456, wherein the encapsulated isolated RNA composition is stored at a temperature of about 5° C., 0° C., −10° C., −20° C., −30° C., −40° C., −50° C., −60° C., −70° C., or −80° C.

E458. The method according to any one of embodiments E423 to E457, wherein the isolated RNA is contacted with the free amino acid before the isolated RNA is encapsulated in the capsule.

E459. The method according to any one of embodiments E423 to E457, wherein the isolated RNA is contacted with the free amino acid during RNA encapsulation in the capsule.

E460. The method according to any one of embodiments E423 to E457, wherein the capsule comprising the isolated RNA is contacted with the free amino acid after the isolated RNA is encapsulated in the capsule.

E461. The method according to any one of embodiments E423 to E460, wherein contacting the capsule comprising the isolated RNA with the free amino acid increases in vitro expression of the isolated RNA compared to in vitro expression of encapsulated isolated RNA that has not been contacted with the solution comprising at least 1 mM of a free amino acid.

E462. The method according to embodiment E461, wherein in vitro expression of the isolated RNA comprised in the composition is increased at least about 20%, 21%, 22%, 23%, 24%, 25%, 26%, 27%, 28%, 29%, 30%, 32%, 33%, 34%, 35%, 36%, 37%, 38%, 39%, or 40%.

E463. The method according to embodiment E461 or E462, wherein in vitro expression of the isolated RNA comprised in the composition is increased at least about 33%.

E464. The method according to any one of embodiments E423 to E463, wherein contacting the capsule comprising the isolated RNA with the free amino acid increases immunogenicity of the composition compared to immunogenicity of a composition comprising encapsulated isolated RNA that has not been contacted with the solution comprising at least 1 mM of a free amino acid.

E465. The method according to embodiment E464, wherein immunogenicity of the composition is increased at least about 15%, 16%, 17%, 18%, 19%, 20%, 21%, 22%, 23%, 24%, 25%, 26%, 27%, 28%, 29%, 30%, 32%, 33%, 34%, or 35%.

E466. The method according to embodiment E464 or E465, wherein immunogenicity of the composition is increased at least about 25%.

It is contemplated that any embodiment discussed in this specification can be implemented with respect to any method or composition of the disclosure, and vice versa. Furthermore, compositions of the disclosure can be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 provides FgenL RNA degradation results (#1) from the TapeStation Assay via gel electrophoresis for: FgenL and various amino acids (20 mM); FgenL and CAPS buffer (50 mM, pH 11); at 0 minutes and 15 minutes at 50° C.

FIG. 2 provides FgenL RNA degradation results (#2) from the TapeStation Assay via gel electrophoresis for: FgenL and various amino acids (20 mM); FgenL and CAPS buffer (50 mM, pH 11); at 0 minutes and 15 minutes at 50° C.

FIG. 3 provides FgenL RNA degradation results from the TapeStation Assay with 20 mM amino acids and FgenL RNA, 50 mM CAPS pH 11 (Avg N=2), as % Control, at 50° C. for 15 minutes.

FIG. 4 provides traces for the TapeStation Assay with 20 mM alanine and FgenL RNA, 50 mM CAPS pH 11.

FIG. 5 provides traces for the TapeStation Assay with 20 mM glutamic acid and FgenL RNA, 50 mM CAPS pH 11.

FIG. 6 provides traces for the TapeStation Assay with 20 mM aspartic acid and FgenL RNA, 50 mM CAPS pH 11.

FIG. 7 provides traces for the TapeStation Assay with 20 mM cysteine and FgenL RNA, 50 mM CAPS pH 11.

FIG. 8 provides traces for the TapeStation Assay with 20 mM glutamine and FgenL RNA, 50 mM CAPS pH 11.

FIG. 9 provides traces for the TapeStation Assay with 20 mM valine and FgenL RNA, 50 mM CAPS pH 11.

FIG. 10 provides chi18 RNA degradation results (#1) from the TapeStation Assay via gel electrophoresis for: chi18 and various amino acids (20 mM); chi18 and CAPS buffer (50 mM, pH 11); at 0 minutes and 15 minutes at 50° C.

FIG. 11 provides chi18 RNA degradation results (#2) from the TapeStation Assay via gel electrophoresis for: chi18 and various amino acids (20 mM); chi18 and CAPS buffer (50 mM, pH11); at 0 minutes and 15 minutes at 50° C.

FIG. 12 provides chi18 RNA degradation results from the TapeStation Assay with 20 mM amino acids and chi18 RNA, 50 mM CAPS pH 11 (Avg N=2), as % Control, at 50° C. for 15 minutes.

FIG. 13 provides FgenL model RNA degradation results from the Size Exclusion Base Hydrolysis Degradation Assay, showing averages (n=2) at time points T2, T10, and T24.

FIGS. 14A-14F provide FgenL model RNA Example Traces from the Size Exclusion Base Hydrolysis Degradation Assay at time points TO, T2, T10, and T24 using CAPS only (FIG. 14A), tyrosine (FIG. 14B), glutamic acid (FIG. 14C), aspartic acid (FIG. 14D), methionine (FIG. 14E), serine (FIG. 14F), or valine (FIG. 14G).

FIG. 15 provides Chi18 model RNA degradation results from the Size Exclusion Base Hydrolysis Degradation Assay, showing averages (n=2) at time points T2, T10, and T24.

FIGS. 16A-16F provide Chi18 model RNA Example Traces from the Size Exclusion Base Hydrolysis Degradation Assay at time points TO, T2, T10, and T24 using CAPS only (FIG. 16A), tyrosine (FIG. 16B), glutamic acid (FIG. 16C), aspartic acid (FIG. 16D), methionine (FIG. 16E), serine (FIG. 16F), or valine (FIG. 16G).

FIGS. 17A-17D provide droplet digital polymerase chain reaction assay plots generated for amino acids and Chi18 model RNA at pH 11.0 and 30° C. for 16 hours. FIG. 17A shows plots for asparagine, aspartic acid, glutamic acid, phenylalanine, isoleucine, and leucine. FIG. 17B shows plots for arginine, glutamine, glycine, cystine, alanine, and cysteine. FIG. 17C shows plots for histidine, proline, serine, valine, lysine, and methionine. FIG. 17D shows plots for threonine, tryptophan, tyrosine, hydroxyproline, glutathione, and TCEP.

FIGS. 18A-18D provide droplet digital polymerase chain reaction assay plots generated for amino acids and FgenL model RNA at pH 11.0 and 44° C. for 16 hours. FIG. 18A shows plots for glutamic acid, asparagine, aspartic acid, glutamine, cysteine, and histidine. FIG. 18B shows plots for alanine, arginine, glycine, isoleucine, leucine, and phenylalanine. FIG. 18C shows plots for proline, serine, threonine, lysine, methionine, and cystine. FIG. 18D shows plots for tryptophan, tyrosine, valine, hydroxyproline, glutathione, and TCEP.

FIGS. 19A-19B show LNP attribute changes (encapsulation efficiency (EE), size/PDI, integrity (FA), and in vitro expression (IVE)) after freeze/thaw for LNPs formulated with PBS and Sucrose in the absence (FIG. 19A) and presence (FIG. 19B) of 20 mM Glutamic Acid.

FIGS. 20A-20B show LNP attribute changes (encapsulation efficiency (EE), size/PDI, integrity (FA), and in vitro expression (IVE)) after freeze/thaw for LNPs formulated with Tris and Sucrose in the absence (FIG. 20A) and presence (FIG. 20B) of 20 mM Glutamic Acid.

FIGS. 21A-21B show LNP attribute changes (encapsulation efficiency (EE), size/PDI, integrity (FA), and in vitro expression (IVE)) after freeze/thaw for LNPs formulated with HEPES and Sucrose in the absence (FIG. 21A) and presence (FIG. 21B) of 20 mM Glutamic Acid.

FIGS. 22A-22B show colloidal stability in terms of LNP size (FIG. 22A) and PDI (FIG. 22B) of LNPs formulated with HEPES and Sucrose when stored at 5° C. post-freeze/thaw.

FIG. 23 shows attribute changes (encapsulation efficiency (EE), size/PDI, integrity (FA), and in vitro expression (IVE)) of LNPs formulated with PBS, Tris, or HEPES and Sucrose when stored at −70° C.

FIG. 24 shows attribute changes (encapsulation efficiency (EE), size/PDI, integrity (FA), and in vitro expression (IVE)) of LNPs formulated with PBS, Tris, or HEPES; Sucrose; and 20 mM Glutamic Acid when stored at −70° C.

FIG. 25 shows attribute changes (encapsulation efficiency (EE), size/PDI, integrity (FA), and in vitro expression (IVE)) of LNPs formulated with PBS, Tris, or HEPES and Sucrose when stored at 5° C.

FIG. 26 shows attribute changes (encapsulation efficiency (EE), size/PDI, integrity (FA), and in vitro expression (IVE)) of LNPs formulated with PBS, Tris, or HEPES; Sucrose; and 20 mM Glutamic Acid when stored at 5° C.

FIG. 27 shows immunogenicity of LNPs formulated with PBS, Tris, or HEPES and Sucrose±20 mM Glutamic Acid. All samples were stored frozen at −80° C. Samples had been stored ˜7-weeks at the time of the in vivo study.

FIG. 28 shows immunogenicity of LNPs formulated with PBS and Sucrose. All samples were stored frozen at −80° C., at −20° C., or at 5° C. Samples had been stored ˜7-weeks at the time of the in vivo study.

FIG. 29 shows immunogenicity of LNPs formulated with Tris and Sucrose±20 mM Glutamic Acid. All samples were stored frozen at −80° C., at −20° C., or at 5° C. Samples had been stored ˜7-weeks at the time of the in vivo study.

### DETAILED DESCRIPTION

### I. Examples of Definitions

Throughout this application, the term “about” is used according to its plain and ordinary meaning in the area of cell and molecular biology to indicate that a value includes the inherent variation or standard deviation of error for the measurement or quantitation method being employed to determine the value. For example, in some embodiments, the term “about” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the measurement or quantitation.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The phrase “essentially all” is defined as “at least 95%”; if essentially all members of a group have a certain property, then at least 95% of members of the group have that property. In some instances, essentially all means equal to any one of, at least any one of, or between any two of 95, 96, 97, 98, 99, or 100% of members of the group have that property.

The compositions and methods for their use can “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that embodiments described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The terms “protect” or “protection” or “protecting” or “protective effect” as used herein regarding RNA, means slowing, reducing, or eliminating RNA degradation. The protective effect may be measured by assays including, but not limited to: Droplet Digital Polymerase Chain Reaction Assay; Size Exclusion Base Hydrolysis Degradation Assay; and TapeStation Assay, as described herein.

The terms “inhibiting” or “reducing” or any variation of these terms includes any measurable decrease or complete inhibition to achieve a desired result. The terms “promote” or “increase” or any variation of these terms includes any measurable increase to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “RNA,” as used herein, means a nucleic acid molecule that includes ribonucleotide residues (such as containing the nucleotide base(s) adenine (A), cytosine (C), guanine (G) and/or uracil (U)). For example, RNA can contain all, or a majority of, ribonucleotide residues. As used herein, the term “ribonucleotide” means a nucleotide with a hydroxyl group at the 2′ position of a β-D-ribofuranosyl group. In one aspect, RNA can be messenger RNA (mRNA) that relates to a RNA transcript which encodes a peptide or protein. As known to those of skill in the art, mRNA generally contains a 5′ untranslated region (5′-UTR), a polypeptide coding region, and a 3′ untranslated region (3′-UTR). Without any limitation, RNA can encompass double stranded RNA, antisense RNA, single stranded RNA, isolated RNA, synthetic RNA, RNA that is recombinantly produced, and modified RNA (modRNA).

An “isolated RNA” is defined as an RNA molecule that can be recombinant or has been isolated from total genomic nucleic acid.

A “modified RNA” or “modRNA” refers to an RNA molecule having at least one addition, deletion, substitution, and/or alteration of one or more nucleotides as compared to naturally occurring RNA. Such alterations can refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide can replace one or more uridine and/or cytidine nucleotides. For example, these replacements can occur for every instance of uridine and/or cytidine in the RNA sequence, or can occur for only select uridine and/or cytidine nucleotides. Such alterations to the standard nucleotides in RNA can include non-standard nucleotides, such as chemically synthesized nucleotides or deoxynucleotides. For example, at least one uridine nucleotide can be replaced with 1-methylpseudouridine in an RNA sequence. Other such altered nucleotides are known to those of skill in the art. Such altered RNAs are considered analogs of naturally-occurring RNA. In some aspects, the RNA is produced by in vitro transcription using a DNA template, where DNA refers to a nucleic acid that contains deoxyribonucleotides. In some aspects, the RNA can be replicon RNA (replicon), in particular self-replicating RNA, or self-amplifying RNA (saRNA).

As contemplated herein, without any limitations, RNA can be used as a therapeutic modality to treat and/or prevent a number of conditions in mammals, including humans. Methods described herein comprise administration of the RNA described herein to a mammal, such as a human. For example, in one aspect such methods of use for RNA include an antigen-coding RNA vaccine to induce robust neutralizing antibodies and accompanying/concomitant T-cell response to achieve protective immunization with preferably minimal vaccine doses. The RNA administered is preferably in vitro transcribed RNA.

For example, such RNA can be used to encode at least one antigen intended to generate an immune response in said mammal. Pathogenic antigens are peptide or protein antigens derived from a pathogen associated with infectious disease which are preferably selected from antigens derived from the pathogens Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 01 57:H7, 01 1 1 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBov), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowled, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B1 9, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

Conditions and/or diseases that can be treated with such RNA therapeutics include, but are not limited to, those caused and/or impacted by viral infection. Such viruses include, but are not limited to, an arenavirus (such as Lassa virus, or lymphocytic choriomeningitis virus (LCMV)); an astrovirus; a bunyavirus (such as a Hantavirus); a calicivirus; a coronavirus (such as a severe acute respiratory syndrome virus (SARS)—e.g. SARS-CoV-2, or a middle east respiratory syndrome (MERS) virus); a filovirus (such as Ebola virus or Marburg virus); a flavivirus (such as Yellow Fever virus, West Nile virus, or Hepatitis C virus (HCV)); a hepadnavirus; a hepevirus; an orthomyxovirus (such as Influenza A virus, Influenza B virus, or Influenza C virus); a paramyxovirus (such as Rubeola virus, or Rubulavirus); a picornavirus (such as Poliovirus, Hepatitis A virus, or Rhinovirus); a reovirus (such as Rotavirus); a retrovirus (such as Human Immunodeficiency Virus (HIV), or Human T-lymphotropic virus (HTLV)); a rhabdovirus (such as Rabies virus or Rabies lyssavirus); a togavirus (such as Sindbis virus (SINV), Eastern Equine Encephalitis virus (EEEV), Western Equine Encephalitis virus (WEEV), or Rubella virus).

The term “RNA degradation,” as used herein, means an original RNA nucleotide sequence is no longer intact. RNA may be degraded under certain conditions including, but not limited to, pH or temperature, or both, over time. In particular, acidic pH conditions, basic pH conditions and/or increased temperatures may accelerate RNA degradation over time.

The term “acidic conditions” or “acidic pH conditions,” as used herein, means a pH less than 7.0.

The term “basic conditions” or “basic pH conditions,” as used herein, means a pH greater than 7.0.

The term “amino acid,” as used herein, means an organic compound comprising an amino and carboxyl group. The term “free amino acid,” as used herein, refers to an amino acid or a salt thereof that exists in a monomeric form in solution. The free amino acid may exist in a fully protonated form, a fully deprotonated form, or any ionic form therebetween.

Several non-amino acids were also tested herein for their ability to protect RNA, such as DTT, TCEP, and glutathione. As used herein the term “DTT” refers to dithiothreitol. As used herein, the term TCEP refers to tris(2-carboxyethyl)phosphine.

The term “pharmaceutically acceptable salt,” as used herein, means those salts within the scope of sound medical judgement that are suitable for use in contact with the tissues of humans and animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66:1-19, herein incorporated by reference. The salts can be prepared in situ during the final isolation and purification of the compounds of the present disclosure or separately by reacting a free base (basic nitrogen) with a suitable organic or inorganic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include, but are not limited to, such inorganic acids as hydrochloric acid, hydrobromic acid, sulphuric acid and phosphoric acid and such organic acids as oxalic acid, maleic acid, succinic acid and citric acid. The term “pharmaceutically acceptable salt,” as used herein, also means salts of carboxylic acid and sulfonic acid groups prepared by reacting the free acid with a positively charged inorganic or organic ion (cation) that is generally considered suitable for human consumption. Examples of pharmaceutically acceptable cations include, but are not limited to, lithium, sodium, potassium, magnesium, calcium, ferrous, ferric, ammonium, alkylammonium, dialkylammonium, trialkylammonium, tetraalkylammonium, diethanolammmonium, and choline. Cations may be interchanged by methods known in the art, such as ion exchange. Where compounds of the present disclosure contain one or more carboxylic acid groups or sulfonic acid groups, addition of a base (such as a hydroxide or a free amine) may yield the appropriate cationic form.

### II. Amino Acids

Suitable amino acids, according to the present disclosure, include, but are not limited to, aspartic acid, aspartate, arginine, glycine, glutamic acid, glutamate, proline, threonine, theanine, cysteine, cystine, alanine, valine, tyrosine, leucine, arabinose, trans-4-hydroxyproline, isoleucine, asparagine, serine, lysine, histidine, ornithine, methionine, carnitine, aminobutyric acid (α-, β-, and/or δ-isomers), glutamine, hydroxyproline, taurine, norvaline, sarcosine, and their salt forms such as sodium or potassium salts or acid salts.

In some embodiments, the amino acids are present in solution. The amino acids, including or or more amino acids, may be present in solution as free amino acids, including one or more free amino acids.

The present disclosure further provides for any mixture of any of the amino acids, such as free amino acids, disclosed herein for the purpose of protecting RNA. Without limitation, the disclosure provides, for example, a mixture of any two, three, four, five, six, or more amino acids as disclosed herein, which may be present as free amino acids. Such mixtures can be present in any amounts/ratios sufficient to produce a protective effect on RNA.

Any of the free amino acids contemplated for use herein may also be in the D- or L-configuration. Additionally, the amino acids may be α-, β-, γ-, and/or δ-isomers if appropriate. Combinations of the foregoing amino acids and their corresponding salts (e.g., sodium, potassium, calcium, magnesium salts, or other alkali or alkaline earth metal salts thereof, or acid salts) also are suitable in some embodiments. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). Non-limiting examples of modified amino acids include amino acid derivatives such as trimethyl glycine, N-methyl-glycine, and N-methyl-alanine. As used herein, modified amino acids encompass both modified and unmodified amino acids. As used herein, amino acids also encompass both peptides and polypeptides (e.g., dipeptides, tripeptides, tetrapeptides, pentapeptides, etc.) such as glutathione and L-alanyl-L-glutamine.

Suitable polyamino acids include poly-L-glutamic acid, poly-L-glutamate, poly-L-aspartic acid, poly-L-aspartate, poly-L-lysine (e.g., poly-L-α-lysine or poly-L-ε-lysine), poly-L-ornithine (e.g., poly-L-α-ornithine or poly-L-ε-ornithine), poly-L-arginine, other polymeric forms of amino acids, and salt forms thereof (e.g., calcium, potassium, sodium, or magnesium salts such as L-glutamic acid mono sodium salt). The poly-amino acid also may be in the D- or L-configuration. Additionally, the poly-amino acids may be α-, β-, γ-, δ-, and ε-isomers if appropriate. Combinations of the foregoing poly-amino acids and their corresponding salts (e.g., sodium, potassium, calcium, magnesium salts, or other alkali or alkaline earth metal salts thereof or acid salts) also are suitable in some embodiments. The poly-amino acids described herein also may comprise co-polymers of different amino acids. The poly-amino acids may be natural or synthetic. The poly-amino acids also may be modified, such that at least one atom has been added, removed, substituted, or combinations thereof (e.g., N-alkyl poly-amino acid or N-acyl poly-amino acid). As used herein, poly-amino acids encompass both modified and unmodified poly-amino acids. For example, modified poly-amino acids include, but are not limited to, poly-amino acids of various molecular weights (MW), such as poly-L-α-lysine with a MW of 1,500, MW of 6,000, MW of 25,200, MW of 63,000, MW of 83,000, or MW of 300,000. The amino acids may be selected from one of the essential or non-essential amino acids selected from glycine, L-lysine, L-alanine, L-phenylalanine, L-leucine, L-isoleucine, L-proline, L-hydroxyproline, L-arginine, L-ornithine, L-methionine, L-aspartic acid, L-aspartate, L-glutamic acid, L-glutamate, L-valine, L-threonine, L-isothreonine, L-histidine, L-tryptophan, L-serine, L-glutamine, L-citrulline, or mixtures thereof or their enantiomeric forms.

In one embodiment, the free amino acid is glutamic acid, glutamate, aspartic acid, aspartate, arginine, lysine, tyrosine, cysteine, glutamine, asparagine, threonine, serine, or any mixtures thereof. In another embodiment, the amino acid is glutamic acid, glutamate, aspartic acid, aspartate, arginine, lysine, or any mixtures thereof. In a specific embodiment, the amino acid is glutamic acid, glutamate, or a pharmaceutically acceptable salt thereof. In a specific embodiment, the amino acid is aspartic acid, aspartate, or a pharmaceutically acceptable salt thereof. In a specific embodiment, the amino acid is arginine. In a specific embodiment, the amino acid is lysine.

The free amino acids of the present disclosure can be present in any amount sufficient to provide a protective effect of the RNA, to increase immunogenicity of the RNA and/or compositions comprising the RNA, and/or to increase in vitro expression of the RNA. For example, those of skill in the art will appreciate that an effective amount can be determined by use of the assays disclosed herein. Without limitation, such amounts of amino acids that provide a protective effect of the RNA include concentrations in excess of the RNA concentration, concentrations within 10× of the total nucleobase concentration of the RNA, and concentrations approaching the solubility limit of the amino acid in the delivery vehicle. Typical concentrations used to protect RNA from degradation, increase immunogenicity of the RNA and/or compositions comprising the RNA, and/or to increase in vitro expression of the RNA can be, for example, between 1 mM and 100 mM, or any range or value derivable therein. For example, the amino acid concentration can be equal to any one of, at least any one of, at most any one of, or between any two of 1 mM, 2 mM, 3 mM, 4 mM, 5 mM, 6 mM, 7 mM, 8 mM, 9 mM, 10 mM, 11 mM, 12 mM, 13 mM, 141 mM, 15 mM, 16 mM, 17 mM, 18 mM, 9 mM, 2 mM, 2 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM. In some embodiments, the amino acid concentration can range from 1 to 10 mM, from 1 to 20 mM, from 1 to 30 mM, from 1 to 40 mM, from 1 to 50 mM, from 1 to 60 mM, from 1 to 70 mM, from 1 to 80 mM, from 1 to 90 mM, or from 1 to 100 mM. In some embodiments, the amino acid concentration can range from 10 to 20 mM, from 20 to 30 mM, from 30 to 40 mM, from 40 to 50 mM, from 50 to 60 mM, from 60 to 70 mM, from 70 to 80 mM, from 80 to 90 mM, or from 90 to 100 mM. In some embodiments, the amino acid concentration can be 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In specific embodiments, the amino acid concentration is 1 mM. In specific embodiments, the amino acid concentration is 5 mM. In specific embodiments, the amino acid concentration is 10 mM. In specific embodiments, the amino acid concentration is 15 mM. In specific embodiments, the amino acid concentration is 20 mM.

### III. RNA Molecules

In some embodiments, the RNA molecule described herein is a coding RNA molecule. Coding RNA includes a functional RNA molecule that may be translated into a peptide or polypeptide. In some embodiments, the coding RNA molecule includes at least one open reading frame coding for at least one peptide or polypeptide. The coding RNA molecule may include one (monocistronic), two (bicistronic) or more (multicistronic) open reading frames (ORFs), which may be a sequence of codons that is translatable into a polypeptide or protein of interest. The coding RNA molecule may be a messenger RNA (mRNA) molecule, viral RNA molecule, or self-amplifying RNA molecule (saRNA, also referred to as a replicon). In some embodiments, the RNA molecule is an mRNA. In some embodiments, the RNA molecule is a saRNA. In some embodiments, the saRNA molecule may be a coding RNA molecule.

The RNA molecule may encode one polypeptide of interest or more, such as an antigen or more than one antigen, e.g., two, three, four, five, six, seven, eight, nine, ten, or more polypeptides. Alternatively, or in addition, one RNA molecule may also encode more than one polypeptide of interest or more, such as an antigen, e.g., a bicistronic, or tricistronic RNA molecule that encodes different or identical antigens.

The sequence of the RNA molecule may be codon optimized or deoptimized for expression in a desired host, such as a human cell.

In some embodiments, the RNA molecule includes equal to any one of, at least any one of, at most any one of, or between any two of from about 20 to about 100,000 nucleotides (e.g., equal to any one of, at least any one of, at most any one of, or between any two of from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000). In some embodiments, the RNA molecule includes at least 100 nucleotides. For example, in some embodiments, the RNA has a length between 100 and 15,000 nucleotides; between 7,000 and 16,000 nucleotides; between 8,000 and 15,000 nucleotides; between 9,000 and 12,500 nucleotides; between 11,000 and 15,000 nucleotides; between 13,000 and 16,000 nucleotides; between 7,000 and 25,000 nucleotides. In some embodiments, the second RNA or the saRNA molecule includes at least about 7000 nucleotides. In some aspects, one or more of the nucleotide size ranges in the list may be excluded.

The sequence of the RNA molecule may be modified if desired, for example to increase the efficacy of expression or replication of the RNA, or to provide additional stability or resistance to degradation. For example, the RNA sequence may be modified with respect to its codon usage, for example, to increase translation efficacy and half-life of the RNA.

In some embodiments, the RNA molecules may include one or more structural and/or chemical modifications or alterations which impart useful properties to the polynucleotide including, in some embodiments, the lack of a substantial induction of the innate immune response of a cell into which the polynucleotide is introduced. As used herein, a “structural” feature or modification is one in which two or more linked nucleotides are inserted, deleted, duplicated, inverted or randomized in an RNA molecule without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some embodiments, the RNA molecule may include one or more modified nucleotides in addition to any 5′ cap structure. Naturally occurring nucleotide modifications are known in the art.

In some embodiments, the RNA molecule does not include modified nucleotides, e.g., does not include modified nucleobases, and all of the nucleotides in the RNA molecule are conventional standard ribonucleotides A, U, G, and C, with the exception of an optional 5′ cap that may include, for example, 7-methylguanosine, which is further described below. In some embodiments, the RNA may include a 5′ cap comprising a 7′-methylguanosine, and the first 1, 2 or 3 5′ ribonucleotides may be methylated at the 2′ position of the ribose.

A. Modified Nucleobases

Modified nucleobases which may be incorporated into modified nucleosides and nucleotides and be present in the RNA molecules include, for example, m5C (5-methylcytidine), m5U (5-methyluridine), m6A (N6-methyladenosine), s2U (2-thiouridine), Um (2′-O-methyluridine), mIA (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-0-ribosyladenosine (phosphate)); I (inosine); mil (1-methylinosine); m′Im (1,2′-0- dimethylinosine); m3C (3-methylcytidine); Cm (2T-0-methylcytidine); s2C (2-thiocytidine); ac4C (N4-acetylcytidine); £5C (5-fonnylcytidine); m5Cm (5,2-0-dimethylcytidine); ac4Cm (N4acetyl2TOmethylcytidine); k2C (lysidine); mIG (1-methylguanosine); m2G (N2-methylguanosine); m7G (7-methylguanosine); Gm (2′-0-methylguanosine); m22G (N2,N2-dimethylguanosine); m2Gm (N2,2′-0-dimethylguanosine); m22Gm (N2,N2,2′-0-trimethylguanosine); Gr(p) (2′-0-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-0-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-0-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-0-methyluridine); cmnm5U (5-carboxymethylaminomethyluridine); cnmm5Um (5-carboxymethy 1 aminomethyl-2-L-Omethyluridine); cmnm5s2U (5-carboxymethylaminomethyl-2-thiouridine); m62A (N6,N6-dimethyladenosine); Tm (2′-0-methylinosine); m4C (N4-methylcytidine); m4Cm (N4,2-0-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-0-dimethyluridine); m5D (5-methyldihydrouridine); f5Cm (5-formyl-2′-0-methylcytidine); mlGm (1,2′-0-dimethylguanosine); m′Am (1,2-0-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-Ce)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. In some aspects, one or more of the modified nucleosides in the list may be excluded.

Additional exemplary modified nucleotides include any one of N-1-methylpseudouridine; pseudouridine, N6-methyladenosine, 5-methylcytidine, and 5-methyluridine. In some embodiments, the modified nucleotide is N-1-methylpseudouridine.

In some embodiments, the RNA molecule may include phosphoramidate, phosphorothioate, and/or methylphosphonate linkages.

In some embodiments, the RNA molecule includes a modified nucleotide selected from any one of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

B. UTRs

The 5′ untranslated regions (UTR) is a regulatory region of DNA situated at the 5′ end of a protein coding sequence that is transcribed into mRNA but not translated into protein. 5′ UTRs may contain various regulatory elements, e.g., 5′ cap structure, stem-loop structure, and an internal ribosome entry site (IRES), which may play a role in the control of translation initiation. The 3′ UTR, situated downstream of a protein coding sequence, may be involved in regulatory processes including transcript cleavage, stability and polyadenylation, translation, and mRNA localization. In some embodiments, the UTR is derived from an mRNA that is naturally abundant in a specific tissue (e.g., lymphoid tissue), to which the mRNA expression is targeted. In some embodiments, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). According, the UTR sequence may prolong protein synthesis in a tissue-specific manner. In some embodiments, the 5′ UTR and the 3′ UTR sequences are computationally derived. In some embodiments, the 5′ UTR and the 3′ UTRs are derived from a naturally abundant mRNA in a tissue. The tissue may be, for example, liver, a stem cell, or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some embodiments, the 5′ UTR and the 3′ UTR are derived from an alphavirus. In some embodiments, the 5′ UTR and the 3′ UTR are from a wild-type alphavirus. Examples of alphaviruses are described below.

In some embodiments, the first RNA molecule includes a 5′ UTR and the 3′ UTR derived from a naturally abundant mRNA in a tissue. In some embodiments, the first RNA molecule includes a 5′ UTR and the 3′ UTR derived from an alphavirus. In some embodiments, the second RNA or the saRNA molecule includes a 5′ UTR and the 3′ UTR derived from an alphavirus. In some embodiments, the second RNA or the saRNA molecule includes a 5′ UTR and the 3′ UTR from a wild-type alphavirus. In some embodiments, the RNA molecule includes a 5′ cap.

C. Open Reading Frame (ORF)

The 5′ and 3′ UTRs may be operably linked to an ORF, which may be a sequence of codons that is capable of being translated into a polypeptide of interest. As stated above, the RNA molecule may include one (monocistronic), two (bicistronic) or more (multicistronic) open reading frames (ORFs).

In some embodiments, the ORF encodes a non-structural viral gene. In some embodiments, the ORF further includes one or more subgenomic promoters. In some embodiments, the RNA molecule includes a subgenomic promoter operably linked to the ORF. In some embodiments, the subgenomic promoter comprises a cis-acting regulatory element. In some embodiments, the cis-acting regulatory element is immediately downstream (5′-3′) of B². In some embodiments, the cis-acting regulatory element is immediately downstream (5′-3′) of a guanine that is immediately downstream of B². In some embodiments, the cis-acting regulatory element is an AU-rich element. In some embodiments, the AU-rich element is au, auaaaagau, auaaaaagau, auag, auauauauau, auauauau, auauauauauau, augaugaugau, augau, auaaaagaua, or auaaaagaug.

In some embodiments, a RNA molecule may include (i) an ORF encoding a replicase which may transcribe RNA from the RNA molecule and (ii) an ORF encoding at least one an antigen or polypeptide of interest. The polymerase may be an alphavirus replicase, e.g., including any one of the non-structural alphavirus proteins nsP1, nsP2, nsP3 and nsP4, or a combination thereof. In some embodiments, the RNA molecule includes alphavirus nonstructural protein nsP1. In some embodiments, the RNA molecule includes alphavirus nonstructural protein nsP2. In some embodiments, the RNA molecule includes alphavirus nonstructural protein nsP3. In some embodiments, the RNA molecule includes alphavirus nonstructural protein nsP4. In some embodiments, the RNA molecule includes alphavirus nonstructural proteins nsP1, nsP2, and nsP3. In some embodiments, the RNA molecule includes alphavirus nonstructural proteins nsP1, nsP2, nsP3, and nsP4. In some embodiments, the RNA molecule includes any combination of nsP1, nsP2, nsP3, and nsP4. In some embodiments, the RNA molecule does not include nsP4.

In some embodiments, the RNA molecule does not encode alphavirus structural proteins. In some embodiments, the RNA molecule may have one or more additional (e.g., downstream) open reading frames, e.g., to encode further antigen(s) or to encode accessory polypeptides.

In some embodiments, a first RNA molecule does not include an ORF encoding any polypeptide of interest, whereas a second RNA or a saRNA molecule includes an ORF encoding a polypeptide of interest. In some embodiments, the first RNA molecule does not include a subgenomic promoter.

In some embodiments, the first RNA molecule includes an ORF for a nonstructural protein derived from an alphavirus. In some embodiments, the ORF encoding a nonstructural protein in the first RNA molecule and in the second RNA or the saRNA molecule are identical. In some embodiments, the second RNA or the saRNA molecule further includes an ORF encoding a polypeptide of interest.

In some embodiments, the second RNA or the saRNA molecule may lead to a production of genomic RNA copies of itself in a cell, but not to the production of RNA-containing virions. In some embodiments, the second RNA or the saRNA molecule cannot perpetuate itself in infectious form. For example, the genes encoding alphavirus structural proteins, which are necessary for perpetuation in wild-type alphaviruses, are absent from the second RNA or saRNA molecules of the present disclosure, and their place is taken by a gene(s) encoding the polypeptide of interest, such that the subgenomic transcript encodes the polypeptide of interest, such as an immunogen, rather than the structural alphavirus virion proteins.

D. Genes of Interest

The RNA molecule described herein may include a gene of interest. The gene of interest can encode a polypeptide of interest selected from, e.g., biologics, antibodies, vaccines, therapeutic polypeptides or peptides, cell penetrating peptides, secreted polypeptides, plasma membrane polypeptides, cytoplasmic or cytoskeletal polypeptides, intracellular membrane bound polypeptides, nuclear polypeptides, polypeptides associated with human disease, targeting moieties or those polypeptides encoded by the human genome for which no therapeutic indication has been identified but which nonetheless have utility in areas of research and discovery. In some instances, the sequence for a particular gene of interest is readily identified by one of skill in the art using public and private databases, e.g., GenBank.

In some embodiments, the RNA molecule comprises an RNAsequence derived from HCV IRES. In some embodiments, the RNA molecule comprises FgenL or Chi18-4 model RNA. FgenL or Chi18-4 model RNA were chosen and designed based on Leija-Martinex et al., with 5′ and 3′ primer annealing sites at the two ends (N. Leija-Martinez et al., Nucleic Acids Res 42, 13963-13968 (2014)). The FgenL and Chi18-4 RNA sequences were derived from a fungal organism named Trichoderma atroviride. The FgenL sequence is available on the fungal genomics resource database with protein ID 258498, and Chi18-4 sequence is available from GenBank with the accession number DQ068751.1.

In some embodiments, the HCV IRES RNA molecule comprises SEQ ID NO: 1. In some embodiments, the HCV IRES RNA molecule is synthesized from a nucleic acid sequence comprising SEQ ID NO: 2, or fragment or variant thereof.

In some embodiments, the FgenL RNA molecule comprises SEQ ID NO: 3. An FgenL RNA molecule including primer annealing sites comprises SEQ ID NO: 4. In some embodiments, the FgenL RNA molecule including primer annealing sites is synthesized from a nucleic acid sequence comprising SEQ ID NO: 5, or fragment or variant thereof.

In some embodiments, the Chi18-4 RNA molecule comprises SEQ ID NO: 6. An Chi18-4 RNA molecule including primer annealing sites comprises SEQ ID NO: 7. In some embodiments, the FgenL RNA molecule including primer annealing sites is synthesized from a nucleic acid sequence comprising SEQ ID NO: 8, or fragment or variant thereof.

In some embodiment, the RNA molecule comprises SEQ ID NO: 9.

In some embodiments, the RNA molecule includes a coding region for an antigen derived from a pathogen associated with infectious disease. In some instances, the antigen is selected from antigens derived from the pathogens Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella burnetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli 01 57:H7, O111 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowled, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioides brasiliensis, Paragonimus spp, Paragonimus westermani, Parvovirus B1 9, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis. In some aspects, one or more of the pathogens in the list may be excluded.

In some embodiments, the RNA molecule encodes a VSV G protein or a fragment or variant thereof. In some embodiments, the RNA molecule encodes a VSV G protein comprising the amino acid sequence according to any one of GenBank Accession Nos.: M27165.1, QQL06423.1, AMB37292.1, QQL06428.1, QQL06418.1, or QQL06413.1, the respective sequences of which are herein incorporated by reference.

In some embodiments, the RNA molecule encodes a VSV G protein comprising the sequence of SEQ ID NO: 10, or fragment or variant thereof.

In some embodiments, the RNA molecule encodes a VSV G protein synthesized from the nucleic acid sequence comprising SEQ ID NO: 11, or fragment or variant thereof.

In some embodiments, the RNA molecule encodes a VZV gE protein or a fragment or a variant thereof. In some embodiments, the RNA molecule encodes a VZV gE protein comprising the amino acid sequence according to any one of GenBank Accession No.: AAG32558.1, ABE03086.1, AAK01047.1, Q9J3M8.1, AEW88548.1, AGY33616.1, AEW89124.1, AIT53150.1, CAA25033.1, NP_040190.1, AKG56356.1, AEW89412.1, ABF21714.1, ABF21714.1, AAT07749.1, AEW88764.1, AAG48520.1, and/or AEW88980.1, the respective sequences of which are herein incorporated by reference. In some embodiments, the RNA molecule encodes a VZV gE protein comprising the amino acid sequence according to GenBank Accession No. AH009994.2, the sequence of which is herein incorporated by reference.

In some embodiments, the RNA molecule encodes a VZV gE glycoprotein comprising the sequence of SEQ ID NO: 12, or fragment or variant thereof.

In some embodiments, the RNA molecule encodes a VZV gE glycoprotein synthesized from the nucleic acid sequence comprising SEQ ID NO: 13, or fragment or variant thereof.

In some embodiments, the RNA molecule comprises the sequence of SEQ ID NO: 14, or fragment or variant thereof.

In some embodiments, the RNA molecule encodes a CMV gB protein or a fragment or a variant thereof. In some embodiments, the RNA molecule encodes a CMV gB protein comprising the amino acid sequence according to any one of GenBank Accession No.: ACS91991.1, AK112129.1, AK122656.1, ACZ79977.1, AFR54884.1, AAA45934.2, AFR55216.1, AK114299.1, ADV04383.1, AK109624.1, AK120990.1, AND81989.1, AAA45932.1, AAA45933.1, AK122156.1, ACT81737.1, ACL51135.1, AFR54557.1, AHB19702.1, AAA45930.1, AFR55719.1, AK112960.1, AK117642.1, AAA45931.1, AAA45920.1, AIC80652.1, AAA45926.1, AK122824.1, AHV84013.1, AZB53179.1, AK111131.1, ABQ23592.1, AAA45925.1, AAA45925.1, AK107947.1, AK109288.1, AAA45923.2, AFR55048.1, AAA45935.1, ADB92600.1, AK113965.1, AHB20033.1, ACS93398.1, ABQ23592.1, AK123324.1, AK120319.1, AGQ47285.1, AK113294.1, AK119483.1, AAA45928.1, AAA45924.1, AK119648.1, AFR55550.1, AK119983.1, AAB07485.1, AK108783.1, AGL96655.1, AK123491.1, ACS92156.1, AFR55885.1, AHJ86153.1, and/or ADB92600.1, the respective sequences of which are herein incorporated by reference.

In some embodiments, the RNA molecule encodes a CMV gB protein comprising the amino acid sequence according to GenBank of Accession No. ABQ23592.1, the sequence of which is herein incorporated by reference.

In some embodiments, the RNA molecule encodes a CMV gB glycoprotein comprising the sequence of SEQ ID NO: 15, or fragment or variant thereof.

In some embodiments, the RNA molecule encodes a CMV gB glycoprotein synthesized from the nucleic acid sequence comprising SEQ ID NO: 16, or fragment or variant thereof.

In some embodiments, the RNA molecule comprises the sequence of SEQ ID NO: 17, or fragment or variant thereof.

In some embodiments, the RNA molecule encodes an RSV F protein or a fragment or a variant thereof. In some embodiments, the RNA molecule encodes an RSV F protein comprising the amino acid sequence according to any one of GenBank Accession No.: AHX57185, AHV80758, 138251, and/or 138250, the respective sequences of which are herein incorporated by reference. The RSV F proteins of subtypes A and B are about 90% identical in amino acid sequence. An example sequence of the F0 precursor polypeptide for the A subtype is provided in SEQ ID NO: 18 (A2 strain; GenBank GI: 138251; Swiss Prot P03420), and for the B subtype is provided in SEQ ID NO: 20 (18537 strain; GenBank GI: 138250; Swiss Prot P13843). SEQ ID NO: 18 and SEQ ID NO: 21 are both 574 amino acid sequences in length.

In some embodiments, the RNA molecule encodes an RSV F protein comprising the amino acid sequence according to Accession No.: AHX57185, AHV80758, 138251, and/or 138250, the respective sequences of which are herein incorporated by reference.

In some embodiments, the RNA molecule encodes a RSV F protein comprising any one of the sequences having SEQ ID NO: 18, SEQ ID NO: 21, SEQ ID NO: 24, SEQ ID NO: 27, SEQ ID NO: 30, SEQ ID NO: 31, SEQ ID NO: 32, SEQ ID NO: 33, SEQ ID NO: 34, or fragments or variants thereof. In another embodiment, the RNA molecule encodes a mutant of a wild-type RSV F protein, which mutant comprises a F1 polypeptide and a F2 polypeptide, wherein the mutant comprises at least one amino acid mutation relative to the amino acid sequence of the wild-type RSV F protein, wherein the introduced amino acid mutation is a pair of cysteine mutations selected from the group consisting of: (1) 55C and 188C; (2) 103C and 148C; (3) 142C and 371C; and (4) 155C and 290C, and wherein amino acid positions are numbered according to SEQ ID NO: 18.

In some embodiments, the RNA molecule encodes a RSV F protein synthesized from any one of the nucleic acid sequences having SEQ ID NO: 19, SEQ ID NO: 22, SEQ ID NO: 25, SEQ ID NO: 28 or SEQ ID NO: 34, SEQ ID NO: 35 or fragments or variants thereof.

In some embodiments, the RNA molecule comprises the sequence of any one of SEQ ID NO: 20, SEQ ID NO: 23, SEQ ID NO: 26, SEQ ID NO: 29 and/or SEQ ID NO: 36, or fragments or variants thereof.

E. 5′ Cap

In some embodiments, the RNA molecule described herein includes a 5′ cap. In some embodiments, the 5′-cap moiety is a natural 5′-cap. A “natural 5′-cap” is defined as a cap that includes 7-methylguanosine connected to the 5′ end of an mRNA molecule through a 5′ to 5′ triphosphate linkage. In some embodiments, the 5′-cap moiety is a 5′-cap analog. In some embodiments, the 5′ end of the RNA is capped with a modified ribonucleotide with the structure m7G (5′) ppp (5′) N (cap 0 structure) or a derivative thereof, which may be incorporated during RNA synthesis (e.g., co-transcriptional capping) or may be enzymatically engineered after RNA transcription (e.g., post-transcriptional capping), wherein “N” is any ribonucleotide. In some embodiments, the 5′ end of the RNA molecule is capped with a modified ribonucleotide via an enzymatic reaction after RNA transcription. In some embodiments, capping is performed after purification, e.g., tangential flow filtration, of the RNA molecule. An exemplary enzymatic reaction for capping may include use of Vaccinia Virus Capping Enzyme (VCE) that includes mRNA triphosphatase, guanylyl-transferase, and guanine-7-methytransferase, which catalyzes the construction of N7-monomethylated cap 0 structures. Cap 0 structure can help maintaining the stability and translational efficacy of the RNA molecule. The 5′ cap of the RNA molecule may be further modified by a 2′-O-Methyltransferase which results in the generation of a cap 1 structure (m7Gppp [m2′-O] N), which may further increase translation efficacy. In some embodiments, the RNA molecule may be enzymatically capped at the 5′ end using Vaccinia guanylyltransferase, guanosine triphosphate, and S-adenosyl-L-methionine to yield cap 0 structure. An inverted 7-methylguanosine cap is added via a 5′ to 5′ triphosphate bridge. Alternatively, use of a 2′O-methyltransferase with Vaccinia guanylyltransferase yields the cap 1 structure where in addition to the cap 0 structure, the 2′OH group is methylated on the penultimate nucleotide. S-adenosyl-L-methionine (SAM) is a cofactor utilized as a methyl transfer reagent. Non-limiting examples of 5′ cap structures are those which, among other things, have enhanced binding of cap binding polypeptides, increased half-life, reduced susceptibility to 5′ endonucleases and/or reduced 5′ decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme may create a canonical 5′-5′-triphosphate linkage between the 5′-terminal nucleotide of an mRNA and a guanine cap nucleotide wherein the cap guanine includes an N7 methylation and the 5′-terminal nucleotide of the mRNA includes a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0) and 7mG(5′)ppp(5′)N1mpNp (cap 1). Cap 0 is a N7-methyl guanosine connected to the 5′ nucleotide through a 5′ to 5′ triphosphate linkage, typically referred to as m7G cap or m7Gppp. In the cell, the cap 0 structure can help provide for efficient translation of the mRNA that carries the cap. An additional methylation on the 2′O position of the initiating nucleotide generates Cap 1, or refers to as m7GpppNm-, wherein Nm denotes any nucleotide with a 2′O methylation. In some embodiments, the 5′ terminal cap includes a cap analog, for example, a 5′ terminal cap may include a guanine analog. Exemplary guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine. In some embodiments, the capping region may include a single cap or a series of nucleotides forming the cap. In this embodiment the capping region may be equal to any one of, at least any one of, at most any one of, or between any two of 1, 2, 3, 4, 5, 6, 7, 8, 9, 10, or at least 2, or 10 or fewer nucleotides in length. In some embodiments, the cap is absent. In some embodiments, the first and second operational regions may be equal to any one of, at least any one of, at most any one of, or between any two of 3 to 40, e.g., 5-30, 10-20, 15, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or at least 4, or 30 or fewer nucleotides in length and may comprise, in addition to a Start and/or Stop codon, one or more signal and/or restriction sequences.

F. Poly-A Tail

As used herein, “poly A tail” refers to a stretch of consecutive adenine residues, which may be attached to the 3′ end of the RNA molecule. The poly-A tail may increase the half-life of the RNA molecule. Poly-A tails may play key regulatory roles in enhancing translation efficiency and regulating the efficiency of mRNA quality control and degradation. Short sequences or hyperpolyadenylation may signal for RNA degradation. Exemplary designs include a poly-A tails of about 40 adenine residues to about 80 adenine residues. In some embodiments, the RNA molecule further includes an endonuclease recognition site sequence immediately downstream of the poly A tail sequence. In some embodiments, such as for the second RNA or the saRNA molecule, the RNA molecule further includes a poly-A polymerase recognition sequence (e.g., AAUAAA) near its 3′ end. A “full length” RNA molecule is one that includes a 5-cap and a poly A tail.

In some embodiments, the poly A tail includes 5-400 nucleotides in length. The poly A tail nucleotide length may be equal to any one of, at least any one of, at most any one of, or between any two of 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, and 400. In some embodiments, the RNA molecule includes a poly A tail that includes about 25 to about 400 adenosine nucleotides, a sequence of about 50 to about 400 adenosine nucleotides, a sequence of about 50 to about 300 adenosine nucleotides, a sequence of about 50 to about 250 adenosine nucleotides, a sequence of about 60 to about 250 adenosine nucleotides, or a sequence of about 40 to about 100 adenosine nucleotides. In some embodiments, the RNA molecule includes a poly A tail includes a sequence of greater than 30 adenosine nucleotides (“As”). In some embodiments, the RNA molecule includes a poly A tail that includes about 40 As. In some embodiments, the RNA molecule includes a poly A tail that includes about 80 As. As used herein, the term “about” refers to a deviation of ±10% of the value(s) to which it is attached. In some embodiments, the 3′ poly-A tail has a stretch of at least 10 consecutive adenosine residues and at most 300 consecutive adenosine residues. In some embodiments, the RNA molecule includes at least 20 consecutive adenosine residues and at most 40 consecutive adenosine residues. In some embodiments, the RNA molecule includes about 40 consecutive adenosine residues. In some embodiments, the RNA molecule includes about 80 consecutive adenosine residues.

In some embodiments, the RNA molecule is purified, e.g., such as by filtration that may occur via, e.g., ultrafiltration, diafiltration, or, e.g., tangential flow ultrafiltration/diafiltration.

G. Self-Amplifying RNA (saRNA)

In some embodiments, the RNA molecule is a saRNA. “saRNA,” “self-amplifying RNA,” and “replicon” refer to RNA with the ability to replicate itself. Self-amplifying RNA molecules may be produced by using replication elements derived from a virus or viruses, e.g., alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNAs. These daughter RNAs, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the protein of interest, e.g., an antigen. The overall result of this sequence of transcriptions is an amplification in the number of the introduced saRNAs and so the encoded gene of interest, e.g., a viral antigen, can become a major polypeptide product of the cells.

In some embodiments, the self-amplifying RNA includes at least one or more genes selected from any one of viral replicases, viral proteases, viral helicases, and other nonstructural viral proteins. In some embodiments, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In some embodiments, the self-amplifying RNA molecule is not encapsulated in a virus-like particle. Self-amplifying RNA molecules described herein may be designed so that the self-amplifying RNA molecule cannot induce production of infectious viral particles. This may be achieved, for example, by omitting one or more viral genes encoding structural proteins that are necessary to produce viral particles in the self-amplifying RNA. For example, when the self-amplifying RNA molecule is based on an alphavirus, such as Sinbis virus (SIN), Semliki forest virus, and Venezuelan equine encephalitis virus (VEE), one or more genes encoding viral structural proteins, such as capsid and/or envelope glycoproteins, may be omitted.

In some embodiments, a self-amplifying RNA molecule described herein encodes (i) an RNA-dependent RNA polymerase that may transcribe RNA from the self-amplifying RNA molecule and (ii) a polypeptide of interest, e.g., a viral antigen. In some embodiments, the polymerase may be an alphavirus replicase, e.g., including any one of alphavirus protein nsP1, nsP2, nsP3, nsP4, and any combination thereof. In some embodiments, the self-amplifying RNA molecules described herein may include one or more modified nucleotides (e.g., pseudouridine, N6-methyladenosine, 5-methylcytidine, 5-methyluridine). In some embodiments, the self-amplifying RNA molecules does not include a modified nucleotide (e.g., pseudouridine, N6-methyladenosine, 5-methylcytidine, 5-methyluridine).

The saRNA construct may encode at least one non-structural protein (NSP), disposed 5′ or 3′ of the sequence encoding at least one peptide or polypeptide of interest. In some embodiments, the sequence encoding at least one NSP is disposed 5′ of the sequences encoding the peptide or polypeptide of interest. Thus, the sequence encoding at least one NSP may be disposed at the 5′ end of the RNA construct. In some embodiments, at least one non-structural protein encoded by the RNA construct may be the RNA polymerase nsP4. In some embodiments, the saRNA construct encodes nsP1, nsP2, nsP3 and, nsP4. As is known in the art, nsP1 is the viral capping enzyme and membrane anchor of the replication complex (RC). nsP2 is an RNA helicase and the protease responsible for the ns polyprotein processing. nsP3 interacts with several host proteins and may modulate protein poly- and mono-ADP-ribosylation. nsP4 is the core viral RNA-dependent RNA polymerase. In some embodiments, the polymerase may be an alphavirus replicase, e.g., comprising one or more of alphavirus proteins nsP1, nsP2, nsP3, and nsP4.

Whereas natural alphavirus genomes encode structural virion proteins in addition to the non-structural replicase polypeptide, in some embodiments, the self-amplifying RNA molecules do not encode alphavirus structural proteins. In some embodiments, the self-amplifying RNA may lead to the production of genomic RNA copies of itself in a cell, but not to the production of RNA that includes virions. Without being bound by theory or mechanism, the inability to produce these virions means that, unlike a wild-type alphavirus, the self-amplifying RNA molecule cannot perpetuate itself in infectious form. The alphavirus structural proteins which are necessary for perpetuation in wild-type viruses can be absent from self-amplifying RNAs of the present disclosure and their place can be taken by gene(s) encoding the immunogen of interest, such that the subgenomic transcript encodes the immunogen rather than the structural alphavirus virion proteins.

In some embodiments, the self-amplifying RNA molecule may have two open reading frames. The first (5) open reading frame can encode a replicase; the second (3) open reading frame can encode a polypeptide comprising an antigen of interest. In some embodiments the RNA may have additional (e.g., downstream) open reading frames, e.g., to encode further antigens or to encode accessory polypeptides.

In some embodiments, the saRNA molecule further includes (1) an alphavirus 5′ replication recognition sequence, and (2) an alphavirus 3′ replication recognition sequence. In some embodiments, the 5′ sequence of the self-amplifying RNA molecule is selected to ensure compatibility with the encoded replicase.

Optionally, self-amplifying RNA molecules described herein may also be designed to induce production of infectious viral particles that are attenuated or virulent, or to produce viral particles that are capable of a single round of subsequent infection.

In some embodiments, the saRNA molecule is alphavirus-based. Alphaviruses include a set of genetically, structurally, and serologically related arthropod-borne viruses of the Togaviridae family. Exemplary viruses and virus subtypes within the alphavirus genus include Sindbis virus (SINV), Semliki Forest virus (SFV), Ross River virus (RRV), and Venezuelan equine encephalitis virus (VEEV). As such, the self-amplifying RNA described herein may incorporate an RNA replicase derived from any one of SFV, SINV, VEEV, RRV, or other viruses belonging to the alphavirus family. In some embodiments, the self-amplifying RNA described herein may incorporate sequences derived from a mutant or wild-type virus sequence, e.g., the attenuated TC83 mutant of VEEV has been used in saRNAs.

Alphavirus-based saRNAs are (+)-stranded saRNAs that may be translated after delivery to a cell, which leads to translation of a replicase (or replicase-transcriptase). The replicase is translated as a polyprotein which auto-cleaves to provide a replication complex which creates genomic (−)-strand copies of the (+)-strand delivered RNA. These (−)-strand transcripts may themselves be transcribed to give further copies of the (+)-stranded parent RNA and also to give a subgenomic transcript which encodes the desired gene product. Translation of the subgenomic transcript thus leads to in situ expression of the desired gene product by the infected cell. Suitable alphavirus saRNAs may use a replicase from a SINV, a SFV, an eastern equine encephalitis virus, a VEEV, or mutant variants thereof.

In some embodiments, the self-amplifying RNA molecule is derived from or based on a virus other than an alphavirus, such as a positive-stranded RNA virus, and in particular a picornavirus, flavivirus, rubivirus, pestivirus, hepacivirus, calicivirus, or coronavirus. Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375). In some aspects, one or more of the alphaviruses in the list may be excluded.

In some embodiments, the self-amplifying RNA molecules described herein are larger than other types of RNA (e.g., mRNA). Typically, the self-amplifying RNA molecules described herein include at least about 4 kb. For example, the self-amplifying RNA may be equal to any one of, at least any one of, at most any one of, or between any two of 3 kb, 4 kb, 5 kb, 6 kb, 7 kb, 8 kb, 9 kb, 10 kb, 11 kb, 12 kb, 13 kb, 14 kb, 15 kb, 16 kb. In some instances the self-amplifying RNA may include at least about 5 kb, at least about 6 kb, at least about 7 kb, at least about 8 kb, at least about 9 kb, at least about 10 kb, at least about 11 kb, at least about 12 kb, or more than 12 kb. In certain examples, the self-amplifying RNA is about 4 kb to about 12 kb, about 5 kb to about 12 kb, about 6 kb to about 12 kb, about 7 kb to about 12 kb, about 8 kb to about 12 kb, about 9 kb to about 12 kb, about 10 kb to about 12 kb, about 11 kb to about 12 kb, about 5 kb to about 11 kb, about 5 kb to about 10 kb, about 5 kb to about 9 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb.

In some embodiments, the self-amplifying RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-amplifying RNA may then be produced as a fusion polypeptide or engineered in such a manner to result in separate polypeptide or peptide sequences.

In some embodiments, the self-amplifying RNA described herein may encode one or more polypeptide antigens that include a range of epitopes. In some embodiments, the self-amplifying RNA described herein may encode epitopes capable of eliciting either a helper T-cell response or a cytotoxic T-cell response or both.

### IV. Nucleic Acids

In certain embodiments, nucleic acid sequences can exist in a variety of instances such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, saRNA, and complementary sequences of the foregoing described herein. Nucleic acids that encode an epitope to which antibodies may bind. Nucleic acids encoding fusion proteins that include these polypeptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that can be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

In certain embodiments, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some embodiments, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

A. Hybridization

The nucleic acids may in some instances hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the polynucleotide.

B. Mutation

Changes can be introduced by mutation into a nucleic acid, thereby leading to changes in the amino acid sequence of a polypeptide (e.g., an antigen or antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. In some instances, however it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, e.g., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing antigens. See, e.g., Gaily Kivi et al., BMC Biotechnol. 16:2 (2016). In some embodiments, the nucleic acid molecules are oligonucleotides. In some embodiments, the oligonucleotides are from the polypeptides of interest, such as antigens.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### V. Oligonucleotide Synthesis

As used herein, “isolated” means altered or removed from the natural state through human intervention. For example, a RNA naturally present in a living animal is not “isolated,” but a synthetic RNA, or a RNA partially or completely separated from the coexisting materials of its natural state is “isolated.” An isolated RNA can exist in substantially purified form, or can exist in a non-native environment such as, for example, a cell into which the RNA has been delivered.

In some embodiments, the RNA molecule is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

Modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a DNA or RNA synthesizer using methodology commonly known to those skilled in DNA or RNA synthesizer art. Generally, the procedure involves functionalizing the sugar moieties of two nucleosides which will be adjacent to one another in the selected sequence. In a 5′ to 3 sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5-hydroxyl and activated for coupling at the 3-hydroxyl with cyanoethyldiisopropyl-phosphite moieties. These compounds can be inserted into any desired sequence by standard, solid phase, automated DNA or RNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some embodiments, the oligonucleotide may be further defined as having one or more locked nucleotides, ethylene bridged nucleotides, peptide nucleic acids, or a 5′(E)-vinyl-phosphonate (VP) modification. In some embodiments, the oligonucleotides has one or more phosphorothioated DNA or RNA bases.

### VI. Characterization and Analysis of RNA Molecule

The RNA molecule described herein may be analyzed and characterized using various methods. Analysis may be performed before or after capping. Alternatively, analysis may be performed before or after poly-A capture-based affinity purification. In another embodiment, analysis may be performed before or after additional purification steps, e.g., anion exchange chromatography and the like. For example, RNA template quality may be determined using Bioanalyzer chip based electrophoresis system. In other embodiments, RNA template purity is analyzed using analytical reverse phase HPLC. Capping efficiency may be analyzed using, e.g., total nuclease digestion followed by MS/MS quantitation of the dinucleotide cap species vs. uncapped GTP species. In vitro efficacy may be analyzed by, e.g., transfecting RNA molecule into a human cell line. Protein expression of the polypeptide of interest may be quantified using methods such as ELISA or flow cytometry. Immunogenicity may be analyzed by, e.g., transfecting RNA molecules into cell lines that indicate innate immune stimulation, e.g., PBMCs. Cytokine induction may be analyzed using, e.g., methods such as ELISA to quantify a cytokine, e.g., Interferon-α.

### VII. Obtaining Encoded Polypeptide Embodiments

In some aspects, there are nucleic acid molecules encoding peptides of interest, e.g., antigens. These nucleic acids may be generated by methods known in the art.

A. Expression

The nucleic acid molecules may be used to express large quantities of the polypeptide of interest, such as an antigen.

1. RNA Molecules

In some aspects, contemplated are RNA molecules comprising a nucleic acid molecule encoding a polypeptide of the desired sequence or a portion thereof (e.g., a fragment containing one or more polypeptides, or antigens). In some aspects, RNA molecules comprising nucleic acid molecules may encode antigens, fusion proteins, modified antibodies, antibody fragments, and probes thereof. In addition to control sequences that govern transcription and translation, the RNA molecules may contain nucleic acid sequences that serve other functions as well.

RNA molecules may be produced by transcription from a DNA template or transcription from an RNA template. Such production methods can be those known in the art.

2. Expression Systems

Numerous expression systems exist that comprise at least a part or all of the RNA molecules discussed above. Prokaryote- and/or eukaryote-based systems or cell free systems can be employed for use with an embodiment to produce nucleic acid sequences, or their cognate polypeptides, proteins and peptides. Commercially and widely available systems include but are not limited to bacterial, mammalian, yeast, insect cell, and cell free systems. Different host cells have characteristic and specific mechanisms for the post-translational processing and modification of proteins. Appropriate cell lines, host systems, or expression systems can be chosen to ensure the correct modification and processing of the nucleic acid or polypeptide(s) expressed. Those skilled in the art are able to express a vector to produce a nucleic acid sequence or its cognate polypeptide, protein, or peptide using an appropriate expression system.

3. Methods of Nucleic Acid Delivery

Suitable methods for nucleic acid delivery to effect expression of compositions are anticipated to include virtually any method by which a particular nucleic acid (e.g., RNA, mRNA, saRNA, DNA) can be introduced into a cell, a tissue or an organism, as described herein or as would be known to one of ordinary skill in the art. Such methods may include, but are not limited to, direct delivery of nucleic acids such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

4. Host Cells

In another aspect, contemplated are the use of host cells into which a RNA molecule has been introduced. RNA can be transfected into cells according to a variety of methods known in the art. RNA can be introduced into prokaryotic or eukaryotic cells via conventional transformation or transfection techniques. Some RNA may employ control sequences that allow it to be replicated and/or expressed in both prokaryotic and eukaryotic cells. In certain aspects, the polypeptide of interest expression construct or RNA replicase can be placed under control of a promoter that is linked to T-cell activation, such as one that is controlled by NFAT-1 or NF-KB, both of which are transcription factors that can be activated upon T-cell activation. Control of expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a RNA molecule, such as a saRNA. Also understood and known are techniques and conditions that would allow large-scale production of RNA molecules, as well as production of the nucleic acids encoded by vectors and their cognate polypeptides, proteins, or peptides.

For transfection of mammalian cells, it is known, depending upon the RNA and transfection technique used, only a small fraction of cells may integrate the foreign RNA into their cells. In order to identify and select these integrants, a selectable marker (e.g., for resistance to antibiotics) is generally introduced into the host cells along with the polypeptide of interest. Cells transfected with the introduced nucleic acid can be identified by drug selection (e.g., cells that have incorporated the selectable marker gene will survive, while the other cells die), among other methods known in the arts.

In particular embodiments, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-l-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21TM supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/mL, μg/mL, mg/mL, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

B. Isolation

The nucleic acid molecule disclosed herein may be obtained from any source that produces nucleic acids. Methods of isolating mRNA are well known in the art. See e.g., Sambrook et al., supra.

### VIII. Detecting a Nucleic Acid or Protein Signatures

Particular embodiments concern the methods of detecting a nucleic acid signature in an individual. In some embodiments, the method for detecting the nucleic acid signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the nucleic acid signature may include fluorescent in situ hybridization, comparative nucleic acid hybridization, arrays, polymerase chain reaction, sequencing, or a combination thereof, for example. The detection of the nucleic acid signature may involve using a particular method to detect one feature of the nucleic acid signature and additionally use the same method or a different method to detect a different feature of the nucleic acid signature. Multiple different methods independently or in combination may be used to detect the same feature or a plurality of features.

A. DNA Sequencing

In some embodiments, DNA may be analyzed by sequencing. In some embodiments, the methods of the disclosure include a sequencing method. The DNA may be prepared for sequencing by any method known in the art, such as library preparation, hybrid capture, sample quality control, product-utilized ligation-based library preparation, or a combination thereof. The DNA may be prepared for any sequencing technique. For example, DNA, including bisulfite-converted DNA could be used for the amplification of the region of interest followed by sequencing. Primers are designed around the CpG island and used for PCR amplification of bisulfite-converted DNA. The resulting PCR products could be cloned and sequenced. Accordingly, aspects of the disclosure may include sequencing nucleic acids to detect methylation of nucleic acids and/or biomarkers. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

Exemplary sequencing methods include those described below.

1. Massively Parallel Signature Sequencing (MPSS)

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

2. Polony Sequencing

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

3. 454 Pyrosequencing

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

4. Illumina (Solexa) Sequencing

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massively parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

5. SOLiD Sequencing

Applied Biosystems' (now a Thermo Fisher Scientific brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

6. Ion Torrent Semiconductor Sequencing

Ion Torrent Systems Inc. (now owned by Thermo Fisher Scientific) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

7. DNA Nanoball Sequencing

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

8. Heliscope Single Molecule Sequencing

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

9. Single Molecule Real Time (SMRT) Sequencing

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.]

B. RNA Sequencing

In some embodiments, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

C. Proteomics

In some embodiments, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool.

D. Additional Assay Methods

In some embodiments, methods involve amplifying and/or sequencing one or more target nucleic acid regions using at least one pair of primers specific to the target nucleic acid regions. In certain embodiments, the primers are heptamers. In other embodiments, enzymes are added such as primases or primase/polymerase combination enzyme to the amplification step to synthesize primers.

In some embodiments, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, dd PCR (digital droplet PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of an oligo of the disclosure, or portion thereof. Typically, primers are oligonucleotides from ten to twenty and/or thirty nucleic acids in length, but longer sequences can be employed. Primers may be provided in double-stranded and/or single-stranded form.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene or RNA under stringent or highly stringent conditions. It is contemplated that probes or primers may have inosine or other design implementations that accommodate recognition of more than one sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific RNA transcripts. It is generally appreciated that conditions can be rendered more stringent by the addition of increasing amounts of formamide.

In one embodiment, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be or may be at least 50, 100, 200, 300, 400, 500 or more probes/cm² (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more polypeptide of interest with respect to diagnostic, prognostic, and treatment methods.

Certain embodiments may involve the use of arrays or data generated from an array. Data may be readily available. Moreover, an array may be prepared in order to generate data that may then be used in correlation studies.

### IX. RNA Transcription and Encapsulation

In some embodiments, the RNA disclosed herein is produced by in vitro transcription. “In vitro transcription” or “IVT” refers to the process whereby transcription occurs in vitro in a non-cellular system to produce a synthetic RNA product for use in various applications, including, e.g., production of protein or polypeptides. Such synthetic RNA products can be translated in vitro or introduced directly into cells, where they can be translated. Such synthetic RNA products include, e.g., but are not limited to mRNAs, saRNAs, antisense RNA molecules, shRNA molecules, long non-coding RNA molecules, ribozymes, aptamers, guide RNAs (e.g., for CRISPR), ribosomal RNAs, small nuclear RNAs, small nucleolar RNAs, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In some embodiments, starting material for IVT can include linearized DNA template, nucleotides, RNase inhibitor, pyrophosphatase, and/or T7 RNA polymerase. In some embodiments, the IVT process is conducted in a bioreactor. The bioreactor can comprise a mixer. In some embodiments, nucleotides can be added into the bioreactor throughout the IVT process.

In some embodiments, one or more post-IVT agents are added into the IVT mixture comprising RNA in the bioreactor after the IVT process. Exemplary post-IVT agents can include DNAse I configured to digest the linearized DNA template, and proteinase K configured to digest DNAse I and T7 RNA polymerase. In some embodiments, the post-IVT agents are incubated with the mixture in the bioreactor after IVT. In some embodiments, the bioreactor can contain any one of, at least any one of, at most any one of, or between any two of 60, 70, 80, 90, 100, 110, 120, 130, 140, 150,160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 or more liters IVT mixture. The IVT mixture can have an RNA concentration at any one of, at least any one of, at most any one of, or between any two of 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0, 10, 11, 12, 13, 14, 15, 16, 17, 18, 19, 20, 30, 40, 50, 60, 70, 80, 90, and 100 mg/mL or more RNA.

In some embodiments, the IVT mixture can include residual spermidine, residual DNA, residual proteins, peptides, HEPES, EDTA, ammonium sulfate, cations (e.g., Mg²⁺, Na⁺, Ca²⁺), RNA fragments, residual nucleotides, free phosphates, or any combinations thereof.

In some embodiments, at least a portion of the IVT mixture is filtered. The IVT mixture can be filtered via ultrafiltration and/or diafiltration to remove at least some impurities from the IVT mixture and/or to change buffer solution for the at least a portion of IVT mixture to produce a concentrated RNA solution as a retentate.

In some embodiments, both “ultrafiltration” and “diafiltration” refer to a membrane filtration process. Ultrafiltration typically uses membranes having pore sizes any one of, at least any one of, at most any one of, or between any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 μm. In some embodiments, ultrafiltration membranes are typically classified by molecular weight cutoff (MWCO) rather than pore size. For example, the MWCO can be any one of, at least any one of, at most any one of, or between any two of 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, and 10000 kDa. A skilled artisan will understand that filtration membranes can be of different suitable materials, including, e.g., polymeric, cellulose, ceramic, etc., depending upon the application. In some embodiments, membrane filtration may be more desirable for large volume purification process.

In some embodiments, ultrafiltration and diafiltration of the IVT mixture for purifying RNA can include (1) Direct Flow Filtration (DFF), also known as “dead-end” filtration, that applies a feed stream perpendicular to the membrane face and attempts to pass 100% of the fluid through the membrane, and/or (2) Tangential Flow Filtration (TFF), also known as crossflow filtration, where a feed stream passes parallel to the membrane face as one portion passes through the membrane (permeate) while the remainder (retentate) is retained and/or recirculated back to the feed tank.

In some embodiments, the filtering of the IVT mixture is conducted via TFF that comprises an ultrafiltration step, a first diafiltration step, and a second diafiltration step. In some embodiments, the first diafiltration step is conducted in the presence of ammonium sulfate. The first diafiltration step can be configured to remove a majority of impurities from the IVT mixture. In some embodiments, the second diafiltration step is conducted without ammonium sulfate. The second diafiltration step can be configured to transfer the RNA into a DS buffer formulation.

A filtration membrane with an appropriate MWCO may be selected for the ultrafiltration in the TFF process. The MWCO of a TFF membrane determines which solutes can pass through the membrane into the filtrate and which are retained in the retentate. The MWCO of a TFF membrane may be selected such that substantially all of the solutes of interest (e.g., desired synthesized RNA species) remains in the retentate, whereas undesired components (e.g., excess ribonucleotides, small nucleic acid fragments such as digested or hydrolyzed DNA template, peptide fragments such as digested proteins and/or other impurities) pass into the filtrate. In some embodiments, the retentate comprising desired synthesized RNA species may be re-circulated to a feed reservoir to be re-filtered in additional cycles. In some embodiments, a TFF membrane may have a MWCO equal to any one of, at least any one of, at most any one of, or between any two of at least 30 kDa, at least 40 kDa, at least 50 kDa, at least 60 kDa, at least 70 kDa, at least 80 kDa, at least 90 kDa, or more. In some embodiments, a TFF membrane may have a MWCO equal to any one of, at least any one of, at most any one of, or between any two of at least 100 kDa, at least 150 kDa, at least 200 kDa, at least 250 kDa, at least 300 kDa, at least 350 kDa, at least 400 kDa, or more. In some embodiments, a TFF membrane may have a MWCO of about 250-350 kDa. In some embodiments, a TFF membrane (e.g., a cellulose-based membrane) may have a MWCO of about 30-300 kDa; in some embodiments about 50-300 kDa, about 100-300 kDa, or about 200-300 kDa.

Diafiltration can be performed either discontinuously, or alternatively, continuously. For example, in continuous diafiltration, a diafiltration solution can be added to a sample feed reservoir at the same rate as filtrate is generated. In this way, the volume in the sample reservoir remains constant but small molecules (e.g., salts, solvents, etc.) that can freely permeate through a membrane are removed. Using solvent removal as an example, each additional diafiltration volume (DV) reduces the solvent concentration further. In discontinuous diafiltration, a solution is first diluted and then concentrated back to the starting volume. This process is then repeated until the desired concentration of small molecules (e.g. salts, solvents, etc.) remaining in the reservoir is reached. Each additional diafiltration volume (DV) reduces the small molecule (e.g., solvent) concentration further. Continuous diafiltration typically requires a minimum volume for a given reduction of molecules to be filtered. Discontinuous diafiltration, on the other hand, permits fast changes of the retentate condition, such as pH, salt content, and the like. In some embodiments, the first diafiltration step is conducted with diavolumes equal to any one of, at least any one of, at most any one of, or between any two of at least 2, at least 3, at least 4, at least 5, at least 6, at least 7, at least 8, at least 9, at least 10, or more. In some embodiments, the second diafiltration step is conducted with diavolumes equal to any one of, at least any one of, at most any one of, or between any two of at least 5, at least 6, at least 7, at least 8, at least 9, at least 10, at least 11, at least 12, at least 13, at least 14, at least 15, at least 16, at least 17, at least 18, at least 19, at least 20, or more. In some embodiments, the first diafiltration step is conducted with 5 diavolumes, and second diafiltration step is conducted with 10 diavolumes.

In some embodiments, for the ultrafiltration and/or diafiltration, the IVT mixture is filtered at a rate equal to any one of, at least any one of, at most any one of, or between any two of at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 500, at least 600, at least 700, at least 800, at least 900, at least 1000 L/m² of filter area per hour, or more. The concentrated RNA solution can comprise any one of, at least any one of, at most any one of, or between any two of 2.0, 2.1, 2.2, 2.3, 2.4, and 2.5 mg/mL single stranded RNA.

The bioburden of the concentrated RNA solution via filtration to obtain an RNA product solution may also be reduced, in some embodiments. The filtration for reducing bioburden can be conducted using one or more filters. The one or more filters can include a filter with a pore size of 0.2 μm, 0.45 μm, 0.65 μm, 0.8 μm, or any other pore size configured to remove bioburdens.

As one example, reducing the bioburden can include draining a retentate tank containing retentate obtained from the ultrafiltration and/or diafiltration to obtain the retentate. Reducing the bioburden can include flushing a filtration system for ultrafiltration and/or diafiltration using a wash buffer solution to obtain a wash pool solution comprising residue RNA remaining in the filtration system. The retentate can be filtered to obtain a filtered retentate. The wash pool solution can be filtered using a first 0.2 μm filter to obtain a filtered wash pool solution. The retentate can be filtered using the first 0.2 μm filter or another 0.2 μm filter.

The filtered wash pool solution and the filtered retentate can be combined to form a combined pool solution. The combined pool solution can be filtered using a second 0.2 μm filter to obtain a filtered combined pool solution, which is further filtered using a third 0.2 μm filter to produce the RNA product solution.

The RNA in the RNA product solution may be encapsulated, and the RNA solution may further comprise at least one encapsulating agent. In one embodiment, the encapsulating agent comprises a lipid, a lipid nanoparticle (LNP), lipoplexes, polymeric particles, polyplexes, and monolithic delivery systems, and a combination thereof. In one embodiment, the encapsulating agent is a lipid, and produced is lipid nanoparticle (LNP)-encapsulated RNA. In some embodiments, LNPs can be designed to protect RNAs (e.g., saRNA, mRNA) from extracellular RNases and/or can be engineered for systemic delivery of the RNA to target cells. In some embodiments, such LNPs may be particularly useful to deliver RNAs (e.g., saRNA, mRNA) when RNAs are intravenously administered to a subject in need thereof. In some embodiments, such LNPs may be particularly useful to deliver RNAs (e.g., saRNA, mRNA) when RNAs are intramuscularly administered to a subject in need thereof.

In some embodiments, provided RNAs (e.g., saRNA, mRNA) may be formulated with LNPs. In various embodiments, such LNPs can have an average size (e.g., mean diameter) equal to any one of, at least any one of, at most any one of, or between any two of about 30 nm to about 150 nm, about 40 nm to about 150 nm, about 50 nm to about 150 nm, about 50 nm to about 130 nm, about 50 nm to about 110 nm, about 50 nm to about 100 nm, about 50 to about 90 nm, or about 60 nm to about 80 nm, or about 60 nm to about 70 nm. In some embodiments, LNPs that may be useful in accordance with the present disclosure can have an average size (e.g., mean diameter) equal to any one of, at least any one of, at most any one of, or between any two of about 50 nm to about 100 nm. In some embodiments, LNPs may have an average size (e.g., mean diameter) of less than 80 nm, less than 75 nm, less than 70 nm, less than 65 nm, less than 60 nm, less than 55 nm, less than 50 nm, or less than 45 nm. In some embodiments, LNPs that may be useful in accordance with the present disclosure can have an average size (e.g., mean diameter) of equal to any one of, at least any one of, at most any one of, or between any two of about 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, nucleic acids (e.g., RNAs), when present in provided LNPs, are resistant in aqueous solution to degradation with a nuclease. In some embodiments, LNPs are liver-targeting lipid nanoparticles. In some embodiments, LNPs are cationic lipid nanoparticles comprising one or more cationic lipids (e.g., ones described herein). In some embodiments, cationic LNPs may comprise at least one cationic lipid, at least one polymer conjugated lipid, and at least one helper lipid (e.g., at least one neutral lipid).

In some embodiments, LNP-encapsulated RNA can be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some embodiments, suitable buffering agents comprise tris, histidine, citrate, acetate, phosphate, or succinate. The pH of a liquid formulation relates to the pKa of the encapsulating agent (e.g. cationic lipid). The pH of the acidifying buffer may be at least half a pH scale less than the pKa of the encapsulating agent (e.g. cationic lipid), and the pH of the final buffer may be at least half a pH scale greater than the pKa of the encapsulating agent (e.g. cationic lipid). In some embodiments, properties of a cationic lipid are chosen such that nascent formation of particles occurs by association with an oppositely charged backbone of a nucleic acid (e.g., RNA). In this way, particles are formed around the nucleic acid, which, for example, in some embodiments, can result in much higher encapsulation efficiency than it is achieved in the absence of interactions between nucleic acids and at least one of the lipid components.

In some embodiments, the LNPs comprise a lipid of Formula (III), a mRNA compound as described above and one or more excipient selected from neutral lipids, steroids and pegylated lipids. In some embodiments, the LNP comprises (i) a cationic lipid of Formula (III):

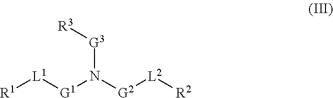

as further defined below or a pharmaceutically acceptable salt, tautomer, prodrug or stereoisomer thereof, and (ii) a mRNA compound comprising an mRNA sequence encoding at least one antigenic peptide or protein, wherein the mRNA compound is encapsulated in or associated with said lipid nanoparticle. In one of the preferred embodiments, the mRNA compound does not comprise a nucleoside modification. In another embodiment, it comprises no base modification. In a further embodiment, it does not comprise a 1-methylpseudouridine modification. In yet a further embodiment the mRNA compound only comprises the natural nucleosides adenine, guanine, cytosine and uracil.

Formula (III) is further defined in that: one of L′ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O—, and the other of L′ or L² is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, —NR^{a}C(═O)NR^{a}—, —OC(═O)NR^{a}— or —NR^{a}C(═O)O— or a direct bond; G¹ and G² are each independently unsubstituted C1-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

In some of the foregoing embodiments of Formula (III), the lipid has one of the following structures (IIIA) or (IIIB):

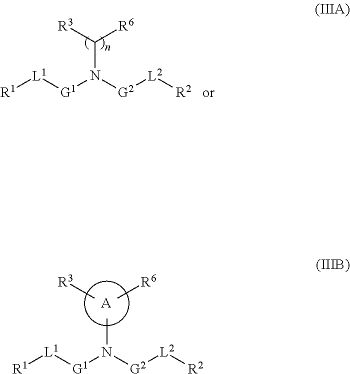

wherein: A is a 3 to 8-membered cycloalkyl or cycloalkylene ring: R³ is, at each occurrence, independently H, OH or C₁-C₂₄ alkyl; n is an integer ranging from 1 to 15. In some of the foregoing embodiments of Formula (III), the lipid has structure (IIIA). and in other embodiments, the lipid has structure (IIIB).

In other embodiments of Formula (III), the lipid has one of the following structures (IIIC) or (IIID):

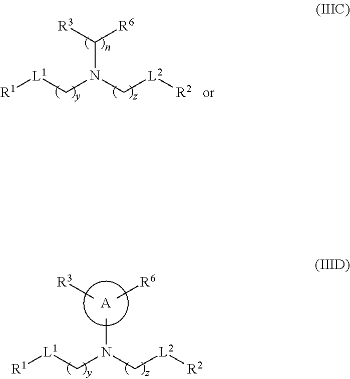

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—. In some different embodiments of Formula (III). the lipid has one of the following structures (IIIE) or (IIIF):

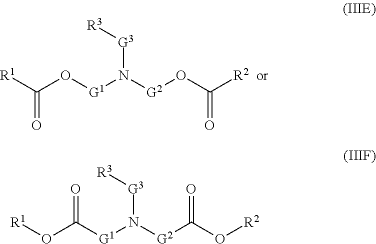

In some of the foregoing embodiments of Formula (III), the lipid has one of the following structures (IIIG), (IIIH) (IIII), or (IIIJ):

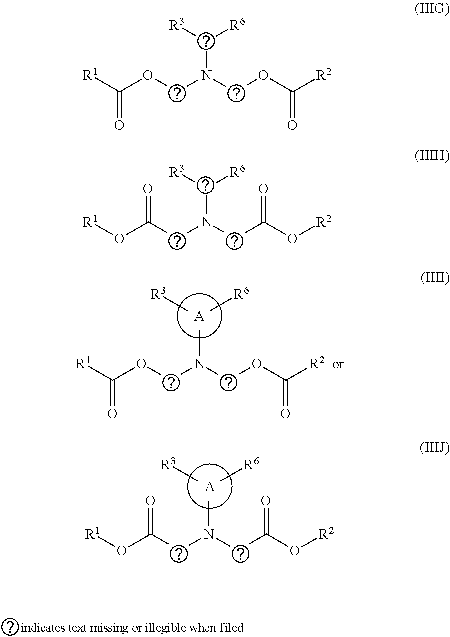

In some of the foregoing embodiments of Formula (III), n is an integer, ranging from 2 to 12, for example from 2 to 8 or from 2 to 4. For example, in some embodiments, n is 3, 4, 5 or, 6. In some embodiments, n is 3. In some embodiments, n is 4. In some embodiments, n is 5. In some embodiments, n is 6. In some other of the foregoing embodiments of Formula (III), y and z are each independently an integer ranging from 2 to 10, For example, in some embodiments, y and z are each independently an integer ranging from 4 to 9 or from 4 to 6. In some of the foregoing embodiments of Formula (III), R⁶ is H. in other of the foregoing embodiments, R⁶ is C₁-C₂₄ alkyl. In other embodiments, R⁶ is OH. In some embodiments of Formula (III), G³ is unsubstituted. In other embodiments, G3 is substituted, In various different embodiments, G³ is linear C₁-C24 alkylene or linear C₁-C₂₄ alkenylene. In some other foregoing embodiments of Formula (III), R′ or R², or both, is C₆-C₂₄ alkenyl. For example, in some embodiments, R³ and R² each, independently have the following structure:

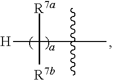

wherein: R^{7a} and R^{7b} are, at each occurrence, independently H or C₁-C₁₂ alkyl; and a is an integer from 2 to 12. wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl

In different embodiments of Formula (III), R′ or R². or both, has one of the following structures:

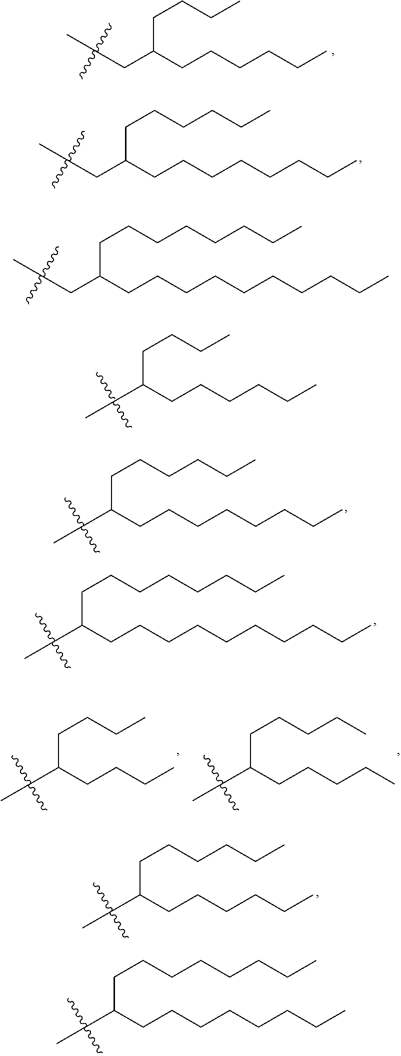

In some of the foregoing embodiments of Formula (III), R³ is OH, CN, —O(═O)OR⁴, —OC(═O)R⁴ or —NHC(═O)R⁴. In some embodiments, R⁴ is methyl or ethyl. In various different embodiments, the cationic lipid of Formula (III) has one of the structures set forth in Table 1 (“Representative Compounds of Formula (III)”) below.

TABLE 1
Representative Compounds of Formula (III)

| No. | Structure |
|---|---|
| III-1 | 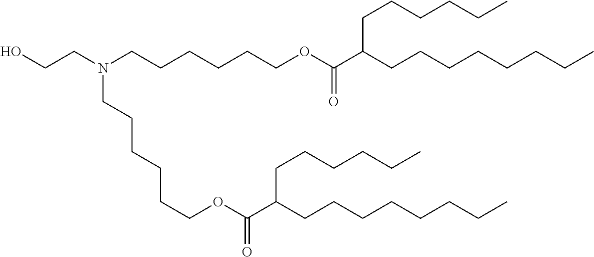 [SMILES: CCCCCCCCC(CCCCCC)C(=O)OCCCCCCN(CCO)CCCCCCOC(=O)C(CCCCCC)CCCCCCCC] |
| III-2 | 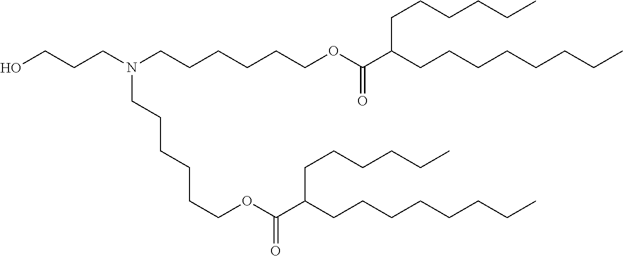 [SMILES: CCCCCCCCC(CCCCCC)C(=O)OCCCCCCN(CCCO)CCCCCCOC(=O)C(CCCCCC)CCCCCCCC] |
| III-3 | 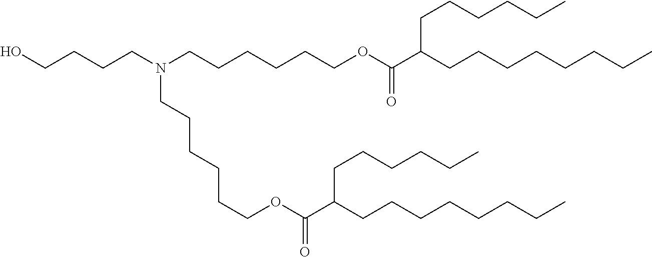 [SMILES: CCCCCCCCC(CCCCCC)C(=O)OCCCCCCN(CCCCO)CCCCCCOC(=O)C(CCCCCC)CCCCCCCC] |
| III-4 | 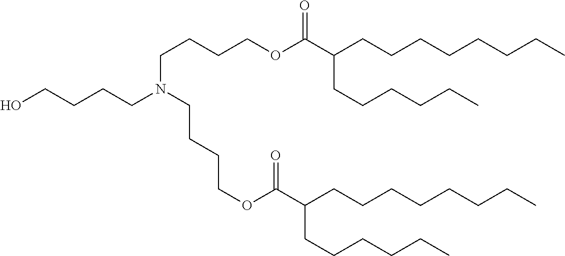 [SMILES: CCCCCCCCC(CCCCCC)C(=O)OCCCCN(CCCCO)CCCCOC(=O)C(CCCCCC)CCCCCCCC] |
| III-5 | 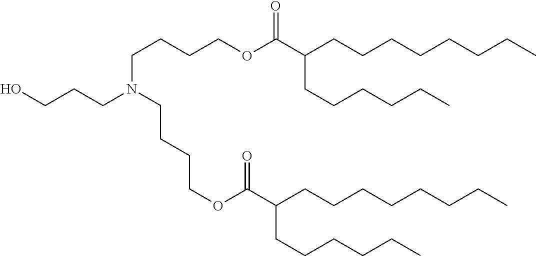 [SMILES: CCCCCCCCC(CCCCCC)C(=O)OCCCCN(CCCO)CCCCOC(=O)C(CCCCCC)CCCCCCCC] |
| III-6 | 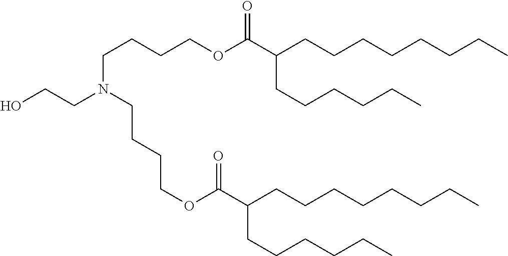 [SMILES: CCCCCCCCC(CCCCCC)C(=O)OCCCCN(CCO)CCCCOC(=O)C(CCCCCC)CCCCCCCC] |
| III-7 | 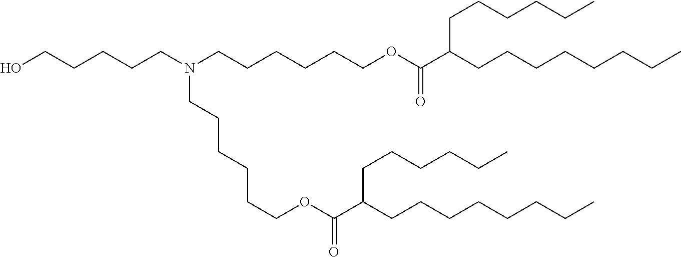 [SMILES: CCCCCCCCC(CCCCCC)C(=O)OCCCCCCN(CCCCCO)CCCCCCOC(=O)C(CCCCCC)CCCCCCCC] |
| III-8 | 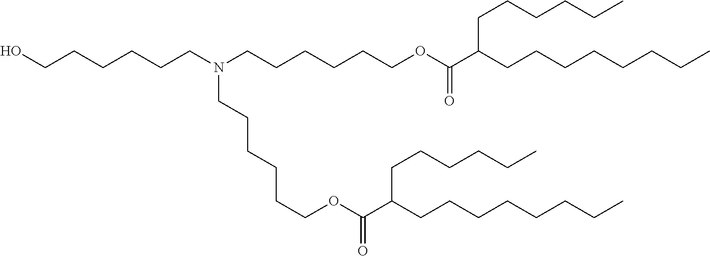 [SMILES: CCCCCCCCC(CCCCCC)C(=O)OCCCCCCN(CCCCCCO)CCCCCCOC(=O)C(CCCCCC)CCCCCCCC] |
| III-9 | 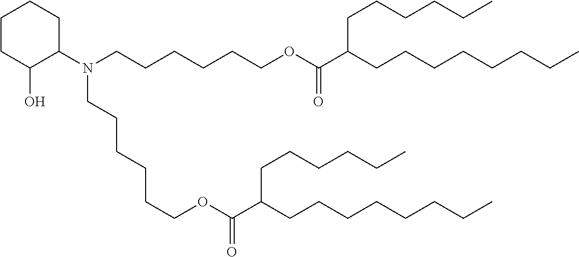 [SMILES: CCCCCCCCC(CCCCCC)C(=O)OCCCCCCN(CCCCCCOC(=O)C(CCCCCC)CCCCCCCC)C1CCCCC1O] |
| III-10 | 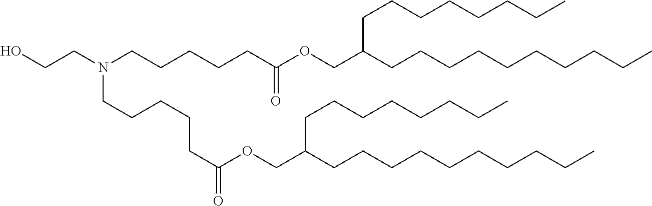 [SMILES: CCCCCCCCCCC(CCCCCCCC)COC(=O)CCCCCN(CCO)CCCCCC(=O)OCC(CCCCCCCC)CCCCCCCCCC] |
| III-11 | 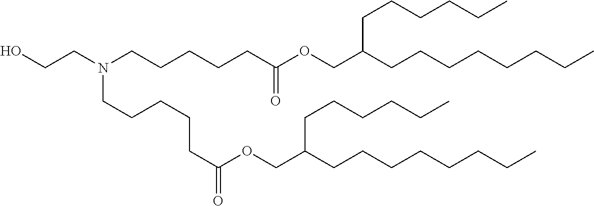 [SMILES: CCCCCCCCC(CCCCCC)COC(=O)CCCCCN(CCO)CCCCCC(=O)OCC(CCCCCC)CCCCCCCC] |
| III-12 | 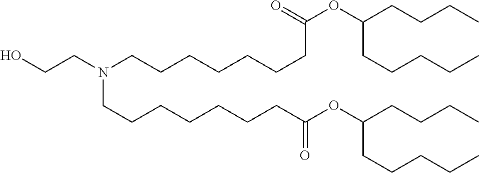 [SMILES: CCCCCC(CCCC)OC(=O)CCCCCCCN(CCO)CCCCCCCC(=O)OC(CCCC)CCCCC] |
| III-13 | 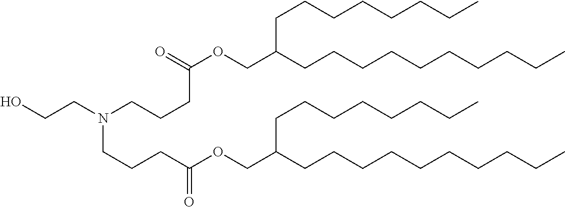 [SMILES: CCCCCCCCCCC(CCCCCCCC)COC(=O)CCCN(CCO)CCCC(=O)OCC(CCCCCCCC)CCCCCCCCCC] |
| III-14 | 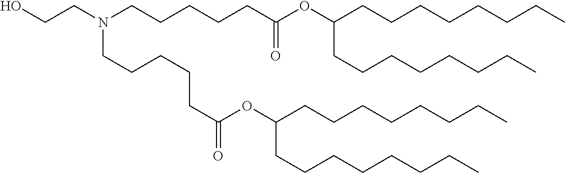 [SMILES: CCCCCCCCC(CCCCCCCC)OC(=O)CCCCCN(CCO)CCCCCC(=O)OC(CCCCCCCC)CCCCCCCC] |
| III-15 | 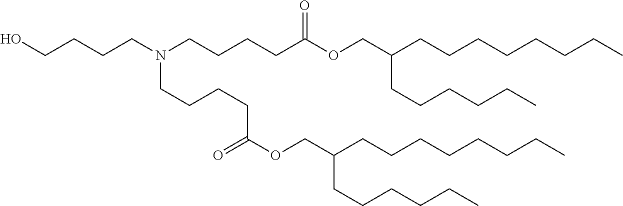 [SMILES: CCCCCCCCC(CCCCCC)COC(=O)CCCCN(CCCCO)CCCCC(=O)OCC(CCCCCC)CCCCCCCC] |
| III-16 | 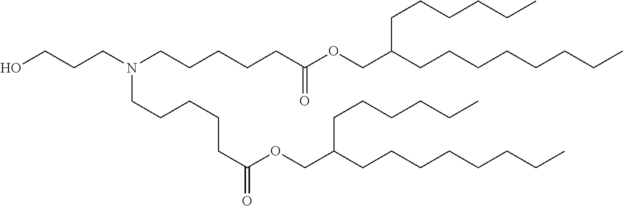 [SMILES: CCCCCCCCC(CCCCCC)COC(=O)CCCCCN(CCCO)CCCCCC(=O)OCC(CCCCCC)CCCCCCCC] |
| III-17 | 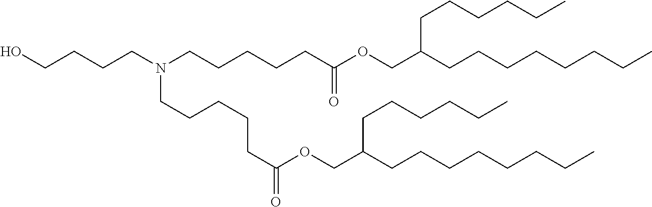 [SMILES: CCCCCCCCC(CCCCCC)COC(=O)CCCCCN(CCCCO)CCCCCC(=O)OCC(CCCCCC)CCCCCCCC] |
| III-18 | 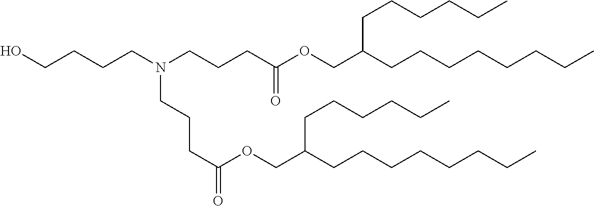 [SMILES: CCCCCCCCC(CCCCCC)COC(=O)CCCN(CCCCO)CCCC(=O)OCC(CCCCCC)CCCCCCCC] |
| III-19 | 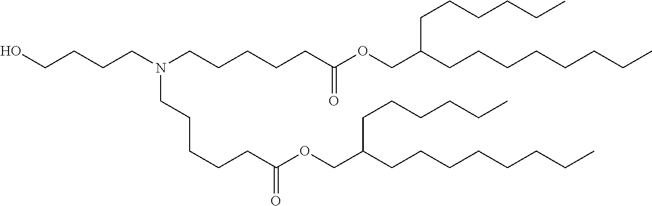 [SMILES: CCCCCCCCC(CCCCCC)COC(=O)CCCCCN(CCCCO)CCCCCC(=O)OCC(CCCCCC)CCCCCCCC] |
| III-20 | 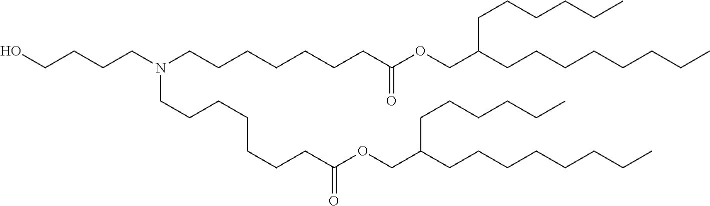 [SMILES: CCCCCCCCC(CCCCCC)COC(=O)CCCCCCCN(CCCCO)CCCCCCCC(=O)OCC(CCCCCC)CCCCCCCC] |
| III-21 | 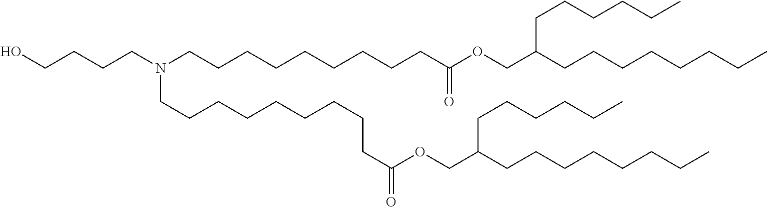 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 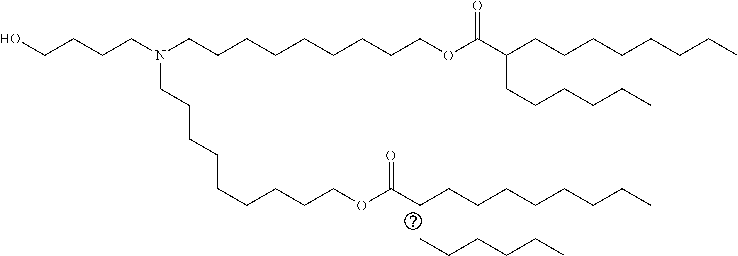 |
| III-23 | 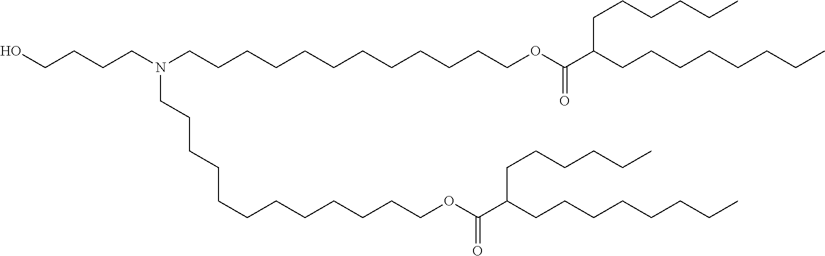 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 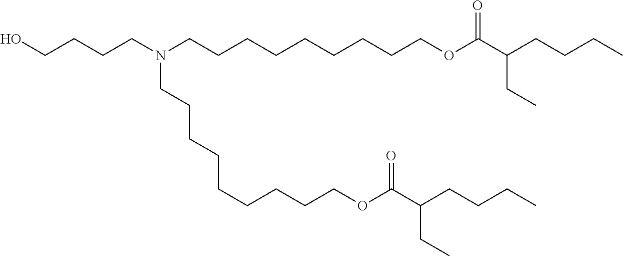 [SMILES: CCCCC(CC)C(=O)OCCCCCCCCCN(CCCCO)CCCCCCCCCOC(=O)C(CC)CCCC] |
| III-25 | 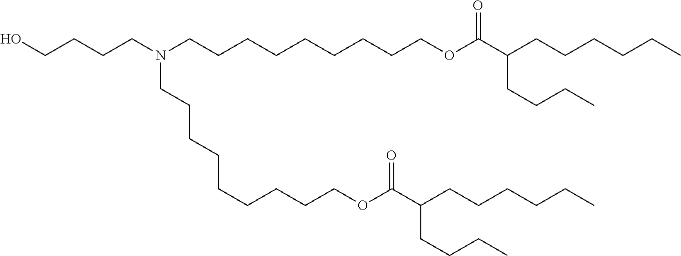 [SMILES: CCCCCCC(CCCC)C(=O)OCCCCCCCCCN(CCCCO)CCCCCCCCCOC(=O)C(CCCC)CCCCCC] |
| III-26 | 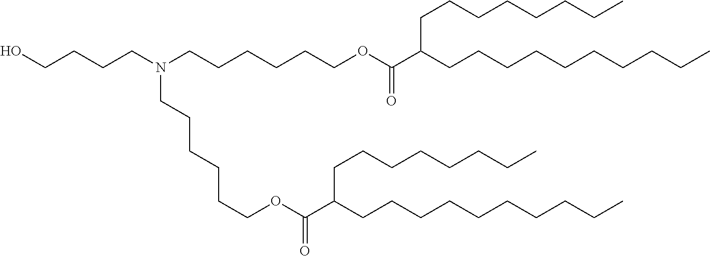 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 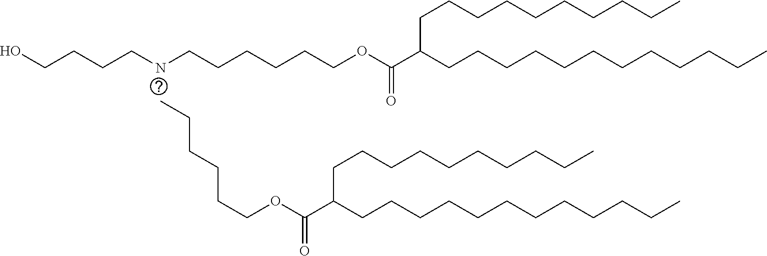 |
| III-28 | 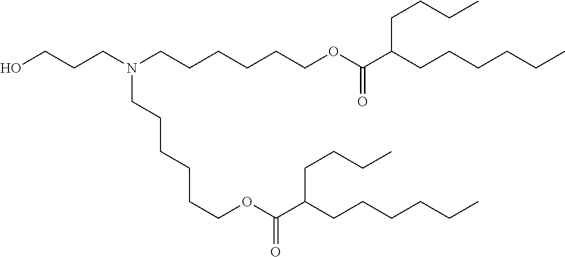 [SMILES: CCCCCCC(CCCC)C(=O)OCCCCCCN(CCCO)CCCCCCOC(=O)C(CCCC)CCCCCC] |
| III-29 | 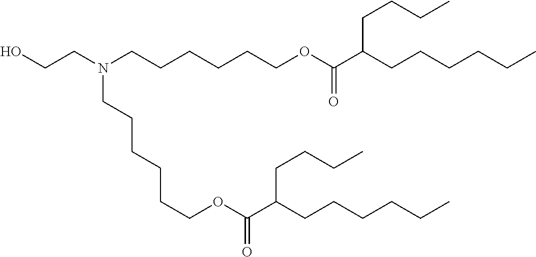 [SMILES: CCCCCCC(CCCC)C(=O)OCCCCCCN(CCO)CCCCCCOC(=O)C(CCCC)CCCCCC] |
| III-30 | 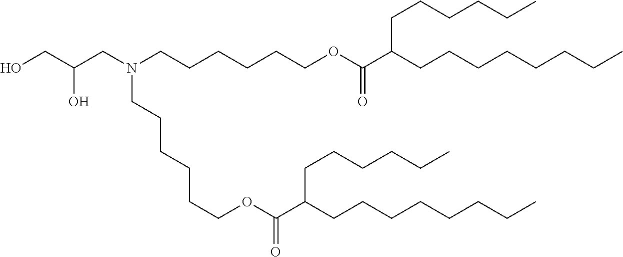 [SMILES: CCCCCCCCC(CCCCCC)C(=O)OCCCCCCN(CCCCCCOC(=O)C(CCCCCC)CCCCCCCC)CC(O)CO] |
| III-31 | 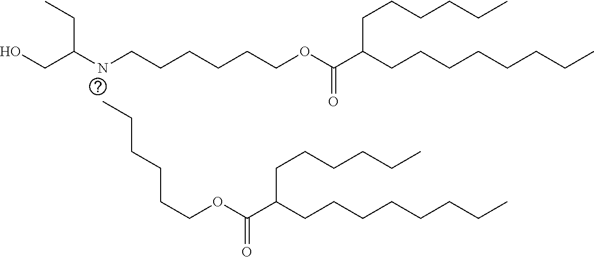 |
| III-32 | 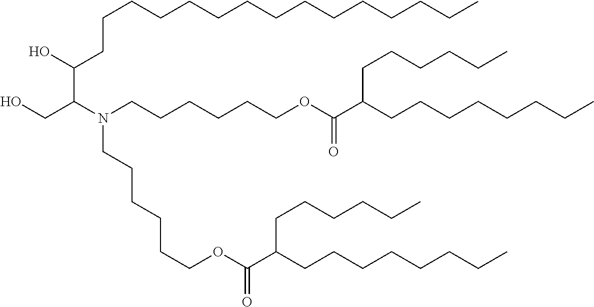 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 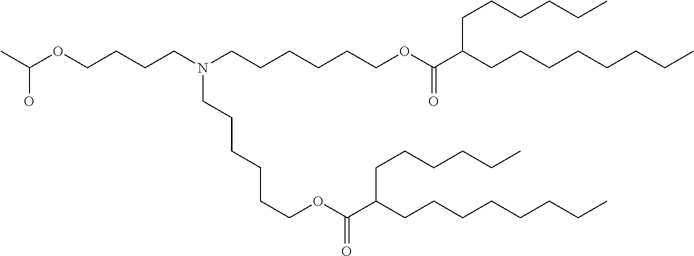 [SMILES: CCCCCCCCC(CCCCCC)C(=O)OCCCCCCN(CCCCCCOC(=O)C(CCCCCC)CCCCCCCC)CCCCOC(C)O] |
| III-34 | 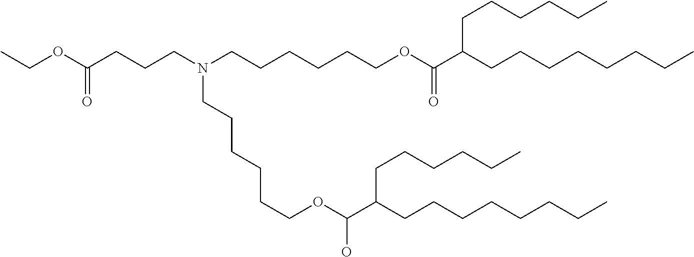 [SMILES: CCCCCCCCC(CCCCCC)C(=O)OCCCCCCN(CCCCCCOC(O)C(CCCCCC)CCCCCCCC)CCCC(=O)OCC] |
| III-35 | 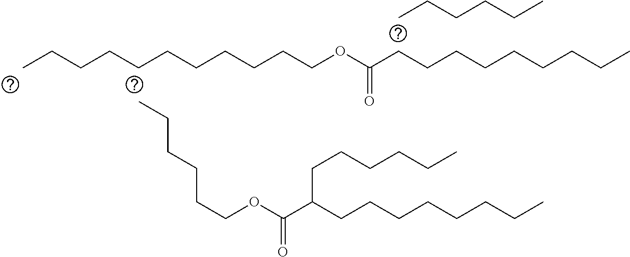 |
| III-36 | 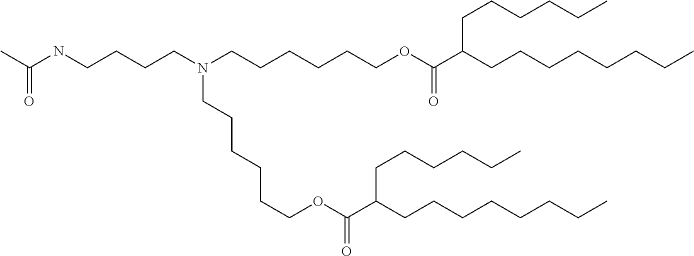 [SMILES: CCCCCCCCC(CCCCCC)C(=O)OCCCCCCN(CCCCCCOC(=O)C(CCCCCC)CCCCCCCC)CCCCNC(C)=O] |

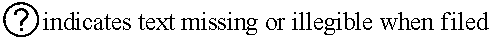

In some embodiments the LNPs comprise a lipid of Formula (III), a mRNA compound as described herein and one or more excipient selected from neutral lipids, steroids and pegylated lipids. In some embodiments the lipid of Formula (III) is compound III-3. In some embodiments the lipid of Formula (III) is compound III-7. Within the context of the present invention LNP-III-3 means a lipid nanoparticle as defined herein comprising the cationic lipid compound III-3, according to the table above. Other lipid nanoparticles are referenced in analogous form. In certain embodiments, the cationic lipid of Formula (III) is present in the LNP in an amount from about 30 to about 95 mole percent, relative to the total lipid content of the LNP. If more than one cationic lipid is incorporated within the LNP, such percentages apply to the combined cationic lipids. In one embodiment, the cationic lipid is present in the LNP in an amount from about 30 to about 70 mole percent. In one embodiment, the cationic lipid is present in the LNP in an amount from about 40 to about 60 more percent. such as about 40, 41, 42, 43, 44, 45, 46, 47, 48, 49, 50, 51, 52, 53, 54, 55, 56, 57, 58, 59 or 60 mole percent, respectively.

In some embodiments of the invention the LNP comprises a combination or mixture of any the lipids described above.

In one of the preferred embodiments, the lipid nanoparticle comprises a cationic lipid selected from the group of:

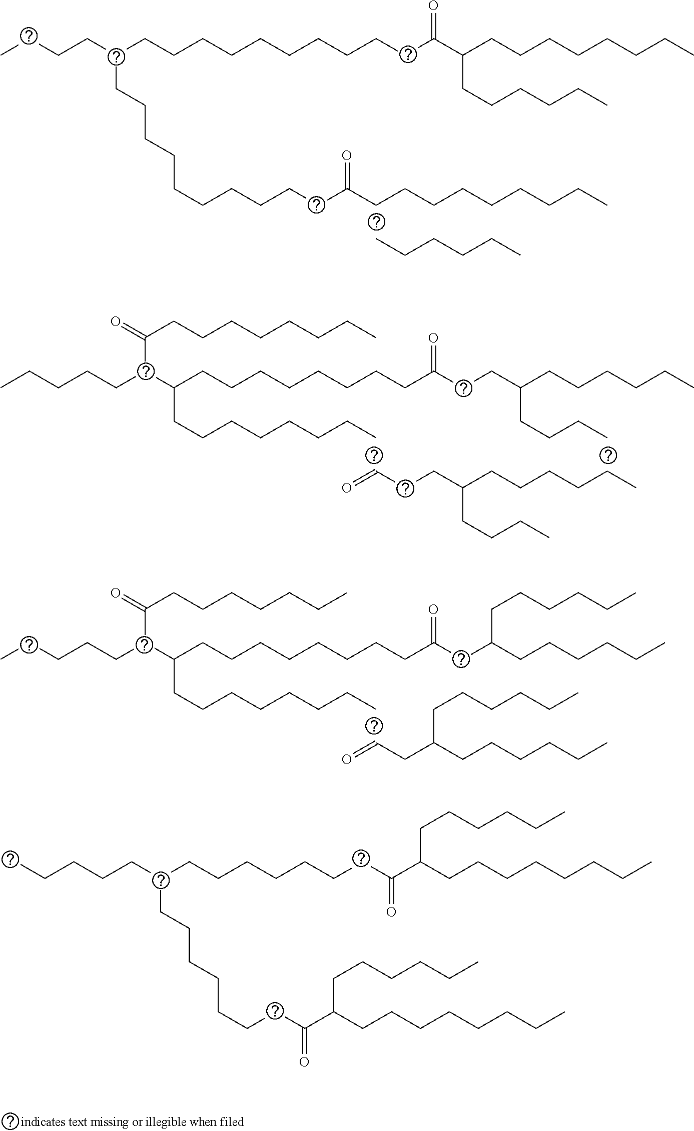

In a further embodiment the invention relates to an mRNA comprising lipid nanoparticle comprising:

(i) a PEG lipid with the formula (IV)

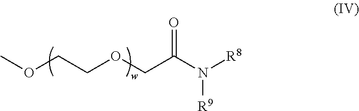

wherein R⁸ and R⁹ are each independently a straight or branched, saturated or unsaturated alkyl chain containing from 10 to 30 carbon atoms, wherein the alkyl chain is optionally interrupted by one or more ester bonds; and w has a mean value ranging from 30 to 60; and (ii) a mRNA compound comprising an mRNA sequence encoding at least one antigenic peptide or protein; wherein the mRNA compound is encapsulated in or associated with said lipid nanoparticle. With respect to the mRNA compound, the mRNA sequence and the antigenic peptide or protein, reference is made to the description of these features including the respective options and preferences above. In one of the preferred embodiments, the mRNA compound does not comprise a nucleoside modification. In another embodiment, it comprises no base modification. In a further embodiment, it does not comprise a 1-methylpseudouridine modification. Moreover, if the PEG lipid is compound (IVa), the lipid nanoparticle is not a lipid nanoparticle comprising compound I-6, DSPC, cholesterol and the PEG lipid (IVa) at a ratio of about 50:10:38.5:1.5 that encapsulates unmodified, 1-methylpseudouridine modified or codon-optimized mRNA encoding an influenza PR8 or Cal/7/2009 hemagglutinin or an HIV-1 CD4-independent R3A envelop protein. In one of the preferred embodiments, the lipid nanoparticle comprises (i) a cationic lipid according to formula (III) as defined above, (ii) a mRNA compound comprising an mRNA sequence encoding at least one antigenic peptide or protein as described herein, and (iii) a PEG lipid of formula (IV); wherein the mRNA compound is encapsulated in or associated with said lipid nanoparticle.

The amount of the permanently cationic lipid or lipidoid should also be selected taking the amount of the nucleic acid cargo into account, In one embodiment, these amounts are selected such as to result in an NIP ratio of the nanoparticle(s) or of the composition in the range from about 0.1 to about 20, in this context, the NIP ratio is defined as the mole ratio of the nitrogen atoms (“N”) of the basic nitrogen-containing groups of the lipid or lipidoid to the phosphate groups (“P”) of the nucleic acid which is used as cargo, The N/P ratio may be calculated on the basis that, for example, 1 μg RNA typically contains about 3 nmol phosphate residues, provided that the RNA exhibits a statistical distribution of bases. The “N”-value of the lipid or lipidoid may be calculated on the basis of its molecular weight and the relative content of permanently cationic and—if present—cationisable groups.

Such low NIP ratios are commonly believed to be detrimental to the performance and in vivo efficacy of such carrier-cargo complexes, or nucleic-acid loaded nanoparticles. However, the inventors found that such N/P ratios are indeed useful in the context of the present invention, in particular when the local or extravascular administration of the nanoparticles is intended. Here, the respectively nanoparticles have been found to be efficacious and at the same time well-tolerated.

In certain embodiments, the LNP comprises one or more additional lipids which stabilize the formation of particles during their formation, Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolarnines, ceramides, sphingomyelins, dihydro sphingonyelins, cephalins, and cerebrosides. Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipamitoylphosphatidylglycerol (DPPG), dioleoyl-phosphatidylethanolamine (DOPE), palnitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-Icarboxylate (DOPE-mal), dipalmitoyl phosphatidyl ethanolamine (DPPE), dimyristoylphosphoethanolamine (DMPE), distearoyl-phosphatidylethanolai-ne (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one embodiment, the neutral lipid is 1,2-distearoyl-sn-glycero-3phosphocholine (DSPC). In some embodiments, the LNPs comprise a neutral lipid selected from DSPC. DPPC. DMPC, DOPC, POPC. DOPE and SM. In various embodiments, the molar ratio of the cationic lipid (e.g., lipid of Formula (I), (II) or (Ill)) to the neutral lipid ranges from about 2:1 to about 8:1.

In various embodiments, the LNPs further comprise a steroid or steroid analogue. A “steroid” is a compound comprising the following carbon skeleton:

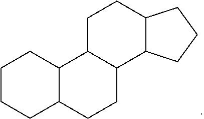 [SMILES: C1CCC2C(C1)CCC1C3CCCC3CCC21]

In certain embodiments, the steroid or steroid analogue is cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP comprises an additional, stabilizing-lipid which is a polyethylene glycol-lipid (pegylated lipid). Suitable polyethylene glycollipids include PEG-modified phosphatidylethanolamine. PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N[(methoxy poly(ethylene glycol)2000)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(rnonomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(o-methoxy(polyethoxy)ethyl)butanedioate (PEG-S-DMG), a pegylated ceramide (PEG-cer), or a PEG dialkoxypropylcarbamate such as mu-methoxy(polyethoxy)ethyl-N-(2,3di(tetradeca noxy)propyl)carba mate or 2,3-di(tetradecanoxy)propyl-N-(w-methoxy(polyethoxy)ethyl)carbamate. In various embodiments, the molar ratio of the cationic lipid to the pegylated lipid ranges from about 100:1 to about 25:1.

As mentioned, the mRNA comprising lipid nanoparticle may comprise a pegylated lipid having the structure of formula (IV):

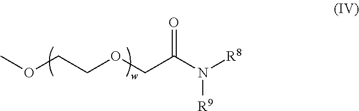

or a pharmaceutically acceptable salt, tautomer or stereoisomer thereof, wherein: R⁸ and R⁹ are each independently a straight or branched, saturated or unsaturated alkyl chain containing from 10 to 30 carbon atoms, wherein the alkyl chain is optionally interrupted by one or more ester bonds; and w has mean value ranging from 30 to 60. In some of the foregoing embodiments of the pegylated lipid (IV), R⁸ and R⁹ are not both n-octadecyl when w is 42. In some other embodiments, R⁸ and R⁹ are each independently a straight or branched, saturated or unsaturated alkyl chain containing from 10 to 18 carbon atoms. In some embodiments, R⁸ and R⁹ are each independently a straight or branched, saturated or unsaturated alkyl chain containing from 12 to 16 carbon atoms. In some embodiments, R⁸ and R⁹ are each independently a straight or branched, saturated or unsaturated alkyl chain containing 12 carbon atoms. In some embodiments, R⁸ and R⁹ are each independently a straight or branched, saturated or unsaturated alkyl chain containing 14 carbon atoms. In other embodiments, R⁸ and R⁹ are each independently a straight or branched, saturated or unsaturated alkyl chain containing 16 carbon atoms. In still more embodiments, R⁸ and R⁹ are each independently a straight or branched, saturated or unsaturated alkyl chain containing 18 carbon atoms. In still other embodiments. R⁸ is a straight or branched, saturated or unsaturated alkyl chain containing 12 carbon atoms and R⁹ is a straight or branched, saturated or unsaturated alkyl chain containing 14 carbon atoms. In various embodiments, w spans a range that is selected such that the PEG portion of (IV) has an average molecular weight of about 400 to about 6000 g/mol. In some embodiments, the average w is about 50. In a preferred embodiment R⁸ and R⁹ are saturated alkyl chains.

In a further preferred embodiment the PEG lipid is of formula (IVa)

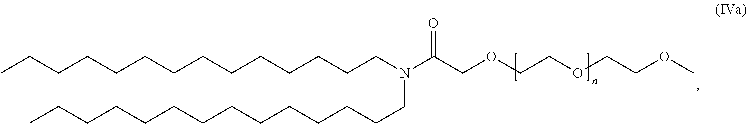

wherein n has a mean value ranging from 30 to 60, such as about 30+2, 32+2, 34+2, 36+2, 38+2, 40+2, 42+2, 44+2, 46+2, 48+2, 50+2, 52+2, 54+2, 56+2, 58+2, or 60+2. In a most preferred embodiment n is about 49.

In other embodiments, the pegylated lipid has one of the following structures:

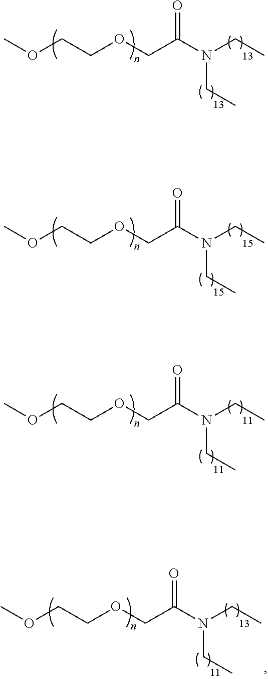

wherein n is an integer selected such that the average molecular weight of the pegylated lipid is about 2500 g/mol, most preferably n is about 49.

In certain embodiments, the PEG lipid is present in the LNP in an amount from about 1 to about 10 mole percent, relative to the total lipid content of the nanoparticle. In one embodiment, the PEG lipid is present in the LNP in an amount from about 1 to about 5 mole percent. In one embodiment, the PEG lipid is present in the LNP in about 1 mole percent or about 1.5 mole percent.

In a particular preferred embodiment, the (pharmaceutical) composition or the vaccine according to the invention Comprising mRNA comprises lipid nanoparticles, which have a molar ratio of approximately 50:10:38.5:1.5, preferably 47.5:10:40.8:1.7 or more preferably 47.4:10:40.9:1.7 (i.e. proportion (mol %) of cationic lipid, DSPC, cholesterol and PEG-lipid; solubilized in ethanol).

In one embodiment, the RNA in the RNA solution is at a concentration of <1 mg/mL. In another embodiment, the RNA is at a concentration of at least about 0.05 mg/mL. In another embodiment, the RNA is at a concentration of at least about 0.5 mg/mL. In another embodiment, the RNA is at a concentration of at least about 1 mg/mL. In another embodiment, the RNA concentration is from about 0.05 mg/mL to about 0.5 mg/mL. In another embodiment, the RNA is at a concentration of at least 10 mg/mL. In another embodiment, the RNA is at a concentration of at least 50 mg/mL. In some embodiments, the RNA is at a concentration of equal to any one of, at least any one of, at most any one of, or between any two of about 0.05 mg/mL, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

In a further embodiment, the RNA solution and the lipid preparation mixture further comprises a stabilizing agent. In some embodiments, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In a specific embodiment, the stabilizing agent is sucrose. In a specific embodiment, the stabilizing agent is trehalose. In a specific embodiment, the stabilizing agent is a combination of sucrose and trehalose. In some embodiments, the stabilizing agent concentration includes, but is not limited to, a concentration of about 10 mg/mL to about 400 mg/mL, about 100 mg/mL to about 200 mg/mL, or about 103 mg/mL to about 200 mg/mL. In some embodiments, the concentration of the stabilizing agent is equal to any one of, at least any one of, at most any one of, or between any two of 10 mg/mL, 20 mg/mL, 50 mg/mL, 103 mg/mL, 150 mg/mL, 200 mg/mL, 300 mg/mL, 400 mg/mL, or more.

In a further embodiment, the mass amount of the stabilizing agent and the mass amount of the RNA are in a specific ratio. In one embodiment, the ratio of the mass amount of the stabilizing agent and the RNA is no greater than 5000. In another embodiment, the ratio of the mass amount of the stabilizing agent and the RNA is no greater than 2000. In another embodiment, the ratio of the mass amount of the stabilizing agent and the RNA is no greater than 1000. In another embodiment, the ratio of the mass amount of the stabilizing agent and the RNA is no greater than 500. In another embodiment, the ratio of the mass amount of the stabilizing agent and the RNA is no greater than 100. In another embodiment, the ratio of the mass amount of the stabilizing agent and the pharmaceutical substance is no greater than 50. In another embodiment, the ratio of the mass amount of the stabilizing agent and the RNA is no greater than 10. In another embodiment, the ratio of the mass amount of the stabilizing agent and the RNA is no greater than 1. In another embodiment, the ratio of the mass amount of the stabilizing agent and the RNA is no greater than 0.5. In another embodiment, the ratio of the mass amount of the stabilizing agent and the RNA is no greater than 0.1. In another embodiment, the stabilizing agent and RNA comprise a mass ratio of about 200-2000 of the stabilizing agent: 1 of the RNA. In a further embodiment, the RNA is saRNA and the stabilizing agent is sucrose.

In some embodiments, the RNA solution and the lipid preparation mixture further comprises a salt. In one embodiment, the salt is a sodium salt. In a specific embodiment, the salt is NaCl. In some embodiments, the RNA solution and the lipid preparation mixture further comprises a surfactant, a preservative, any other excipient, or a combination thereof. As used herein, “any other excipient” includes, but is not limited to, antioxidants, glutathione, EDTA, methionine, desferal, antioxidants, metal scavengers, or free radical scavengers. In one aspect, the surfactant, preservative, excipient or combination thereof is selected from sterile water for injection (sWFI), bacteriostatic water for injection (BWFI), saline, dextrose solution, polysorbates, poloxamers, Triton, divalent cations, Ringer's lactate, amino acids, sugars, polyols, polymers or cyclodextrins.

In some embodiments, the RNA solution and/or the lipid preparation mixture further comprises at least one free amino acid. In certain cases, the at least one free amino acid is internally loaded in the LNP-encapsulated RNAs. For example, in some cases, the at least one free amino acid is soluble in water and is combined with an RNA solution described herein (e.g., the RNA product solution). In some cases, the at least one free amino acid is soluble in ethanol and is combined with a lipid preparation described herein (comprising, e.g., at least one cationic lipid and optionally one or more other lipid components, in an organic solvent). In some cases, the at least one free amino acid is soluble in water and/or ethanol and is combined with both an RNA solution described herein (e.g., the RNA product solution) and a lipid preparation described herein (comprising, e.g., at least one cationic lipid and optionally one or more other lipid components, in an organic solvent). In this way, the at least one free amino acid is comprised in the LNP-encapsulated RNAs.

Alternatively, or in addition to, internally loading the LNP-encapsulated RNAs with the at least one free amino acid, pre-formed LNP-encapsulated RNAs may be externally loaded with the at least one free amino acid. For example, in some cases, the LNP-encapsulated RNAs produced according to the methods described herein are combined with the at least one free amino acid.

In some embodiments, each of a buffer, stabilizing agent, salt, surfactant, preservative, and excipient are included in the RNA solution and the lipid preparation mixture. In other embodiments, any one or more of a buffer, stabilizing agent, salt, surfactant, preservative, and excipient may be excluded from the RNA solution and the lipid preparation mixture.

### X. Compositions

In some instances, the compositions described herein include at least one isolated RNA molecule as described herein. In some instances, the compositions described herein further include at least one free amino acid, as described herein.

In some embodiments, the RNA molecule, which includes a modified nucleotide, is capable of evading an innate immune response of a cell into which the first RNA molecule is introduced. In some embodiments, the RNA molecule does not further include any one of a cap, UTR, and poly A tail while being capable of evading an innate immune response of the cell. In some embodiments, the RNA molecule does not include a subgenomic promoter and is not a self-amplifying RNA molecule. In some embodiments, the RNA molecule includes a non-coding region. In some embodiments, the RNA molecule includes any one of a 5′ cap, a 5′ UTR, an open reading frame, a 3′ UTR, and a poly A sequence, or any combination thereof. In some embodiments, the RNA molecule includes a 5′ cap moiety. In some embodiments, the RNA molecule includes a 5′ UTR and a 3′UTR. In some embodiments, the RNA molecule includes a 5′ UTR, an open reading frame, a 3′UTR, and does not further include a 5′ cap. In some embodiments, the RNA molecule includes a 5′ cap moiety, 5′ UTR, coding region, 3′ UTR, and a 3′ poly A sequence. In some embodiments, the RNA molecule includes a 5′ cap moiety, 5′ UTR, noncoding region, 3′ UTR, and a 3′ poly A sequence. In some embodiments, the RNA molecule includes a noncoding region and does not further comprise any one of a 5′ cap moiety, 5′ UTR, 3′ UTR, and a 3′ poly A sequence. In some embodiments, the RNA molecule includes a 5′ cap moiety, a 5′ untranslated region (5′ UTR), a modified nucleotide, an open reading frame, a 3′ untranslated region (3′ UTR), and a 3′ poly A sequence.

In some embodiments, equal to any one of, at least any one of, at most any one of, or between any two of 50%, 55%, 60%, 65%, 70%, 71%, 72%, 73%, 74%,75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the total RNA molecules (capped and uncapped) in the composition are capped.

In some embodiments, equal to any one of, at least any one of, at most any one of, or between any two of 35%, 40%, 45%, 50%, 55%, 60%, 65%, 70%, 71%, 72%, 73%, 74%,75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the total RNA molecules in the composition are full length RNA transcripts. Purity may be determined as described herein, e.g., via reverse phase HPLC or Bioanalyzer chip-based electrophoresis and measure by, e.g., peak area of full-length RNA molecule relative to total peak. In some embodiments, a fragment analyzer (FA) may be used to quantify and purify the RNA. The fragment analyzer automates capillary electrophoresis and HPLC.

In some embodiments, the composition is substantially free of one or more impurities or contaminants including the linear DNA template and/or reverse complement transcription products and, for instance, includes RNA molecules that are equal to any one of, at least any one of, at most any one of, or between any two of 90%, 91%, 92%, 93%, 94%, 95%, 96%, 97%, 98%, or 99% pure; at least 98% pure, or at least 99% pure.

In some embodiments, the RNA of the compositions is protected from degradation upon inclusion of the at least one free amino acid in the composition compared to a composition lacking the at least one free amino acid. In some embodiments, immunogenicity of RNA and/or the compositions including the RNA is increased upon inclusion of the at least one free amino acid in the composition compared to immunogenicity of RNA and/or compositions lacking the at least one free amino acid. In some embodiments, in vitro expression of the RNA of the compositions is increased upon inclusion of the at least one free amino acid in the composition compared to in vitro expression of RNA in a composition lacking the at least one free amino acid.

In one embodiment of the compositions described herein, the at least one free amino acid is glutamic acid, glutamate, aspartic acid, aspartate, arginine, lysine, tyrosine, cysteine, glutamine, asparagine, threonine, serine, pharmaceutically acceptable salts thereof, or any mixtures thereof. In another embodiment of the compositions described herein, the at least one free amino acid is glutamic acid, glutamate, aspartic acid, aspartate, arginine, lysine, pharmaceutically acceptable salts thereof, or any mixtures thereof. In a specific embodiment of the compositions described herein, the at least one free amino acid is glutamic acid, glutamate, or a pharmaceutically acceptable salt thereof. In a specific embodiment of the compositions described herein, the at least one free amino acid is aspartic acid, aspartate, or a pharmaceutically acceptable salt thereof. In a specific embodiment of the compositions described herein, the at least one free amino acid is arginine, or a pharmaceutically acceptable salt thereof. In a specific embodiment of the compositions described herein, the at least one free amino acid is lysine, or a pharmaceutically acceptable salt thereof.

The compositions described herein include at least 1 mM of at least one free amino acid. In some embodiments, the at least one free amino acid is comprised in a solution. In some embodiments, the amount of at least one free amino acid used to protect RNA from degradation, increase immunogenicity of the RNA and/or compositions comprising the RNA, and/or increase in vitro expression of the RNA can be in concentrations in excess of the RNA concentration. For example, the at least one free amino acid can be present in concentrations up to 10× (or more) of the total nucleobase concentration of the RNA, and can be as high as concentrations that approach the solubility limit of the amino acid in the delivery vehicle (including drug product). Concentrations of the at least one free amino acid used to protect RNA from degradation, increase immunogenicity of the RNA and/or compositions comprising the RNA, and/or to increase in vitro expression of the RNA can be, for example, between 1 mM and 100 mM (or the solubility limit), or any range or value derivable therein. For example, the at least one free acid concentration can be equal to any one of, at least any one of, at most any one of, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM, or any range or value derivable therein. In some embodiments, the at least one free amino acid concentration can range from 1 to 10 mM, from 1 to 20 mM, from 1 to 30 mM, from 1 to 40 mM, from 1 to 50 mM, from 1 to 60 mM, from 1 to 70 mM, from 1 to 80 mM, from 1 to 90 mM, or from 1 to 100 mM. In some embodiments, the at least one free amino acid concentration can range from 10 to 20 mM, from 20 to 30 mM, from 30 to 40 mM, from 40 to 50 mM, from 50 to 60 mM, from 60 to 70 mM, from 70 to 80 mM, from 80 to 90 mM, or from 90 to 100 mM. In some embodiments, the at least one free amino acid concentration can be 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM. In specific embodiments, the at least one free amino acid concentration is 1 mM. In specific embodiments, the at least one free amino acid concentration is 5 mM. In specific embodiments, the at least one free amino acid concentration is 10 mM. In specific embodiments, the at least one free amino acid concentration is 15 mM. In specific embodiments, the at least one free amino acid concentration is 20 mM.

In some embodiments, the composition further includes a lipid-based delivery system, which delivers an RNA molecule to the interior of a cell, where it can then replicate, inhibit protein expression of interest, and/or express the encoded polypeptide of interest. The delivery system may have adjuvant effects which enhance the immunogenicity of an encoded antigen. In some embodiments, the composition further includes neutral lipids, cationic lipids, cholesterol, and polyethylene glycol (PEG), and forms nanoparticles that encompass, or encapsulate, the RNA molecules. In some embodiments, the composition further includes any one of a cationic lipid, a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, and a cationic nanoemulsion. In some embodiments, the RNA molecule is encapsulated in, bound to or adsorbed on any one of a cationic lipid, a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, and a cationic nanoemulsion, or a combination thereof. As used herein, “encapsulate,” “encapsulated,” “encapsulation,” and grammatically comparable variants thereof mean that at least a portion of a substance is enclosed or surrounded by another material or another substance in a composition. In some embodiments, a substance, such as RNA, can be fully enclosed or surrounded by another material or another substance in a composition, such as a lipid.

In some embodiments, encapsulated RNA of the compositions is protected from degradation upon inclusion of the at least one free amino acid in the composition compared to a composition lacking the at least one free amino acid. In some embodiments, immunogenicity of encapsulated RNA and/or the compositions including the encapsulated RNA is increased upon inclusion of the at least one free amino acid in the composition compared to immunogenicity of compositions including encapsulated RNA that the at least one free amino acid. In some embodiments, in vitro expression of the encapsulated RNA of the compositions is increased upon inclusion of the at least one free amino acid in the composition compared to in vitro expression of encapsulated RNA in a composition lacking the at least one free amino acid.

The compositions disclosed herein may be produced by methods including: (a) combining the RNA and the at least one free amino acid, optionally, before the RNA is encapsulated; (b) combining the RNA and the at least one free amino acid during RNA encapsulation; or (c) combining the encapsulated RNA and the at least one free amino acid after the RNA is encapsulated.

In some embodiments in which the RNA is not encapsulated, the at least one free amino acid directly contacts the RNA. The RNA may be contacted with the at least one free amino acid before the RNA is encapsulated. In some embodiments in which the RNA is encapsulated, it is the capsule that encapsulates the RNA that contacts the at least one free amino acid. In some embodiments in which the RNA is encapsulated, though the capsule contacts the at least one amino acid, the capsule does not encapsulate the at least one free amino acid, and the capsule comprising the RNA is contacted with the at least one amino acid after the RNA is encapsulated in the capsule. In some embodiments in which the RNA is encapsulated, the capsule that encapsulates the RNA further encapsulates the at least one free amino acid.

In some embodiments, the compositions further comprise one or more stabilizing agents and one or more buffers. A RNA molecule, e.g., a naked or encapsulated RNA, as disclosed herein may be comprised in a solution comprising the one or more stabilizing agents and one or more buffers. In some embodiments, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In some embodiments, the stabilizing agent is a disaccharide, or sugar. In one embodiment, the stabilizing agent is sucrose. In another embodiment, the stabilizing agent is trehalose. In a further embodiment, the stabilizing agent is a combination of sucrose and trehalose. In some embodiments, the total concentration of the stabilizing agent(s) in the composition is about 5% to about 10% w/v. For example, the total concentration of the stabilizing agent can be equal to any one of, at least any one of, at most any one of, or between any two of 1%, 2%, 3%, 4%, 5%, 6%, 7%, 8%, 9%, 10%, 11%, 12%, 13%, 14%, 15%, 16%, 17%, 18%, 19%, or 20% w/v or any range or value derivable therein. In specific embodiments, the total concentration of the stabilizing agent(s) in the composition is 10% w/v. In specific embodiments, the amino acid concentration is 5% w/v.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some embodiments, the buffer is a HEPES buffer, a Tris buffer, or a PBS buffer. In one embodiment, the buffer is Tris buffer. In another embodiment, the buffer is a HEPES buffer. In a further embodiment, the buffer is a PBS buffer. In some embodiments, the concentration of the buffer in the composition is about 10 mM. For example, the buffer concentration can be equal to any one of, at least any one of, at most any one of, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. In specific embodiments, the buffer concentration is 10 mM. The buffer can be at a neutral pH, pH 6.5 to 8.5, pH 7.0 to pH 8.0, or pH 7.2 to pH 7.6. For example, the buffer can be at pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific embodiments, the buffer is at pH 7.4.

The compositions may further include one or more salts and/or one or more pharmaceutically acceptable surfactants, preservatives, carriers, diluents, and/or excipients, in some cases. In some embodiments, the composition further includes a pharmaceutically acceptable vehicle. In some embodiments, each of a buffer, stabilizing agent, salt, surfactant, preservative, and excipient are included in the compositions. In other embodiments, any one or more of a buffer, stabilizing agent, salt, surfactant, preservative, excipient, carrier, diluent, or vehicle may be excluded from compositions.

Examples of salts include but not limited to sodium salts and/or potassium salts. In some embodiments, the sodium salt comprises sodium chloride. In some embodiments, the potassium salt comprises potassium chloride. The concentration of the salts in the composition can be about 70 mM to about 140 mM. For example, the salt concentration can be equal to any one of, at least any one of, at most any one of, or between any two of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM, or any range or value derivable therein. In specific embodiments, the salt concentration is 70 mM. In specific embodiments, the salt concentration is 140 mM. The salt can be at a neutral pH, pH 6.5 to 8.5, pH 7.0 to pH 8.0, or pH 7.2 to pH 7.6. For example, the salt can be at a pH equal to any one of, at least any one of, at most any one of, or between any two of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein.

Examples of excipients, which refer to ingredients in the compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, or colorants. Preservatives for use in the compositions disclosed herein include but are not limited to benzalkonium chloride, chlorobutanol, paraben and thimerosal. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato; and celluloses such as microcrystalline cellulose. The amount of diluent in the composition can range from about 10% to about 90% by weight of the total composition, about 25% to about 75%, about 30% to about 60% by weight, or about 12% to about 60%.

The pH and exact concentration of the various components in a pharmaceutical composition are adjusted according to well-known parameters. The use of such media and agents for pharmaceutical active substances is well known in the art. Except insofar as any conventional media or agent is incompatible with the active ingredients, its use in immunogenic and therapeutic compositions is contemplated.

Administration of the compositions described herein can be carried out via any of the accepted modes of administration of agents for serving similar utilities. Pharmaceutical compositions may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection, or infusion techniques. Pharmaceutical compositions described herein are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. The composition to be administered will, in any event, contain a therapeutically effective amount of a compound within the scope of this disclosure, or a pharmaceutically acceptable salt thereof, for treatment of a disease or condition of interest in accordance with the teachings described herein.

A pharmaceutical composition within the scope of this disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalator administration. When intended for oral administration, the pharmaceutical composition is preferably in either solid or liquid form, where semi-solid, semi-liquid, suspension, and gel forms are included within the forms considered herein as either solid or liquid. As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate, or orange flavoring; and a coloring agent. When the pharmaceutical composition is in the form of a capsule, for example, a gelatin capsule, it may contain, in addition to materials of the above type, a liquid carrier such as polyethylene glycol or oil. The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant, and flavor enhancer. In a composition intended to be administered by injection, one or more of a surfactant, preservative, wetting agent, dispersing agent, suspending agent, buffer, stabilizer, and isotonic agent may be included or exclude.

A liquid pharmaceutical composition, whether they be solutions, suspensions, or other like form, may include or exclude one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation can be enclosed in ampoules, disposable syringes, or multiple dose vials made of glass or plastic. Physiological saline is a preferred adjuvant. An injectable pharmaceutical composition is preferably sterile.

A liquid pharmaceutical composition intended for either parenteral or oral administration should contain an amount of a compound such that a suitable dosage will be obtained.

The pharmaceutical compositions may be prepared by methodology well known in the pharmaceutical art. For example, a pharmaceutical composition intended to be administered by injection can be prepared by combining the RNA and amino acid(s) with sterile, distilled water or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

The compositions within the scope of the disclosure are administered in a therapeutically effective amount, which will vary depending upon a variety of factors including the activity of the specific therapeutic agent employed; the metabolic stability and length of action of the therapeutic agent; the age, body weight, general health, gender, and diet of the patient; the mode and time of administration; the rate of excretion; the drug combination; the severity of the particular disorder or condition; and the subject undergoing therapy.

### XI. Methods of Use

In one aspect, the disclosure relates to a method of protecting an isolated RNA from degradation by contacting the isolated RNA with at least one free amino acid. The method includes the steps of contacting the isolated RNA with at least one free amino acid as described herein. In some embodiments, the isolated RNA is encapsulated in a capsule, and the method includes the steps of contacting the capsule encapsulating the isolated RNA with at least one free amino acid as described herein. In some instances, the RNA molecule is comprised in any one of the compositions as described herein. In some instances, the at least one free amino acid is comprised in any one of the compositions as described herein. In some instances, the composition includes both the RNA molecule as described herein and the at least one free amino acid as described herein. The method can be a method to protect the RNA, which may be encapsulated, in an amount that is, when measured under identical conditions, greater than a method that does not comprise contacting the RNA molecule, whether or not encapsulated, with at least one free amino acid.

In one aspect, the present disclosure relates to a method of protecting RNA or encapsulated RNA such that the method can influence and/or dictate physical (e.g., stability), chemical (e.g., nucleic acid stability), and/or biological (e.g. efficacy, intracellular delivery, immunogenicity) properties of the RNA or encapsulated RNA or a composition thereof. In some embodiments, the methods of the present disclosure mitigate an undesired property change from the produced RNA and/or compositions thereof. In some embodiments, the methods of the present disclosure mitigate an undesired property change from the produced RNA and/or compositions thereof as compared to a RNA or composition thereof produced by a comparable method (e.g., a method without one or more of the steps as disclosed herein).

In some embodiments, the undesired property change is caused by a stress upon the RNA compositions or RNA therein. In some embodiments, the stress is induced during producing, purifying, packing, storing, transporting, and/or using the RNA and/or compositions thereof. In some embodiments, the stress is heat, shear, excessive agitation, membrane concentration polarization (change in charge state), dehydration, freezing stress, drying stress, stress due to crystallization of excipients during freezing, drying or storage, freeze/thaw stress, and/or nebulization stress. In some embodiments, the stress is induced during freezing or lyophilizing encapsulated RNA.

In some embodiments, the undesired property change is a reduction of the physical stability of the RNA and/or compositions thereof. In some embodiments, the methods of the present disclosure mitigate a reduction of the physical stability (e.g., an increase in the degradation of the RNA) from the RNA and/or compositions thereof as compared to RNA and/or compositions thereof produced by comparable methods to those disclosed herein that do not utilize at least one free amino acid.

In some embodiments, the undesired property change is a reduction of the chemical stability of the RNA and/or compositions thereof. In some embodiments, the undesired property change is a reduction of the integrity of the RNA in the compositions. In some embodiments, the undesired property change is a reduction of the biological property of the RNA and/or compositions thereof. In some embodiments, the undesired property change is a reduction of efficacy, intracellular delivery, and/or immunogenicity of the RNA and/or compositions thereof.

In some embodiments, the RNA and/or compositions thereof produced by the methods of the present disclosure have an efficacy, intracellular delivery, and/or immunogenicity higher than the efficacy, intracellular delivery, and/or immunogenicity of RNA and/or compositions thereof produced by comparable methods to those disclosed herein that do not utilize at least one free amino acid.

In some embodiments, the RNA and/or compositions thereof produced by the methods of the present disclosure have an efficacy, intracellular delivery, and/or immunogenicity being higher than the efficacy, intracellular delivery, and/or immunogenicity of RNA and/or compositions thereof produced by a comparable method that does not utilize at least one free amino acid by equal to any one of, at least any one of, at most any one of, or between any two of about 5% or higher, about 10% or more, about 15% or more, about 20% or more, about 30% or more, about 40% or more, about 50% or more, about 60% or more, about 70% or more, about 80% or more, about 90% or more, about 1-fold or more, about 2-fold or more, about 3-fold or more, about 4-fold or more, about 5-fold or more, about 10-fold or more, about 20-fold or more, about 30-fold or more, about 40-fold or more, about 50-fold or more, about 100-fold or more, about 200-fold or more, about 300-fold or more, about 400-fold or more, about 500-fold or more, about 1000-fold or more, about 2000-fold or more, about 3000-fold or more, about 4000-fold or more, about 5000-fold or more, or about 10000-fold or more.

In some embodiments, the RNA and/or compositions thereof produced by the methods of the present disclosure exhibit a nucleic acid expression (e.g., the RNA expression) higher than the nucleic acid expression (e.g., the RNA expression) of RNA and/or compositions thereof produced by a comparable method that does not utilize at least one free amino acid. As used herein, “expression” of a nucleic acid refers to production of an RNA template from a DNA sequence (e.g., by transcription), processing of an RNA transcript (e.g., by splicing, editing, 5′ cap formation, and/or 3′ end formation), translation of an RNA into a polypeptide or protein, and/or post-translational modification of a polypeptide or protein.

In some embodiments, the RNA and/or compositions thereof produced by the methods of the present disclosure exhibit a nucleic acid expression (e.g., the RNA expression) higher than the nucleic acid expression (e.g., the RNA expression) of RNA and/or compositions thereof produced by a comparable method that does not utilize at least one free amino acid by equal to any one of, at least any one of, at most any one of, or between any two of about 5% or higher, about 10% or more, about 15% or more, about 20% or more, about 30% or more, about 40% or more, about 50% or more, about 60% or more, about 70% or more, about 80% or more, about 90% or more, about 1-fold or more, about 2-fold or more, about 3-fold or more, about 4-fold or more, about 5-fold or more, about 10-fold or more, about 20-fold or more, about 30-fold or more, about 40-fold or more, about 50-fold or more, about 100-fold or more, about 200-fold or more, about 300-fold or more, about 400-fold or more, about 500-fold or more, about 1000-fold or more, about 2000-fold or more, about 3000-fold or more, about 4000-fold or more, about 5000-fold or more, or about 10000-fold or more.

In some cases, protection of the RNA is assessed by measuring in vitro expression of the RNA, and contacting the RNA or encapsulated RNA with the at least one free amino acid increases in vitro expression of the RNA compared to in vitro expression of RNA or encapsulated RNA that has not been contacted with the at least one free amino acid. In some cases, in vitro expression of the RNA is increased at least equal to any one of, at least any one of, at most any one of, or between any two of about 20%, 21%, 22%, 23%, 24%, 25%, 26%, 27%, 28%, 29%, 30%, 32%, 33%, 34%, 35%, 36%, 37%, 38%, 39%, or 40%. In some cases, in vitro expression of the RNA is increased at least about 33%.

In some cases, protection of the RNA is assessed by measuring immunogenicity of the isolated RNA, and contacting the RNA or encapsulated RNA with the at least one free amino acid increases immunogenicity of the RNA, encapsulated RNA, or compositions thereof compared to immunogenicity of the RNA, encapsulated RNA, or compositions thereof that has not been contacted with the at least one free amino acid. In some cases, immunogenicity of the RNA, encapsulated RNA, or compositions thereof is increased at least equal to any one of, at least any one of, at most any one of, or between any two of about 15%, 16%, 17%, 18%, 19%, 20%, 21%, 22%, 23%, 24%, 25%, 26%, 27%, 28%, 29%, 30%, 32%, 33%, 34%, or 35%. In some cases, immunogenicity of the RNA, encapsulated RNA, or compositions thereof is increased at least about 25%.

The method can further include the steps of storing the RNA, which may be encapsulated, after contacting the RNA or capsule encapsulating the RNA with the at least one free amino acid. The RNA or encapsulated RNA may be stored for at least about 2 hours to 12 weeks. In some embodiments, the RNA or encapsulated RNA is stored for equal to any one of, at least any one of, at most any one of, or between any two of at least about 2 hours, 4 hours to 8 weeks, 6 hours to seven weeks, 12 hours, 1 day, 2 days, 3 days, 4 days, 5 days, 6 days, 7 days, 2 weeks, 3 weeks, four weeks, five weeks, six weeks, seven weeks, 8 weeks, 9 weeks, 10 weeks, 11 weeks, 12 weeks, or any range or value derivable therein. The RNA or encapsulated RNA may be stored at a temperature of about room temperature to about −90° C. For example, the RNA or encapsulated RNA may be stored at a temperature below room temperature, at or below 4° C., at or below 0° C., at or below −20° C., at or below −60° C., at or below −70° C., at or below −80° C., or at or below −90° C. In some embodiments, the RNA or encapsulated RNA is stored at a temperature of equal to any one of, at least any one of, at most any one of, or between any two of about 20° C., 15° C., 10° C., 5° C., 0° C., −10° C., −20° C., −30° C., −40° C., −50° C., −60° C., −70° C., −80° C., or −90° C.

In another aspect, the disclosure relates to a method of expressing a polypeptide in a mammalian cell. The method includes the steps of administering to the mammalian cell a composition as described herein. In some instances, the composition includes a RNA molecule as described herein. In some instances, the composition includes at least one free amino acid as described herein. In some instances, the composition includes a RNA molecule as described herein and at least one free amino acid as described herein.

The method can be a method to protect the RNA of the composition in an amount that is, when measured under identical conditions, greater than a method that comprises administering to the mammalian cell a composition that includes the RNA molecule in the absence of the at least one free amino acid. The method can be a method to increase the immunogenicity of the RNA, the composition comprising the RNA, and/or the polypeptide of interest expressed from the RNA in an amount that is, when measured under identical conditions, greater than a method that comprises administering to the mammalian cell a composition that includes a RNA molecule but that does not include at least one free amino acid as described herein.

In another aspect, the disclosure relates to a method of inducing an immune response in a subject. The method includes administering to the mammalian cell an effective amount of a composition as described herein.

In another aspect, the disclosure relates to a method of vaccinating a subject. The method includes administering to the subject in need thereof an effective amount of a composition described herein.

In another aspect, the disclosure relates to a method of treating or preventing an infectious disease. The method includes administering to the subject an effective amount of a composition as described herein.

In another aspect, disclosure relates to a method of treating or preventing an infectious disease in a subject by, for example, inducing an immune response to an infectious disease in the subject. In some embodiments, the method includes administering a priming composition that includes an effective amount of a composition described herein, and administering a booster composition including an effective amount of an adenoviral vector encoding an antigen. In another embodiment, the method includes administering a priming composition including an effective amount of an adenoviral vector encoding an antigen, and administering a booster composition that includes an effective amount of a composition described herein. In some embodiments, the composition elicits an immune response including an antibody response. In some embodiments, the composition elicits an immune response including a T cell response.

### EXAMPLES

The following examples are included to demonstrate embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

To identify amino acids that showed utility in protecting RNA from degradation, several functional assays were used with multiple RNA constructs. All functional assays were performed under conditions that accelerate RNA degradation; notably incubation of RNA in basic pH buffer at high temperature over time. These assays were performed with amino acids and compared to a no-degradation baseline control to gauge degree of protection imparted by the amino acid. Multiple functional assay approaches were used where differing endpoints were measured including a droplet digital PCR (ddPCR) assay where intact RNA sequence was quantitated, a size exclusion base hydrolysis degradation assay that used a size exclusion column to determine the degree of degradation occurring to an mRNA construct, and finally, a TapeStation assay that utilizes capillary electrophoresis and optical detection to measure intact RNA. Amino acids that showed protection may be further characterized using assays that assess binding to demonstrate direct interaction of the amino acid binding to RNA. Affinity selection by mass spectrometry (ASMS), native electrospray ionization mass spectrometry (nESI-MS) and nuclear magnetic resonance (NMR) may be used as several complementary approaches to ascertain compound binding to RNA. The latter techniques utilize MS to measure mass of bound compound alone (ASMS), or mass of RNA and bound compound (nESI-MS). The nESI-MS and NMR studies may be conducted to provide direct analytical quantification of resulting complex via measurement of mass (nESI-MS) or chemical shifts (NMR). The ASMS experiment is conducted to determine saturation binding of the compound to RNA where resulting apparent binding constants (K_{D,App}) may be generated. Further descriptions of the functional and binding assays are provided in the section below.

A. TapeStation Assay

The Agilent 4150 TapeStation bioanalyzer is an automated platform for electrophoresis of nucleic acids. In this case, the system was used to measure the integrity of RNA samples after various degradation conditions and show protection from degradation by compounds. In principle, samples are loaded to the instrument where electrophoresis separates component RNA's on the basis of mass, and subsequently analyzes these optically as they travel through the capillary.

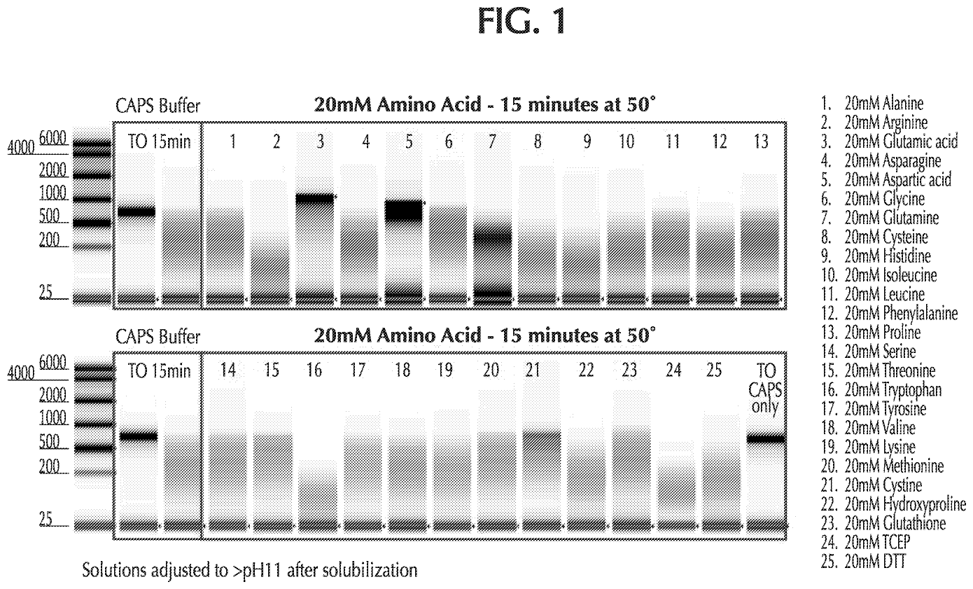

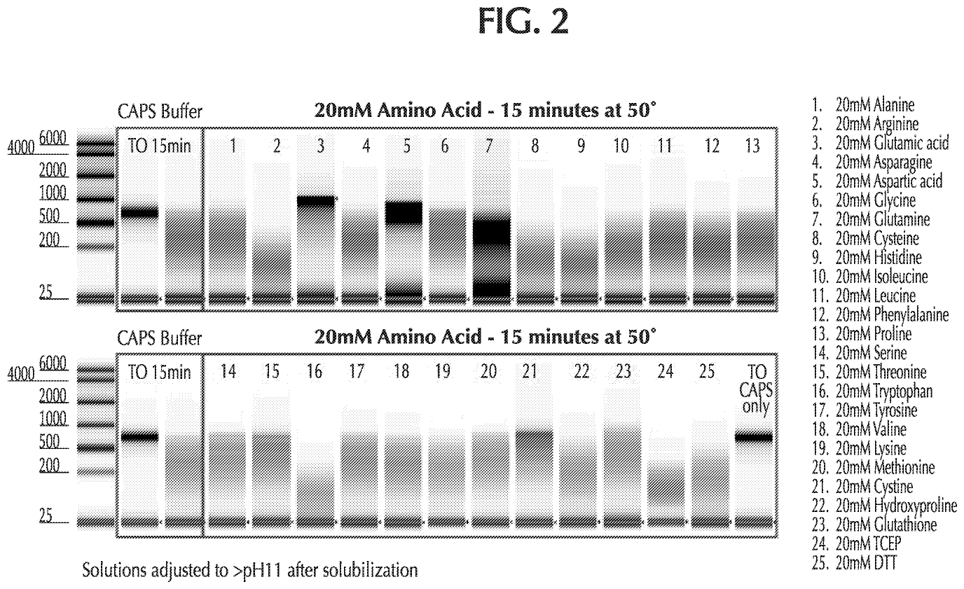

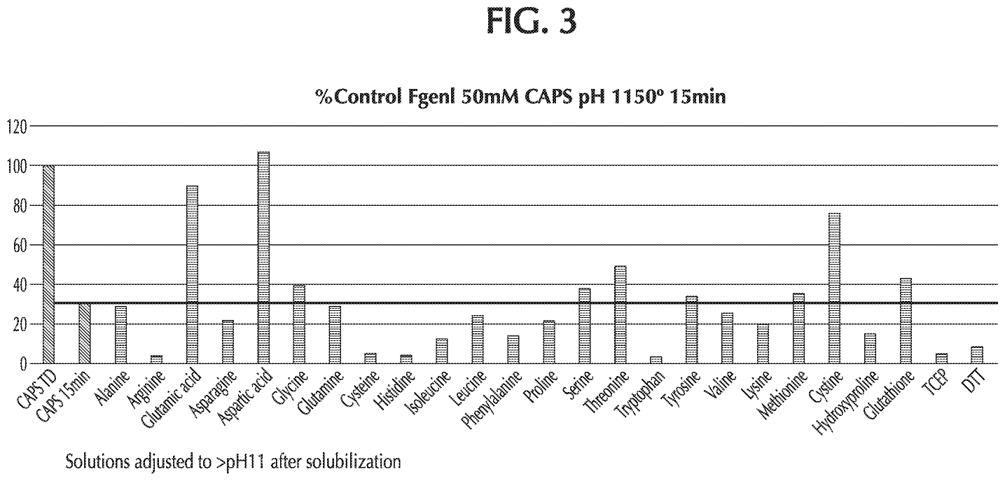

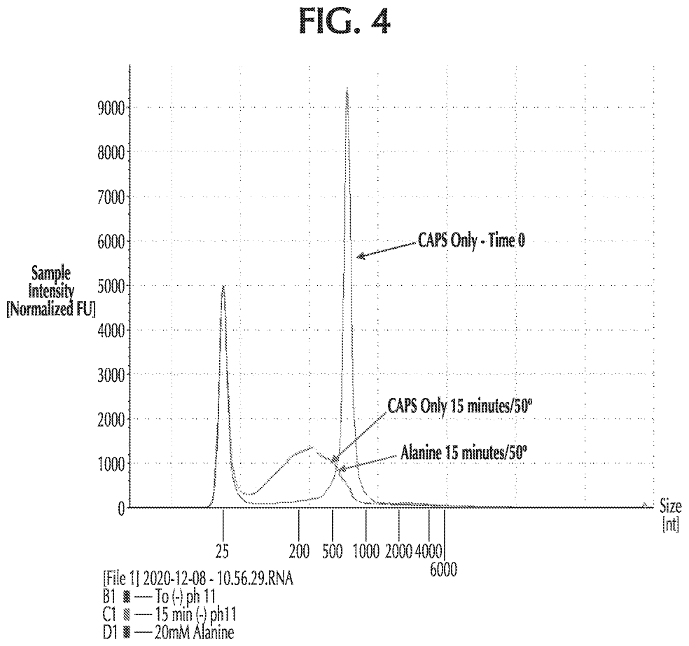

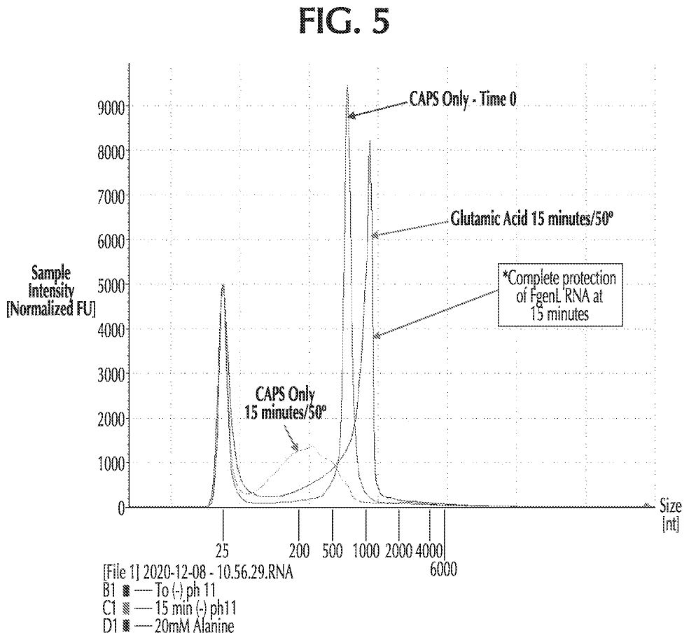

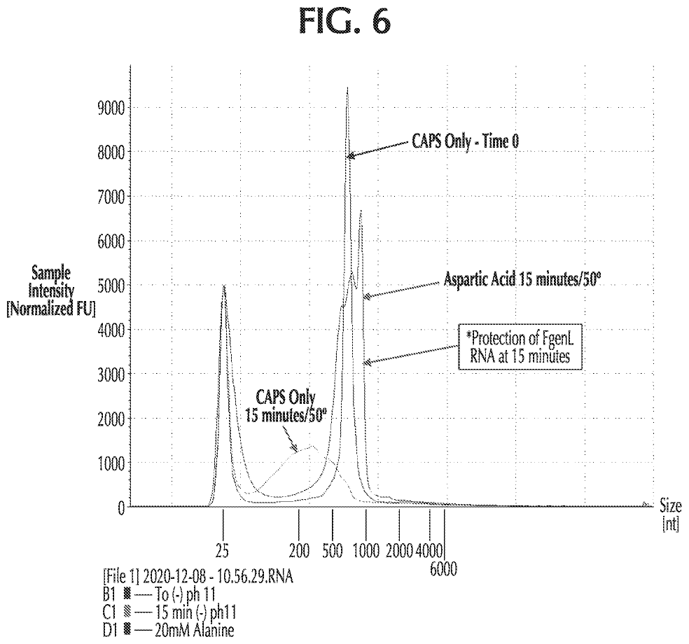

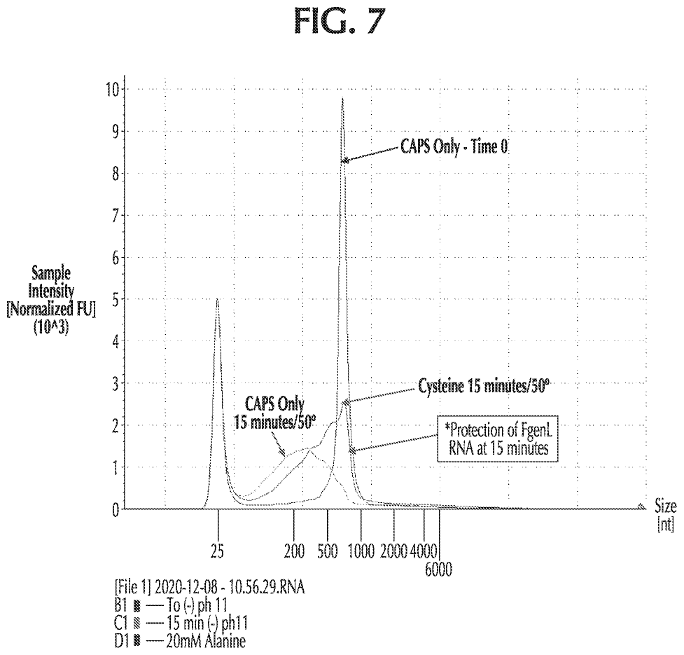

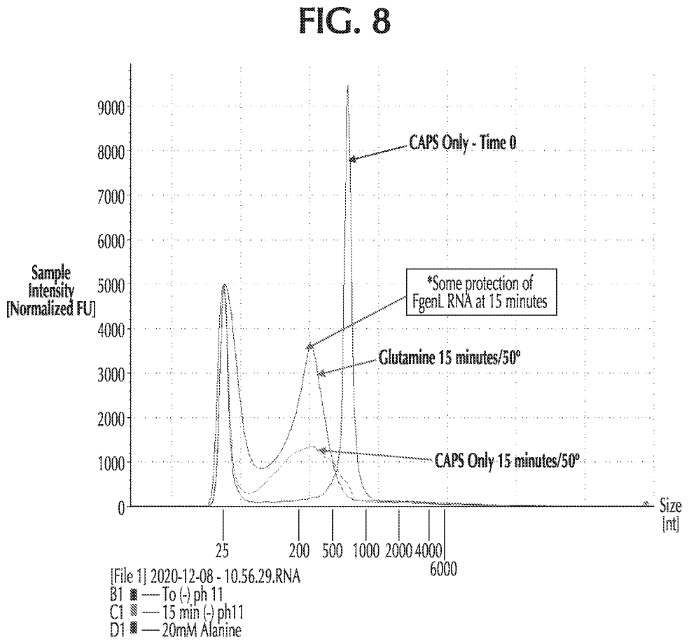

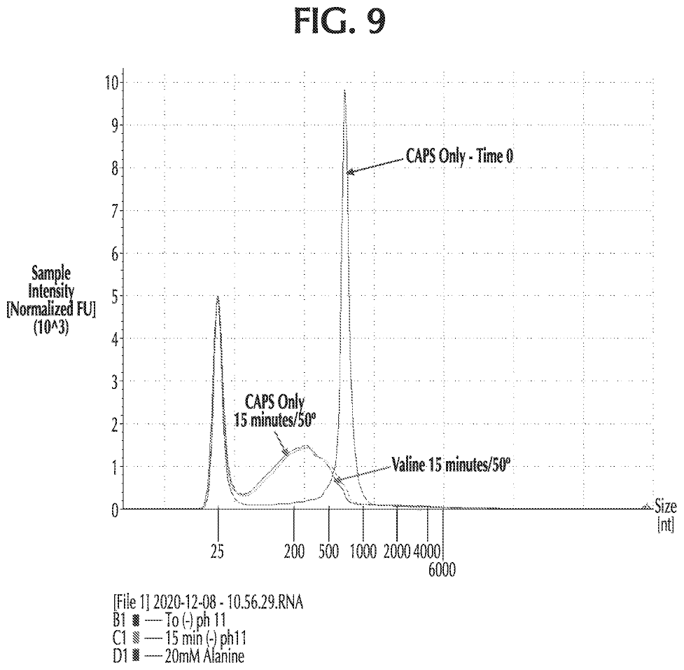

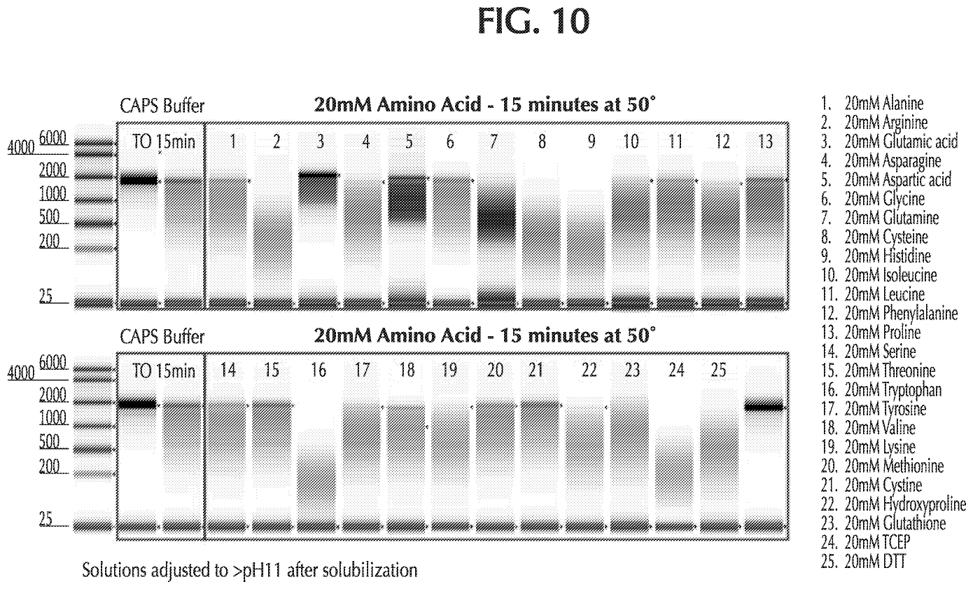

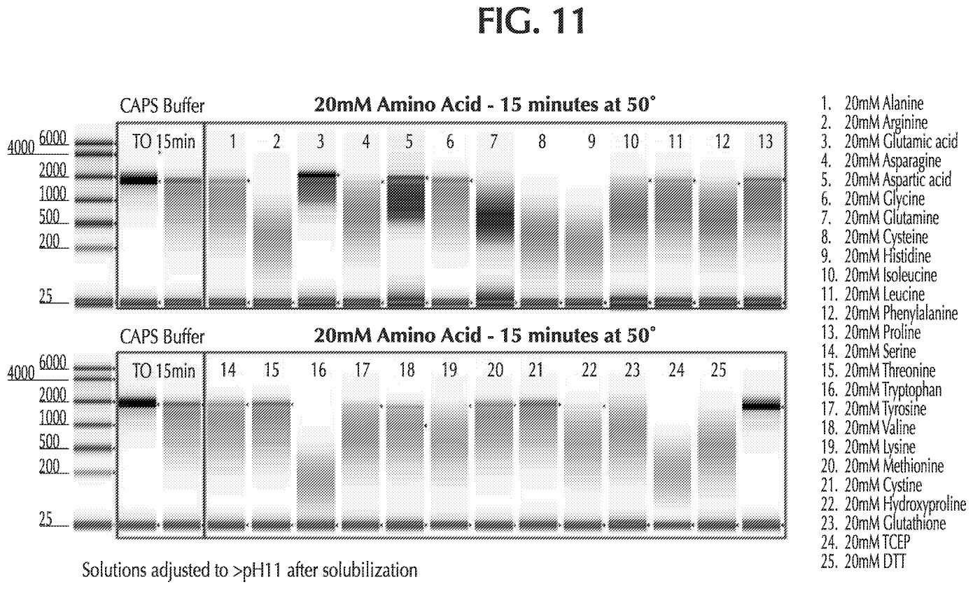

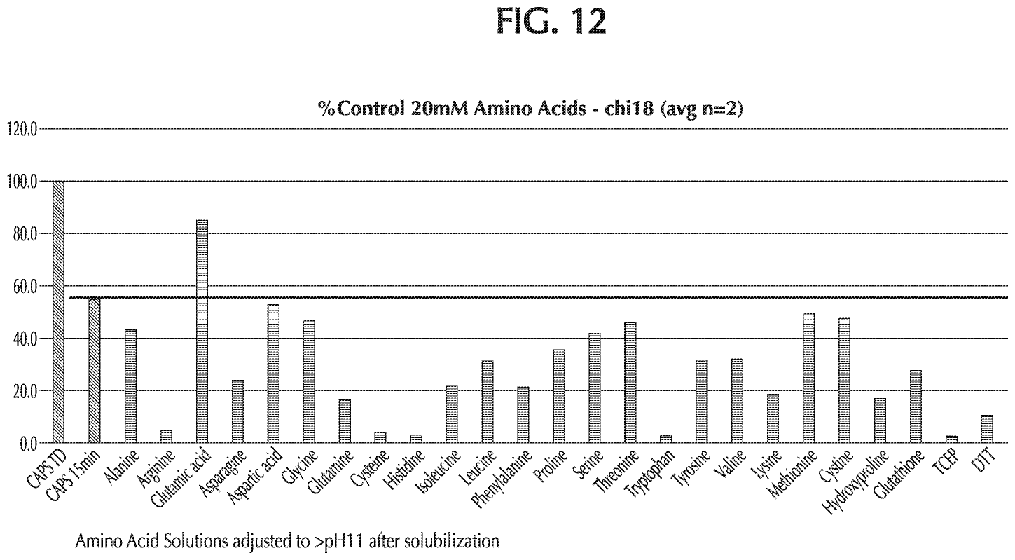

RNA samples were diluted to the appropriate concentration to fall within the linear range of detection for the RNA ScreenTape Assay. Model Chi18 or FgenL RNAs were diluted to 50 ng/μL in 50 mM CAPS buffer pH 11. Amino acid stocks were made up in 50 mM CAPS pH 11 and re-adjusted to >pH 11 after solubilization. Amino acid stocks were diluted to 250 mM in 50 mM CAPS pH 11, then diluted 1:10+RNA for a final concentration of 25 mM. The samples were aliquoted to PCR tubes for each time point (Time 0 or 15 minutes). Degradation reaction: Samples were incubated at 50° C. for 0 to 15 minutes in a thermocycler, removed at the appropriate time point and placed on ice. Time 0 samples remained on ice. Samples were prepared for the TapeStation according to the RNA ScreenTape Quick Guide and run on the Agilent 4150 TapeStation using the RNA ScreenTape Assay. Using the TapeStation software, a region representing the intact RNA size (e.g. 600-1000 nt for FgenL) was set to quantitate the starting (Time 0) and degraded (15 mm/50° C. RNA concentrations, and the results represented as the % Control (e.g. [degraded concentration/Time 01*100). Representative data from the TapeStation Assay are shown in FIGS. 1 to 12, and in Tables 2 and 3 below.

TABLE 2
20 mM amino acids in 50 mM CAPS pH 11 with FgenL model RNA

| | Conc. | Conc. | % Control | | |
|---|---|---|---|---|---|
| | FgenL#1 | FgenL#2 | N = 1 | N = 2 | Average |
| CAPS T0 | 34.5 | 34.7 | 100.0 | 100.0 | 100.0 |
| CAPS 15 min | 10.3 | 7.75 | 29.9 | 22.3 | 26.1 |
| Alanine | 9.93 | 8.05 | 28.8 | 23.2 | \|26.0 |
| Arginine | 1.46 | 1.15 | 4.2 | 3.3 | 3.8 |
| Glutamic acid | 31 | 26.4 | 89.9 | 76.1 | 83.0 |
| Asparagine | 7.56 | 5.19 | 21.9 | 15.0 | 18.4 |
| Aspartic acid | 36.9 | 31.6 | 107.0 | 91.1 | 99.0 |
| Glycine | 13.6 | 12.9 | 39.4 | 37.2 | 38.3 |
| Glutamine | 10 | 3.57 | 29.0 | 10.3 | 19.6 |
| Cysteine | 1.7 | 2.05 | 4.9 | 5.9 | 5.4 |
| Histidine | 1.46 | 1.3 | 4.2 | 3.7 | 4.0 |
| Isoleucine | 4.22 | 3.28 | 12.2 | 9.5 | 10.8 |
| Leucine | 8.37 | 5.85 | 24.3 | 16.9 | 20.6 |
| Phenylalanine | 4.76 | 2.4 | 13.8 | 6.9 | 10.4 |
| Proline | 7.52 | 7.03 | 21.8 | 20.3 | 21.0 |
| Serine | 13 | 10.9 | 37.7 | 31.4 | 34.5 |
| Threonine | 17 | 12 | 49.3 | 34.6 | 41.9 |
| Tryptophan | 1.21 | 0.51 | 3.5 | 1.5 | 2.5 |
| Tyrosine | 11.7 | 7.59 | 33.9 | 21.9 | 27.9 |
| Valine | 8.77 | 6.38 | 25.4 | 18.4 | 21.9 |
| Lysine | 6.85 | 6.12 | 19.9 | 17.6 | 18.7 |
| Methionine | 12.2 | 11.3 | 35.4 | 32.6 | 34.0 |
| Cystine | 26.2 | 22.4 | 75.9 | 64.6 | 70.2 |
| Hydroxyproline | 5.12 | 3.73 | 14.8 | 10.7 | 12.8 |
| Glutathione | 14.8 | 12.4 | 42.9 | 35.7 | 39.3 |
| TCEP | 1.71 | 1.38 | 5.0 | 4.0 | 4.5 |
| DTT | 2.86 | 3.13 | 8.3 | 9.0 | 8.7 |

TABLE 3
20 mM amino acids in 50 mM CAPS pH 11 with Chi18 model RNA

| | Conc. | Conc. | % Control | | |
|---|---|---|---|---|---|
| | chi18#1 | chi18#2 | N = 1 | N = 2 | Avg. |
| CAPS T0 | 53.3 | 52.9 | 100.0 | 100.0 | 100.0 |
| CAPS 15 min | 22.6 | 35.7 | 42.4 | 67.5 | 54.9 |
| Alanine | 18.8 | 27.2 | 35.3 | 51.4 | 43.3 |
| Arginine | 2.78 | 2.46 | 5.2 | 4.7 | 4.9 |
| Glutamic acid | 40.5 | 50 | 76.0 | 94.5 | 85.3 |
| Asparagine | 11.2 | 14.1 | 21.0 | 26.7 | 23.8 |
| Aspartic acid | 28 | 28.3 | 52.5 | 53.5 | 53.0 |
| Glycine | 22.4 | 27.2 | 42.0 | 51.4 | 46.7 |
| Glutamine | 8.46 | 9.42 | 15.9 | 17.8 | 16.8 |
| Cysteine | 2.39 | 1.85 | 4.5 | 3.5 | 4.0 |
| Histidine | 1.58 | 1.68 | 3.0 | 3.2 | 3.1 |
| Isoleucine | 10.3 | 12.8 | 19.3 | 24.2 | 21.8 |
| Leucine | 16.4 | 16.9 | 30.8 | 31.9 | 31.4 |
| Phenylalanine | 9.19 | 13.4 | 17.2 | 25.3 | 21.3 |
| Proline | 19.7 | 18.3 | 37.0 | 34.6 | 35.8 |
| Serine | 22.5 | | 42.2 | 42.2 | 42.2 |
| Threonine | 27.4 | 21.6 | 51.4 | 40.8 | 46.1 |
| Tryptophan | 1.57 | | 2.9 | 2.9 | 2.9 |
| Tyrosine | 17 | | 31.9 | 31.9 | 31.9 |
| Valine | 17.3 | | 32.5 | 32.5 | 32.5 |
| Lysine | 11.7 | 8.18 | 22.0 | 15.5 | 18.7 |
| Methionine | 26.5 | | 49.7 | 49.7 | 49.7 |
| Cystine | 28.3 | 22.6 | 53.1 | 42.7 | 47.9 |
| Hydroxyproline | 9.2 | | 17.3 | 17.3 | 17.3 |
| Glutathione | 15.6 | 14.1 | 29.3 | 26.7 | 28.0 |
| TCEP | 1.41 | | 2.6 | 2.6 | 2.6 |
| DTT | 5.68 | | 10.7 | 10.7 | 10.7 |

B. Size Exclusion Base Hydrolysis Degradation Assay

Waters H-Class UPLC system with bioinert components, fitted with a Sepax SRT-500 500 Å 5 μm 4.6×150 mm size exclusion column was used to determine the degree of degradation occurring to an mRNA construct when incubated with a small molecule protector (in this case an amino acid) under specific conditions, and hence the degree of stabilization of the mRNA construct.

Basic assay procedure: RNA samples were diluted to 1.0 mg/mL in DNAase/RNAase free water. Amino acids were dissolved in water to a stock solution of 100 mM and a solution of 500 mM CAPS pH 11 was used. The two mRNA constructs tested were FgenL and Chi18. Each individual sample volume was 50 μL. To determine the amount of 1 M NaOH needed to adjust the final pH to 11.0 after the addition of all sample components, the sample volume was increased 100 fold (5 mL), the volume of 1 M NaOH recorded and this value was translated back down to the 50 μL sample volume. Each individual sample consisted of the following final concentrations: mRNA 0.1 mg/mL, 25 mM Amino acid, 50 mM CAPS, adjusted to pH 11 with 1M NaOH (made up to 50 μL with water).

The mRNA and amino acid were allowed to incubate for one hour prior to the addition of CAPS pH 11. All steps of the assay were conducted at room temperature.

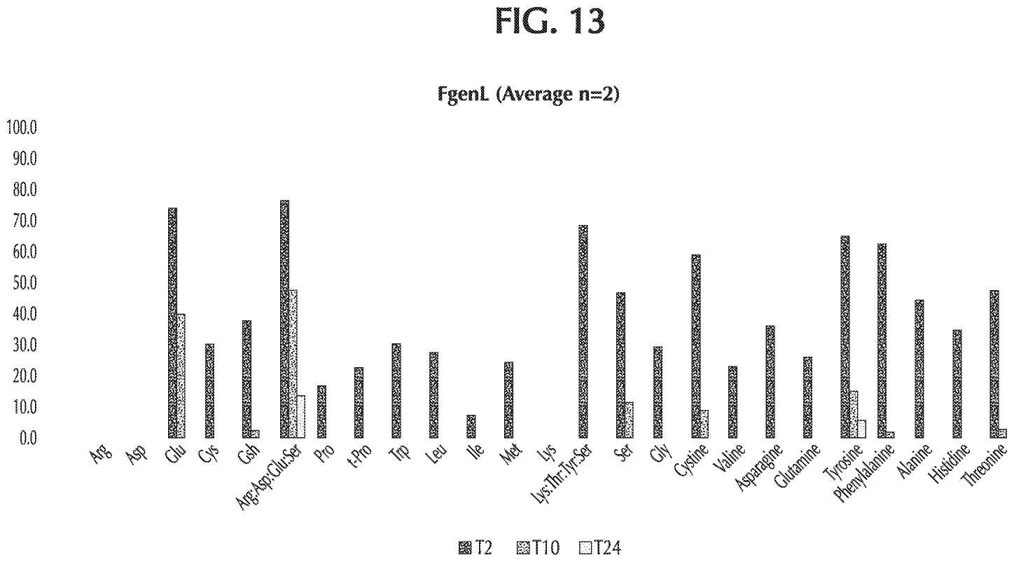

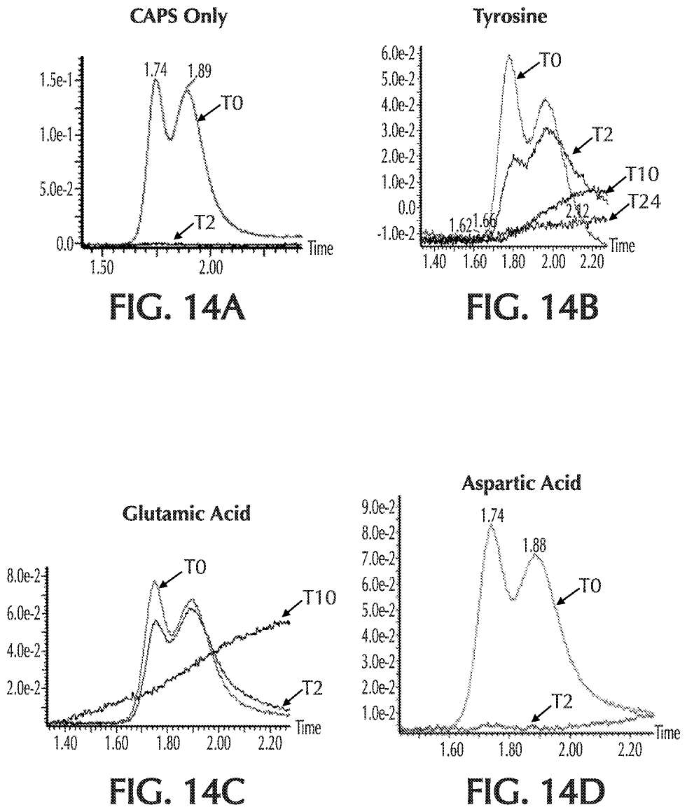

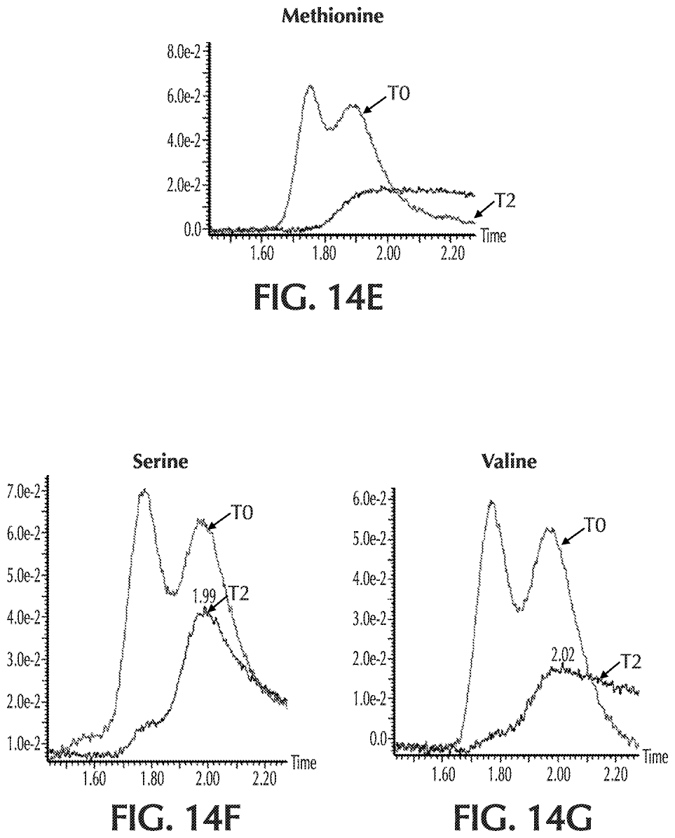

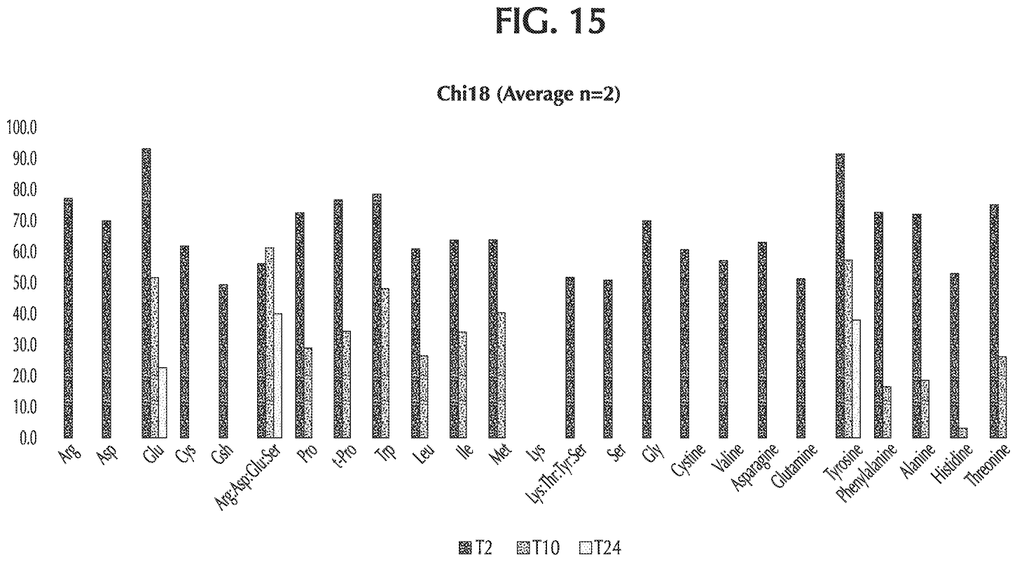

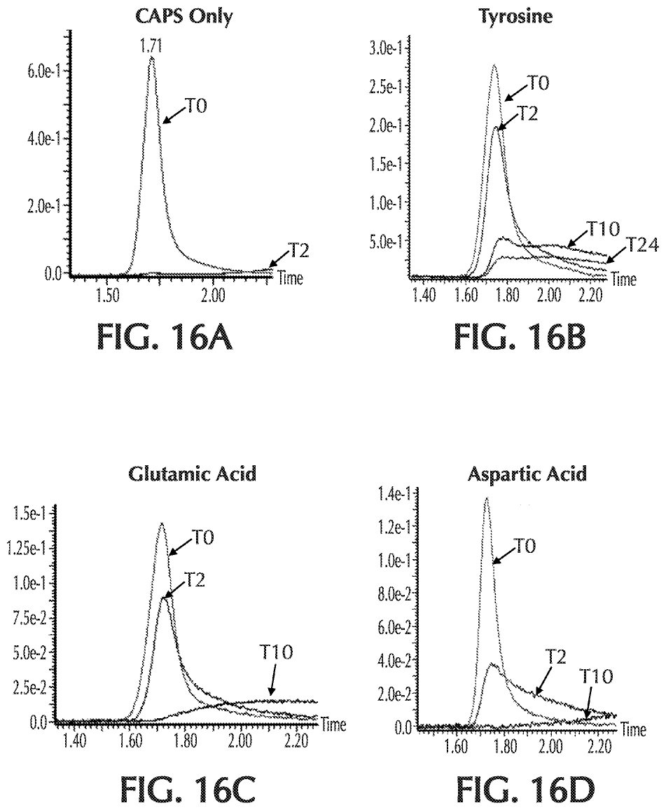

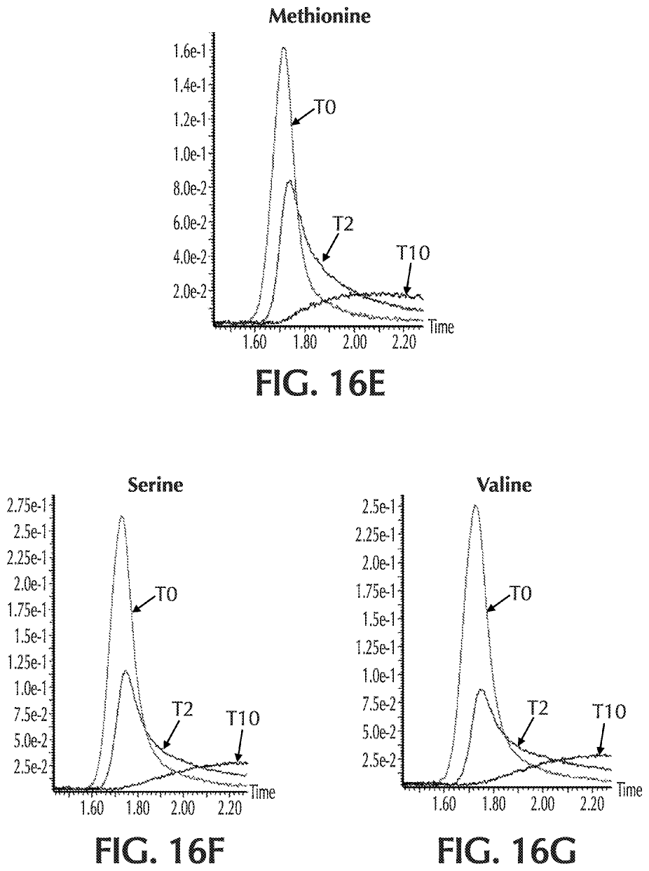

Degradation reaction: After the CAPS addition, the samples were injected onto the Waters H-Class UPLC system to record the first timepoint, T=O. This resulting chromatogram was processed by integrating the area of absorbance at 250 nm for the mRNA construct. Immediately following this timepoint, the pH was adjusted to 11. Subsequent time points (2 hrs, 10 hrs, and 24 hrs) recorded the Absorbance Units (peak area) at 250 nm as a percentage of the peak area determined at T=O. Integration of the chromatogram for subsequent timepoints was conducted by analyzing the area of absorbance at 250 nm contained within the same time window in which the peak eluted in T=0 for the 100% intact mRNA. Representative data for this assay are shown in FIGS. 13 to 16, and in Tables 4 and 5, below.

TABLE 4
Size Exclusion Base Hydrolysis Assay - FgenL Raw Data

| Amino | T0 | | T2 | | T10 | | T24 | | Amino | T0 | T2 | T10 | T24 |
|---|---|---|---|---|---|---|---|---|---|---|---|---|---|
| Acid | R1 | R2 | R1 | R2 | R1 | R2 | R1 | R2 | Acid | Avg | Avg | Avg | Avg |
| Arg | 14624 | 15089 | 0 | 0 | 0 | 0 | 0 | 0 | Arg | 100.0 | 0.0 | 0.0 | 0.0 |
| Asp | 17497 | 17759 | 0 | 0 | 0 | 0 | 0 | 0 | Asp | 100.0 | 0.0 | 0.0 | 0.0 |
| Glu | 14529 | 15463 | 10330 | 11820 | 8449 | 3450 | 0 | 0 | Glu | 100.0 | 73.9 | 39.7 | 0.0 |
| Cys | 18041 | 14850 | 6085 | 3823 | 0 | 0 | 0 | 0 | Cys | 100.0 | 30.1 | 0.0 | 0.0 |
| Glutathione | 16850 | 17517 | 4627 | 22 | 0 | 786 | 0 | 0 | Glutathione | 100.0 | 37.7 | 2.3 | 0.0 |
| Arg:Asp:Glu:Ser | 13375 | 15976 | 13239 | 9159 | 8989 | 4970 | 3952 | 0 | Arg:Asp:Glu:Ser | 100.0 | 76.3 | 47.6 | 13.5 |
| Pro | 17907 | 13985 | 3380 | 1928 | 0 | 0 | 0 | 0 | Pro | 100.0 | 16.6 | 0.0 | 0.0 |
| t-Pro | 16626 | 15223 | 5048 | 2094 | 0 | 0 | 0 | 0 | t-Pro | 100.0 | 22.4 | 0.0 | 0.0 |
| Trp | 15191 | 11700 | 4991 | 3138 | 0 | 0 | 0 | 0 | Trp | 100.0 | 30.2 | 0.0 | 0.0 |
| Leu | 13599 | 13125 | 4569 | 2727 | 0 | 0 | 0 | 0 | Leu | 100.0 | 27.3 | 0.0 | 0.0 |
| Ile | 14903 | 8515 | 1687 | 0 | 0 | 0 | 0 | 0 | Ile | 100.0 | 7.2 | 0.0 | 0.0 |
| Met | 15213 | 14794 | 3658 | 3601 | 0 | 0 | 0 | 0 | Met | 100.0 | 24.2 | 0.0 | 0.0 |
| Lys | 17400 | 18213 | 0 | 0 | 0 | 0 | 0 | 0 | Lys | 100.0 | 0.0 | 0.0 | 0.0 |
| Lys:Thr:Tyr:Ser | 15539 | | 10622 | | 0 | | 0 | | Lys:Thr:Tyr:Ser | 100.0 | 68.4 | 0.0 | 0.0 |
| Ser | 17044 | 14971 | 7928 | 6982 | 0 | 3606 | 0 | 0 | Ser | 100.0 | 46.6 | 11.3 | 0.0 |
| Gly | 18897 | 17170 | 3657 | 6895 | 0 | 0 | 0 | 0 | Gly | 100.0 | 29.3 | 0.0 | 0.0 |
| Cys | 23851 | 22663 | 17633 | 9702 | 3767 | 328 | 0 | 0 | Cys | 100.0 | 58.8 | 8.8 | 0.0 |
| Val | 17745 | 19832 | 4305 | 4266 | 0 | 0 | 0 | 0 | Val | 100.0 | 22.8 | 0.0 | 0.0 |
| Asn | 14320 | 17262 | 6791 | 4553 | 0 | 0 | 0 | 0 | Asn | 100.0 | 35.9 | 0.0 | 0.0 |
| Gln | 15510 | 18175 | 5779 | 2962 | 0 | 0 | 0 | 0 | Gln | 100.0 | 25.9 | 0.0 | 0.0 |
| Tyr | 22082 | 19139 | 13039 | 13633 | 1912 | 4267 | 912 | 1402 | Tyr | 100.0 | 64.7 | 15.0 | 5.6 |
| Phe | 18152 | 17936 | 12662 | 9788 | 605 | 0 | 0 | 0 | Phe | 100.0 | 62.2 | 1.7 | 0.0 |
| Ala | 17478 | 17419 | 7051 | 8399 | 0 | 0 | 0 | 0 | Ala | 100.0 | 44.3 | 0.0 | 0.0 |
| His | 18949 | 19416 | 8325 | 4903 | 0 | 0 | 0 | 0 | His | 100.0 | 34.5 | 0.0 | 0.0 |
| Thr | 17711 | 17633 | 8436 | 8292 | 904 | 0 | 0 | 0 | Thr | 100.0 | 47.3 | 2.6 | 0.0 |

TABLE 5
Size Exclusion Base Hydrolysis Assay - Chi18 Raw Data

| Amino | T0 | | T2 | | T10 | | T24 | | Amino | T0 | T2 | T10 | T24 |
|---|---|---|---|---|---|---|---|---|---|---|---|---|---|
| Acid | R1 | R2 | R1 | R2 | R1 | R2 | R1 | R2 | Acid | Avg | Avg | Avg | Avg |
| Arg | 20698 | 14916 | 15655 | 11667 | 0 | 0 | 0 | 0 | Arg | 100.0 | 76.7 | 0.0 | 0.0 |
| Asp | 11079 | 14735 | 7643 | 10278 | 0 | 0 | 0 | 0 | Asp | 100.0 | 69.4 | 0.0 | 0.0 |
| Glu | 19221 | 16192 | 16514 | 16342 | 6324 | 11807 | 0 | 7890 | Glu | 100.0 | 92.8 | 151.2 | 22.3 |
| Cys | 20358 | 15096 | 14715 | 7054 | 0 | 0 | 0 | 0 | Cys | 100.0 | 61.4 | 0.0 | 0.0 |
| Glutathione | 13157 | 21375 | 7747 | 9070 | 0 | 0 | 0 | 0 | Glutathione | 100.0 | 48.7 | 0.0 | 0.0 |
| Arg:Asp:Glu:Ser | 16515 | 15545 | 7933 | 9897 | 15540 | 3993 | 12780 | 0 | Arg:Asp:Glu:Ser | 100.0 | 55.6 | 60.9 | 39.9 |
| Pro | 12033 | 15095 | 7330 | 12208 | 2712 | 5024 | 0 | 0 | Pro | 100.0 | 72.0 | 28.5 | 0.0 |
| t-Pro | 13659 | 26735 | 8540 | 22235 | 3280 | 10531 | 0 | 0 | t-Pro | 100.0 | 76.2 | 34.2 | 0.0 |
| Trp | 8974 | 20722 | 7000 | 16175 | 3243 | 10924 | 0 | 0 | Trp | 100.0 | 78.0 | 47.7 | 0.0 |
| Leu | 43979 | 15204 | 23370 | 12518 | 10474 | 5016 | 0 | 0 | Leu | 100.0 | 60.6 | 26.2 | 0.0 |
| Ile | 15361 | 29558 | 7533 | 20878 | 4699 | 10500 | 0 | 0 | Ile | 100.0 | 63.2 | 33.8 | 0.0 |
| Met | 17259 | 17101 | 8392 | 13421 | 6837 | 6929 | 0 | 0 | Met | 100.0 | 63.5 | 40.1 | 0.0 |
| Lys | 24122 | 22023 | 0 | 0 | 0 | 0 | 0 | 0 | Lys | 100.0 | 0.0 | 0.0 | 0.0 |
| Lys:Thr:Tyr:Ser | 35030 | | 17990 | | 0 | | 0 | | Lys:Thr:Tyr:Ser | 100.0 | 51.4 | 0.0 | 0.0 |
| Ser | 31778 | 53454 | 19639 | 23301 | 0 | 0 | 0 | 0 | Ser | 100.0 | 150.4 | 0.0 | 0.0 |
| Gly | 32571 | 24952 | 21950 | 18073 | 0 | 0 | 0 | 0 | Gly | 100.0 | 69.6 | 0.0 | 0.0 |
| Cys | 39458 | 23604 | 25387 | 12672 | 0 | 0 | 0 | 0 | Cys | 100.0 | 60.4 | 0.0 | 0.0 |
| Val | 29804 | 24568 | 12369 | 18453 | 0 | 0 | 0 | 0 | Val | 100.0 | 56.7 | 0.0 | 0.0 |
| Asn | 25494 | 24113 | 12115 | 18973 | 0 | 0 | 0 | 0 | Asn | 100.0 | 62.7 | 0.0 | 0.0 |
| Gln | 25301 | 34767 | 10030 | 20636 | 0 | 0 | 0 | 0 | Gln | 100.0 | 51.1 | 0.0 | 0.0 |
| Tyr | 21493 | 34065 | 22110 | 28401 | 14946 | 16624 | 10375 | 10525 | Tyr | 100.0 | 90.9 | 56.8 | 37.6 |
| Phe | 30537 | 18192 | 22175 | 12956 | 3615 | 4315 | 0 | 0 | Phe | 100.0 | 72.1 | 16.3 | 0.0 |
| Ala | 19606 | 13180 | 13211 | 10264 | 2730 | 3215 | 0 | 0 | Ala | 100.0 | 71.6 | 18.1 | 0.0 |
| His | 27170 | 21961 | 14612 | 11328 | 1391 | 0 | 0 | 0 | His | 100.0 | 152.8 | 2.8 | 0.0 |
| Thr | 39202 | 14708 | 28759 | 11473 | 10963 | 3030 | 0 | 0 | Thr | 100.0 | 74.6 | 26.0 | 0.0 |

C. Droplet Digital Polymerase Chain Reaction (ddPCR) Assay

The ddPCR assays measure RNA integrity by converting intact RNA to cDNA by reverse transcription, then performing digital PCR in thousands of nanoliter-sized droplets for each sample. PCR is performed within each droplet, which allows for more sensitive detection of subtle changes in RNA concentration compared to traditional PCR or qPCR. Additionally, primers/probes specific to the 5′ end of the RNA/cDNA transcript(s) are used to assess fragment integrity—e.g. degraded RNA will have fewer 5′ positive droplets. The BioRad QX200 Droplet Digital PCR System was used for the ddPCR workflow.

Basic assay procedure: RNA samples (Chi18 or FgenL fragments) were diluted to 25 fg/μL or 10 fg/μL, respectively, in 50 mM Tris buffer pH 11.0. Amino acid stocks were made up in 50 mM CAPS buffer, pH 11.0 and subsequently re-adjusted to pH 11.0, post solubilization. Amino acid stocks were tested in a dose-response format to determine EC50. An amino acid dilution plate containing a ˜300 mM (or appropriate) top-concentration of amino acid in 50 mM CAPS buffer was serially (half-log) diluted for 10 times. 0.6 μL of this compound dilution plate was spotted into a 96-well PCR plate (Thermo). Sample RNAs were added (6 μL) and incubated with test amino acid as described below. Final amino acid concentrations ranged from −30 mM to 0.949 μM.

In addition, each PCR plate also contained positive (non-degraded) and negative (degraded) control wells to define the upper and lower limits for the assay signal. These control wells did not contain test amino acid but instead received 6 μL of RNA samples treated with 50 mM CAPS buffer, pH 11 on the same day as the assay (100% protection, non-degraded control) or 6 μL of RNA samples treated with 50 mM CAPS buffer, pH 11 on the same day as compound treatment to define 0% protection, degraded control.

Degradation reaction: Treated RNAs were incubated for 16 hours at 30° C. for Chi18 samples or 44° C. for FgenL samples. Upon completion of the degradation reaction, cDNA was made from the intact RNA by Reverse Transcription and ddPCR run for each sample (96 well plates). Positive 5′ droplet values were divided by the total accepted droplet values for each sample and a ratio value generated for each sample.

Data was imported into the company database, where percent (%) protection and compound EC50 were calculated and curated. % protection was calculated by using the following formula: % protection=100×(ZPE−sample)/(ZPE−HPE) where ZPE (zero % protection) is the signal observed in wells that contain RNA with no amino acid degraded over night for 16 hours under conditions described previously, HPE (high percent effect) is the signal observed in wells that contain RNA that was not subjected to degradation conditions overnight, and ‘sample’ is the signal observed in wells that contain the indicated concentrations of amino acid. EC50 was determined by using a four parameter logistic equation: Y=A+((B−A)/(1+(C/X)D)); where Y is percent protection, X is compound concentration, A and B are minimum and maximum asymptotes, respectively, C is EC50, and D is Hill slope.

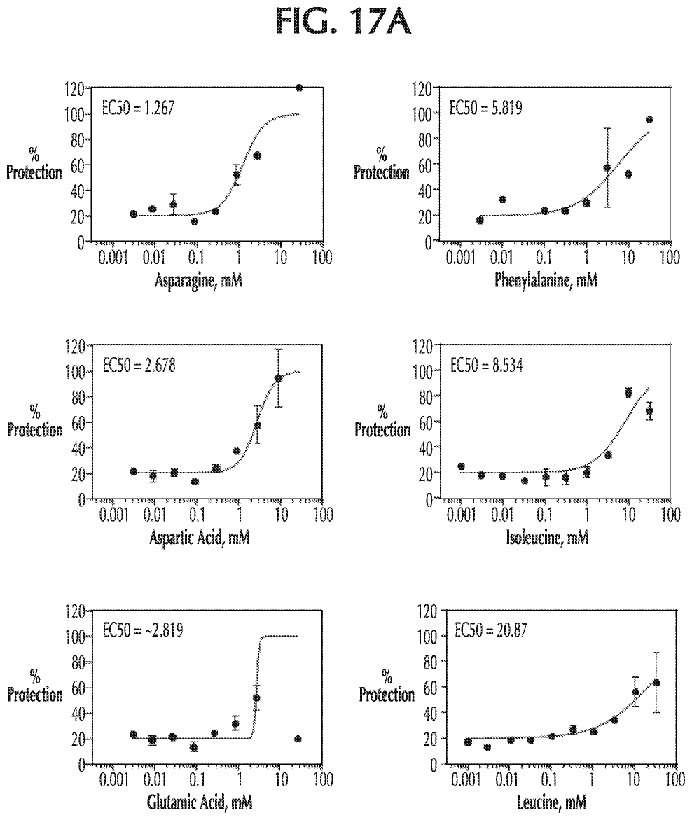

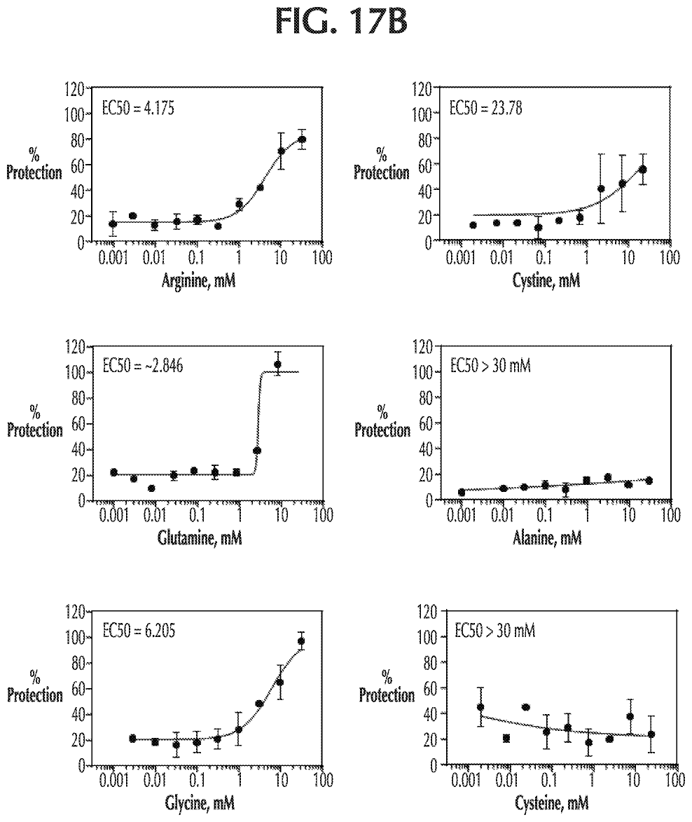

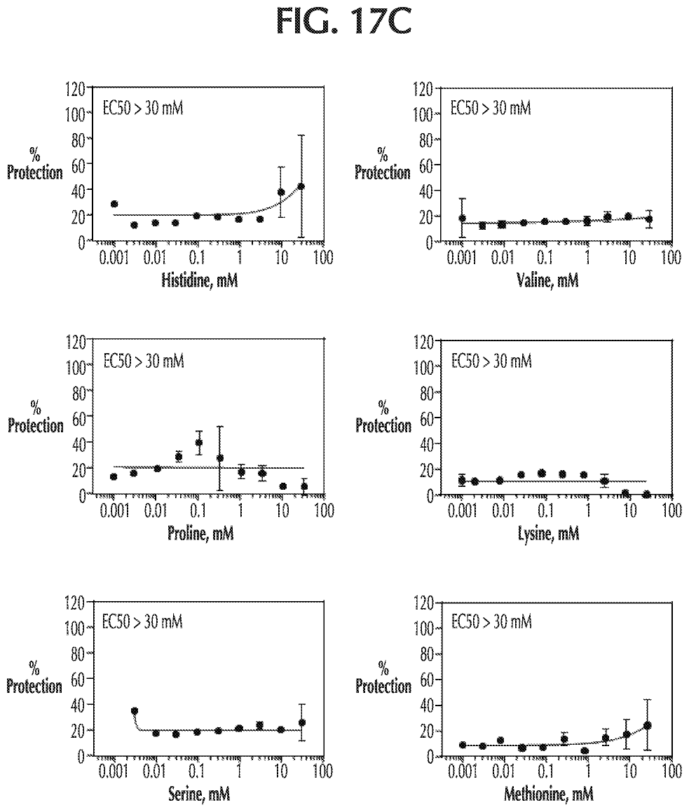

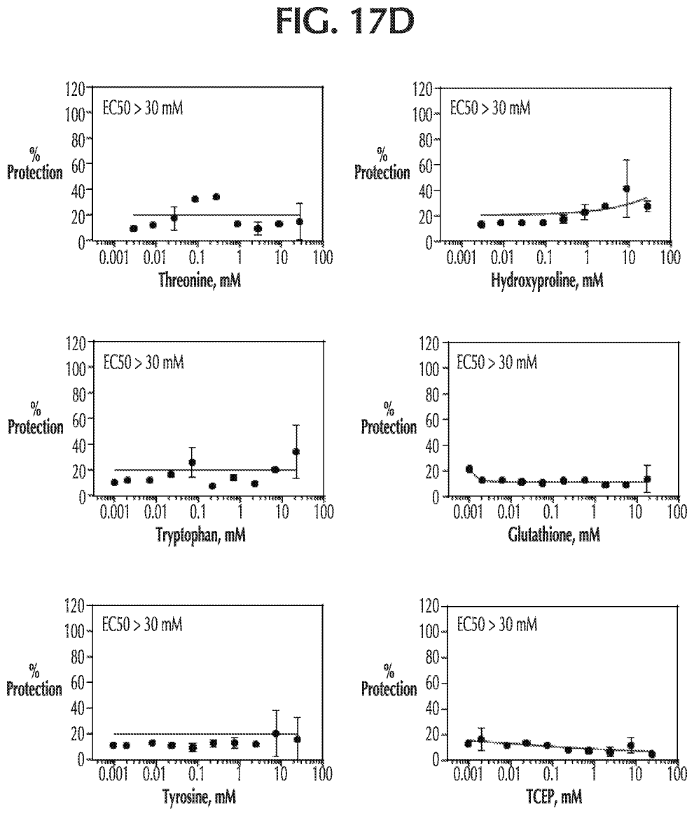

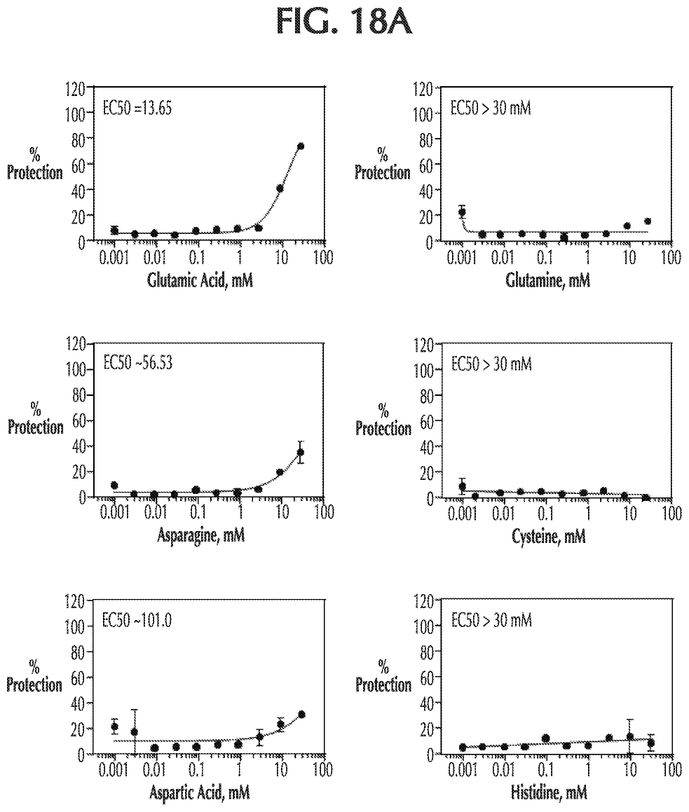

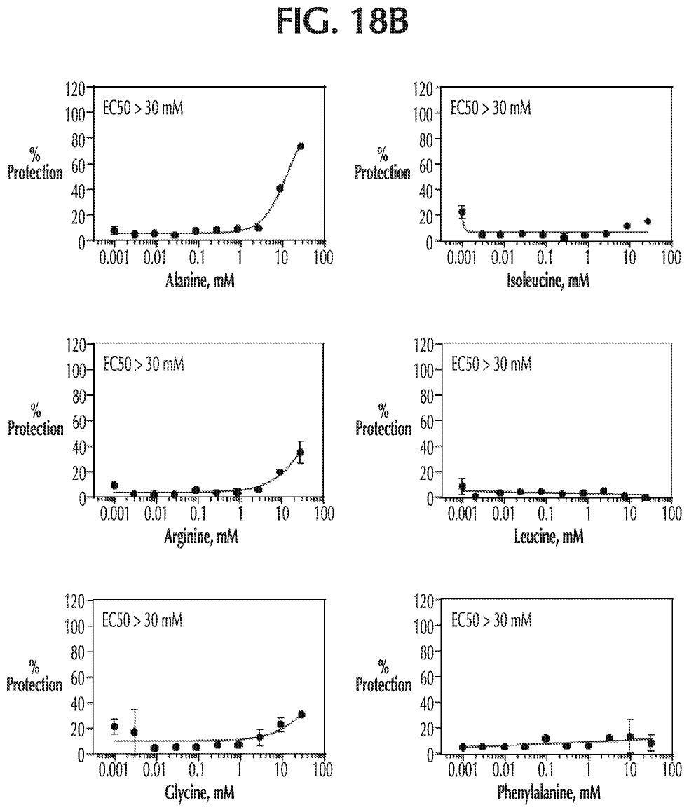

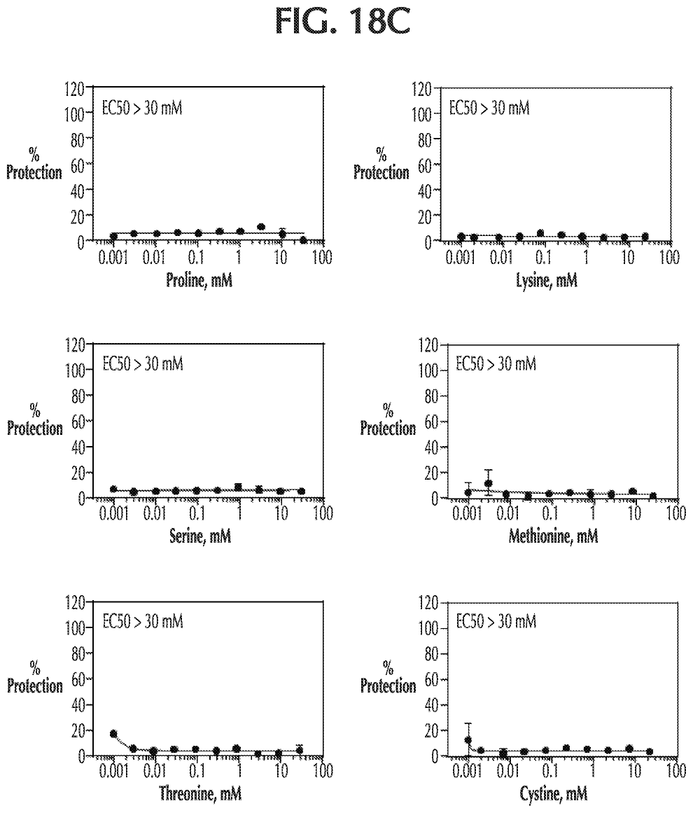

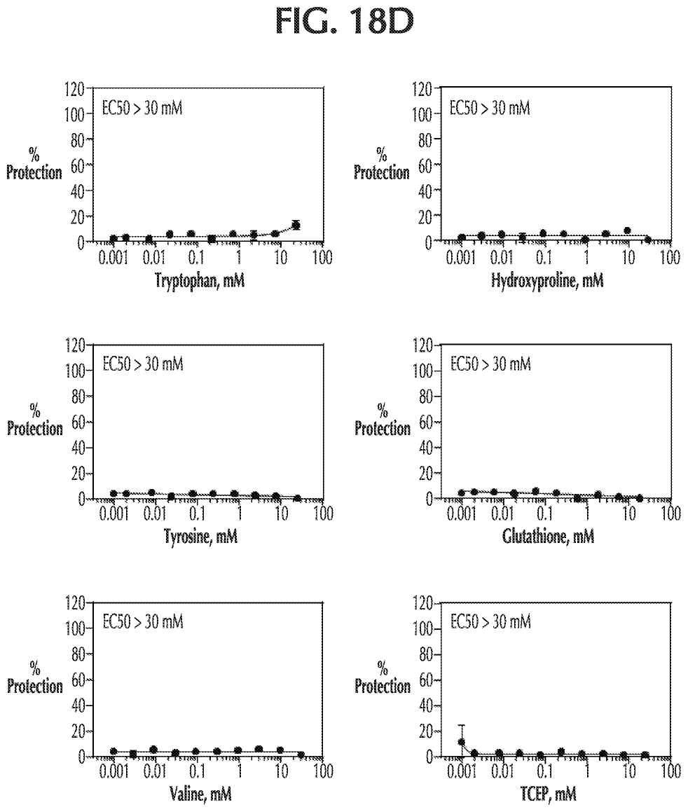

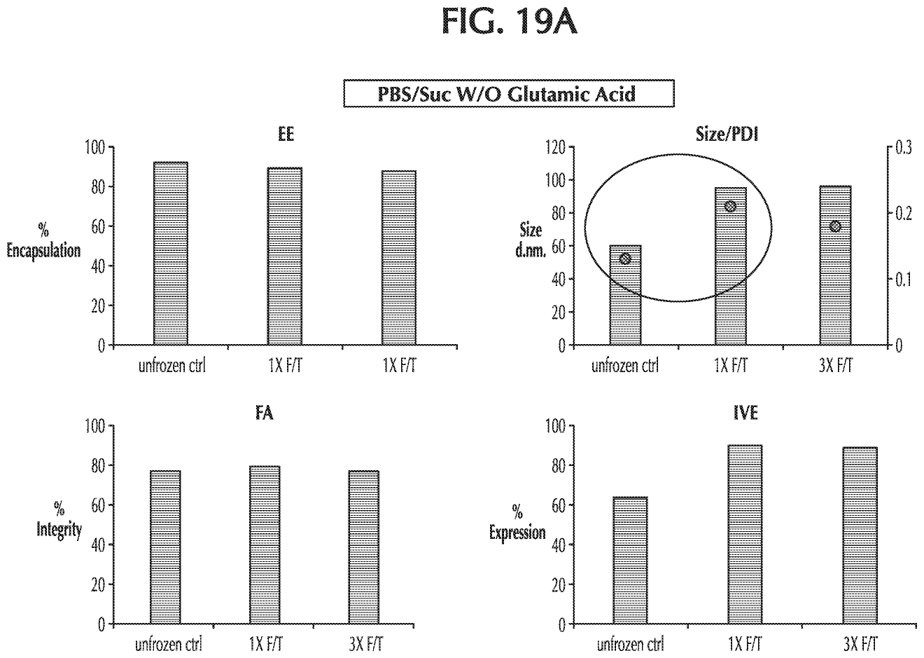

Representative data for this assay are shown in FIGS. 17 and 18, and in Table 6, below.

TABLE 6
Amino Acid ddPCR Summary: Average (n = 2) EC50 values for amino
acids tested with Chi18 and FgenL model RNAs.

| Test Sample | Chi18 Avg EC50 [mM] | FgenL Avg EC50 [mM] |
|---|---|---|
| Asparagine | 4.9 | 76 |
| Aspartic acid | 6.1 | 214 |
| Glutamic acid | 6.7 | 19 |
| Arginine | 7.6 | >30 |
| Glutamine | 8.8 | >30 |
| Glycine | 15 | >30 |
| Alanine | >30 | >30 |
| Cysteine | >30 | >30 |
| Histidine | >30 | >30 |
| Proline | >30 | >30 |
| Serine | >30 | >30 |
| Threonine | >30 | >30 |
| Tryptophan | >30 | >30 |
| Tyrosine | >30 | >30 |
| Valine | >30 | >30 |
| Lysine | >30 | >30 |
| Methionine | >30 | >30 |
| Hydroxyproline | >30 | >30 |
| Glutathione | >30 | >30 |
| TCEP | >30 | >30 |
| Phenylalanine | >18 | >30 |
| Isoleucine | >19 | >30 |
| Leucine | >25 | >30 |
| Cystine | >29 | >30 |

Note,
Avg EC50 = mean potency average across two separate experiments, n = 2; and > 30 indicates an EC50 greater than the top-concentration tested.

### Example 2

Current RNA vaccines have limited shelf life under normal refrigeration due to limitations on lipid nanoparticle physical stability and RNA chemical stability. In particular, these vaccines exhibit poor colloidal stability upon freeze/thaw cycles at RNA concentrations of less than 0.2 mg/mL, with LNP particles increasing in size and polydispersity index (PDI) and poor colloidal stability upon long-term storage at, for example, 5° C. As such, small molecules were screened for their ability to stabilize RNA as measured by analysis of RNA directly or through functional readouts like in vitro expression and in vivo immunogenicity.

Initial screening efforts focused on several benzoxaboroles and amino acid excipients as potential stabilizers. Many of these agents exhibited limited translation across various assays, and 20 mM glutamic acid was selected as an excipient with LNPs for the examples disclosed herein. The effect of glutamic acid on the in vitro expression and in vivo immunogenicity of self-amplifying VSV G (vesicular stomatitis virus G) self-amplifying RNA as a model antigen (see SEQ ID NO: 37 for the corresponding DNA sequence) encapsulated by LNP matrices utilizing alternative buffers was assessed. In vitro transcription of RNA is known in the art. According to the present disclosure, the manufacture of polynucleotides and/or parts or regions thereof may be accomplished utilizing the methods taught in International Publication WO2017053297, entitled “Compositions and Methods for Synthesizing 5-Capped RNAs,” the contents of which is incorporated herein by reference in its entirety. The LNP matrices tested included: 1) 0.75× PBS and Sucrose, 20 mM Glutamic Acid, pH 7.4; 2) 10 mM Tris and 10% Sucrose, 20 mM Glutamic Acid, pH 7.4; and 3) 10 mM HEPES/10% Sucrose, 20 mM Glutamic Acid, pH 7.4.

The VSV G self-amplifying RNA The VSV G self-amplifying RNA concentration was 0.06 mg/mL for all LNP formulations. An amino lipid was used as the functional cationic lipid component and contains a titratable tertiary amino head group linked via ester bonds to two saturated alkyl chains which, when incorporated in LNP, confer distinct physicochemical properties that regulate particle formation, cellular uptake, fusogenicity and endosomal release of the RNA. N/P=6 for all LNP formulations.

Exemplary LNP Formulation. Lipid nanoparticles, cationic lipids and polymer conjugated lipids (PEG-lipid) were prepared and tested. Lipid nanoparticle (LNP)-formulated mRNA was prepared using an ionizable amino lipid (cationic lipid), phospholipid, cholesterol and a PEGylated lipid. Cationic lipid, DSPC, cholesterol and PEG-lipid were solubilized in ethanol at a molar ratio of approximately 47.5:10:40.8:1.7. LNPs for the Examples included, for example, cationic lipid compound III-3 and the foregoing components. Lipid nanoparticle particle diameter size was 60-90 nm.

A. In Vitro Expression

The LNP formulations were tested for in vitro protein expression (IVE). Expression of VSV G protein from LNP formulations was measured using a characterization assay. First, the RNA-containing lipid nanoparticles (LNPs) were added to a HEK-293T cell monolayer (˜4×10⁵ cells) in a single well of a 12-well plate at a non-saturating RNA dose level of 10 ng per well.

Protein expression was measured using an anti-VSV G antibody. Prior to analysis by flow cytometry, cells were labeled with a Live/Dead dye, and to eliminate background signal, the percentage of live cells expressing the protein was enumerated. Expression was measured by quantifying the number of cells having a positive signal for bound anti-VSV G antibody.

As shown below in Table 7, glutamic acid improved in vitro expression (IVE) of the Tris/Sucrose LNP formulation. In particular, IVE of the Tris/Sucrose LNP formulation increased from 42% to 70% upon inclusion of glutamic acid in the formulation. No significant changes were observed for RNA concentration, encapsulation efficiency (EE), integrity (fragment analyzer, FA), and size/PDI. All other attributes including lipid concentration, N/P ratio, pH, residual ethanol were as expected.

TABLE 7
Physical and Functional Characteristics of LNP Formulations

| | RNA | | | Size | IVE |
|---|---|---|---|---|---|
| | Conc. | EE | FA | (nm)/ | (%) |
| LNP matrix | (mg/mL) | % | % | PDI | (10 ng) |
| 0.75X PBS/10% Suc, | 60 | ≥92 | 77 | 60/0.13 | 64 |
| pH 7.4 | | | | | |
| 0.75X PBS/10% Suc, | 51 | ≥90 | 76 | 61/0.14 | 64 |
| pH 7.4 + | | | | | |
| 20 mM Glutamic Acid | | | | | |
| 10 mM Tris/10% Suc, | 60 | ≥92 | 71 | 62/0.14 | 42 |
| pH 7.4 | | | | | |
| 10 mM Tris/10% Suc, | 60 | ≥92 | 73 | 61/0.12 | 70 |
| pH 7.4 + | | | | | |
| 20 mM Glutamic Acid | | | | | |
| 10 mM HEPES/10% | 55 | ≥91 | 70 | 70/0.12 | 92 |
| Suc, pH 7.4 | | | | | |
| 10 mM HEPES/10% Suc, | 64 | ≥92 | 72 | 64/0.09 | 90 |
| pH 7.4 + | | | | | |
| 20 mM Glutamic Acid | | | | | |

B. LNP Particle Size

The chemical and physical characteristics of the LNP formulations were also tested after one or more freeze/thaw cycles as compared to unfrozen controls. Characteristics measured include in vitro expression (IVE), encapsulation efficiency (EE), integrity (fragment analyzer, FA), and size/PDI. The LNP formulations were subject to either one freeze/thaw cycle or three freeze/thaw cycles from −70° C. to ambient temperature.

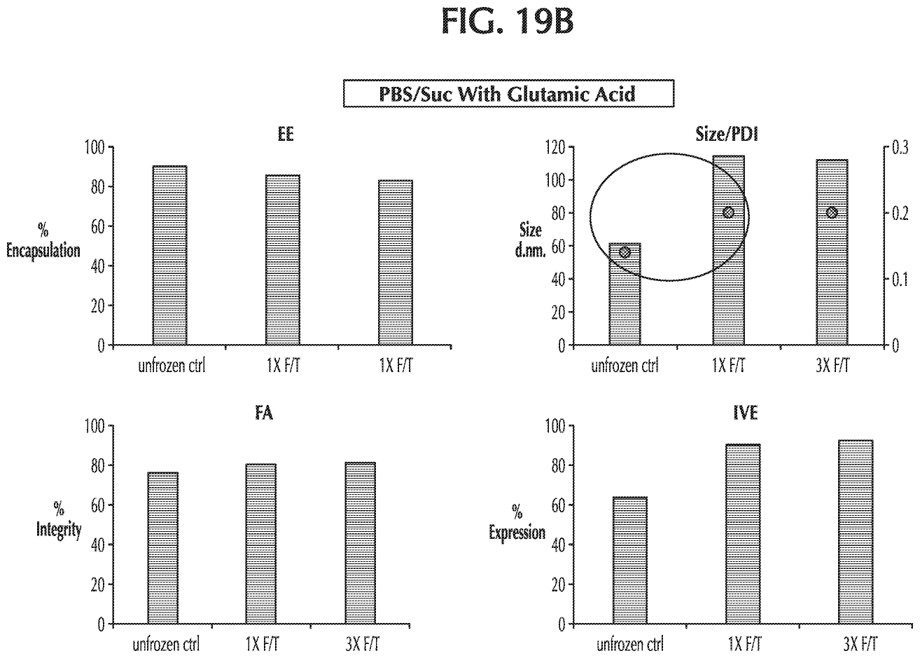

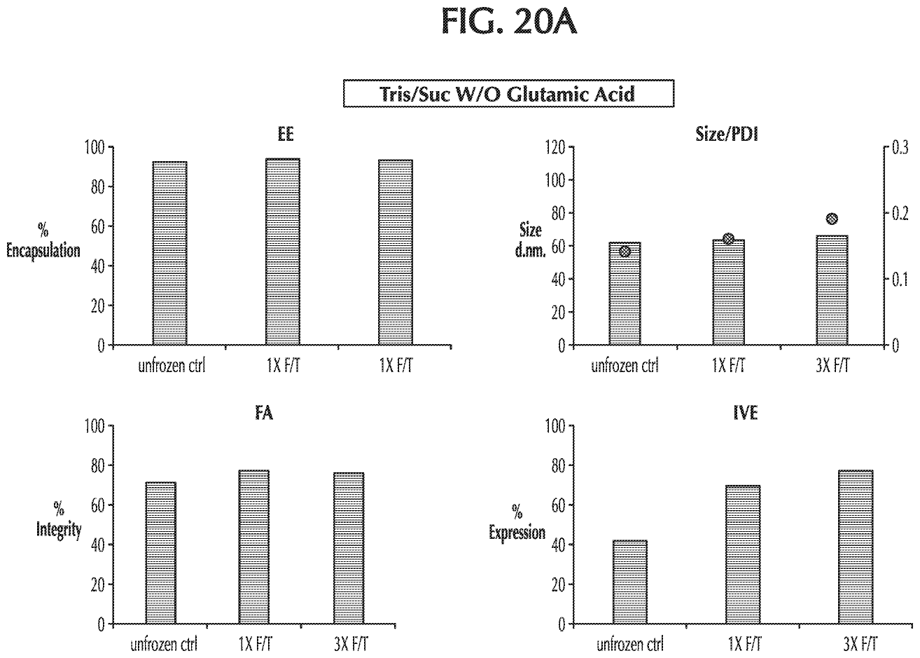

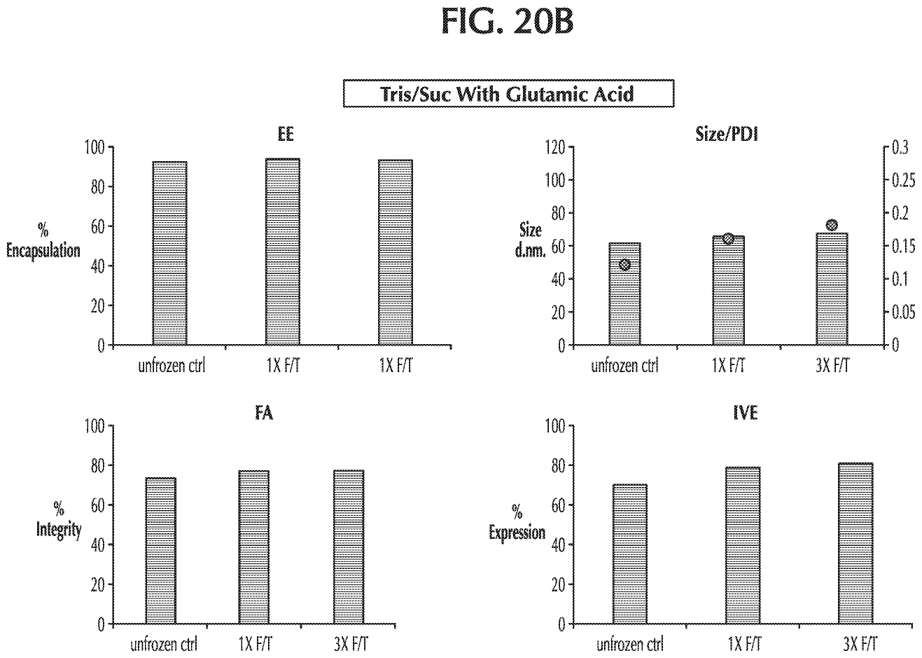

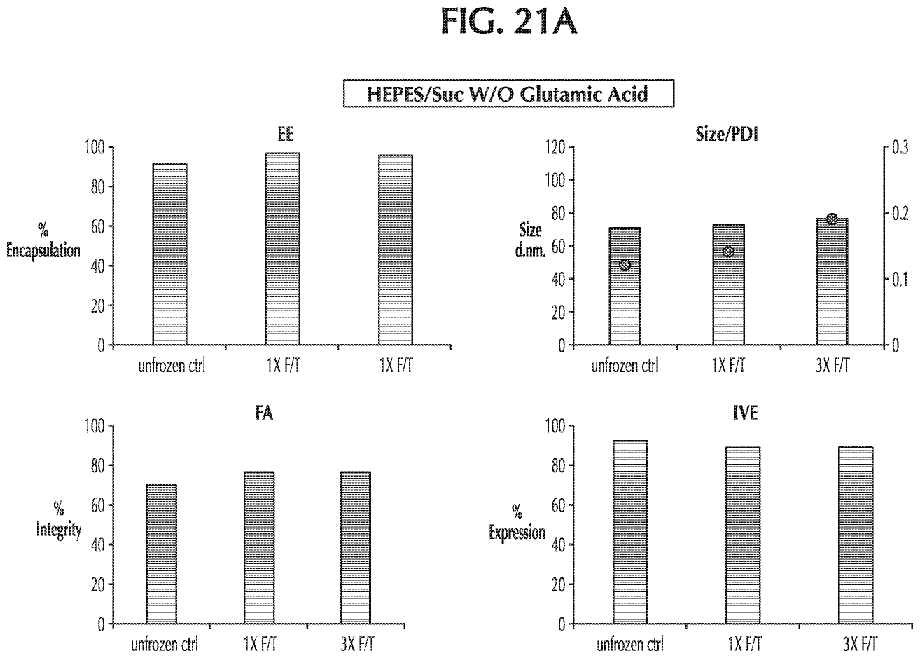

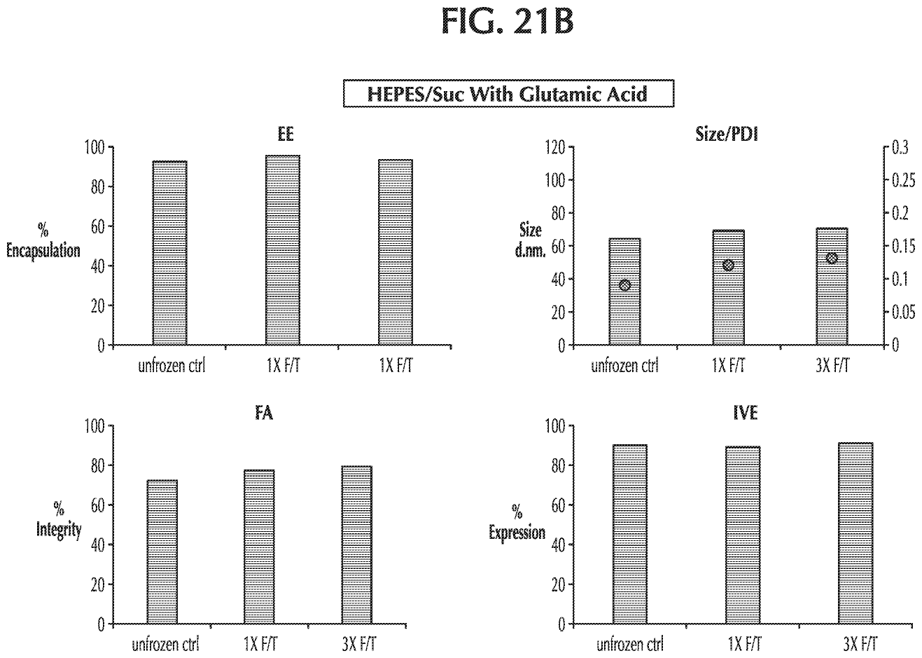

As shown in FIG. 19, freeze/thaw leads to LNP particle size increase in PBS/Sucrose±20 mM Glutamic Acid formulation but has no significant impact on other attributes. As shown in FIG. 20, freeze/thaw results in negligible impact on LNP attributes for the Tris/Sucrose±20 mM Glutamic Acid formulation. Finally, as shown in FIG. 21, freeze/thaw results in negligible impact on LNP attributes for the HEPES/Sucrose±20 mM Glutamic Acid formulation.

C. LNP Stability

Colloidal stability of the LNP formulations after storage at 5° C. post-freeze/thaw (F/T) was also examined by using dynamic light scattering techniques to monitor size and polydispersity index (PDI).

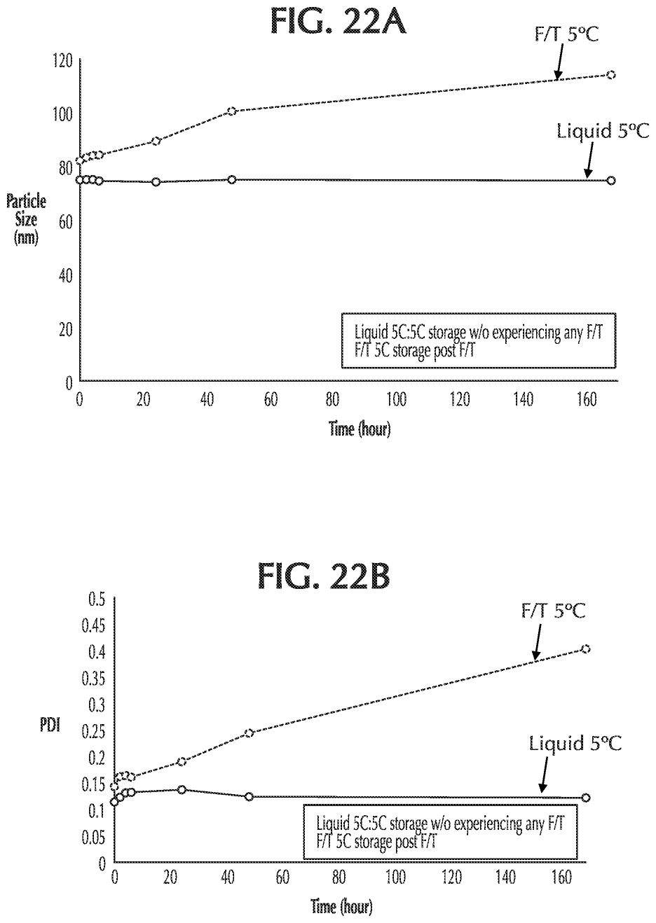

As shown below in Tables 8 and 9 and in FIG. 22, the HEPES/Sucrose formulation (no Glutamic Acid) was not colloidally stable when stored at 5° C. post-freeze/thaw. All other formulations were colloidally stable when stored 5° C. post-freeze/thaw (data not shown).

TABLE 8
Size of HEPES/Sucrose LNPs after
Storage at 5° C. Post-Freeze/Thaw

| | Liquid 5° C. | F/T followed by |
|---|---|---|
| Time (hour) | (nm) | 5° C. storage |
| 0 | 75 | 82 |
| 2 | 75 | 83 |
| 4 | 75 | 84 |
| 6 | 75 | 84 |
| 24 | 74 | 89 |
| 48 | 75 | 101 |
| 168 | 75 | 114 |

TABLE 9
PDI of HEPES/Sucrose LNPs after
Storage at 5° C. Post-Freeze/Thaw

| | Liquid | F/T followed by |
|---|---|---|
| Time (hour) | 5° C. | 5° C. storage |
| 0 | 0.114 | 0.143 |
| 2 | 0.123 | 0.162 |
| 4 | 0.131 | 0.164 |
| 6 | 0.132 | 0.161 |
| 24 | 0.137 | 0.190 |
| 48 | 0.124 | 0.244 |
| 168 | 0.122 | 0.403 |

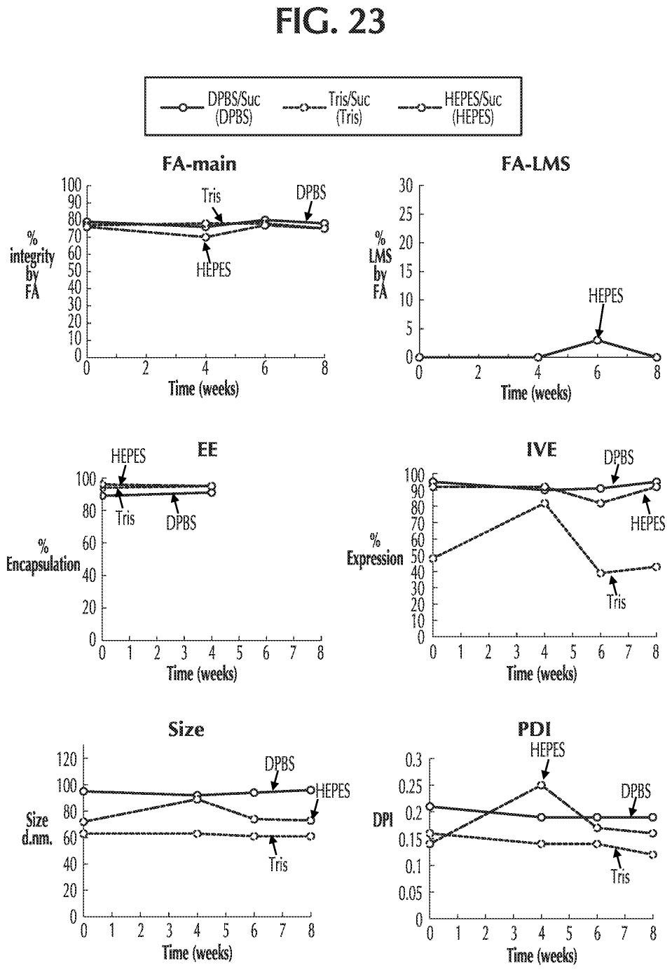

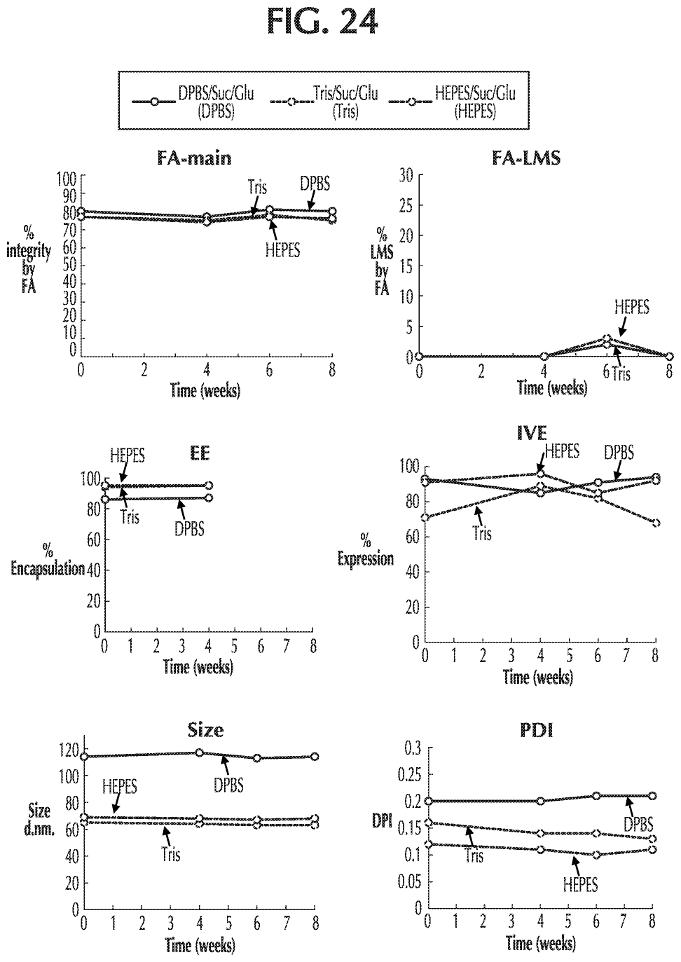

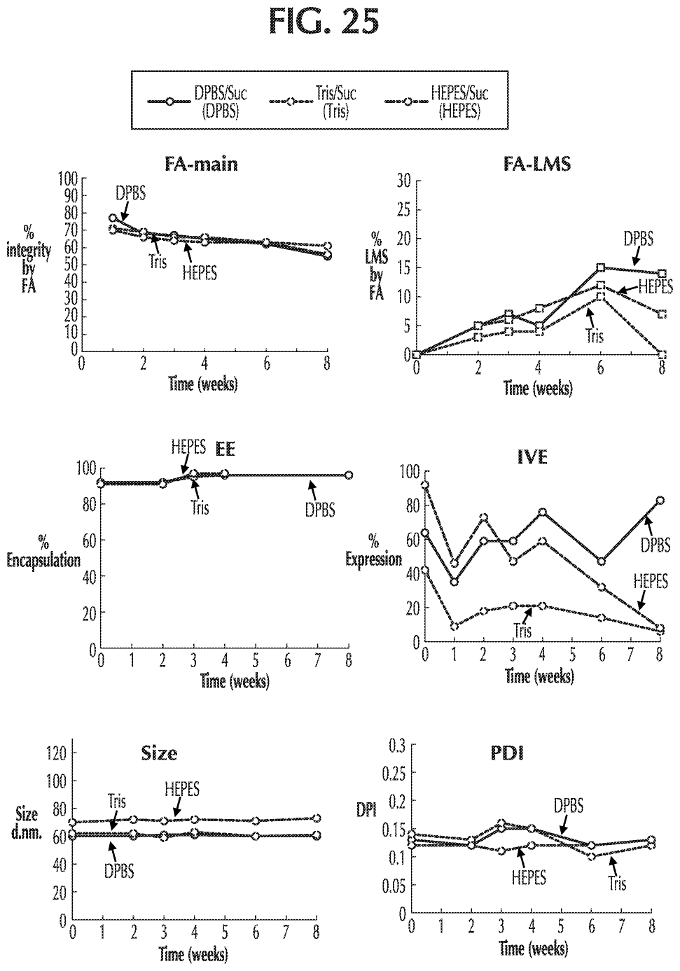

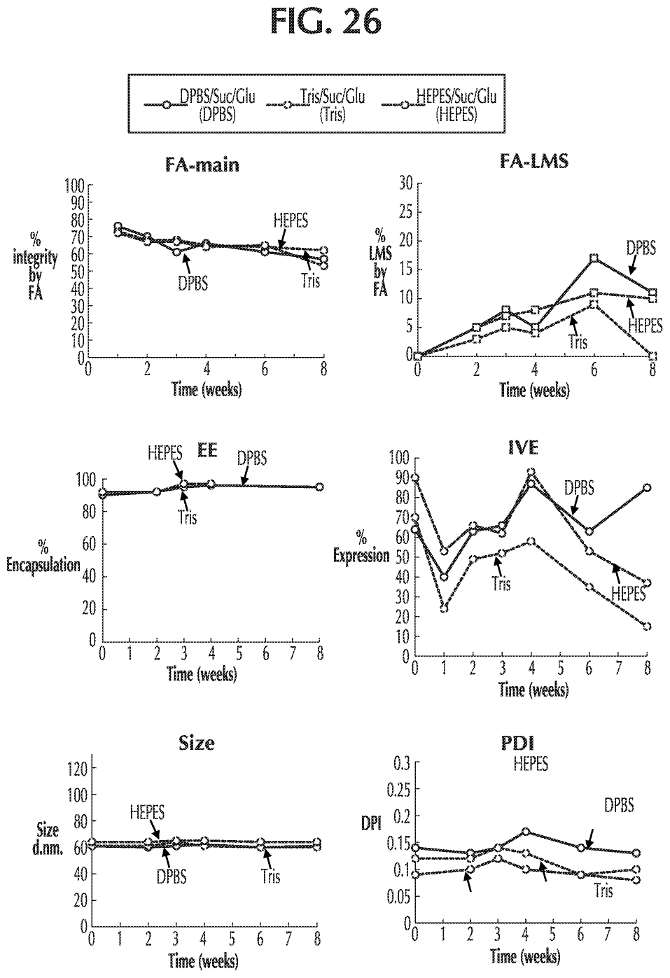

Stability in the absence of freezing and thawing was also assessed by measuring in vitro expression (IVE), encapsulation efficiency (EE), integrity (fragment analyzer, FA), and size/PDI of the LNP formulations. FIGS. 23 and 24 show that all three LNP formulations (±Glutamic Acid) appear stable after storage for up to 8 weeks at −70° C. FIGS. 25 and 26 show stability of the LNP formulations (±Glutamic Acid) after storage for up to 8 weeks at 5° C. At 5° C., the Tris/Sucrose formulation formulations (±Glutamic Acid) demonstrates the slowest RNA chemical degradation.

D. LNP Immunogenicity

The LNP formulations were tested for in vivo immunogenicity. The LNP formations were added to cells at a nonsaturating concentration, and protein expression was measured. BALB/c mice (n=10/group) were immunized on day 0 and day 28 with the LNP formulations (±Glutamic Acid) at a 0.02 μg dose level. Immunizations were given intramuscularly (i.m.) in a dose volume of 20 μL. Blood was collected after three and six weeks (days 21 and 42) to measure the antibody immune response by ELISA.

In particular, a VSV G pseudotyped virus microneutralization assay (MNT) was used to quantitatively measure functional antibodies in serum that neutralize pseudovirus activity, preventing infection of a host cell monolayer. A neutralization reaction occurs when the pseudovirus is incubated with serum samples; this reaction mixture is then applied to a monolayer of Vero cells to measure the extent of neutralization. Neutralizing titers are reported as the reciprocal of the dilution that results in 50% or 90% reduction in infection when compared to a no serum control.

All LNP formulations were stored frozen at −80° C. Samples had been stored ˜7-weeks at the time of the in vivo study. Stability of the stored LNP formulations was assessed by measuring in vitro expression (IVE), encapsulation efficiency (EE), integrity (fragment analyzer, FA), and size/PDI of the LNP formulations.

Experimental groups, descriptions of the LNP formulations tested, physical and chemical stability measurements, and immunogenicity (50% Neut (PD1) and 90% Neut (PD1) are provided in Table 10 below.

TABLE 10
In vivo Study of Stored LNP Formulations

| | | Description | | | | | | | | |
|---|---|---|---|---|---|---|---|---|---|---|
| | | of | | | | | FA | IVE | 50% | 90% |
| Gp | | saRNA | Vax | Bleed | Dose | Size/PDI | (main/ | (10 | Neut | Neut |
| # | Mice | LNP | (Day) | (Day) | (μg) | (nm/PDI) | LMS) | ng) | (PD1) | (PD1) |
| 1 | 10 | Saline | 0, 28 | 21, 42 | — | — | — | — | — | — |
| 2 | 10 | Tris/Suc − | 0, 28 | 21, 42 | 0.02 μg | 71/0.25 | 53/4 | 9 | 8133 | 1715 |
| | | 1W25 C. | | | | | | | | |
| 3 | 10 | Tris/Suc − | 0, 28 | 21, 42 | 0.02 μg | 59/0.16 | 66/4 | 21 | 10113 | 2007 |
| | | 3W5 C. | | | | | | | | |
| 4 | 10 | Tris/Suc − | 0, 28 | 21, 42 | 0.02 μg | 60/0.10 | 63/10 | 14 | 9365 | 1915 |
| | | 6W5 C. | | | | | | | | |
| 5 | 10 | Tris/Suc − | 0, 28 | 21, 42 | 0.02 μg | 65/0.18 | 64/15 | 69 | 20039 | 4066 |
| | | 6W-20 C. | | | | | | | | |
| 6 | 10 | Tris/Suc − −80 C. | 0, 28 | 21, 42 | 0.02 μg | 62/0.14 | 77 | 48 | 29242 | 5386 |
| | | control | | | | | | | | |
| 7 | 10 | Tris/Suc + | 0, 28 | 21, 42 | 0.02 μg | 64/0.14 | 68/5 | 52 | 25517 | 4660 |
| | | Glu − | | | | | | | | |
| | | 3W5 C. | | | | | | | | |
| 8 | 10 | Tris/Suc + | 0, 28 | 21, 42 | 0.02 μg | 63/0.14 | 77 | 71 | 38654 | 5996 |
| | | Glu − −80 C. | | | | | | | | |
| | | control | | | | | | | | |
| 9 | 10 | DPBS/Suc − | 0, 28 | 21, 42 | 0.02 μg | 61/0.16 | 66/8 | 48 | 10577 | 2009 |
| | | 7W5 C. | | | | | | | | |
| | | (liquid) | | | | | | | | |
| 10 | 10 | DPBS/Suc − | 0, 28 | 21, 42 | 0.02 μg | 123/0.27 | 81 | 87 | 18771 | 4578 |
| | | 7W-20 C. | | | | | | | | |
| 11 | 10 | DPBS/Suc − −80° | 0, 28 | 21, 42 | 0.02 μg | 92/0.18 | 79 | 95 | 47543 | 9911 |
| | | C. | | | | | | | | |
| | | control | | | | | | | | |
| 12 | 10 | DPBS/Suc + | 0, 28 | 21, 42 | 0.02 μg | 110/0.20 | 80 | 93 | 37419 | 8645 |
| | | Glu − −80 C. | | | | | | | | |
| | | control | | | | | | | | |
| 13 | 10 | HEPES/Suc − | 0, 28 | 21, 42 | 0.02 μg | 72/0.14 | 76 | 92 | 28196 | 5782 |
| | | −80 C. | | | | | | | | |
| | | control | | | | | | | | |
| 14 | 10 | HEPES/Suc + | 0, 28 | 21, 42 | 0.02 μg | 69/0.11 | 77 | 91 | 28861 | 4960 |
| | | Glu − −80 C. | | | | | | | | |
| | | control | | | | | | | | |

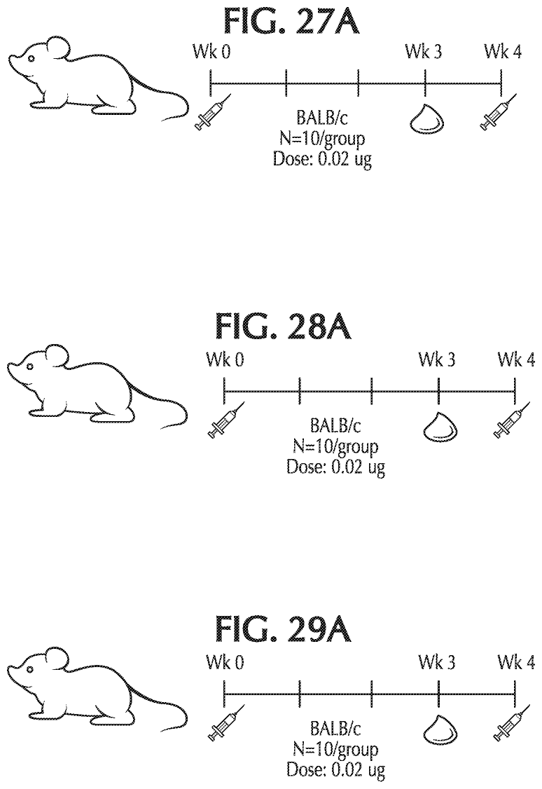

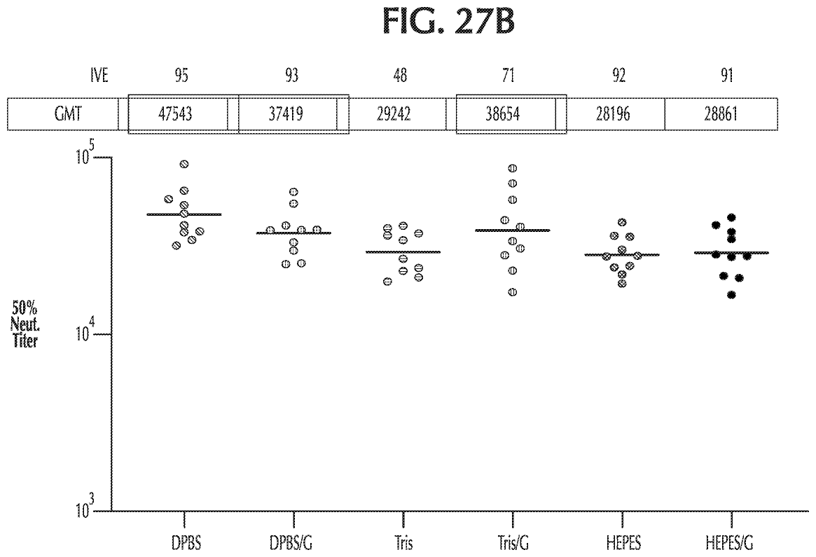

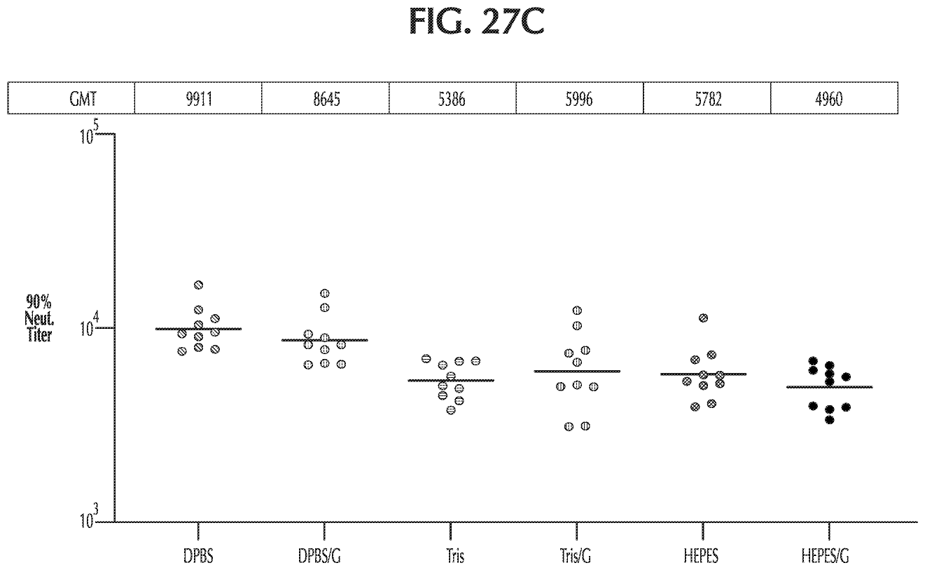

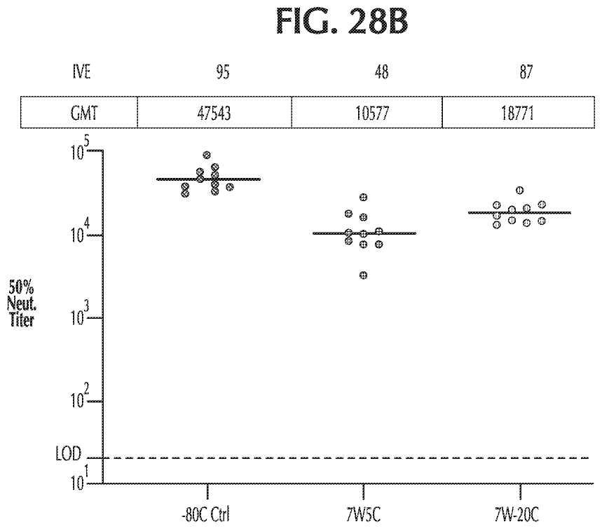

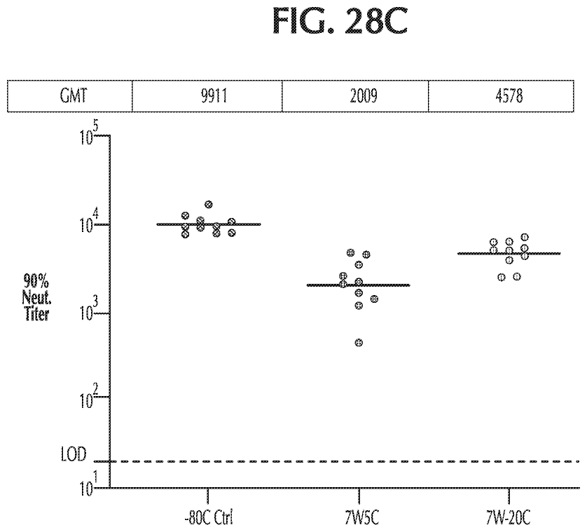

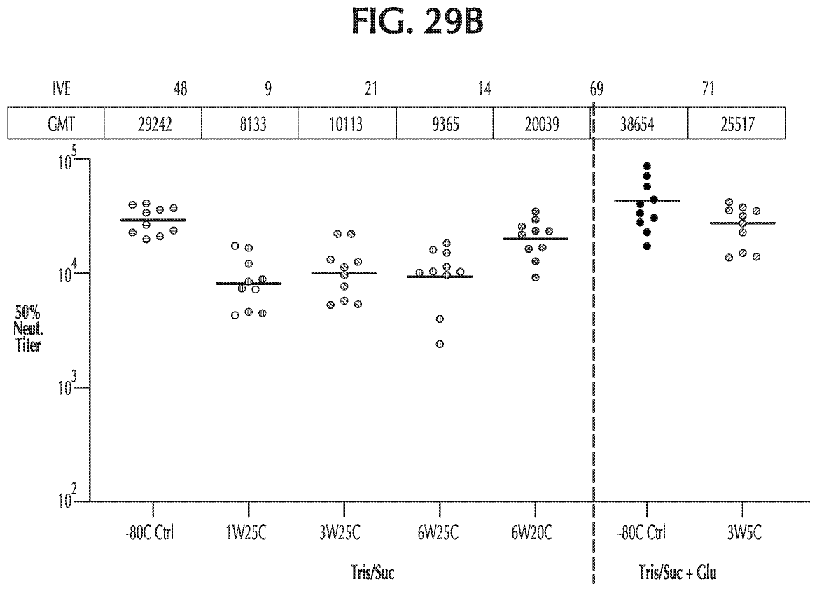

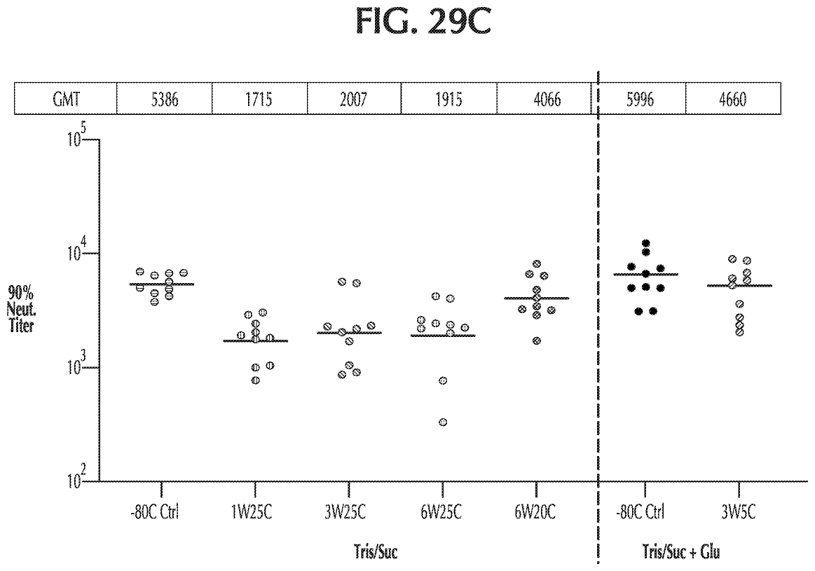

Including 20 mM glutamic acid as an excipient with LNPs encapsulating self-amplifying VSV G RNA as a model antigen resulted in significant stabilization as measured by functional immunogenic response in vivo after 3 months of storage of the formulations at 5° C. As shown in FIG. 27, the PBS/Sucrose, PBS/Sucrose/Glutamic Acid, and Tris/Sucrose/Glutamic Acid formulations were slightly more immunogenic than the remaining formulations. FIG. 28 shows that aging of PBS/Sucrose formulation led to a rapid 80% loss of immunogenicity upon 7 weeks' storage at 5° C. Finally, FIG. 29 shows that immunogenicity loss in Tris/Sucrose/Glutamic Acid formulation is slower than in the Tris/Suc formulation, indicating that inclusion of glutamic acid in the formulation improves immunogenicity. In particular, 6 weeks and 3 weeks of storage at 5° C. led to ˜65% loss of immunogenicity for the Tris/Sucrose formulation, while the Tris/Sucrose/Glutamic acid formulation exhibited a ˜30% loss of immunogenicity upon 3 weeks of storage at 5° C. storage. In this experiment, almost no loss of 50% neutralization titer was observed from vaccines treated with glutamic acid in Tris/Sucrose buffer, whereas the untreated vaccine in the same buffer showed a nearly 50-fold reduction of neutralization titer when compared with controls stored at −80° C.

SEQUENCES
This application includes a Sequence Listing in computer readable form. The computer
readable form is incorporated herein by reference in its entirety.

| SEQ ID NO: | Sequence |
|---|---|
| RNA sequence | GCCAGCCCCCGAUUGGGGGCGACACUCCACCAUAGAUCACUCCCCU |
| derived from HCV | GUGAGGAACUACUGUCUUCACGCAGAAAGCGUCUAGCCAUGGCGUU |
| IRES | AGUAUGAGUGUCGUGCAGCCUCCAGGACCCCCCCUCCCGGGAGAGC |
| SEQ ID NO: 1 | CAUAGUGGUCUGCGGAACCGGUGAGUACACCGGAAUUGCCAGGACG |
| | ACCGGGUCCUUUCUUGGAUCAACCCGCUCAAUGCCUGGAGAUUUGG |
| | GCGUGCCCCCGCGAGACUGCUAGCCGaAGUAGUGUUGGGUCGCGAA |
| | AGGCCUUGUGGUACUGCCUGAUAGGGUGCUUGCGAGUGCCCCGGGA |
| | GGUCUCGUAGACCGUGCACCAUGAGCACGAAUCCUAAACCUCAAAG |
| | AAAAACCAAACGUAACACCAACCGCCGCCCACAGGACGUCUGAGGC |
| | GGCCGCCGCCUCAGACGUCCUGUGGGUUUAUUGCAUCCCGC |
| DNA transcript, | GGATCCTAATACGACTCACTATAGCCAGCCCCCGATTGGGGGCGACA |
| derived from HCV | CTCCACCATAGATCACTCCCCTGTGAGGAACTACTGTCTTCACGCAGA |
| IRES (T7 promoter | AAGCGTCTAGCCATGGCGTTAGTATGAGTGTCGTGCAGCCTCCAGGA |
| in bold; site in | CCCCCCCTCCCGGGAGAGCCATAGTGGTCTGCGGAACCGGTGAGTAC |
| italics) | ACCGGAATTGCCAGGACGACCGGGTCCTTTCTTGGATCAACCCGCTC |
| SEQ ID NO: 2 | AATGCCTGGAGATTTGGGCGTGCCCCCGCGAGACTGCTAGCCGAGTA |
| | GTGTTGGGTCGCGAAAGGCCTTGTGGTACTGCCTGATAGGGTGCTTGC |
| | GAGTGCCCCGGGAGGTCTCGTAGACCGTGCACCATGAGCACGAATCC |
| | TAAACCTCAAAGAAAAACCAAACGTAACACCAACCGCCGCCCACAG |
| | GACGTCTGAGGCGGCCGCCGCCTCAGACGTCCTGTGGGTTTATTGCAT |
| | CCCGCCTCGAGTCTAGA |
| FgenL model RNA | UCUCUCGCUAUCUCGGAAUCGAGGGGUCUGGCCUACGCUGCGAUUG |
| synthesized RNA | CUUCUUCAGAGCAUCCUUCUUCAGCCUUGUGCCCUCUACAGUGGCA |
| sequence | GCCUCACACAAACUAUCAACAUGGCUUCCGAAUCCAGGCUCUACCA |
| SEQ ID NO: 3 | GAUCUCCGGCGAGACCAAGUCUCAUCUGCUCAAGUUCCGCUUGACG |
| | ACAUCUCGGGCCAGCAAACCUCAGGCAGUGAUUUAUUUGAUUGAU |
| | AAAAACACUCACGAAAUCCGCCAAGACGAUGACAAGACUGUAUACA |
| | CCUCCCUCGACGAGAUAGCCGACGACCUCCCCGACAGCACCCCUCG |
| | AUUCAUCUUCCUCAGCUAUCCCUUGACGAUGCCCGAUGGACGGCUA |
| | UCCGUGCCUUACACCAUGAUCUACUACCUCCCCAUCAACUGCAAUG |
| | CCGCAACAAGGAUGCUCUACGCCGGCGCAAAAGAGCUGAUACGCAA |
| | CACGGCCGAGGUGAACAAGGUUAUCGAUAUAGAGUCUGCAGAGGA |
| | UCUGGAAGAUAUUCCAAAGCAGUUGAGCGGAUAGAAAUAAUAAUA |
| | AAAGAAAUAACAUGUUCGCUAUGCC |
| FgenL model RNA | UCCACCAUGAAUCACUCCUCUCUCGCUAUCUCGGAAUCGAGGGGU |
| synthesized RNA | CUGGCCUACGCUGCGAUUGCUUCUUCAGAGCAUCCUUCUUCAGCCU |
| sequence, with | UGUGCCCUCUACAGUGGCAGCCUCACACAAACUAUCAACAUGGCUU |
| primer annealing | CCGAAUCCAGGCUCUACCAGAUCUCCGGCGAGACCAAGUCUCAUCU |
| sites bolded | GCUCAAGUUCCGCUUGACGACAUCUCGGGCCAGCAAACCUCAGGCA |
| SEQ ID NO: 4 | GUGAUUUAUUUGAUUGAUAAAAACACUCACGAAAUCCGCCAAGAC |
| | GAUGACAAGACUGUAUACACCUCCCUCGACGAGAUAGCCGACGACC |
| | UCCCCGACAGCACCCCUCGAUUCAUCUUCCUCAGCUAUCCCUUGAC |
| | GAUGCCCGAUGGACGGCUAUCCGUGCCUUACACCAUGAUCUACUAC |
| | CUCCCCAUCAACUGCAAUGCCGCAACAAGGAUGCUCUACGCCGGCG |
| | CAAAAGAGCUGAUACGCAACACGGCCGAGGUGAACAAGGUUAUCG |
| | AUAUAGAGUCUGCAGAGGAUCUGGAAGAUAUUCCAAAGCAGUUGA |
| | GCGGAUAGAAAUAAUAAUAAAAGAAAUAACAUGUUCGCUAUGCCG |
| | AAAAACCAAACGUAACACC |
| DNA template of | TCCACCATGAATCACTCCTCTCTCGCTATCTCGGAATCGAGGGGTCT |
| FgenL model | GGCCTACGCTGCGATTGCTTCTTCAGAGCATCCTTCTTCAGCCTTGTG |
| RNA, with primer | CCCTCTACAGTGGCAGCCTCACACAAACTATCAACATGGCTTCCGAAT |
| annealing sites | CCAGGCTCTACCAGATCTCCGGCGAGACCAAGTCTCATCTGCTCAAGT |
| bolded | TCCGCTTGACGACATCTCGGGCCAGCAAACCTCAGGCAGTGATTTATT |
| SEQ ID NO:5 | TGATTGATAAAAACACTCACGAAATCCGCCAAGACGATGACAAGACT |
| | GTATACACCTCCCTCGACGAGATAGCCGACGACCTCCCCGACAGCAC |
| | CCCTCGATTCATCTTCCTCAGCTATCCCTTGACGATGCCCGATGGACG |
| | GCTATCCGTGCCTTACACCATGATCTACTACCTCCCCATCAACTGCAA |
| | TGCCGCAACAAGGATGCTCTACGCCGGCGCAAAAGAGCTGATACGCA |
| | ACACGGCCGAGGTGAACAAGGTTATCGATATAGAGTCTGCAGAGGAT |
| | CTGGAAGATATTCCAAAGCAGTTGAGCGGATAGAAATAATAATAAAA |
| | GAAATAACATGTTCGCTATGCCGAAAAACCAAACGTAACACC |
| Chi18-4 model | UACGCCAGACAAUAGAUUCAGCUCGAAUAAUGAAGCCGAUUGGUC |
| RNA synthesized | ACUGAUUAACUUGGUGUAUCUACUGGGUAGCAAAGGACCGAUCUU |
| RNA sequence | CACCUCGCAUGACCGUUGAAUGUGGCGCAAUUCUAGUACUUGAAUG |
| SEQ ID NO: 6 | GCCUGGCCAAGCUUAUAGAACAAUCGAGACGAAUUGAACACCACUA |
| | UCAAAAUCCAAAUUAUGCGGUUUUCAAUCAUACAUGUGGCUUUAU |
| | GGCUGGCCAUGGCGGCCGGUUCAUUUACAGCUAACGCUUACGGAGC |
| | UGUUCGAUGCGUCAUGUAUCUCACAGGGCAACACGUGGUAGUCCCU |
| | UCAGAACCUCAUCUCGUGGAUUCCAUAACCCAUUUGAUACUGGCUU |
| | UUAUGCGCUCUGAUGUCUUCAAUGUGGACAAAACGCCUGCUGAAU |
| | UCCCGCUCUUCGCAUCAGUUGCUGAGACGCGCGAGAAGUUCAACGC |
| | AAAUACCAAGAUCAUGGUCGCAAUCGGCGGUUGGGGAGACGCAGG |
| | AUUUGAAGAAGCUGCGCGUGACGAUUCAUCGAGAAAGCGGUGGGC |
| | UGGCCACGUCAAGGCCAUGGUUGACCAGACAGGAGCCGAUGGCAUU |
| | GACAUUGACUGGGAAUAUCCGGGAGGGAACCGUGACGAUUAUAAA |
| | CUCAUUCCGAACUCUCAGCGGGAAUGGGAGAUUCAGGCAUUCGUGC |
| | UCCUUCUUCAAGAAUUGCGUUCCGCCUUGGGACAAACGAAACUGCU |
| | CUCAAUUGCGGUGCCAGCGCUGGAACGAGAUUUGAUGGCCUUCACA |
| | AACUCGACUAUUCCGUCCAUUGUGGAUCAAGUUGACUUUAUCAGU |
| | GUAAUGACUUACGACAUGAUGAACCGACGCGACAACGUUGUCAAGC |
| | ACCACAGCGGCGUGGCUGACUCCUGGGAAGCAAUGAAGCGAUAUAU |
| | AGAUCGCGGCGUCCCUCCGCACAAGCUGAACUUUGGACUUGGUUAC |
| | UAUGCCAAAUGGUUCAUGACUGAACAAUGCGAUGUACAGCACCCAU |
| | UAGGAUGCCGCACUCAACUGCUAGAAGACCCCGCCAAUGGAGCCGA |
| | UCUUGGCAAGACUGCAGCUUUUAGCUGGCACGACGAGGUUCCCGCA |
| | GAAAUGGCCAAUUCCUUUGGCAAAGCCCAUGCUCAUGGCCGCUACU |
| | AUGAAGAUGGAAGCUACGGCUAUUGGGACGAUGAAGAGAAGAGAU |
| | GGUGGUCCUACGACACGCCUCUCACCAUCAAAGCCAAAGUGCCUCG |
| | GCUUCUCGGCGAGCUGCAGCUGGGCGGGGUGUUUGCCUGGGGGCUG |
| | GGCGAGGACGCUCCGCAGUUUCUGCACCUGAAGGCGACUGUUGACG |
| | GCAUUCGGGUUCUGCGCGGAGAUGACGAUGCUGUGAAGGAUGAGC |
| | UGUAACAAGCAGCGGCGUGUGUACGUCUGGAGUUUGGCGACAUCA |
| | UUGAAGGCGUGGGAUGCAAUUCAAGCCAUUGAUACUGCAGAUAAG |
| | UUGAAGCAGCAACAGCCAAAACUCUGCACGUGGCCCGAGACUUCCG |
| | UCUUAUAGCAGGGAACAGACAGGAUGGUGCUGACGGAAUAUGCAG |
| | AGCUCGAGCUCAACCGGUAGGCUAGCUUGAAAACCCCAUGAGCUAG |
| | CAGUUAACCUUGCAGUUAAGGGUAACCAGAGCUUAAACACGUCUG |
| | AUUUGGUGCAAUGAAUACAGGCUGC |
| Chi18-4 model | UCCACCAUGAAUCACUCCUACGCCAGACAAUAGAUUCAGCUCGAA |
| RNA synthesized | UAAUGAAGCCGAUUGGUCACUGAUUAACUUGGUGUAUCUACUGGG |
| RNA sequence, | UAGCAAAGGACCGAUCUUCACCUCGCAUGACCGUUGAAUGUGGCGC |
| primer annealing | AAUUCUAGUACUUGAAUGGCCUGGCCAAGCUUAUAGAACAAUCGA |
| sites in bold | GACGAAUUGAACACCACUAUCAAAAUCCAAAUUAUGCGGUUUUCA |
| SEQ ID NO: 7 | AUCAUACAUGUGGCUUUAUGGCUGGCCAUGGCGGCCGGUUCAUUU |
| | ACAGCUAACGCUUACGGAGCUGUUCGAUGCGUCAUGUAUCUCACAG |
| | GGCAACACGUGGUAGUCCCUUCAGAACCUCAUCUCGUGGAUUCCAU |
| | AACCCAUUUGAUACUGGCUUUUAUGCGCUCUGAUGUCUUCAAUGU |
| | GGACAAAACGCCUGCUGAAUUCCCGCUCUUCGCAUCAGUUGCUGAG |
| | ACGCGCGAGAAGUUCAACGCAAAUACCAAGAUCAUGGUCGCAAUCG |
| | GCGGUUGGGGAGACGCAGGAUUUGAAGAAGCUGCGCGUGACGAUU |
| | CAUCGAGAAAGCGGUGGGCUGGCCACGUCAAGGCCAUGGUUGACCA |
| | GACAGGAGCCGAUGGCAUUGACAUUGACUGGGAAUAUCCGGGAGG |
| | GAACCGUGACGAUUAUAAACUCAUUCCGAACUCUCAGCGGGAAUGG |
| | GAGAUUCAGGCAUUCGUGCUCCUUCUUCAAGAAUUGCGUUCCGCCU |
| | UGGGACAAACGAAACUGCUCUCAAUUGCGGUGCCAGCGCUGGAACG |
| | AGAUUUGAUGGCCUUCACAAACUCGACUAUUCCGUCCAUUGUGGAU |
| | CAAGUUGACUUUAUCAGUGUAAUGACUUACGACAUGAUGAACCGA |
| | CGCGACAACGUUGUCAAGCACCACAGCGGCGUGGCUGACUCCUGGG |
| | AAGCAAUGAAGCGAUAUAUAGAUCGCGGCGUCCCUCCGCACAAGCU |
| | GAACUUUGGACUUGGUUACUAUGCCAAAUGGUUCAUGACUGAACA |
| | AUGCGAUGUACAGCACCCAUUAGGAUGCCGCACUCAACUGCUAGAA |
| | GACCCCGCCAAUGGAGCCGAUCUUGGCAAGACUGCAGCUUUUAGCU |
| | GGCACGACGAGGUUCCCGCAGAAAUGGCCAAUUCCUUUGGCAAAGC |
| | CCAUGCUCAUGGCCGCUACUAUGAAGAUGGAAGCUACGGCUAUUGG |
| | GACGAUGAAGAGAAGAGAUGGUGGUCCUACGACACGCCUCUCACCA |
| | UCAAAGCCAAAGUGCCUCGGCUUCUCGGCGAGCUGCAGCUGGGCGG |
| | GGUGUUUGCCUGGGGGCUGGGCGAGGACGCUCCGCAGUUUCUGCAC |
| | CUGAAGGCGACUGUUGACGGCAUUCGGGUUCUGCGCGGAGAUGAC |
| | GAUGCUGUGAAGGAUGAGCUGUAACAAGCAGCGGCGUGUGUACGU |
| | CUGGAGUUUGGCGACAUCAUUGAAGGCGUGGGAUGCAAUUCAAGC |
| | CAUUGAUACUGCAGAUAAGUUGAAGCAGCAACAGCCAAAACUCUGC |
| | ACGUGGCCCGAGACUUCCGUCUUAUAGCAGGGAACAGACAGGAUGG |
| | UGCUGACGGAAUAUGCAGAGCUCGAGCUCAACCGGUAGGCUAGCUU |
| | GAAAACCCCAUGAGCUAGCAGUUAACCUUGCAGUUAAGGGUAACCA |
| | GAGCUUAAACACGUCUGAUUUGGUGCAAUGAAUACAGGCUGCGAA |
| | AAACCAAACGUAACACC |
| DNA transcript for | TCCACCATGAATCACTCCTACGCCAGACAATAGATTCAGCTCGAAT |
| Chi18-4 model | AATGAAGCCGATTGGTCACTGATTAACTTGGTGTATCTACTGGGTAGC |
| RNA synthesized | AAAGGACCGATCTTCACCTCGCATGACCGTTGAATGTGGCGCAATTCT |
| RNA sequence, | AGTACTTGAATGGCCTGGCCAAGCTTATAGAACAATCGAGACGAATT |
| primer annealing | GAACACCACTATCAAAATCCAAATTATGCGGTTTTCAATCATACATGT |
| sites in bold | GGCTTTATGGCTGGCCATGGCGGCCGGTTCATTTACAGCTAACGCTTA |
| SEQ ID NO: 8 | CGGAGCTGTTCGATGCGTCATGTATCTCACAGGGCAACACGTGGTAG |
| | TCCCTTCAGAACCTCATCTCGTGGATTCCATAACCCATTTGATACTGG |
| | CTTTTATGCGCTCTGATGTCTTCAATGTGGACAAAACGCCTGCTGAAT |
| | TCCCGCTCTTCGCATCAGTTGCTGAGACGCGCGAGAAGTTCAACGCA |
| | AATACCAAGATCATGGTCGCAATCGGCGGTTGGGGAGACGCAGGATT |
| | TGAAGAAGCTGCGCGTGACGATTCATCGAGAAAGCGGTGGGCTGGCC |
| | ACGTCAAGGCCATGGTTGACCAGACAGGAGCCGATGGCATTGACATT |
| | GACTGGGAATATCCGGGAGGGAACCGTGACGATTATAAACTCATTCC |
| | GAACTCTCAGCGGGAATGGGAGATTCAGGCATTCGTGCTCCTTCTTCA |
| | AGAATTGCGTTCCGCCTTGGGACAAACGAAACTGCTCTCAATTGCGG |
| | TGCCAGCGCTGGAACGAGATTTGATGGCCTTCACAAACTCGACTATTC |
| | CGTCCATTGTGGATCAAGTTGACTTTATCAGTGTAATGACTTACGACA |
| | TGATGAACCGACGCGACAACGTTGTCAAGCACCACAGCGGCGTGGCT |
| | GACTCCTGGGAAGCAATGAAGCGATATATAGATCGCGGCGTCCCTCC |
| | GCACAAGCTGAACTTTGGACTTGGTTACTATGCCAAATGGTTCATGAC |
| | TGAACAATGCGATGTACAGCACCCATTAGGATGCCGCACTCAACTGC |
| | TAGAAGACCCCGCCAATGGAGCCGATCTTGGCAAGACTGCAGCTTTT |
| | AGCTGGCACGACGAGGTTCCCGCAGAAATGGCCAATTCCTTTGGCAA |
| | AGCCCATGCTCATGGCCGCTACTATGAAGATGGAAGCTACGGCTATT |
| | GGGACGATGAAGAGAAGAGATGGTGGTCCTACGACACGCCTCTCACC |
| | ATCAAAGCCAAAGTGCCTCGGCTTCTCGGCGAGCTGCAGCTGGGCGG |
| | GGTGTTTGCCTGGGGGCTGGGCGAGGACGCTCCGCAGTTTCTGCACCT |
| | GAAGGCGACTGTTGACGGCATTCGGGTTCTGCGCGGAGATGACGATG |
| | CTGTGAAGGATGAGCTGTAACAAGCAGCGGCGTGTGTACGTCTGGAG |
| | TTTGGCGACATCATTGAAGGCGTGGGATGCAATTCAAGCCATTGATA |
| | CTGCAGATAAGTTGAAGCAGCAACAGCCAAAACTCTGCACGTGGCCC |
| | GAGACTTCCGTCTTATAGCAGGGAACAGACAGGATGGTGCTGACGGA |
| | ATATGCAGAGCTCGAGCTCAACCGGTAGGCTAGCTTGAAAACCCCAT |
| | GAGCTAGCAGTTAACCTTGCAGTTAAGGGTAACCAGAGCTTAAACAC |
| | GTCTGATTTGGTGCAATGAATACAGGCTGCGAAAAACCAAACGTAA |
| | CACC |
| RNA FP | GGAAUCUCUCUCACGAACUGACGUAAUCUU |
| Stabilization Assay | |
| SEQ ID NO: 9 | |
| VSV G protein | MKCLLYLAFLFIGVNCKFTIVFPHNQKGNWKNVPSNYHYCPSSSDLNWH |
| amino acid | NDLIGTALQVKMPKSHKAIQADGWMCHASKWVTTCDFRWYGPKYITH |
| SEQ ID NO: 10 | SIRSFTPSVEQCKESIEQTKQGTWLNPGFPPQSCGYATVTDAEAVIVQVTP |
| | HHVLVDEYTGEWVDSQFINGKCSNYICPTVHNSTTWHSDYKVKGLCDS |
| | NLISMDITFFSEDGELSSLGKEGTGFRSNYFAYETGGKACKMQYCKHWG |
| | VRLPSGVWFEMADKDLFAAARFPECPEGSSISAPSQTSVDVSLIQDVERIL |
| | DYSLCQETWSKIRAGLPISPVDLSYLAPKNPGTGPAFTIINGTLKYFETRYI |
| | RVDIAAPILSRMVGMISGTTTERELWDDWAPYEDVEIGPNGVLRTSSGY |
| | KFPLYMIGHGMLDSDLHLSSKAQVFEHPHIQDAASQLPDDESLFFGDTGL |
| | SKNPIELVEGWFSSWKSSIASFFFIIGLIIGLFLVLRVGIHLCIKLKHTKKRQ |
| | IYTDIEMNRLGK |
| VSV G protein | ATGAAGTGCCTGCTGTACCTGGCCTTCCTGTTCATCGGCGTGAACTGC |
| DNA | AAGTTTACCATCGTGTTCCCTCACAACCAGAAAGGCAACTGGAAGAA |
| SEQ ID NO: 11 | CGTGCCCAGCAACTACCACTACTGCCCCAGCAGCAGCGACCTGAACT |
| | GGCACAATGACCTGATCGGCACAGCCCTGCAAGTGAAGATGCCCAAG |
| | AGCCACAAGGCCATCCAGGCCGATGGATGGATGTGCCACGCCAGCAA |
| | ATGGGTCACCACCTGTGACTTCCGTTGGTACGGCCCCAAGTACATCAC |
| | CCACAGCATCCGCAGCTTCACCCCTAGCGTGGAACAGTGCAAAGAGA |
| | GCATCGAGCAGACCAAGCAAGGCACCTGGCTGAACCCTGGATTCCCA |
| | CCTCAGAGCTGTGGCTACGCCACTGTGACAGATGCCGAGGCCGTGAT |
| | CGTGCAAGTGACACCTCATCACGTGCTGGTGGACGAGTACACAGGCG |
| | AGTGGGTCGACAGCCAGTTCATCAACGGCAAGTGCTCCAACTACATC |
| | TGCCCCACCGTGCACAACAGCACCACCTGGCACAGCGACTACAAAGT |
| | GAAGGGCCTGTGCGACAGCAACCTGATCAGCATGGACATCACCTTTT |
| | TCAGCGAGGACGGCGAGCTGAGCAGCCTGGGAAAAGAAGGCACCGG |
| | CTTCAGAAGCAACTACTTCGCCTATGAGACAGGCGGCAAGGCCTGCA |
| | AGATGCAGTATTGCAAGCACTGGGGCGTCAGACTGCCTAGCGGAGTT |
| | TGGTTCGAGATGGCCGACAAGGACCTGTTTGCCGCCGCTAGATTCCCT |
| | GAGTGTCCTGAGGGCAGCAGCATCTCTGCCCCTAGCCAGACATCTGT |
| | GGACGTGTCCCTGATCCAGGACGTGGAACGGATCCTGGACTACAGCC |
| | TGTGCCAAGAGACATGGTCCAAGATCAGAGCCGGCCTGCCTATCAGC |
| | CCTGTGGACCTGTCTTATCTGGCCCCTAAGAACCCTGGCACCGGACCT |
| | GCCTTCACCATCATCAATGGCACCCTGAAGTACTTTGAGACACGGTAC |
| | ATCCGCGTGGACATTGCCGCTCCTATCCTGAGCAGAATGGTCGGAAT |
| | GATCAGCGGCACCACCACCGAGAGGGAACTGTGGGATGATTGGGCCC |
| | CTTACGAGGATGTGGAAATCGGCCCTAACGGCGTGCTGAGAACAAGC |
| | AGCGGCTACAAGTTCCCACTGTACATGATCGGCCACGGCATGCTGGA |
| | CAGCGATCTGCACCTGTCTAGCAAGGCCCAGGTGTTCGAGCACCCTC |
| | ACATTCAGGATGCCGCCAGCCAGCTGCCTGATGACGAGAGCCTGTTC |
| | TTTGGCGATACCGGCCTGAGCAAGAACCCCATCGAACTGGTGGAAGG |
| | CTGGTTCAGCAGCTGGAAGTCCTCTATCGCCAGCTTCTTCTTCATCAT |
| | CGGGCTGATTATCGGCCTGTTCCTGGTGCTGAGAGTGGGCATCCACCT |
| | GTGCATCAAGCTGAAGCACACCAAGAAGCGGCAGATCTACACCGACA |
| | TCGAGATGAACCGGCTGGGCAAGTGA |
| VZV gE amino | MGTVNKPVVGVLMGFGIITGTLRITNPVRASVLRYDDFHIDEDKLDTNSV |
| acid | YEPYYHSDHAESSWVNRGESSRKAYDHNSPYIWPRNDYDGFLENAHEH |
| SEQ ID NO: 12 | HGVYNQGRGIDSGERLMQPTQMSAQEDLGDDTGIHVIPTLNGDDRHKIV |
| | NVDQRQYGDVFKGDLNPKPQGQRLIEVSVEENHPFTLRAPIQRIYGVRYT |
| | ETWSFLPSLTCTGDAAPAIQHICLKHTTCFQDVVVDVDCAENTKEDQLA |
| | EISYRFQGKKEADQPWIVVNTSTLFDELELDPPEIEPGVLKVLRTEKQYL |
| | GVYIWNMRGSDGTSTYATFLVTWKGDEKTRNPTPAVTPQPRGAEFHM |
| | WNYHSHVFSVGDTFSLAMHLQYKIHEAPFDLLLEWLYVPIDPTCQPMRL |
| | YSTCLYHPNAPQCLSHMNSGCTFTSPHLAQRVASTVYQNCEHADNYTA |
| | YCLGISHMEPSFGLILHDGGTTLKFVDTPESLSGLYVFVVYFNGHVEAVA |
| | YTVVSTVDHFVNAIEERGFPPTAGQPPATTKPKEITPVNPGTSPLIRYAAW |
| | TGGLAAVVLLCLVIFLICTAKRMRVKAYRVDKSPYNQSMYYAGLPVDD |
| | FEDSESTDTEEEFGNAIGGSHGGSSYTVYIDKTR |
| VZV gE DNA | ATGGGGACAGTTAATAAACCTGTGGTGGGGGTATTGATGGGGTTCGG |
| sequence | AATTATCACGGGAACGTTGCGTATAACGAATCCGGTCAGAGCATCCG |
| SEQ ID NO: 13 | TCTTGCGATACGATGATTTTCACATCGATGAAGACAAACTGGATACA |
| | AACTCCGTATATGAGCCTTACTACCATTCAGATCATGCGGAGTCTTCA |
| | TGGGTAAATCGGGGAGAGTCTTCGCGAAAAGCGTACGATCATAACTC |
| | ACCTTATATATGGCCACGTAATGATTATGATGGATTTTTAGAGAACGC |
| | ACACGAACACCATGGGGTGTATAATCAGGGCCGTGGTATCGATAGCG |
| | GGGAACGGTTAATGCAACCCACACAAATGTCTGCACAGGAGGATCTT |
| | GGGGACGATACGGGCATCCACGTTATCCCTACGTTAAACGGCGATGA |
| | CAGACATAAAATTGTAAATGTGGACCAACGTCAATACGGTGACGTGT |
| | TTAAAGGAGATCTTAATCCAAAACCCCAAGGCCAAAGACTCATTGAG |
| | GTGTCAGTGGAAGAAAATCACCCGTTTACTTTACGCGCACCGATTCA |
| | GCGGATTTATGGAGTCCGGTACACCGAGACTTGGAGCTTTTTGCCGTC |
| | ATTAACCTGTACGGGAGACGCAGCGCCCGCCATCCAGCATATATGCT |
| | TAAAACATACAACATGCTTTCAAGACGTGGTGGTGGATGTGGATTGC |
| | GCGGAAAATACTAAAGAGGATCAGTTGGCCGAAATCAGTTACCGTTT |
| | TCAAGGTAAGAAGGAAGCGGACCAACCGTGGATTGTTGTAAACACGA |
| | GCACACTGTTTGATGAACTCGAATTAGACCCCCCCGAGATTGAACCG |
| | GGTGTCTTGAAAGTACTTCGGACAGAAAAACAATACTTGGGTGTGTA |
| | CATTTGGAACATGCGCGGCTCCGATGGTACGTCTACCTACGCCACGTT |
| | TTTGGTCACCTGGAAAGGGGATGAAAAAACAAGAAACCCTACGCCCG |
| | CAGTAACTCCTCAACCAAGAGGGGCTGAGTTTCATATGTGGAATTAC |
| | CACTCGCATGTATTTTCAGTTGGTGATACGTTTAGCTTGGCAATGCAT |
| | CTTCAGTATAAGATACATGAAGCGCCATTTGATTTGCTGTTAGAGTGG |
| | TTGTATGTCCCCATCGATCCTACATGTCAACCAATGCGGTTATATTCT |
| | ACGTGTTTGTATCATCCCAACGCACCCCAATGCCTCTCTCATATGAAT |
| | TCCGGTTGTACATTTACCTCGCCACATTTAGCCCAGCGTGTTGCAAGC |
| | ACAGTGTATCAAAATTGTGAACATGCAGATAACTACACCGCATATTG |
| | TCTGGGAATATCTCATATGGAGCCTAGCTTTGGTCTAATCTTACACGA |
| | CGGGGGCACCACGTTAAAGTTTGTAGATACACCCGAGAGTTTGTCGG |
| | GATTATACGTTTTTGTGGTGTATTTTAACGGGCATGTTGAAGCCGTAG |
| | CATACACTGTTGTATCCACAGTAGATCATTTTGTAAACGCAATTGAAG |
| | AGCGTGGATTTCCGCCAACGGCCGGTCAGCCACCGGCGACTACTAAA |
| | CCCAAGGAAATTACCCCCGTAAACCCCGGAACGTCACCACTTATACG |
| | ATATGCCGCATGGACCGGAGGGCTTGCAGCAGTAGTACTTTTATGTCT |
| | CGTAATATTTTTAATCTGTACGGCTAAACGAATGAGGGTTAAAGCCTA |
| | TAGGGTAGACAAGTCCCCGTATAACCAAAGCATGTATTACGCTGGCC |
| | TTCCAGTGGACGATTTCGAGGACTCGGAATCTACGGATACGGAAGAA |
| | GAGTTTGGTAACGCGATTGGAGGGAGTCACGGGGGTTCGAGTTACAC |
| | GGTGTATATAGATAAGACCCGGTGA |
| VZV gE RNA | AUGGGGACAGUUAAUAAACCUGUGGUGGGGGUAUUGAUGGGGUUC |
| SEQ ID NO: 14 | GGAAUUAUCACGGGAACGUUGCGUAUAACGAAUCCGGUCAGAGCA |
| | UCCGUCUUGCGAUACGAUGAUUUUCACAUCGAUGAAGACAAACUG |
| | GAUACAAACUCCGUAUAUGAGCCUUACUACCAUUCAGAUCAUGCGG |
| | AGUCUUCAUGGGUAAAUCGGGGAGAGUCUUCGCGAAAAGCGUACG |
| | AUCAUAACUCACCUUAUAUAUGGCCACGUAAUGAUUAUGAUGGAU |
| | UUUUAGAGAACGCACACGAACACCAUGGGGUGUAUAAUCAGGGCC |
| | GUGGUAUCGAUAGCGGGGAACGGUUAAUGCAACCCACACAAAUGU |
| | CUGCACAGGAGGAUCUUGGGGACGAUACGGGCAUCCACGUUAUCCC |
| | UACGUUAAACGGCGAUGACAGACAUAAAAUUGUAAAUGUGGACCA |
| | ACGUCAAUACGGUGACGUGUUUAAAGGAGAUCUUAAUCCAAAACC |
| | CCAAGGCCAAAGACUCAUUGAGGUGUCAGUGGAAGAAAAUCACCCG |
| | UUUACUUUACGCGCACCGAUUCAGCGGAUUUAUGGAGUCCGGUACA |
| | CCGAGACUUGGAGCUUUUUGCCGUCAUUAACCUGUACGGGAGACGC |
| | AGCGCCCGCCAUCCAGCAUAUAUGCUUAAAACAUACAACAUGCUUU |
| | CAAGACGUGGUGGUGGAUGUGGAUUGCGCGGAAAAUACUAAAGAG |
| | GAUCAGUUGGCCGAAAUCAGUUACCGUUUUCAAGGUAAGAAGGAA |
| | GCGGACCAACCGUGGAUUGUUGUAAACACGAGCACACUGUUUGAU |
| | GAACUCGAAUUAGACCCCCCCGAGAUUGAACCGGGUGUCUUGAAAG |
| | UACUUCGGACAGAAAAACAAUACUUGGGUGUGUACAUUUGGAACA |
| | UGCGCGGCUCCGAUGGUACGUCUACCUACGCCACGUUUUUGGUCAC |
| | CUGGAAAGGGGAUGAAAAAACAAGAAACCCUACGCCCGCAGUAACU |
| | CCUCAACCAAGAGGGGCUGAGUUUCAUAUGUGGAAUUACCACUCGC |
| | AUGUAUUUUCAGUUGGUGAUACGUUUAGCUUGGCAAUGCAUCUUC |
| | AGUAUAAGAUACAUGAAGCGCCAUUUGAUUUGCUGUUAGAGUGGU |
| | UGUAUGUCCCCAUCGAUCCUACAUGUCAACCAAUGCGGUUAUAUUC |
| | UACGUGUUUGUAUCAUCCCAACGCACCCCAAUGCCUCUCUCAUAUG |
| | AAUUCCGGUUGUACAUUUACCUCGCCACAUUUAGCCCAGCGUGUUG |
| | CAAGCACAGUGUAUCAAAAUUGUGAACAUGCAGAUAACUACACCGC |
| | AUAUUGUCUGGGAAUAUCUCAUAUGGAGCCUAGCUUUGGUCUAAU |
| | CUUACACGACGGGGGCACCACGUUAAAGUUUGUAGAUACACCCGAG |
| | AGUUUGUCGGGAUUAUACGUUUUUGUGGUGUAUUUUAACGGGCAU |
| | GUUGAAGCCGUAGCAUACACUGUUGUAUCCACAGUAGAUCAUUUU |
| | GUAAACGCAAUUGAAGAGCGUGGAUUUCCGCCAACGGCCGGUCAGC |
| | CACCGGCGACUACUAAACCCAAGGAAAUUACCCCCGUAAACCCCGG |
| | AACGUCACCACUUAUACGAUAUGCCGCAUGGACCGGAGGGCUUGCA |
| | GCAGUAGUACUUUUAUGUCUCGUAAUAUUUUUAAUCUGUACGGCU |
| | AAACGAAUGAGGGUUAAAGCCUAUAGGGUAGACAAGUCCCCGUAU |
| | AACCAAAGCAUGUAUUACGCUGGCCUUCCAGUGGACGAUUUCGAGG |
| | ACUCGGAAUCUACGGAUACGGAAGAAGAGUUUGGUAACGCGAUUG |
| | GAGGGAGUCACGGGGGUUCGAGUUACACGGUGUAUAUAGAUAAGA |
| | CCCGGUGA |
| CMV gB protein | MESRIWCLVVCVNLCIVCLGAAVSSSSTRGTSATHSHHSSHTTSAAHSRS |
| SEQ ID NO: 15 | GSVSQRVTSSQTVSHGVNETIYNTTLKYGDVVGVNTTKYPYRVCSMAQ |
| | GTDLIRFERNIVCTSMKPINEDLDEGIMVVYKRNIVAHTFKVRVYQKVLT |
| | FRRSYAYIHTTYLLGSNTEYVAPPMWEIHHINSHSQCYSSYSRVIAGTVF |
| | VAYHRDSYENKTMQLMPDDYSNTHSTRYVTVKDQWHSRGSTWLYRET |
| | CNLNCMVTITTARSKYPYHFFATSTGDVVDISPFYNGTNRNASYFGENA |
| | DKFFIFPNYTIVSDFGRPNSALETHRLVAFLERADSVISWDIQDEKNVTCQ |
| | LTFWEASERTIRSEAEDSYHFSSAKMTATFLSKKQEVNMSDSALDCVRD |
| | EAINKLQQIFNTSYNQTYEKYGNVSVFETTGGLVVFWQGIKQKSLVELE |
| | RLANRSSLNLTHNRTKRSTDGNNATHLSNMESVHNLVYAQLQFTYDTL |
| | RGYINRALAQIAEAWCVDQRRTLEVFKELSKINPSAILSAIYNKPIAARFM |
| | GDVLGLASCVTINQTSVKVLRDMNVKESPGRCYSRPVVIFNFANSSYVQ |
| | YGQLGEDNEILLGNHRTEECQLPSLKIFIAGNSAYEYVDYLFKRMIDLSSI |
| | STVDSMIALDIDPLENTDFRVLELYSQKELRSSNVFDLEEIMREFNSYKQR |
| | VKYVEDKVVDPLPPYLKGLDDLMSGLGAAGKAVGVAIGAVGGAVASV |
| | VEGVATFLKNPFGAFTIILVAIAVVIIIYLIYTRQRRLCMQPLQNLFPYLVS |
| | ADGTTVTSGNTKDTSLQAPPSYEESVYNSGRKGPGPPSSDASTAAPPYTN |
| | EQAYQMLLALVRLDAEQRAQQNGTDSLDGQTGTQDKGQKPNLLDRLR |
| | HRKNGYRHLKDSDEEENV |
| CMV gB DNA | ATGGAATCCAGGATCTGGTGCCTGGTAGTCTGCGTTAACTTGTGTATC |
| SEQ ID NO: 16 | GTCTGTCTGGGTGCTGCGGTTTCCTCATCTTCTACTCGTGGAACTTCTG |
| | CTACTCACAGTCACCATTCCTCTCATACGACGTCTGCTGCTCATTCTC |
| | GATCCGGTTCAGTCTCTCAACGCGTAACTTCTTCCCAAACGGTCAGCC |
| | ATGGTGTTAACGAGACCATCTACAACACTACCCTCAAGTACGGAGAT |
| | GTGGTGGGGGTCAACACCACCAAGTACCCCTATCGCGTGTGTTCTATG |
| | GCACAGGGTACGGATCTTATTCGCTTTGAACGTAATATCGTCTGCACC |
| | TCGATGAAGCCCATCAATGAAGACCTGGACGAGGGCATCATGGTGGT |
| | CTACAAACGCAACATCGTCGCGCACACCTTTAAGGTACGAGTCTACC |
| | AGAAGGTTTTGACGTTTCGTCGTAGCTACGCTTACGTCCACACCACTT |
| | ATCTGCTGGGCAGCAACACGGAATACGTGGCGCCTCCTATGTGGGAG |
| | ATTCATCATATCAACAGTCACAGTCAGTGCTACAGTTCCTACAGCCGC |
| | GTTATAGCAGGCACGGTTTTCGTGGCTTATCATAGGGACAGCTATGA |
| | AAACAAAACCATGCAATTAATGCCCGACGATTATTCCAACACCCACA |
| | GTACCCGTTACGTGACGGTCAAGGATCAATGGCACAGCCGCGGCAGC |
| | ACCTGGCTCTATCGTGAGACCTGTAATCTGAATTGTATGGTGACCATC |
| | ACTACTGCGCGCTCCAAGTATCCCTATCATTTTTTCGCAACTTCCACG |
| | GGTGATGTGGTTGACATTTCTCCTTTCTACAACGGAACTAATCGCAAT |
| | GCCAGCTATTTTGGAGAAAACGCCGACAAGTTTTTCATTTTTCCGAAC |
| | TACACTATCGTCTCCGACTTTGGAAGACCGAATTCTGCGTTAGAGACC |
| | CACAGGTTGGTGGCTTTTCTTGAACGTGCGGACTCAGTGATCTCCTGG |
| | GATATACAGGACGAGAAGAATGTTACTTGTCAACTCACTTTCTGGGA |
| | AGCCTCGGAACGCACCATTCGTTCCGAAGCCGAGGACTCGTATCACT |
| | TTTCTTCTGCCAAAATGACCGCCACTTTCTTATCTAAGAAGCAAGAGG |
| | TGAACATGTCCGACTCTGCGCTGGACTGTGTACGTGATGAGGCCATA |
| | AATAAGTTACAGCAGATTTTCAATACTTCATACAATCAAACATATGA |
| | AAAATATGGAAACGTGTCCGTCTTTGAAACCACTGGTGGTTTGGTGGT |
| | GTTCTGGCAAGGTATCAAGCAAAAATCTCTGGTGGAACTCGAACGTT |
| | TGGCCAACCGCTCCAGTCTGAATCTTACTCATAATAGAACCAAAAGA |
| | AGTACAGATGGCAACAATGCAACTCATTTATCCAACATGGAGTCGGT |
| | GCACAATCTGGTCTACGCCCAGCTGCAGTTCACCTATGACACGTTGCG |
| | CGGTTACATCAACCGGGCGCTGGCGCAAATCGCAGAAGCCTGGTGTG |
| | TGGATCAACGGCGCACCCTAGAGGTCTTCAAGGAACTTAGCAAGATC |
| | AACCCGTCAGCTATTCTCTCGGCCATCTACAACAAACCGATTGCCGCG |
| | CGTTTCATGGGCGATGTCCTGGGTCTGGCCAGCTGCGTGACCATTAAC |
| | CAAACCAGCGTCAAGGTGCTGCGTGATATGAATGTGAAGGAATCGCC |
| | AGGACGCTGCTACTCACGACCAGTGGTCATCTTTAATTTCGCCAACAG |
| | CTCGTACGTGCAGTACGGTCAACTGGGCGAGGATAACGAAATCCTGT |
| | TGGGCAACCACCGCACTGAGGAATGTCAGCTTCCCAGCCTCAAGATC |
| | TTCATCGCCGGCAACTCGGCCTACGAGTACGTGGACTACCTCTTCAAA |
| | CGCATGATTGACCTCAGCAGCATCTCCACCGTCGACAGCATGATCGC |
| | CCTAGACATCGACCCGCTGGAAAACACCGACTTCAGGGTACTGGAAC |
| | TTTACTCGCAGAAAGAATTGCGTTCCAGCAACGTTTTTGATCTCGAGG |
| | AGATCATGCGCGAGTTCAATTCGTATAAGCAGCGGGTAAAGTACGTG |
| | GAGGACAAGGTAGTCGACCCGCTGCCGCCCTACCTCAAGGGTCTGGA |
| | CGACCTCATGAGCGGCCTGGGCGCCGCGGGAAAGGCCGTTGGCGTAG |
| | CCATTGGGGCCGTGGGTGGCGCGGTGGCCTCCGTGGTCGAAGGCGTT |
| | GCCACCTTCCTCAAAAACCCCTTCGGAGCCTTCACCATCATCCTCGTG |
| | GCCATAGCCGTCGTCATTATCATTTATTTGATCTATACTCGACAGCGG |
| | CGTCTCTGCATGCAGCCGCTGCAGAACCTCTTTCCCTATCTGGTGTCC |
| | GCCGACGGGACCACCGTGACGTCGGGCAACACCAAAGACACGTCGTT |
| | ACAGGCTCCGCCTTCCTACGAGGAAAGTGTCTATAATTCTGGTCGCAA |
| | AGGACCGGGACCACCGTCG |
| | TCTGATGCATCCACGGCGGCTCCGCCTTACACCAACGAGCAGGCTTA |
| | CCAGATGCTTCTGGCCCTGGTCCGTCTGGACGCAGAGCAGCGAGCGC |
| | AGCAGAACGGTACAGATTCTTTGGACGGACAGACTGGCACGCAGGAC |
| | AAGGGACAGAAGCCCAACCTGCTAGACCGACTGCGACACCGCAAAA |
| | ACGGCTACCGACACTTGAAAGACTCCGACGAAGAAGAGAACGTCTGA |
| CMV gB RNA | AUGGAAUCCAGGAUCUGGUGCCUGGUAGUCUGCGUUAACUUGUGU |
| SEQ ID NO: 17 | AUCGUCUGUCUGGGUGCUGCGGUUUCCUCAUCUUCUACUCGUGGAA |
| | CUUCUGCUACUCACAGUCACCAUUCCUCUCAUACGACGUCUGCUGC |
| | UCAUUCUCGAUCCGGUUCAGUCUCUCAACGCGUAACUUCUUCCCAA |
| | ACGGUCAGCCAUGGUGUUAACGAGACCAUCUACAACACUACCCUCA |
| | AGUACGGAGAUGUGGUGGGGGUCAACACCACCAAGUACCCCUAUCG |
| | CGUGUGUUCUAUGGCACAGGGUACGGAUCUUAUUCGCUUUGAACG |
| | UAAUAUCGUCUGCACCUCGAUGAAGCCCAUCAAUGAAGACCUGGAC |
| | GAGGGCAUCAUGGUGGUCUACAAACGCAACAUCGUCGCGCACACCU |
| | UUAAGGUACGAGUCUACCAGAAGGUUUUGACGUUUCGUCGUAGCU |
| | ACGCUUACGUCCACACCACUUAUCUGCUGGGCAGCAACACGGAAUA |
| | CGUGGCGCCUCCUAUGUGGGAGAUUCAUCAUAUCAACAGUCACAGU |
| | CAGUGCUACAGUUCCUACAGCCGCGUUAUAGCAGGCACGGUUUUCG |
| | UGGCUUAUCAUAGGGACAGCUAUGAAAACAAAACCAUGCAAUUAA |
| | UGCCCGACGAUUAUUCCAACACCCACAGUACCCGUUACGUGACGGU |
| | CAAGGAUCAAUGGCACAGCCGCGGCAGCACCUGGCUCUAUCGUGAG |
| | ACCUGUAAUCUGAAUUGUAUGGUGACCAUCACUACUGCGCGCUCCA |
| | AGUAUCCCUAUCAUUUUUUCGCAACUUCCACGGGUGAUGUGGUUG |
| | ACAUUUCUCCUUUCUACAACGGAACUAAUCGCAAUGCCAGCUAUUU |
| | UGGAGAAAACGCCGACAAGUUUUUCAUUUUUCCGAACUACACUAUC |
| | GUCUCCGACUUUGGAAGACCGAAUUCUGCGUUAGAGACCCACAGGU |
| | UGGUGGCUUUUCUUGAACGUGCGGACUCAGUGAUCUCCUGGGAUA |
| | UACAGGACGAGAAGAAUGUUACUUGUCAACUCACUUUCUGGGAAG |
| | CCUCGGAACGCACCAUUCGUUCCGAAGCCGAGGACUCGUAUCACUU |
| | UUCUUCUGCCAAAAUGACCGCCACUUUCUUAUCUAAGAAGCAAGAG |
| | GUGAACAUGUCCGACUCUGCGCUGGACUGUGUACGUGAUGAGGCCA |
| | UAAAUAAGUUACAGCAGAUUUUCAAUACUUCAUACAAUCAAACAU |
| | AUGAAAAAUAUGGAAACGUGUCCGUCUUUGAAACCACUGGUGGUU |
| | UGGUGGUGUUCUGGCAAGGUAUCAAGCAAAAAUCUCUGGUGGAAC |
| | UCGAACGUUUGGCCAACCGCUCCAGUCUGAAUCUUACUCAUAAUAG |
| | AACCAAAAGAAGUACAGAUGGCAACAAUGCAACUCAUUUAUCCAAC |
| | AUGGAGUCGGUGCACAAUCUGGUCUACGCCCAGCUGCAGUUCACCU |
| | AUGACACGUUGCGCGGUUACAUCAACCGGGCGCUGGCGCAAAUCGC |
| | AGAAGCCUGGUGUGUGGAUCAACGGCGCACCCUAGAGGUCUUCAAG |
| | GAACUUAGCAAGAUCAACCCGUCAGCUAUUCUCUCGGCCAUCUACA |
| | ACAAACCGAUUGCCGCGCGUUUCAUGGGCGAUGUCCUGGGUCUGGC |
| | CAGCUGCGUGACCAUUAACCAAACCAGCGUCAAGGUGCUGCGUGAU |
| | AUGAAUGUGAAGGAAUCGCCAGGACGCUGCUACUCACGACCAGUGG |
| | UCAUCUUUAAUUUCGCCAACAGCUCGUACGUGCAGUACGGUCAACU |
| | GGGCGAGGAUAACGAAAUCCUGUUGGGCAACCACCGCACUGAGGAA |
| | UGUCAGCUUCCCAGCCUCAAGAUCUUCAUCGCCGGCAACUCGGCCU |
| | ACGAGUACGUGGACUACCUCUUCAAACGCAUGAUUGACCUCAGCAG |
| | CAUCUCCACCGUCGACAGCAUGAUCGCCCUAGACAUCGACCCGCUG |
| | GAAAACACCGACUUCAGGGUACUGGAACUUUACUCGCAGAAAGAA |
| | UUGCGUUCCAGCAACGUUUUUGAUCUCGAGGAGAUCAUGCGCGAG |
| | UUCAAUUCGUAUAAGCAGCGGGUAAAGUACGUGGAGGACAAGGUA |
| | GUCGACCCGCUGCCGCCCUACCUCAAGGGUCUGGACGACCUCAUGA |
| | GCGGCCUGGGCGCCGCGGGAAAGGCCGUUGGCGUAGCCAUUGGGGC |
| | CGUGGGUGGCGCGGUGGCCUCCGUGGUCGAAGGCGUUGCCACCUUC |
| | CUCAAAAACCCCUUCGGAGCCUUCACCAUCAUCCUCGUGGCCAUAG |
| | CCGUCGUCAUUAUCAUUUAUUUGAUCUAUACUCGACAGCGGCGUCU |
| | CUGCAUGCAGCCGCUGCAGAACCUCUUUCCCUAUCUGGUGUCCGCC |
| | GACGGGACCACCGUGACGUCGGGCAACACCAAAGACACGUCGUUAC |
| | AGGCUCCGCCUUCCUACGAGGAAAGUGUCUAUAAUUCUGGUCGCAA |
| | AGGACCGGGACCACCGUCGUCUGAUGCAUCCACGGCGGCUCCGCCU |
| | UACACCAACGAGCAGGCUUACCAGAUGCUUCUGGCCCUGGUCCGUC |
| | UGGACGCAGAGCAGCGAGCGCAGCAGAACGGUACAGAUUCUUUGG |
| | ACGGACAGACUGGCACGCAGGACAAGGGACAGAAGCCCAACCUGCU |
| | AGACCGACUGCGACACCGCAAAAACGGCUACCGACACUUGAAAGAC |
| | UCCGACGAAGAAGAGAACGUCUGA |
| RSV Full Length | MELLILKANAITTILTAVTFCFASGQNITEEFYQSTCSAVSKGYLSALRTG |
| F0 of Native RSV | WYTSVITIE |
| A2 (GenBank GI: | LSNIKENKCNGTDAKVKLIKQELDKYKNAVTELQLLMQSTPPTNNRARR |
| 138251; Swiss Prot | ELPRFMNYTLNNAKKTNVTLSKKRKRRFLGFLLGVGSAIASGVAVSKVL |
| P03420) | HLEGEVNKIKSALLSTNKAVVSLSNGVSVLTSKVLDLKNYIDKQLLPIVN |
| SEQ ID NO: 18 | KQSCSISNIETVIEFQQKNNRLLEITREFSVNAGVTTPVSTYMLTNSELLSL |
| | INDMPITNDQKKLMSNNVQIVRQQSYSIMSIIKEEVLAYVVQLPLYGVID |
| | TPCWKLHTSPLCTTNTKEGSNICLTRTDRGWYCDNAGSVSFFPQAETCK |
| | VQSNRVFCDTMNSLTLPSEINLCNVDIFNPKYDCKIMTSKTDVSSSVITSL |
| | GAIVSCYGKTKCTASNKNRGIIKTFSNGCDYVSNKGMDTVSV |
| | GNTLYYVNKQEGKSLYVKGEPIINFYDPLVFPSDEFDASISQVNEKINQSL |
| | AFIRKSDELLHNVNAGKSTTNIMITTIIIVIIVILLSLIAVGLLLYCKARSTPV |
| | TLSKDQLSGINNIAFSN |
| DNA encoding | ATGGAGTTGCTAATCCTCAAAGCAAATGCAATTACCACAATCCTCACT |
| RSV Full Length | GCAGTCACATTTTGTTTTGCTTCTGGTCAAAACATCACTGAAGAATTT |
| F0 of Native RSV | TATCAATCAACATGCAGTGCAGTTAGCAAAGGCTATCTTAGTGCTCTG |
| A2 (GenBank GI: | AGAACTGGTTGGTATACCAGTGTTATAACTATAGAATTAAGTAATATC |
| 138251; Swiss Prot | AAGGAAAATAAGTGTAATGGAACAGATGCTAAGGTAAAATTGATAA |
| P03420) | AACAAGAATTAGATAAATATAAAAATGCTGTAACAGAATTGCAGTTG |
| SEQ ID NO: 19 | CTCATGCAAAGCACACCACCAACAAACAATCGAGCCAGAAGAGAAC |
| | TACCAAGGTTTATGAATTATACACTCAACAATGCCAAAAAAACCAAT |
| | GTAACATTAAGCAAGAAAAGGAAAAGAAGATTTCTTGGTTTTTTGTT |
| | AGGTGTTGGATCTGCAATCGCCAGTGGCGTTGCTGTATCTAAGGTCCT |
| | GCACCTAGAAGGGGAAGTGAACAAGATCAAAAGTGCTCTACTATCCA |
| | CAAACAAGGCTGTAGTCAGCTTATCAAATGGAGTTAGTGTCTTAACC |
| | AGCAAAGTGTTAGACCTCAAAAACTATATAGATAAACAATTGTTACC |
| | TATTGTGAACAAGCAAAGCTGCAGCATATCAAATATAGAAACTGTGA |
| | TAGAGTTCCAACAAAAGAACAACAGACTACTAGAGATTACCAGGGA |
| | ATTTAGTGTTAATGCAGGTGTAACTACACCTGTAAGCACTTACATGTT |
| | AACTAATAGTGAATTATTGTCATTAATCAATGATATGCCTATAACAAA |
| | TGATCAGAAAAAGTTAATGTCCAACAATGTTCAAATAGTTAGACAGC |
| | AAAGTTACTCTATCATGTCCATAATAAAAGAGGAAGTCTTAGCATAT |
| | GTAGTACAATTACCACTATATGGTGTTATAGATACACCCTGTTGGAAA |
| | CTACACACATCCCCTCTATGTACAACCAACACAAAAGAAGGGTCCAA |
| | CATCTGTTTAACAAGAACTGACAGAGGATGGTACTGTGACAATGCAG |
| | GATCAGTATCTTTCTTCCCACAAGCTGAAACATGTAAAGTTCAATCAA |
| | ATCGAGTATTTTGTGACACAATGAACAGTTTAACATTACCAAGTGAA |
| | ATAAATCTCTGCAATGTTGACATATTCAACCCCAAATATGATTGTAAA |
| | ATTATGACTTCAAAAACAGATGTAAGCAGCTCCGTTATCACATCTCTA |
| | GGAGCCATTGTGTCATGCTATGGCAAAACTAAATGTACAGCATCCAA |
| | TAAAAATCGTGGAATCATAAAGACATTTTCTAACGGGTGCGATTATG |
| | TATCAAATAAAGGGATGGACACTGTGTCTGTAGGTAACACATTATAT |
| | TATGTAAATAGCAAGAAGGTAAAAGTCTCTATGTAAAAGGTGAACCA |
| | ATAATAAATTTCTATGACCCATTAGTATTCCCCTCTGATGAATTTGAT |
| | GCATCAATATCTCAAGTCAACGAGAAGATTAACCAGAGCCTAGCATT |
| | TATTCGTAAATCCGATGAATTATTACATAATGTAAATGCTGGTAAATC |
| | CACCACAAATATCATGATAACTACTATAATTATAGTGATTATAGTAAT |
| | ATTGTTATCATTAATTGCTGTTGGACTGCTCTTATACTGTAAGGCCAG |
| | AAGCACACCAGTCACACTAAGCAAAGATCAACTGAGTGGTATAAATA |
| | ATATTGCATTTAGTAACTAA |
| RNA encoding | AUGGAGUUGCUAAUCCUCAAAGCAAAUGCAAUUACCACAAUCCUCA |
| RSV Full Length | CUGCAGUCACAUUUUGUUUUGCUUCUGGUCAAAACAUCACUGAAG |
| F0 of Native RSV | AAUUUUAUCAAUCAACAUGCAGUGCAGUUAGCAAAGGCUAUCUUA |
| A2 (GenBank GI: | GUGCUCUGAGAACUGGUUGGUAUACCAGUGUUAUAACUAUAGAAU |
| 138251; Swiss Prot | UAAGUAAUAUCAAGGAAAAUAAGUGUAAUGGAACAGAUGCUAAGG |
| P03420) | UAAAAUUGAUAAAACAAGAAUUAGAUAAAUAUAAAAAUGCUGUAA |
| SEQ ID NO: 20 | CAGAAUUGCAGUUGCUCAUGCAAAGCACACCACCAACAAACAAUCG |
| | AGCCAGAAGAGAACUACCAAGGUUUAUGAAUUAUACACUCAACAA |
| | UGCCAAAAAAACCAAUGUAACAUUAAGCAAGAAAAGGAAAAGAAG |
| | AUUUCUUGGUUUUUUGUUAGGUGUUGGAUCUGCAAUCGCCAGUGG |
| | CGUUGCUGUAUCUAAGGUCCUGCACCUAGAAGGGGAAGUGAACAA |
| | GAUCAAAAGUGCUCUACUAUCCACAAACAAGGCUGUAGUCAGCUUA |
| | UCAAAUGGAGUUAGUGUCUUAACCAGCAAAGUGUUAGACCUCAAA |
| | AACUAUAUAGAUAAACAAUUGUUACCUAUUGUGAACAAGCAAAGC |
| | UGCAGCAUAUCAAAUAUAGAAACUGUGAUAGAGUUCCAACAAAAG |
| | AACAACAGACUACUAGAGAUUACCAGGGAAUUUAGUGUUAAUGCA |
| | GGUGUAACUACACCUGUAAGCACUUACAUGUUAACUAAUAGUGAA |
| | UUAUUGUCAUUAAUCAAUGAUAUGCCUAUAACAAAUGAUCAGAAA |
| | AAGUUAAUGUCCAACAAUGUUCAAAUAGUUAGACAGCAAAGUUAC |
| | UCUAUCAUGUCCAUAAUAAAAGAGGAAGUCUUAGCAUAUGUAGUA |
| | CAAUUACCACUAUAUGGUGUUAUAGAUACACCCUGUUGGAAACUA |
| | CACACAUCCCCUCUAUGUACAACCAACACAAAAGAAGGGUCCAACA |
| | UCUGUUUAACAAGAACUGACAGAGGAUGGUACUGUGACAAUGCAG |
| | GAUCAGUAUCUUUCUUCCCACAAGCUGAAACAUGUAAAGUUCAAUC |
| | AAAUCGAGUAUUUUGUGACACAAUGAACAGUUUAACAUUACCAAG |
| | UGAAAUAAAUCUCUGCAAUGUUGACAUAUUCAACCCCAAAUAUGA |
| | UUGUAAAAUUAUGACUUCAAAAACAGAUGUAAGCAGCUCCGUUAU |
| | CACAUCUCUAGGAGCCAUUGUGUCAUGCUAUGGCAAAACUAAAUG |
| | UACAGCAUCCAAUAAAAAUCGUGGAAUCAUAAAGACAUUUUCUAA |
| | CGGGUGCGAUUAUGUAUCAAAUAAAGGGAUGGACACUGUGUCUGU |
| | AGGUAACACAUUAUAUUAUGUAAAUAGCAAGAAGGUAAAAGUCUC |
| | UAUGUAAAAGGUGAACCAAUAAUAAAUUUCUAUGACCCAUUAGUA |
| | UUCCCCUCUGAUGAAUUUGAUGCAUCAAUAUCUCAAGUCAACGAGA |
| | AGAUUAACCAGAGCCUAGCAUUUAUUCGUAAAUCCGAUGAAUUAU |
| | UACAUAAUGUAAAUGCUGGUAAAUCCACCACAAAUAUCAUGAUAA |
| | CUACUAUAAUUAUAGUGAUUAUAGUAAUAUUGUUAUCAUUAAUUG |
| | CUGUUGGACUGCUCUUAUACUGUAAGGCCAGAAGCACACCAGUCAC |
| | ACUAAGCAAAGAUCAACUGAGUGGUAUAAAUAAUAUUGCAUUUAG |
| | UAACUAA |
| RSV Full Length | MELLIHRSSAIFLTLAVNALYLTSSQNITEEFYQSTCSAVSRGYFSALRTG |
| F0 of Native RSV | WYTSVITIE |
| B (18537 strain; | LSNIKETKCNGTDTKVKLIKQELDKYKNAVTELQLLMONTPAANNRAR |
| GenBank GI: | REAPQYMNYTINTTKNLNVSISKKRKRRFLGFLLGVGSAIASGIAVSKVL |
| 138250; Swiss Prot | HLEGEVNKIKNALLSTNKAVVSLSNGVSVLTSKVLDLKNYINNRLLPIVN |
| P13843) | QQSCRISNIETVIEFQQMNSRLLEITREFSVN |
| SEQ ID NO: 21 | AGVTTPLSTYMLTNSELLSLINDMPITNDQKKLMSSNVQIVRQQSYSIMSI |
| | IKEEVLAYV |
| | VQLPIYGVIDTPCWKLHTSPLCTTNIKEGSNICLTRTDRGWYCDNAGSVS |
| | FFPQADTCKVQSNRVFCDTMNSLTLPSEVSLCNTDIFNSKYDCKIMTSKT |
| | DISSSVITSLGAIVSCYGKTKCTASNKNRGIIKTFSNGCDYVSNKGVDTVS |
| | V |
| | GNTLYYVNKLEGKNLYVKGEPIINYYDPLVFPSDEFDASISQVNEKINQS |
| | LAFIRRSDELLHNVNTGKSTTNIMITTIIIVIIVVLLSLIAIGLLLYCKAKNTP |
| | VTLSKDQLSGINNIAFSK |
| DNA encoding | ATGGAGTTGCTGATCCACAGGTCAAGTGCAATCTTCCTAACTCTTGCT |
| RSV Full Length | GTTAATGCATTGTACCTCACCTCAAGTCAGAACATAACTGAGGAGTTT |
| F0 of Native RSV | TACCAATCGACATGTAGTGCAGTTAGCAGAGGTTATTTTAGTGCTTTA |
| B (18537 strain; | AGAACAGGTTGGTATACCAGTGTCATAACAATAGAATTAAGTAATAT |
| GenBank GI: | AAAAGAAACCAAATGCAATGGAACTGACACTAAAGTAAAACTTATA |
| 138250; Swiss Prot | AAACAAGAATTAGATAAGTATAAGAATGCAGTAACAGAATTACAGCT |
| P13843) | ACTTATGCAAAACACGCCAGCTGCCAACAACCGGGCCAGAAGAGAA |
| SEQ ID NO: 22 | GCACCACAGTACATGAACTACACAATCAATACCACAAAAAACCTAAA |
| | TGTATCAATAAGCAAGAAAAGGAAACGAAGATTTCTGGGCTTCTTGT |
| | TAGGTGTAGGATCTGCAATAGCAAGTGGTATAGCTGTATCCAAAGTT |
| | TTACACCTTGAAGGAGAAGTGAACAAAATCAAAAATGCTTTGTTGTC |
| | TACAAACAAAGCTGTAGTCAGTCTATCAAATGGGGTCAGTGTTTTAA |
| | CCAGCAAAGTGTTAGATCTCAAGAATTACATAAATAACCGATTATTA |
| | CCCATAGTAAATCAACAGAGCTGTCGCATCTCCAACATTGAAACAGT |
| | TATAGAATTCCAGCAGATGAATAGCAGATTGTTGGAAATCACCAGAG |
| | AATTTAGTGTTAATGCAGGTGTAACAACACCTTTAAGCACTTACATGT |
| | TAACAAACAGTGAGTTACTATCATTGATCAATGATATGCCTATAACA |
| | AATGACCAGAAAAAATTAATGTCAAGCAATGTTCAGATAGTAAGGCA |
| | ACAAAGTTATTCTATCATGTCTATAATAAAGGAAGAAGTCCTTGCATA |
| | TGTTGTACAGCTACCTATCTATGGTGTAATAGATACACCTTGCTGGAA |
| | ATTACACACATCACCTCTATGCACCACCAACATCAAAGAAGGATCAA |
| | ATATTTGTTTAACAAGGACTGATAGAGGATGGTATTGTGATAATGCA |
| | GGATCAGTATCCTTCTTCCCACAGGCTGATACTTGCAAAGTACAGTCC |
| | AATCGAGTATTTTGTGACACTATGAACAGTTTAACATTACCAAGTGAA |
| | GTCAGCCTTTGTAACACTGACATATTCAATTCCAAGTATGACTGCAAA |
| | ATTATGACATCAAAAACAGACATAAGCAGCTCAGTAATTACTTCTCTT |
| | GGAGCTATAGTGTCATGCTATGGAAAAACTAAATGCACTGCATCCAA |
| | TAAAAATCGTGGGATTATAAAGACATTTTCTAATGGTTGTGACTATGT |
| | GTCAAACAAAGGAGTAGATACTGTGTCAGTGGGCAACACTTTATACT |
| | ATGTAAACAAGCTGGAAGGCAAAAACCTTTATGTAAAAGGGGAACCT |
| | ATAATAAATTACTATGATCCTCTAGTGTTTCCTTCTGATGAGTTTGAT |
| | GCATCAATATCTCAAGTCAATGAAAAAATCAATCAAAGTTTAGCTTTT |
| | ATTCGTAGATCTGATGAATTACTACATAATGTAAATACTGGCAAATCT |
| | ACTACAAATATTATGATAACTACAATTATTATAGTAATCATTGTAGTA |
| | TTGTTATCATTAATAGCTATTGGTTTACTGTTGTATTGCAAAGCCAAA |
| | AACACACCAGTTACACTAAGCAAAGACCAACTAAGTGGAATCAATAA |
| | TATTGCATTCAGCAAATAG |
| RNA encoding | AUGGAGUUGCUGAUCCACAGGUCAAGUGCAAUCUUCCUAACUCUUG |
| RSV Full Length | CUGUUAAUGCAUUGUACCUCACCUCAAGUCAGAACAUAACUGAGGA |
| F0 of Native RSV | GUUUUACCAAUCGACAUGUAGUGCAGUUAGCAGAGGUUAUUUUAG |
| B (18537 strain; | UGCUUUAAGAACAGGUUGGUAUACCAGUGUCAUAACAAUAGAAUU |
| GenBank GI: | AAGUAAUAUAAAAGAAACCAAAUGCAAUGGAACUGACACUAAAGU |
| 138250; Swiss Prot | AAAACUUAUAAAACAAGAAUUAGAUAAGUAUAAGAAUGCAGUAAC |
| P13843) | AGAAUUACAGCUACUUAUGCAAAACACGCCAGCUGCCAACAACCGG |
| SEQ ID NO: 23 | GCCAGAAGAGAAGCACCACAGUACAUGAACUACACAAUCAAUACCA |
| | CAAAAAACCUAAAUGUAUCAAUAAGCAAGAAAAGGAAACGAAGAU |
| | UUCUGGGCUUCUUGUUAGGUGUAGGAUCUGCAAUAGCAAGUGGUA |
| | UAGCUGUAUCCAAAGUUUUACACCUUGAAGGAGAAGUGAACAAAA |
| | UCAAAAAUGCUUUGUUGUCUACAAACAAAGCUGUAGUCAGUCUAU |
| | CAAAUGGGGUCAGUGUUUUAACCAGCAAAGUGUUAGAUCUCAAGA |
| | AUUACAUAAAUAACCGAUUAUUACCCAUAGUAAAUCAACAGAGCU |
| | GUCGCAUCUCCAACAUUGAAACAGUUAUAGAAUUCCAGCAGAUGA |
| | AUAGCAGAUUGUUGGAAAUCACCAGAGAAUUUAGUGUUAAUGCAG |
| | GUGUAACAACACCUUUAAGCACUUACAUGUUAACAAACAGUGAGU |
| | UACUAUCAUUGAUCAAUGAUAUGCCUAUAACAAAUGACCAGAAAA |
| | AAUUAAUGUCAAGCAAUGUUCAGAUAGUAAGGCAACAAAGUUAUU |
| | CUAUCAUGUCUAUAAUAAAGGAAGAAGUCCUUGCAUAUGUUGUAC |
| | AGCUACCUAUCUAUGGUGUAAUAGAUACACCUUGCUGGAAAUUAC |
| | ACACAUCACCUCUAUGCACCACCAACAUCAAAGAAGGAUCAAAUAU |
| | UUGUUUAACAAGGACUGAUAGAGGAUGGUAUUGUGAUAAUGCAGG |
| | AUCAGUAUCCUUCUUCCCACAGGCUGAUACUUGCAAAGUACAGUCC |
| | AAUCGAGUAUUUUGUGACACUAUGAACAGUUUAACAUUACCAAGU |
| | GAAGUCAGCCUUUGUAACACUGACAUAUUCAAUUCCAAGUAUGAC |
| | UGCAAAAUUAUGACAUCAAAAACAGACAUAAGCAGCUCAGUAAUU |
| | ACUUCUCUUGGAGCUAUAGUGUCAUGCUAUGGAAAAACUAAAUGC |
| | ACUGCAUCCAAUAAAAAUCGUGGGAUUAUAAAGACAUUUUCUAAU |
| | GGUUGUGACUAUGUGUCAAACAAAGGAGUAGAUACUGUGUCAGUG |
| | GGCAACACUUUAUACUAUGUAAACAAGCUGGAAGGCAAAAACCUU |
| | UAUGUAAAAGGGGAACCUAUAAUAAAUUACUAUGAUCCUCUAGUG |
| | UUUCCUUCUGAUGAGUUUGAUGCAUCAAUAUCUCAAGUCAAUGAA |
| | AAAAUCAAUCAAAGUUUAGCUUUUAUUCGUAGAUCUGAUGAAUUA |
| | CUACAUAAUGUAAAUACUGGCAAAUCUACUACAAAUAUUAUGAUA |
| | ACUACAAUUAUUAUAGUAAUCAUUGUAGUAUUGUUAUCAUUAAUA |
| | GCUAUUGGUUUACUGUUGUAUUGCAAAGCCAAAAACACACCAGUU |
| | ACACUAAGCAAAGACCAACUAAGUGGAAUCAAUAAUAUUGCAUUC |
| | AGCAAAUAG |
| RSV RSVA/Homo | MELPILKTNAITTILAAVTLCFASSQNITEEFYQSTCSAVSKGYLSALRTG |
| sapiens/USA/LA2_ | WYTSVITIELSNIKENKCNGTDAKVKLIKQELDKYKNAVTELQLLMQSTP |
| 21/2013 F | AANSRARRELPRFMNYTLNNTKNTNVTLSKKRKRRFLGFLLGVGSAIAS |
| (Ontario) Native | GIAVSKVLHLEGEVNKIKSALLSTNKAVVSLSNGVSVLTSKVLDLKNYID |
| Amino Acid | KQLLPIVNKQSCSISNIETVIEFQQKNNRLLEITREFSVNAGVTTPVSTYML |
| Sequence | TNSELLSLINDMPITNDQKKLMSSNVQIVRQQSYSIMSIIKEEVLAYVVQL |
| (GenBank GI: | PLYGVIDTPCWKLHTSPLCTTNTKEGSNICLTRTDRGWYCDNAGSVSFFP |
| AHX57185) | QAETCKVQSNRVFCDTMNSLTLPSEVNLCNIDIFNPKYDCKIMTSKTDVS |
| SEQ ID NO: 24 | SSVITSLGAIVSCYGKTKCTASNKNRGIIKTFSNGCDYVSNKGVDTVSV |
| | GNTLYYVNKQEGKSLYVKGEPIINFYDPLVFPSDEFDASISQVNEKINQSL |
| | AFIRKSDELLHNVNAGKSTTNIMITTIIIVIIVILLALIAVGLLLYCKARSTP |
| | VTLSKDQLSGINNIAFSN |
| DNA encoding | ATGGAGTTGCCAATCCTCAAAACAAATGCTATTACCACAATCCTTGCT |
| RSVA/Homo | GCAGTCACACTCTGCTTCGCTTCCAGTCAAAACATCACTGAAGAATTT |
| sapiens/USA/LA2_ | TATCAATCAACATGCAGTGCAGTTAGCAAAGGCTATCTTAGTGCTCTA |
| 21/2013 F | AGAACTGGTTGGTATACTAGTGTTATAACTATAGAATTAAGTAATATC |
| (Ontario) Native | AAGGAAAATAAGTGTAATGGTACAGATGCTAAGGTAAAATTAATAAA |
| Amino Acid | ACAAGAATTAGATAAATATAAAAATGCTGTAACAGAATTGCAGTTGC |
| Sequence | TCATGCAAAGCACACCAGCAGCCAACAGTCGAGCCAGAAGAGAACT |
| (GenBank GI: | GCCAAGATTTATGAATTATACACTCAACAATACCAAAAACACCAATG |
| AHX57185) | TAACATTAAGTAAGAAAAGGAAAAGAAGATTTCTTGGATTTTTGTTA |
| SEQ ID NO: 25 | GGTGTTGGATCTGCAATCGCCAGTGGCATTGCCGTATCCAAGGTCCTG |
| | CACCTAGAAGGGGAAGTGAACAAAATCAAAAGTGCTCTACTATCCAC |
| | AAACAAGGCTGTAGTCAGCTTATCTAATGGAGTCAGTGTCTTAACCA |
| | GCAAGGTGTTAGACCTCAAAAACTATATAGATAAACAGTTGTTACCT |
| | ATTGTTAACAAGCAAAGCTGCAGCATATCAAACATTGAAACTGTGAT |
| | AGAGTTCCAACAAAAGAACAACAGACTACTAGAGATTACCAGAGAA |
| | TTTAGTGTTAATGCAGGTGTAACTACACCTGTAAGCACTTATATGTTA |
| | ACTAATAGTGAGTTATTATCATTAATCAATGATATGCCTATAACAAAT |
| | GATCAGAAAAAGTTAATGTCCAGCAATGTTCAAATAGTTAGACAGCA |
| | AAGTTACTCTATCATGTCAATAATAAAAGAGGAAGTCTTAGCATATG |
| | TAGTACAATTACCACTATATGGTGTAATAGATACTCCTTGTTGGAAAC |
| | TACACACATCCCCTCTATGTACAACCAACACAAAGGAAGGATCCAAC |
| | ATCTGCTTAACAAGAACCGACAGAGGATGGTACTGTGACAATGCAGG |
| | ATCAGTATCCTTTTTCCCACAAGCTGAAACATGTAAAGTTCAATCGAA |
| | TCGGGTATTTTGTGACACAATGAACAGTTTAACATTACCAAGTGAGGT |
| | AAATCTCTGCAACATTGACATATTCAACCCCAAATATGATTGCAAAAT |
| | TATGACTTCAAAAACAGATGTAAGCAGCTCCGTTATCACATCTCTAGG |
| | AGCCATTGTGTCATGCTATGGCAAAACCAAATGTACAGCATCCAATA |
| | AAAATCGTGGGATCATAAAGACATTCTCTAACGGGTGTGATTATGTA |
| | TCAAATAAGGGGGTGGATACTGTGTCTGTAGGTAATACATTATATTAT |
| | GTAAATAAGCAAGAAGGCAAAAGTCTCTATGTAAAAGGTGAACCAAT |
| | AATAAATTTCTATGATCCATTAGTGTTCCCCTCTGATGAATTTGATGC |
| | ATCAATATCTCAAGTCAATGAGAAAATTAATCAGAGTCTAGCATTTAT |
| | CCGTAAATCAGATGAATTATTACATAATGTAAATGCTGGTAAATCCA |
| | CCACAAATATCATGATAACTACCATAATTATAGTAATTATAGTAATAT |
| | TGTTAGCATTAATTGCAGTTGGACTGCTTCTATACTGCAAGGCCAGAA |
| | GCACACCAGTCACATTAAGTAAGGATCAACTGAGTGGTATAAATAAT |
| | ATTGCATTTAGTAACTGA |
| RNA encoding | AUGGAGUUGCCAAUCCUCAAAACAAAUGCUAUUACCACAAUCCUUG |
| RSVA/Homo | CUGCAGUCACACUCUGCUUCGCUUCCAGUCAAAACAUCACUGAAGA |
| sapiens/USA/LA2_ | AUUUUAUCAAUCAACAUGCAGUGCAGUUAGCAAAGGCUAUCUUAG |
| 21/2013 F | UGCUCUAAGAACUGGUUGGUAUACUAGUGUUAUAACUAUAGAAUU |
| (Ontario) Native | AAGUAAUAUCAAGGAAAAUAAGUGUAAUGGUACAGAUGCUAAGGU |
| Amino Acid | AAAAUUAAUAAAACAAGAAUUAGAUAAAUAUAAAAAUGCUGUAAC |
| Sequence | AGAAUUGCAGUUGCUCAUGCAAAGCACACCAGCAGCCAACAGUCGA |
| (GenBank GI: | GCCAGAAGAGAACUGCCAAGAUUUAUGAAUUAUACACUCAACAAU |
| AHX57185) | ACCAAAAACACCAAUGUAACAUUAAGUAAGAAAAGGAAAAGAAGA |
| SEQ ID NO: 26 | UUUCUUGGAUUUUUGUUAGGUGUUGGAUCUGCAAUCGCCAGUGGC |
| | AUUGCCGUAUCCAAGGUCCUGCACCUAGAAGGGGAAGUGAACAAA |
| | AUCAAAAGUGCUCUACUAUCCACAAACAAGGCUGUAGUCAGCUUAU |
| | CUAAUGGAGUCAGUGUCUUAACCAGCAAGGUGUUAGACCUCAAAA |
| | ACUAUAUAGAUAAACAGUUGUUACCUAUUGUUAACAAGCAAAGCU |
| | GCAGCAUAUCAAACAUUGAAACUGUGAUAGAGUUCCAACAAAAGA |
| | ACAACAGACUACUAGAGAUUACCAGAGAAUUUAGUGUUAAUGCAG |
| | GUGUAACUACACCUGUAAGCACUUAUAUGUUAACUAAUAGUGAGU |
| | UAUUAUCAUUAAUCAAUGAUAUGCCUAUAACAAAUGAUCAGAAAA |
| | AGUUAAUGUCCAGCAAUGUUCAAAUAGUUAGACAGCAAAGUUACU |
| | CUAUCAUGUCAAUAAUAAAAGAGGAAGUCUUAGCAUAUGUAGUAC |
| | AAUUACCACUAUAUGGUGUAAUAGAUACUCCUUGUUGGAAACUAC |
| | ACACAUCCCCUCUAUGUACAACCAACACAAAGGAAGGAUCCAACAU |
| | CUGCUUAACAAGAACCGACAGAGGAUGGUACUGUGACAAUGCAGG |
| | AUCAGUAUCCUUUUUCCCACAAGCUGAAACAUGUAAAGUUCAAUCG |
| | AAUCGGGUAUUUUGUGACACAAUGAACAGUUUAACAUUACCAAGU |
| | GAGGUAAAUCUCUGCAACAUUGACAUAUUCAACCCCAAAUAUGAU |
| | UGCAAAAUUAUGACUUCAAAAACAGAUGUAAGCAGCUCCGUUAUC |
| | ACAUCUCUAGGAGCCAUUGUGUCAUGCUAUGGCAAAACCAAAUGU |
| | ACAGCAUCCAAUAAAAAUCGUGGGAUCAUAAAGACAUUCUCUAAC |
| | GGGUGUGAUUAUGUAUCAAAUAAGGGGGUGGAUACUGUGUCUGUA |
| | GGUAAUACAUUAUAUUAUGUAAAUAAGCAAGAAGGCAAAAGUCUC |
| | UAUGUAAAAGGUGAACCAAUAAUAAAUUUCUAUGAUCCAUUAGUG |
| | UUCCCCUCUGAUGAAUUUGAUGCAUCAAUAUCUCAAGUCAAUGAG |
| | AAAAUUAAUCAGAGUCUAGCAUUUAUCCGUAAAUCAGAUGAAUUA |
| | UUACAUAAUGUAAAUGCUGGUAAAUCCACCACAAAUAUCAUGAUA |
| | ACUACCAUAAUUAUAGUAAUUAUAGUAAUAUUGUUAGCAUUAAUU |
| | GCAGUUGGACUGCUUCUAUACUGCAAGGCCAGAAGCACACCAGUCA |
| | CAUUAAGUAAGGAUCAACUGAGUGGUAUAAAUAAUAUUGCAUUUA |
| | GUAACUGA |
| RSV RSVB/Homo | MELLIHRSSAIFLTLAINALYLTSSQNITEEFYQSTCSAVSRGYFSALRTG |
| sapiens/PER/ | WYTSVITIELSNIKETKCNGTDTKVKLIKQELDKYKNAVTELQLLMQNTP |
| FPP00592/2011 F | AANNRARREAPQYMNYTINTTKNLNVSISKKRKRRFLGFLLGVGSAIAS |
| (Buenos Aires) | GIAVSKVLHLEGEVNKIKNALLSTNKAVVSLSNGVSVLTSKVLDLKNYIN |
| Native Amino | NQLLPIVNQQSCRISNIETVIEFQQKNSRLLEITREFSVNAGVTTPLSTYML |
| Acid Sequence | TNSELLSLINDMPITNDQKKLMSSNVQIVRQQSYSIMSIIKEEVLAYVVQL |
| (GenBank GI: | PIYGVIDTPCWKLHTSPLCTTNIKEGSNICLTRTDRGWYCDNAGSVSFFPQ |
| AHV80758) | ADTCKVQSNRVFCDTMNSLTLPSEVSLCNTDIFNSKYDCKIMTSKTDISSS |
| SEQ ID NO: 27 | VITSLGAIVSCYGKTKCTASNKNRGIIKTFSNGCDYVSNKGVDTVSV |
| | GNTLYYVNKLEGKNLYVKGEPIINYYDPLVFPSDEFDASISQVNEKINQS |
| | LAFIRRSDELLHNVNTGKSTTNIMITAIIIVIIVVLLSLIAIGLLLYCKAKNTP |
| | VTLSKDQLSGINNIAFSK |
| DNA encoding | ATGGAGTTGCTGATCCATAGATCAAGTGCAATCTTCCTAACTCTTGCT |
| RSV RSVB/Homo | ATTAATGCATTGTACCTCACCTCAAGTCAGAACATAACTGAGGAGTTT |
| sapiens/PER/ | TACCAATCGACATGTAGTGCAGTTAGCAGAGGTTACTTTAGTGCTTTA |
| FPP00592/2011 F | AGAACAGGTTGGTATACCAGTGTCATAACAATAGAATTAAGTAATAT |
| (Buenos Aires) | AAAAGAAACCAAATGCAATGGAACTGACACTAAAGTAAAACTTATA |
| Native Amino | AAACAAGAATTAGATAAGTATAAGAATGCAGTAACAGAATTACAGTT |
| Acid Sequence | ACTTATGCAAAACACACCAGCTGCCAACAACCGAGCCAGAAGAGAA |
| (GenBank GI: | GCACCACAGTATATGAACTACACAATCAATACCACTAAAAACCTAAA |
| AHV80758) | TGTATCAATAAGCAAGAAGAGGAAACGAAGATTTCTGGGCTTCTTGT |
| SEQ ID NO: 28 | TAGGTGTAGGATCTGCAATAGCAAGTGGCATAGCTGTATCCAAAGTT |
| | CTACACCTTGAAGGAGAAGTGAACAAGATCAAAAATGCTTTGCTGTC |
| | TACAAACAAAGCTGTAGTCAGTCTATCAAATGGGGTCAGTGTTTTAA |
| | CCAGCAAAGTGTTAGATCTCAAGAATTATATAAACAACCAATTATTA |
| | CCTATAGTAAATCAACAGAGTTGTCGCATCTCCAACATTGAAACAGTT |
| | ATAGAATTCCAGCAGAAGAACAGCAGATTGTTGGAAATCACCAGAGA |
| | ATTTAGTGTCAATGCAGGTGTAACGACACCTTTAAGCACTTACATGTT |
| | AACAAACAGTGAGTTACTATCATTAATCAATGATATGCCTATAACAA |
| | ATGATCAGAAGAAATTAATGTCAAGCAATGTTCAGATAGTAAGGCAA |
| | CAAAGTTATTCTATCATGTCTATAATAAAGGAAGAAGTCCTCGCATAT |
| | GTTGTACAGCTACCTATCTATGGTGTAATAGATACACCTTGCTGGAAA |
| | TTACACACATCACCTCTGTGCACCACCAACATCAAAGAAGGATCAAA |
| | TATTTGTTTAACAAGGACTGATAGAGGATGGTACTGTGATAATGCAG |
| | GATCAGTATCCTTCTTTCCACAGGCTGACACTTGTAAAGTACAGTCCA |
| | ATCGAGTATTTTGTGACACTATGAACAGTTTGACATTACCAAGTGAAG |
| | TCAGCCTTTGTAACACTGACATATTCAATTCCAAGTATGACTGCAAAA |
| | TTATGACATCAAAAACAGACATAAGCAGCTCAGTAATTACTTCTCTA |
| | GGAGCTATAGTGTCATGCTATGGTAAAACTAAATGCACTGCATCCAA |
| | CAAAAATCGTGGAATTATAAAGACATTTTCTAATGGTTGTGATTATGT |
| | GTCAAACAAAGGAGTAGATACTGTATCAGTAGGCAACACTTTATACT |
| | ATGTCAACAAGCTGGAAGGCAAAAACCTTTATGTAAAAGGGGAACCT |
| | ATAATAAATTACTATGACCCTCTAGTGTTTCCTTCTGATGAGTTTGAT |
| | GCATCAATATCTCAAGTCAATGAAAAAATTAATCAAAGTTTAGCTTTT |
| | ATTCGTAGATCCGATGAATTATTACATAATGTAAATACTGGAAAATCT |
| | ACTACAAATATTATGATAACTGCAATTATTATAGTAATCATTGTAGTA |
| | TTGTTATCATTAATAGCTATTGGTTTACTGTTGTATTGCAAAGCCAAA |
| | AACACACCAGTTACACTAAGCAAAGACCAACTAAGTGGAATCAACAA |
| | CATTGCATTCAGCAAATAG |
| RNA encoding | AUGGAGUUGCUGAUCCAUAGAUCAAGUGCAAUCUUCCUAACUCUU |
| RSV RSVB/Homo | GCUAUUAAUGCAUUGUACCUCACCUCAAGUCAGAACAUAACUGAGG |
| sapiens/PER/ | AGUUUUACCAAUCGACAUGUAGUGCAGUUAGCAGAGGUUACUUUA |
| FPP00592/2011 F | GUGCUUUAAGAACAGGUUGGUAUACCAGUGUCAUAACAAUAGAAU |
| (Buenos Aires) | UAAGUAAUAUAAAAGAAACCAAAUGCAAUGGAACUGACACUAAAG |
| Native Amino | UAAAACUUAUAAAACAAGAAUUAGAUAAGUAUAAGAAUGCAGUAA |
| Acid Sequence | CAGAAUUACAGUUACUUAUGCAAAACACACCAGCUGCCAACAACCG |
| (GenBank GI: | AGCCAGAAGAGAAGCACCACAGUAUAUGAACUACACAAUCAAUACC |
| AHV80758) | ACUAAAAACCUAAAUGUAUCAAUAAGCAAGAAGAGGAAACGAAGA |
| SEQ ID NO: 29 | UUUCUGGGCUUCUUGUUAGGUGUAGGAUCUGCAAUAGCAAGUGGC |
| | AUAGCUGUAUCCAAAGUUCUACACCUUGAAGGAGAAGUGAACAAG |
| | AUCAAAAAUGCUUUGCUGUCUACAAACAAAGCUGUAGUCAGUCUA |
| | UCAAAUGGGGUCAGUGUUUUAACCAGCAAAGUGUUAGAUCUCAAG |
| | AAUUAUAUAAACAACCAAUUAUUACCUAUAGUAAAUCAACAGAGU |
| | UGUCGCAUCUCCAACAUUGAAACAGUUAUAGAAUUCCAGCAGAAG |
| | AACAGCAGAUUGUUGGAAAUCACCAGAGAAUUUAGUGUCAAUGCA |
| | GGUGUAACGACACCUUUAAGCACUUACAUGUUAACAAACAGUGAG |
| | UUACUAUCAUUAAUCAAUGAUAUGCCUAUAACAAAUGAUCAGAAG |
| | AAAUUAAUGUCAAGCAAUGUUCAGAUAGUAAGGCAACAAAGUUAU |
| | UCUAUCAUGUCUAUAAUAAAGGAAGAAGUCCUCGCAUAUGUUGUA |
| | CAGCUACCUAUCUAUGGUGUAAUAGAUACACCUUGCUGGAAAUUA |
| | CACACAUCACCUCUGUGCACCACCAACAUCAAAGAAGGAUCAAAUA |
| | UUUGUUUAACAAGGACUGAUAGAGGAUGGUACUGUGAUAAUGCAG |
| | GAUCAGUAUCCUUCUUUCCACAGGCUGACACUUGUAAAGUACAGUC |
| | CAAUCGAGUAUUUUGUGACACUAUGAACAGUUUGACAUUACCAAG |
| | UGAAGUCAGCCUUUGUAACACUGACAUAUUCAAUUCCAAGUAUGA |
| | CUGCAAAAUUAUGACAUCAAAAACAGACAUAAGCAGCUCAGUAAU |
| | UACUUCUCUAGGAGCUAUAGUGUCAUGCUAUGGUAAAACUAAAUG |
| | CACUGCAUCCAACAAAAAUCGUGGAAUUAUAAAGACAUUUUCUAA |
| | UGGUUGUGAUUAUGUGUCAAACAAAGGAGUAGAUACUGUAUCAGU |
| | AGGCAACACUUUAUACUAUGUCAACAAGCUGGAAGGCAAAAACCU |
| | UUAUGUAAAAGGGGAACCUAUAAUAAAUUACUAUGACCCUCUAGU |
| | GUUUCCUUCUGAUGAGUUUGAUGCAUCAAUAUCUCAAGUCAAUGA |
| | AAAAAUUAAUCAAAGUUUAGCUUUUAUUCGUAGAUCCGAUGAAUU |
| | AUUACAUAAUGUAAAUACUGGAAAAUCUACUACAAAUAUUAUGAU |
| | AACUGCAAUUAUUAUAGUAAUCAUUGUAGUAUUGUUAUCAUUAAU |
| | AGCUAUUGGUUUACUGUUGUAUUGCAAAGCCAAAAACACACCAGU |
| | UACACUAAGCAAAGACCAACUAAGUGGAAUCAACAACAUUGCAUUC |
| | AGCAAAUAG |
| RSV F2 | QNITEEFYQSTCSAVSKGYLSALRTGWYTSVITIELSNIKENKCNGTDAK |
| polypeptide of F | VKLIKQELDKYKNAVTELQLLMQSTPACNSRARR |
| protein of RSV | |
| subtype A | |
| SEQ ID NO: 30 | |
| RSV F1 | FLGFLLGVGSACASGIAVSKVLHLEGEVNKIKSALLSTNKAVVSLSNGVS |
| polypeptide of F | VLTIKVLDLKNYIDKQLLPIVNKQSCSISNIETVIEFQQKNNRLLEITREFS |
| protein of RSV | VNAGVTTPVSTYMLTNSELLSLINDMPITNDQKKLMSSNVQIVRQQSYSI |
| subtype A | MSIIKEEVLAYVVQLPLYGVIDTPCWKLHTSPLCTTNTKEGSNICLTRTDR |
| SEQ ID NO: 31 | GWYCDNAGSVSFFPQAETCKVQSNRVFCDTMNSLTLPSEVNLCNIDIFNP |
| | KYDCKIMTSKTDVSSSVITSLGAIVSCYGKTKCTASNKNRGIIKTFSNGCD |
| | YVSNKGVDTVSV |
| | GNTLYYVNKQEGKSLYVKGEPIINFYDPLVFPSSEFDASISQVNEKINQSL |
| | AFIRKSDELL |
| RSV F2 | QNITEEFYQSTCSAVSRGYFSALRTGWYTSVITIELSNIKETKCNGTDTKV |
| polypeptide of F | KLIKQELDKYKNAVTELQLLMQNTPACNNRARR |
| protein of RSV | |
| subtype B | |
| SEQ ID NO: 32 | |
| RSV F1 | FLGFLLGVGSACASGIAVSKVLHLEGEVNKIKNALLSTNKAVVSLSNGVS |
| polypeptide of F | VLTIKVLDLKNYINNQLLPIVNQQSCRISNIETVIEFQQKNSRLLEITREFSV |
| protein of RSV | NAGVTTPLSTYMLTNSELLSLINDMPITNDQKKLMSSNVQIVRQQSYSIM |
| subtype B | SIIKEEVLAYVVQLPIYGVIDTPCWKLHTSPLCTTNIKEGSNICLTRTDRG |
| SEQ ID NO: 33 | WYCDNAGSVSFFPQADTCKVQSNRVFCDTMNSLTLPSEVSLCNTDIFNS |
| | KYDCKIMTSKTDISSSVITSLGAIVSCYGKTKCTASNKNRGIIKTFSNGCD |
| | YVSNKGVDTVSV |
| | GNTLYYVNKLEGKNLYVKGEPIINYYDPLVFPSSEFDASISQVNEKINQSL |
| | AFIRRSDELL |
| RSV F Protein | MELLILKANAITTILTAVTFCFASGQNITEEFYQSTCSAVSKGYLSALRTG |
| Precursor | WYTSVITIELSNIKENKCNGTDAKVKLIKQELDKYKNAVTELQLLMQSTP |
| Polypeptide | ACNNRARRELPRFMNYTLNNAKKTNVTLSKKRKRRFLGFLLGVGSACA |
| [F1: residues 137- | SGVAVSKVLHLEGEVNKIKSALLSTNKAVVSLSNGVSVLTIKVLDLKNYI |
| 513; F2: residues | DKQLLPIVNKQSCSISNIETVIEFQQKNNRLLEITREFSVNAGVTTPVSTY |
| 26-109] | MLTNSELLSLINDMPITNDQKKLMSNNVQIVRQQSYSIMSIIKEEVLAYV |
| SEQ ID NO: 34 | VQLPLYGVIDTPCWKLHTSPLCTTNTKEGSNICLTRTDRGWYCDNAGSV |
| | SFFPQAETCKVQSNRVFCDTMNSLTLPSEVNLCNVDIFNPKYDCKIMTSK |
| | TDVSSSVITSLGAIVSCYGKTKCTASNKNRGIIKTFSNGCDYVSNKGVDT |
| | VSV |
| | GNTLYYVNKQEGKSLYVKGEPIINFYDPLVFPSSEFDASISQVNEKINQSL |
| | AFIRKSDELLSAIGGYIPEAPRDGQAYVRKDGEWVLLSTFLGGLVPRGSH |
| | HHHHHGSWSHPQFEK |
| DNA encoding | ATGGAACTTCTGATCCTGAAAGCCAACGCGATTACCACTATCCTGACT |
| RSV F Protein | GCCGTCACCTTCTGCTTCGCATCGGGACAGAACATTACCGAGGAGTTC |
| Precursor | TACCAGTCCACCTGTTCGGCGGTGTCCAAGGGTTACCTCTCGGCCCTG |
| Polypeptide | AGAACTGGCTGGTACACCAGCGTGATTACTATCGAGCTGAGCAACAT |
| [F1: bp 409-1539; | CAAGGAGAACAAGTGCAATGGAACGGACGCGAAGGTCAAGCTGATT |
| F2: bp 76-327] | AAGCAGGAACTCGATAAGTACAAGAACGCCGTGACCGAGCTCCAGCT |
| SEQ ID NO: 35 | GCTGATGCAATCGACCCCTGCCTGTAACAACAGAGCTCGCCGGGAAC |
| | TGCCGCGCTTCATGAATTACACCCTCAACAACGCGAAGAAAACCAAC |
| | GTGACCCTGTCCAAGAAGCGCAAGCGGAGGTTCCTGGGATTCCTGTT |
| | GGGCGTGGGCTCCGCATGTGCATCCGGAGTGGCCGTGTCCAAAGTGC |
| | TGCATCTGGAGGGGGAAGTGAACAAGATCAAGTCCGCCCTCCTGTCA |
| | ACTAATAAGGCGGTGGTGTCCCTGAGCAACGGAGTCAGCGTGCTGAC |
| | AATCAAGGTCCTGGACCTCAAGAACTACATCGACAAGCAGCTGTTGC |
| | CCATCGTCAACAAGCAGTCATGCTCGATTAGCAATATCGAAACCGTG |
| | ATTGAGTTCCAGCAGAAGAACAACAGACTGCTCGAAATTACCCGGGA |
| | GTTTTCCGTGAACGCCGGAGTGACCACTCCTGTGTCCACCTACATGCT |
| | TACGAACTCCGAACTGCTCAGCCTCATCAACGATATGCCGATCACTA |
| | ACGACCAGAAGAAGTTGATGAGCAACAATGTGCAGATCGTGCGCCAA |
| | CAGTCCTACTCAATCATGTCAATTATCAAGGAGGAAGTGCTCGCCTAT |
| | GTGGTGCAATTGCCTCTGTACGGAGTCATCGACACACCCTGCTGGAA |
| | GCTGCACACTAGCCCACTCTGTACGACCAACACCAAGGAAGGTTCCA |
| | ACATCTGCCTGACTAGGACCGATCGGGGCTGGTATTGCGATAATGCT |
| | GGGTCCGTGAGCTTCTTCCCGCAAGCCGAGACTTGCAAAGTGCAGTC |
| | AAACCGCGTGTTCTGTGACACCATGAACAGCCTGACCCTGCCATCCG |
| | AAGTCAACCTCTGCAACGTGGACATCTTTAACCCGAAATACGACTGC |
| | AAGATTATGACCTCCAAGACCGACGTCAGCAGCTCTGTCATCACTAG |
| | CCTGGGAGCTATTGTGTCCTGCTACGGAAAGACCAAATGCACTGCCT |
| | CGAACAAGAACAGAGGCATCATCAAGACCTTCAGCAACGGCTGTGAC |
| | TACGTGTCCAACAAGGGAGTGGACACCGTGTCCGTCGGGAACACCCT |
| | GTACTACGTGAACAAGCAGGAGGGGAAGTCGCTCTACGTCAAGGGG |
| | GAACCGATTATCAATTTCTACGACCCCCTGGTGTTCCCTTCCTCCGAG |
| | TTCGATGCCTCCATATCCCAAGTCAACGAGAAGATCAACCAGTCTCTT |
| | GCCTTCATCCGGAAGTCGGACGAACTGCTGTCCGCCATCGGTGGCTAT |
| | ATTCCGGAAGCCCCCAGGGATGGACAGGCCTACGTGCGGAAGGATGG |
| | AGAATGGGTGCTTTTGTCCACCTTCCTGGGCGGTCTGGTGCCCCGCGG |
| | CTCACACCATCATCACCACCACGGTTCGTGGTCCCACCCTCAATTTGA |
| | GAAGTGA |
| RNA encoding | AUGGAACUUCUGAUCCUGAAAGCCAACGCGAUUACCACUAUCCUGA |
| RSV F Protein | CUGCCGUCACCUUCUGCUUCGCAUCGGGACAGAACAUUACCGAGGA |
| Precursor | GUUCUACCAGUCCACCUGUUCGGCGGUGUCCAAGGGUUACCUCUCG |
| Polypeptide | GCCCUGAGAACUGGCUGGUACACCAGCGUGAUUACUAUCGAGCUGA |
| [F1: bp 409-1539; | GCAACAUCAAGGAGAACAAGUGCAAUGGAACGGACGCGAAGGUCA |
| F2: bp 76-327] | AGCUGAUUAAGCAGGAACUCGAUAAGUACAAGAACGCCGUGACCG |
| SEQ ID NO: 36 | AGCUCCAGCUGCUGAUGCAAUCGACCCCUGCCUGUAACAACAGAGC |
| | UCGCCGGGAACUGCCGCGCUUCAUGAAUUACACCCUCAACAACGCG |
| | AAGAAAACCAACGUGACCCUGUCCAAGAAGCGCAAGCGGAGGUUCC |
| | UGGGAUUCCUGUUGGGCGUGGGCUCCGCAUGUGCAUCCGGAGUGGC |
| | CGUGUCCAAAGUGCUGCAUCUGGAGGGGGAAGUGAACAAGAUCAA |
| | GUCCGCCCUCCUGUCAACUAAUAAGGCGGUGGUGUCCCUGAGCAAC |
| | GGAGUCAGCGUGCUGACAAUCAAGGUCCUGGACCUCAAGAACUACA |
| | UCGACAAGCAGCUGUUGCCCAUCGUCAACAAGCAGUCAUGCUCGAU |
| | UAGCAAUAUCGAAACCGUGAUUGAGUUCCAGCAGAAGAACAACAG |
| | ACUGCUCGAAAUUACCCGGGAGUUUUCCGUGAACGCCGGAGUGACC |
| | ACUCCUGUGUCCACCUACAUGCUUACGAACUCCGAACUGCUCAGCC |
| | UCAUCAACGAUAUGCCGAUCACUAACGACCAGAAGAAGUUGAUGA |
| | GCAACAAUGUGCAGAUCGUGCGCCAACAGUCCUACUCAAUCAUGUC |
| | AAUUAUCAAGGAGGAAGUGCUCGCCUAUGUGGUGCAAUUGCCUCU |
| | GUACGGAGUCAUCGACACACCCUGCUGGAAGCUGCACACUAGCCCA |
| | CUCUGUACGACCAACACCAAGGAAGGUUCCAACAUCUGCCUGACUA |
| | GGACCGAUCGGGGCUGGUAUUGCGAUAAUGCUGGGUCCGUGAGCU |
| | UCUUCCCGCAAGCCGAGACUUGCAAAGUGCAGUCAAACCGCGUGUU |
| | CUGUGACACCAUGAACAGCCUGACCCUGCCAUCCGAAGUCAACCUC |
| | UGCAACGUGGACAUCUUUAACCCGAAAUACGACUGCAAGAUUAUG |
| | ACCUCCAAGACCGACGUCAGCAGCUCUGUCAUCACUAGCCUGGGAG |
| | CUAUUGUGUCCUGCUACGGAAAGACCAAAUGCACUGCCUCGAACAA |
| | GAACAGAGGCAUCAUCAAGACCUUCAGCAACGGCUGUGACUACGUG |
| | UCCAACAAGGGAGUGGACACCGUGUCCGUCGGGAACACCCUGUACU |
| | ACGUGAACAAGCAGGAGGGGAAGUCGCUCUACGUCAAGGGGGAAC |
| | CGAUUAUCAAUUUCUACGACCCCCUGGUGUUCCCUUCCUCCGAGUU |
| | CGAUGCCUCCAUAUCCCAAGUCAACGAGAAGAUCAACCAGUCUCUU |
| | GCCUUCAUCCGGAAGUCGGACGAACUGCUGUCCGCCAUCGGUGGCU |
| | AUAUUCCGGAAGCCCCCAGGGAUGGACAGGCCUACGUGCGGAAGGA |
| | UGGAGAAUGGGUGCUUUUGUCCACCUUCCUGGGCGGUCUGGUGCCC |
| | CGCGGCUCACACCAUCAUCACCACCACGGUUCGUGGUCCCACCCUC |
| | AAUUUGAGAAGUGA |
| RMM048_pColEl- | ORIGIN |
| TC83_VSV-G_ | 1 taatacgact cactatagat aggcggcgca tgagagaagc ccagaccaat tacctaccca |
| CO13476 bp ds-DNA | 61 aaatggagaa agttcacgtt gacatcgagg aagacagccc attcctcaga gctttgcagc |
| SEQ ID NO: 37 | 121 ggagcttccc gcagtttgag gtagaagcca agcaggtcac tgataatgac catgctaatg |
| | 181 ccagagcgtt ttcgcatctg gcttcaaaac tgatcgaaac ggaggtggac ccatccgaca |
| | 241 cgatccttga cattggaagt gcgcccgccc gcagaatgta ttctaagcac aagtatcatt |
| | 301 gtatctgtcc gatgagatgt gcggaagatc cggacagatt gtataagtat gcaactaagc |
| | 361 tgaagaaaaa ctgtaaggaa ataactgata aggaattgga caagaaaatg aaggagctcg |
| | 421 ccgccgtcat gagcgaccct gacctggaaa ctgagactat gtgcctccac gacgacgagt |
| | 481 cgtgtcgcta cgaagggcaa gtcgctgttt accaggatgt atacgcggtt gacggaccga |
| | 541 caagtctcta tcaccaagcc aataagggag ttagagtcgc ctactggata ggctttgaca |
| | 601 ccaccccttt tatgtttaag aacttggctg gagcatatcc atcatactct accaactggg |
| | 661 ccgacgaaac cgtgttaacg gctcgtaaca taggcctatg cagctctgac gttatggagc |
| | 721 ggtcacgtag agggatgtcc attcttagaa agaagtattt gaaaccatcc aacaatgttc |
| | 781 tattctctgt tggctcgacc atctaccacg agaagaggga cttactgagg agctggcacc |
| | 841 tgccgtctgt atttcactta cgtggcaagc aaaattacac atgtcggtgt gagactatag |
| | 901 ttagttgcga cgggtacgtc gttaaaagaa tagctatcag tccaggcctg tatgggaagc |
| | 961 cttcaggcta tgctgctacg atgcaccgcg agggattctt gtgctgcaaa gtgacagaca |
| | 1021 cattgaacgg ggagagggtc tcttttcccg tgtgcacgta tgtgccagct acattgtgtg |
| | 1081 accaaatgac tggcatactg gcaacagatg tcagtgcgga cgacgcgcaa aaactgctgg |
| | 1141 ttgggctcaa ccagcgtata gtcgtcaacg gtcgcaccca gagaaacacc aataccatga |
| | 1201 aaaattacct tttgcccgta gtggcccagg catttgctag gtgggcaaag gaatataagg |
| | 1261 aagatcaaga agatgaaagg ccactaggac tacgagatag acagttagtc atggggtgtt |
| | 1321 gttgggcttt tagaaggcac aagataacat ctatttataa gcgcccggat acccaaacca |
| | 1381 tcatcaaagt gaacagcgat ttccactcat tcgtgctgcc caggataggc agtaacacat |
| | 1441 tggagatcgg gctgagaaca agaatcagga aaatgttaga ggagcacaag gagccgtcac |
| | 1501 ctctcattac cgccgaggac gtacaagaag ctaagtgcgc agccgatgag gctaaggagg |
| | 1561 tgcgtgaagc cgaggagttg cgcgcagctc taccaccttt ggcagctgat gttgaggagc |
| | 1621 ccactctgga agccgatgtc gacttgatgt tacaagaggc tggggccggc tcagtggaga |
| | 1681 cacctcgtgg cttgataaag gttaccagct acgatggcga ggacaagatc ggctcttacg |
| | 1741 ctgtgctttc tccgcaggct gtactcaaga gtgaaaaatt atcttgcatc caccctctcg |
| | 1801 ctgaacaagt catagtgata acacactctg gccgaaaagg gcgttatgcc gtggaaccat |
| | 1861 accatggtaa agtagtggtg ccagagggac atgcaatacc cgtccaggac tttcaagctc |
| | 1921 tgagtgaaag tgccaccatt gtgtacaacg aacgtgagtt cgtaaacagg tacctgcacc |
| | 1981 atattgccac acatggagga gcgctgaaca ctgatgaaga atattacaaa actgtcaagc |
| | 2041 ccagcgagca cgacggcgaa tacctgtacg acatcgacag gaaacagtgc gtcaagaaag |
| | 2101 aactagtcac tgggctaggg ctcacaggcg agctggtgga tcctcccttc catgaattcg |
| | 2161 cctacgagag tctgagaaca cgaccagccg ctccttacca agtaccaacc ataggggtgt |
| | 2221 atggcgtgcc aggatcaggc aagtctggca tcattaaaag cgcagtcacc aaaaaagatc |
| | 2281 tagtggtgag cgccaagaaa gaaaactgtg cagaaattat aagggacgtc aagaaaatga |
| | 2341 aagggctgga cgtcaatgcc agaactgtgg actcagtgct cttgaatgga tgcaaacacc |
| | 2401 ccgtagagac cctgtatatt gacgaagctt ttgcttgtca tgcaggtact ctcagagcgc |
| | 2461 tcatagccat tataagacct aaaaaggcag tgctctgcgg ggatcccaaa cagtgcggtt |
| | 2521 tttttaacat gatgtgcctg aaagtgcatt ttaaccacga gatttgcaca caagtcttcc |
| | 2581 acaaaagcat ctctcgccgt tgcactaaat ctgtgacttc ggtcgtctca accttgtttt |
| | 2641 acgacaaaaa aatgagaacg acgaatccga aagagactaa gattgtgatt gacactaccg |
| | 2701 gcagtaccaa acctaagcag gacgatctca ttctcacttg tttcagaggg tgggtgaagc |
| | 2761 agttgcaaat agattacaaa ggcaacgaaa taatgacggc agctgcctct caagggctga |
| | 2821 cccgtaaagg tgtgtatgcc gttcggtaca aggtgaatga aaatcctctg tacgcaccca |
| | 2881 cctcagaaca tgtgaacgtc ctactgaccc gcacggagga ccgcatcgtg tggaaaacac |
| | 2941 tagccggcga cccatggata aaaacactga ctgccaagta ccctgggaat ttcactgcca |
| | 3001 cgatagagga gtggcaagca gagcatgatg ccatcatgag gcacatcttg gagagaccgg |
| | 3061 accctaccga cgtcttccag aataaggcaa acgtgtgttg ggccaaggct ttagtgccgg |
| | 3121 tgctgaagac cgctggcata gacatgacca ctgaacaatg gaacactgtg gattattttg |
| | 3181 aaacggacaa agctcactca gcagagatag tattgaacca actatgcgtg aggttctttg |
| | 3241 gactcgatct ggactccggt ctattttctg cacccactgt tccgttatcc attaggaata |
| | 3301 atcactggga taactccccg tcgcctaaca tgtacgggct gaataaagaa gtggtccgtc |
| | 3361 agctctctcg caggtaccca caactgcctc gggcagttgc cactggaaga gtctatgaca |
| | 3421 tgaacactgg tacactgcgc aattatgatc cgcgcataaa cctagtacct gtaaacagaa |
| | 3481 gactgcctca tgctttagtc ctccaccata atgaacaccc acagagtgac ttttcttcat |
| | 3541 tcgtcagcaa attgaagggc agaactgtcc tggtggtcgg ggaaaagttg tccgtcccag |
| | 3601 gcaaaatggt tgactggttg tcagaccggc ctgaggctac cttcagagct cggctggatt |
| | 3661 taggcatccc aggtgatgtg cccaaatatg acataatatt tgttaatgtg aggaccccat |
| | 3721 ataaatacca tcactatcag cagtgtgaag accatgccat taagcttagc atgttgacca |
| | 3781 agaaagcttg tctgcatctg aatcccggcg gaacctgtgt cagcataggt tatggttacg |
| | 3841 ctgacagggc cagcgaaagc atcattggtg ctatagcgcg gcagttcaag ttttcccggg |
| | 3901 tatgcaaacc gaaatcctca cttgaagaga cggaagttct gtttgtattc attgggtacg |
| | 3961 atcgcaaggc ccgtacgcac aatccttaca agctttcatc aaccttgacc aacatttata |
| | 4021 caggttccag actccacgaa gccggatgtg caccctcata tcatgtggtg cgaggggata |
| | 4081 ttgccacggc caccgaagga gtgattataa atgctgctaa cagcaaagga caacctggcg |
| | 4141 gaggggtgtg cggagcgctg tataagaaat tcccggaaag cttcgattta cagccgatcg |
| | 4201 aagtaggaaa agcgcgactg gtcaaaggtg cagctaaaca tatcattcat gccgtaggac |
| | 4261 caaacttcaa caaagtttcg gaggttgaag gtgacaaaca gttggcagag gcttatgagt |
| | 4321 ccatcgctaa gattgtcaac gataacaatt acaagtcagt agcgattcca ctgttgtcca |
| | 4381 ccggcatctt ttccgggaac aaagatcgac taacccaatc attgaaccat ttgctgacag |
| | 4441 ctttagacac cactgatgca gatgtagcca tatactgcag ggacaagaaa tgggaaatga |
| | 4501 ctctcaagga agcagtggct aggagagaag cagtggagga gatatgcata tccgacgact |
| | 4561 cttcagtgac agaacctgat gcagagctgg tgagggtgca tccgaagagt tctttggctg |
| | 4621 gaaggaaggg ctacagcaca agcgatggca aaactttctc atatttggaa gggaccaagt |
| | 4681 ttcaccaggc ggccaaggat atagcagaaa ttaatgccat gtggcccgtt gcaacggagg |
| | 4741 ccaatgagca ggtatgcatg tatatcctcg gagaaagcat gagcagtatt aggtcgaaat |
| | 4801 gccccgtcga agagtcggaa gcctccacac cacctagcac gctgccttgc ttgtgcatcc |
| | 4861 atgccatgac tccagaaaga gtacagcgcc taaaagcctc acgtccagaa caaattactg |
| | 4921 tgtgctcatc ctttccattg ccgaagtata gaatcactgg tgtgcagaag atccaatgct |
| | 4981 cccagcctat attgttctca ccgaaagtgc ctgcgtatat tcatccaagg aagtatctcg |
| | 5041 tggaaacacc accggtagac gagactccgg agccatcggc agagaaccaa tccacagagg |
| | 5101 ggacacctga acaaccacca cttataaccg aggatgagac caggactaga acgcctgagc |
| | 5161 cgatcatcat cgaagaggaa gaagaggata gcataagttt gctgtcagat ggcccgaccc |
| | 5221 accaggtgct gcaagtcgag gcagacattc acgggccgcc ctctgtatct agctcatcct |
| | 5281 ggtccattcc tcatgcatcc gactttgatg tggacagttt atccatactt gacaccctgg |
| | 5341 agggagctag cgtgaccagc ggggcaacgt cagccgagac taactcttac ttcgcaaaga |
| | 5401 gtatggagtt tctggcgcga ccggtgcctg cgcctcgaac agtattcagg aaccctccac |
| | 5461 atcccgctcc gcgcacaaga acaccgtcac ttgcacccag cagggcctgc tcgagaacca |
| | 5521 gcctagtttc caccccgcca ggcgtgaata gggtgatcac tagagaggag ctcgaggcgc |
| | 5581 ttaccccgtc acgcactcct agcaggtcgg tctcgagaac cagcctggtc tccaacccgc |
| | 5641 caggcgtaaa tagggtgatt acaagagagg agtttgaggc gttcgtagca caacaacaat |
| | 5701 gacggtttga tgcgggtgca tacatctttt cctccgacac cggtcaaggg catttacaac |
| | 5761 aaaaatcagt aaggcaaacg gtgctatccg aagtggtgtt ggagaggacc gaattggaga |
| | 5821 tttcgtatgc cccgcgcctc gaccaagaaa aagaagaatt actacgcaag aaattacagt |
| | 5881 taaatcccac acctgctaac agaagcagat accagtccag gaaggtggag aacatgaaag |
| | 5941 ccataacagc tagacgtatt ctgcaaggcc tagggcatta tttgaaggca gaaggaaaag |
| | 6001 tggagtgcta ccgaaccctg catcctgttc ctttgtattc atctagtgtg aaccgtgcct |
| | 6061 tttcaagccc caaggtcgca gtggaagcct gtaacgccat gttgaaagag aactttccga |
| | 6121 ctgtggcttc ttactgtatt attccagagt acgatgccta tttggacatg gttgacggag |
| | 6181 cttcatgctg cttagacact gccagttttt gccctgcaaa gctgcgcagc tttccaaaga |
| | 6241 aacactccta tttggaaccc acaatacgat cggcagtgcc ttcagcgatc cagaacacgc |
| | 6301 tccagaacgt cctggcagct gccacaaaaa gaaattgcaa tgtcacgcaa atgagagaat |
| | 6361 tgcccgtatt ggattcggcg gcctttaatg tggaatgctt caagaaatat gcgtgtaata |
| | 6421 atgaatattg ggaaacgttt aaagaaaacc ccatcaggct tactgaagaa aacgtggtaa |
| | 6481 attacattac caaattaaaa ggaccaaaag ctgctgctct ttttgcgaag acacataatt |
| | 6541 tgaatatgtt gcaggacata ccaatggaca ggtttgtaat ggacttaaag agagacgtga |
| | 6601 aagtgactcc aggaacaaaa catactgaag aacggcccaa ggtacaggtg atccaggctg |
| | 6661 ccgatccgct agcaacagcg tatctgtgcg gaatccaccg agagctggtt aggagattaa |
| | 6721 atgcggtcct gcttccgaac attcatacac tgtttgatat gtcggctgaa gactttgacg |
| | 6781 ctattatagc cgagcacttc cagcctgggg attgtgttct ggaaactgac atcgcgtcgt |
| | 6841 ttgataaaag tgaggacgac gccatggctc tgaccgcgtt aatgattctg gaagacttag |
| | 6901 gtgtggacgc agagctgttg acgctgattg aggcggcttt cggcgaaatt tcatcaatac |
| | 6961 atttgcccac taaaactaaa tttaaattcg gagccatgat gaaatctgga atgttcctca |
| | 7021 cactgtttgt gaacacagtc attaacattg taatcgcaag cagagtgttg agagaacggc |
| | 7081 taaccggatc accatgtgca gcattcattg gagatgacaa tatcgtgaaa ggagtcaaat |
| | 7141 cggacaaatt aatggcagac aggtgcgcca cctggttgaa tatggaagtc aagattatag |
| | 7201 atgctgtggt gggcgagaaa gcgccttatt tctgtggagg gtttattttg tgtgactccg |
| | 7261 tgaccggcac agcgtgccgt gtggcagacc ccctaaaaag gctgtttaag cttggcaaac |
| | 7321 ctctggcagc agacgatgaa catgatgatg acaggagaag ggcattgcat gaagagtcaa |
| | 7381 cacgctggaa ccgagtgggt attctttcag agctgtgcaa ggcagtagaa tcaaggtatg |
| | 7441 aaaccgtagg aacttccatc atagttatgg ccatgactac tctagctagc agtgttaaat |
| | 7501 cattcagcta cctgagaggg gcccctataa ctctctacgg ctaacctgaa tggactacga |
| | 7561 catagtctag tccgccaaga tatcgcacca tgaagtgcct gctgtacctg gccttcctgt |
| | 7621 tcatcggcgt gaactgcaag tttaccatcg tgttccctca caaccagaaa ggcaactgga |
| | 7681 agaacgtgcc cagcaactac cactactgcc ccagcagcag cgacctgaac tggcacaatg |
| | 7741 acctgatcgg cacagccctg caagtgaaga tgcccaagag ccacaaggcc atccaggccg |
| | 7801 atggatggat gtgccacgcc agcaaatggg tcaccacctg tgacttccgt tggtacggcc |
| | 7861 ccaagtacat cacccacagc atccgcagct tcacccctag cgtggaacag tgcaaagaga |
| | 7921 gcatcgagca gaccaagcaa ggcacctggc tgaaccctgg attcccacct cagagctgtg |
| | 7981 gctacgccac tgtgacagat gccgaggccg tgatcgtgca agtgacacct catcacgtgc |
| | 8041 tggtggacga gtacacaggc gagtgggtcg acagccagtt catcaacggc aagtgctcca |
| | 8101 actacatctg ccccaccgtg cacaacagca ccacctggca cagcgactac aaagtgaagg |
| | 8161 gcctgtgcga cagcaacctg atcagcatgg acatcacctt tttcagcgag gacggcgagc |
| | 8221 tgagcagcct gggaaaagaa ggcaccggct tcagaagcaa ctacttcgcc tatgagacag |
| | 8281 gcggcaaggc ctgcaagatg cagtattgca agcactgggg cgtcagactg cctagcggag |
| | 8341 tttggttcga gatggccgac aaggacctgt ttgccgccgc tagattccct gagtgtcctg |
| | 8401 agggcagcag catctctgcc cctagccaga catctgtgga cgtgtccctg atccaggacg |
| | 8461 tggaacggat cctggactac agcctgtgcc aagagacatg gtccaagatc agagccggcc |
| | 8521 tgcctatcag ccctgtggac ctgtcttatc tggcccctaa gaaccctggc accggacctg |
| | 8581 ccttcaccat catcaatggc accctgaagt actttgagac acggtacatc cgcgtggaca |
| | 8641 ttgccgctcc tatcctgagc agaatggtcg gaatgatcag cggcaccacc accgagaggg |
| | 8701 aactgtggga tgattgggcc ccttacgagg atgtggaaat cggccctaac ggcgtgctga |
| | 8761 gaacaagcag cggctacaag ttcccactgt acatgatcgg ccacggcatg ctggacagcg |
| | 8821 atctgcacct gtctagcaag gcccaggtgt tcgagcaccc tcacattcag gatgccgcca |
| | 8881 gccagctgcc tgatgacgag agcctgttct ttggcgatac cggcctgagc aagaacccca |
| | 8941 tcgaactggt ggaaggctgg ttcagcagct ggaagtcctc tatcgccagc ttcttcttca |
| | 9001 tcatcgggct gattatcggc ctgttcctgg tgctgagagt gggcatccac ctgtgcatca |
| | 9061 agctgaagca caccaagaag cggcagatct acaccgacat cgagatgaac cggctgggca |
| | 9121 agtgatgaat acagcagcaa ttggcaagct gcttacatag aactcgcggc gattggcatg |
| | 9181 ccgccttaaa atttttattt tatttttctt ttcttttccg aatcggattt tgtttttaat |
| | 9241 atttcaaaaa aaaaaaaaaa aaaaaaaaaa aaaaaaaaaa aaaaagaaga gcatgcaagc |
| | 9301 ttggctgttt tggcggatga gagaagattt tcagcctgat acagattaaa tcagaacgca |
| | 9361 gaagcggtct gataaaacag aatttgcctg gcggcagtag cgcggtggtc ccacctgacc |
| | 9421 ccatgccgaa ctcagaagtg aaacgccgta gcgccgatgg tagtgtgggg tctccccatg |
| | 9481 cgagagtagg gaactgccag gcatcaaata aaacgaaagg ctcagtcgaa agactgggcc |
| | 9541 tttcgtttta tctgttgttt gtcggtgaac gctctcctga gtaggacaaa tccgccggga |
| | 9601 gcggatttga acgttgcgaa gcaacggccc ggagggtggc gggcaggacg cccgccataa |
| | 9661 actgccaggc atcaaattaa gcagaaggcc atcctgacgg atggcctttt tgcgtttcta |
| | 9721 caaactcttt tgtttatttt tctaaataca ttcaaatatg tatccgctca tgagacaata |
| | 9781 accctgataa atgcttcaat aatattgaaa aaggaagagt atgagtattc aacatttccg |
| | 9841 tgtcgccctt attccctttt ttgcggcatt ttgccttcct gtttttgctc acccagaaac |
| | 9901 gctggtgaaa gtaaaagatg ctgaagatca gttgggtgca cgagtgggtt acatcgaact |
| | 9961 ggatctcaac agcggtaaga tccttgagag ttttcgcccc gaagaacgtt ttccaatgat |
| | 10021 gagcactttt aaagttctgc tatgtggcgc ggtattatcc cgtgttgacg ccgggcaaga |
| | 10081 gcaactcggt cgccgcatac actattctca gaatgacttg gttgagtact caccagtcac |
| | 10141 agaaaagcat cttacggatg gcatgacagt aagagaatta tgcagtgctg ccataaccat |
| | 10201 gagtgataac actgcggcca acttacttct gacaacgatc ggaggaccga aggagctaac |
| | 10261 cgcttttttg cacaacatgg gggatcatgt aactcgcctt gatcgttggg aaccggagct |
| | 10321 gaatgaagcc ataccaaacg acgagcgtga caccacgatg cctgcagcaa tggcaacaac |
| | 10381 gttgccgtgc tgctgaagtt gcccgcaaca gagagtggaa ccaaccggtg ataccacgat |
| | 10441 actatgactg agagtcaacg ccatgagcgg cctcatttct tattctgagt tacaacagtc |
| | 10501 cgcaccgctg tccggtagct ccttccggtg ggcgcggggc atgactatcg tcgccgcact |
| | 10561 tatgactgtc ttctttatca tgcaactcgt aggacaggtg ccggcagcgc ccaacagtcc |
| | 10621 cccggccacg gggcctgcca ccatacccac gccgaaacaa gcgccctgca ccattatgtt |
| | 10681 ccggatctgc atcgcaggat gctgctggct accctgtgga acacctacat ctgtattaac |
| | 10741 gaagcgctaa ccgtttttat caggctctgg gaggcagaat aaatgatcat atcgtcaatt |
| | 10801 attacctcca cggggagagc ctgagcaaac tggcctcagg catttgagaa gcacacggtc |
| | 10861 acactgcttc cggtagtcaa taaaccggta aaccagcaat agacataagc ggctatttaa |
| | 10921 cgaccctgcc ctgaaccgac gaccgggtcg aatttgcttt cgaatttctg ccattcatcc |
| | 10981 gcttattatc acttattcag gcgtagcacc aggcgtttaa gggcaccaat aactgcctta |
| | 11041 aaaaaattac gccccgccct gccactcatc gcagtactgt tgtaattcat taagcattct |
| | 11101 gccgacatgg aagccatcac agacggcatg atgaacctga atcgccagcg gcatcagcac |
| | 11161 cttgtcgcct tgcgtataat atttgcccat ggtgaaaacg ggggcgaaga agttgtccat |
| | 11221 attggccacg tttaaatcaa aactggtgaa actcacccag ggattggctg agacgaaaaa |
| | 11281 catattctca ataaaccctt tagggaaata ggccaggttt tcaccgtaac acgccacatc |
| | 11341 ttgcgaatat atgtgtagaa actgccggaa atcgtcgtgg tattcactcc agagcgatga |
| | 11401 aaacgtttca gtttgctcat ggaaaacggt gtaacaaggg tgaacactat cccatatcac |
| | 11461 cagctcaccg tctttcattg ccatacggaa ttccggatga gcattcatca ggcgggcaag |
| | 11521 aatgtgaata aaggccggat aaaacttgtg cttatttttc tttacggtct ttaaaaaggc |
| | 11581 cgtaatatcc agctgaacgg tctggttata ggtacattga gcaactgact gaaatgcctc |
| | 11641 aaaatgttct ttacgatgcc attgggatat atcaacggtg gtatatccag tgattttttt |
| | 11701 ctccatttta gcttccttag ctcctgaaaa tctcgataac tcaaaaaata cgcccggtag |
| | 11761 tgatcttatt tcattatggt gaaagttgga acctcttacg caaactatta actggcgaac |
| | 11821 tacttactct agcttcccgg caacaattaa tagactggat ggaggcggat aaagttgcag |
| | 11881 gaccacttct gcgctcggcc cttccggctg gctggtttat tgctgataaa tctggagccg |
| | 11941 gtgagcgtgg gtctcgcggt atcattgcag cactggggcc agatggtaag ccctcccgta |
| | 12001 tcgtagttat ctacacgacg gggagtcagg caactatgga tgaacgaaat agacagatcg |
| | 12061 ctgagatagg tgcctcactg attaagcatt ggtaactgtc agaccaagtt tactcatata |
| | 12121 tactttagat tgatttacgc gccctgtagc ggcgcattaa gcgcggcggg tgtggtggtt |
| | 12181 acgcgcagcg tgaccgctac acttgccagc gccctagcgc ccgctccttt cgctttcttc |
| | 12241 ccttcctttc tcgccacgtt cgccggcttt ccccgtcaag ctctaaatcg ggggctccct |
| | 12301 ttagggttcc gatttagtgc tttacggcac ctcgacccca aaaaacttga tttgggtgat |
| | 12361 ggttcacgta gtgggccatc gccctgatag acggtttttc gccctttgac gttggagtcc |
| | 12421 acgttcttta atagtggact cttgttccaa acttgaacaa cactcaaccc tatctcgggc |
| | 12481 tattcttttg atttataagg gattttgccg atttcggcct attggttaaa aaatgagctg |
| | 12541 atttaacaaa aatttaacgc gaattttaac aaaatattaa cgtttacaat ttaaaaggat |
| | 12601 ctaggtgaag atcctttttg ataatctcat gaccaaaatc ccttaacgtg agttttcgtt |
| | 12661 ccactgagcg tcagaccccg tagaaaagat caaaggatct tcttgagatc ctttttttct |
| | 12721 gcgcgtaatc tgctgcttgc aaacaaaaaa accaccgcta ccagcggtgg tttgtttgcc |
| | 12781 ggatcaagag ctaccaactc tttttccgaa ggtaactggc ttcagcagag cgcagatacc |
| | 12841 aaatactgtc cttctagtgt agccgtagtt aggccaccac ttcaagaact ctgtagcacc |
| | 12901 gcctacatac ctcgctctgc taatcctgtt accagtggct gctgccagtg gcgataagtc |
| | 12961 gtgtcttacc gggttggact caagacgata gttaccggat aaggcgcagc ggtcgggctg |
| | 13021 aacggggggt tcgtgcacac agcccagctt ggagcgaacg acctacaccg aactgagata |
| | 13081 cctacagcgt gagctatgag aaagcgccac gcttcccgaa gggagaaagg cggacaggta |
| | 13141 tccggtaagc ggcagggtcg gaacaggaga gcgcacgagg gagcttccag ggggaaacgc |
| | 13201 ctggtatctt tatagtcctg tcgggtttcg ccacctctga cttgagcgtc gatttttgtg |
| | 13261 atgctcgtca ggggggcgga gcctatggaa aaacgccagc aacgcggcct ttttacggtt |
| | 13321 cctggccttt tgctggcctt ttgctcacat gttctttcct gcgttatccc ctgattctgt |
| | 13381 ggataaccgt attaccgcct ttgagtgagc tgataccgct cgccgcagcc gaacgaccga |
| | 13441 gcgcagcgag tcagtgagcg aggaagcgac gttaat |
| | // |

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A composition comprising in solution:
at least 1 mM of a free amino acid; and
a capsule comprising isolated RNA.

2. The composition according to claim 1, wherein the RNA is selected from the group consisting of messenger RNA (mRNA) and self-amplifying RNA (saRNA).

3. (canceled)

4. The composition according to claim 1, wherein the RNA comprises:
a 5′ Cap represented by Formula I, 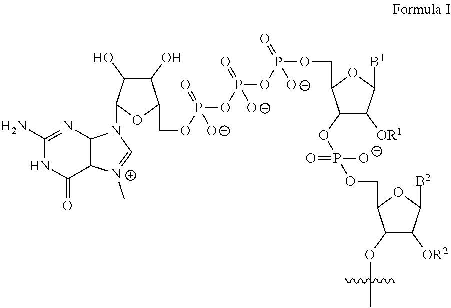
where R¹ and R² are each independently H or Me; and
B¹ and B² are each independently guanine, adenine, or uracil;
a 5′ untranslated region;
a coding region for a nonstructural protein derived from an alphavirus;
a subgenomic promoter derived from an alphavirus;
an open reading frame encoding a gene of interest;
a 3′ untranslated region; and
a 3′ poly A sequence.

5. The composition according to claim 1, wherein the RNA is modified RNA (modRNA).

6. The composition according to claim 1, wherein the RNA is viral RNA.

7. The composition according to claim 1, wherein the RNA encodes a viral antigen.

8. The composition according to claim 1, wherein the RNA is part of a vaccine.

9. (canceled)

10. The composition according to claim 1, wherein the capsule comprises a cationic lipid, a liposome, a lipid nanoparticle, a polyplex, a cochleate, a virosome, an immune-stimulating complex, a microparticle, a microsphere, a nanosphere, a unilamellar vesicle, a multilamellar vesicle, an oil-in-water emulsion, a water-in-oil emulsion, an emulsome, a polycationic peptide, or a cationic nanoemulsion.

11. The composition according to claim 1, wherein the capsule comprising the isolated RNA further encapsulates the at least 1 mM of a free amino acid.

12. The composition according to claim 1, wherein the capsule comprising the isolated RNA does not encapsulate the at least 1 mM of a free amino acid.

13. The composition according to claim 1, wherein the free amino acid is selected from the group consisting of glutamic acid, glutamate, aspartic acid, aspartate, arginine, lysine, tyrosine, cysteine, glutamine, asparagine, threonine, and serine, or a pharmaceutically acceptable salt thereof.

14. (canceled)

15. (canceled)

16. (canceled)

17. The composition according to claim 1, wherein the solution further comprises a sugar and/or buffer.

18. The composition according to claim 17, wherein the sugar is at a concentration of about 10% w/v.

19. The composition according to claim 17, wherein the buffer is at a concentration of about 10 mM.

20. The composition according to claim 17, wherein the buffer is at a pH of about 7.4.

21. (canceled)

22. (canceled)

23. A method of producing a composition in solution, comprising combining an isolated RNA and at least 1 mM of a free amino acid before the isolated RNA is encapsulated in a capsule.

24. (canceled)

25. (canceled)

26. A method for treating or preventing an infectious disease, comprising administering to a subject in need thereof an effective amount of a composition comprising in solution: at least 1 mM of a free amino acid; and a capsule comprising isolated RNA.

27. The method according to claim 26, wherein immunogenicity of the composition is increased compared to immunogenicity of a composition in which the capsule comprising the isolated RNA has not been contacted with the at least 1 mM of a free amino acid.

28. The method according to claim 26, wherein the free amino acid is selected from the group consisting of glutamic acid, glutamate, aspartic acid, aspartate, arginine, glutamine, and asparagine, or a pharmaceutically acceptable salt thereof.

29. The method according to claim 13, wherein the free amino acid is selected from the group consisting of glutamic acid, glutamate, aspartic acid, aspartate, arginine, glutamine, and asparagine, or a pharmaceutically acceptable salt thereof.


SEQUENCE LISTING (37 sequences)

SEQ ID NO: 1 - RNA, 455 nt, Artificial Sequence
  Synthetic nucleotide sequence
gccagcccccgauugggggcgacacuccaccauagaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggaucaacccgcucaaugccuggagauuugggcgugcccccgcgagacugcuagccgaaguaguguugggucgcgaaaggccuugugguacugccugauagggugcuugcgagugccccgggaggucucguagaccgugcaccaugagcacgaauccuaaaccucaaagaaaaaccaaacguaacaccaaccgccgcccacaggacgucugaggcggccgccgccucagacguccuguggguuuauugcaucccgc

SEQ ID NO: 2 - DNA, 489 bp, Artificial Sequence
  Synthetic nucleotide sequence
ggatcctaatacgactcactatagccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtctgaggcggccgccgcctcagacgtcctgtgggtttattgcatcccgcctcgagtctaga

SEQ ID NO: 3 - RNA, 574 nt, Artificial Sequence
  Synthetic nucleotide sequence
ucucucgcuaucucggaaucgaggggucuggccuacgcugcgauugcuucuucagagcauccuucuucagccuugugcccucuacaguggcagccucacacaaacuaucaacauggcuuccgaauccaggcucuaccagaucuccggcgagaccaagucucaucugcucaaguuccgcuugacgacaucucgggccagcaaaccucaggcagugauuuauuugauugauaaaaacacucacgaaauccgccaagacgaugacaagacuguauacaccucccucgacgagauagccgacgaccuccccgacagcaccccucgauucaucuuccucagcuaucccuugacgaugcccgauggacggcuauccgugccuuacaccaugaucuacuaccuccccaucaacugcaaugccgcaacaaggaugcucuacgccggcgcaaaagagcugauacgcaacacggccgaggugaacaagguuaucgauauagagucugcagaggaucuggaagauauuccaaagcaguugagcggauagaaauaauaauaaaagaaauaacauguucgcuaugcc

SEQ ID NO: 4 - RNA, 612 nt, Artificial Sequence
  Synthetic nucleotide sequence
uccaccaugaaucacuccucucucgcuaucucggaaucgaggggucuggccuacgcugcgauugcuucuucagagcauccuucuucagccuugugcccucuacaguggcagccucacacaaacuaucaacauggcuuccgaauccaggcucuaccagaucuccggcgagaccaagucucaucugcucaaguuccgcuugacgacaucucgggccagcaaaccucaggcagugauuuauuugauugauaaaaacacucacgaaauccgccaagacgaugacaagacuguauacaccucccucgacgagauagccgacgaccuccccgacagcaccccucgauucaucuuccucagcuaucccuugacgaugcccgauggacggcuauccgugccuuacaccaugaucuacuaccuccccaucaacugcaaugccgcaacaaggaugcucuacgccggcgcaaaagagcugauacgcaacacggccgaggugaacaagguuaucgauauagagucugcagaggaucuggaagauauuccaaagcaguugagcggauagaaauaauaauaaaagaaauaacauguucgcuaugccgaaaaaccaaacguaacacc

SEQ ID NO: 5 - DNA, 612 bp, Artificial Sequence
  Synthetic nucleotide sequence
tccaccatgaatcactcctctctcgctatctcggaatcgaggggtctggcctacgctgcgattgcttcttcagagcatccttcttcagccttgtgccctctacagtggcagcctcacacaaactatcaacatggcttccgaatccaggctctaccagatctccggcgagaccaagtctcatctgctcaagttccgcttgacgacatctcgggccagcaaacctcaggcagtgatttatttgattgataaaaacactcacgaaatccgccaagacgatgacaagactgtatacacctccctcgacgagatagccgacgacctccccgacagcacccctcgattcatcttcctcagctatcccttgacgatgcccgatggacggctatccgtgccttacaccatgatctactacctccccatcaactgcaatgccgcaacaaggatgctctacgccggcgcaaaagagctgatacgcaacacggccgaggtgaacaaggttatcgatatagagtctgcagaggatctggaagatattccaaagcagttgagcggatagaaataataataaaagaaataacatgttcgctatgccgaaaaaccaaacgtaacacc

SEQ ID NO: 6 - RNA, 1667 nt, Artificial Sequence
  Synthetic nucleotide sequence
uacgccagacaauagauucagcucgaauaaugaagccgauuggucacugauuaacuugguguaucuacuggguagcaaaggaccgaucuucaccucgcaugaccguugaauguggcgcaauucuaguacuugaauggccuggccaagcuuauagaacaaucgagacgaauugaacaccacuaucaaaauccaaauuaugcgguuuucaaucauacauguggcuuuauggcuggccauggcggccgguucauuuacagcuaacgcuuacggagcuguucgaugcgucauguaucucacagggcaacacgugguagucccuucagaaccucaucucguggauuccauaacccauuugauacuggcuuuuaugcgcucugaugucuucaauguggacaaaacgccugcugaauucccgcucuucgcaucaguugcugagacgcgcgagaaguucaacgcaaauaccaagaucauggucgcaaucggcgguuggggagacgcaggauuugaagaagcugcgcgugacgauucaucgagaaagcggugggcuggccacgucaaggccaugguugaccagacaggagccgauggcauugacauugacugggaauauccgggagggaaccgugacgauuauaaacucauuccgaacucucagcgggaaugggagauucaggcauucgugcuccuucuucaagaauugcguuccgccuugggacaaacgaaacugcucucaauugcggugccagcgcuggaacgagauuugauggccuucacaaacucgacuauuccguccauuguggaucaaguugacuuuaucaguguaaugacuuacgacaugaugaaccgacgcgacaacguugucaagcaccacagcggcguggcugacuccugggaagcaaugaagcgauauauagaucgcggcgucccuccgcacaagcugaacuuuggacuugguuacuaugccaaaugguucaugacugaacaaugcgauguacagcacccauuaggaugccgcacucaacugcuagaagaccccgccaauggagccgaucuuggcaagacugcagcuuuuagcuggcacgacgagguucccgcagaaauggccaauuccuuuggcaaagcccaugcucauggccgcuacuaugaagauggaagcuacggcuauugggacgaugaagagaagagauggugguccuacgacacgccucucaccaucaaagccaaagugccucggcuucucggcgagcugcagcugggcgggguguuugccugggggcugggcgaggacgcuccgcaguuucugcaccugaaggcgacuguugacggcauucggguucugcgcggagaugacgaugcugugaaggaugagcuguaacaagcagcggcguguguacgucuggaguuuggcgacaucauugaaggcgugggaugcaauucaagccauugauacugcagauaaguugaagcagcaacagccaaaacucugcacguggcccgagacuuccgucuuauagcagggaacagacaggauggugcugacggaauaugcagagcucgagcucaaccgguaggcuagcuugaaaaccccaugagcuagcaguuaaccuugcaguuaaggguaaccagagcuuaaacacgucugauuuggugcaaugaauacaggcugc

SEQ ID NO: 7 - RNA, 1705 nt, Artificial Sequence
  Synthetic nucleotide sequence
uccaccaugaaucacuccuacgccagacaauagauucagcucgaauaaugaagccgauuggucacugauuaacuugguguaucuacuggguagcaaaggaccgaucuucaccucgcaugaccguugaauguggcgcaauucuaguacuugaauggccuggccaagcuuauagaacaaucgagacgaauugaacaccacuaucaaaauccaaauuaugcgguuuucaaucauacauguggcuuuauggcuggccauggcggccgguucauuuacagcuaacgcuuacggagcuguucgaugcgucauguaucucacagggcaacacgugguagucccuucagaaccucaucucguggauuccauaacccauuugauacuggcuuuuaugcgcucugaugucuucaauguggacaaaacgccugcugaauucccgcucuucgcaucaguugcugagacgcgcgagaaguucaacgcaaauaccaagaucauggucgcaaucggcgguuggggagacgcaggauuugaagaagcugcgcgugacgauucaucgagaaagcggugggcuggccacgucaaggccaugguugaccagacaggagccgauggcauugacauugacugggaauauccgggagggaaccgugacgauuauaaacucauuccgaacucucagcgggaaugggagauucaggcauucgugcuccuucuucaagaauugcguuccgccuugggacaaacgaaacugcucucaauugcggugccagcgcuggaacgagauuugauggccuucacaaacucgacuauuccguccauuguggaucaaguugacuuuaucaguguaaugacuuacgacaugaugaaccgacgcgacaacguugucaagcaccacagcggcguggcugacuccugggaagcaaugaagcgauauauagaucgcggcgucccuccgcacaagcugaacuuuggacuugguuacuaugccaaaugguucaugacugaacaaugcgauguacagcacccauuaggaugccgcacucaacugcuagaagaccccgccaauggagccgaucuuggcaagacugcagcuuuuagcuggcacgacgagguucccgcagaaauggccaauuccuuuggcaaagcccaugcucauggccgcuacuaugaagauggaagcuacggcuauugggacgaugaagagaagagauggugguccuacgacacgccucucaccaucaaagccaaagugccucggcuucucggcgagcugcagcugggcgggguguuugccugggggcugggcgaggacgcuccgcaguuucugcaccugaaggcgacuguugacggcauucggguucugcgcggagaugacgaugcugugaaggaugagcuguaacaagcagcggcguguguacgucuggaguuuggcgacaucauugaaggcgugggaugcaauucaagccauugauacugcagauaaguugaagcagcaacagccaaaacucugcacguggcccgagacuuccgucuuauagcagggaacagacaggauggugcugacggaauaugcagagcucgagcucaaccgguaggcuagcuugaaaaccccaugagcuagcaguuaaccuugcaguuaaggguaaccagagcuuaaacacgucugauuuggugcaaugaauacaggcugcgaaaaaccaaacguaacacc

SEQ ID NO: 8 - DNA, 1705 bp, Artificial Sequence
  Synthetic nucleotide sequence
tccaccatgaatcactcctacgccagacaatagattcagctcgaataatgaagccgattggtcactgattaacttggtgtatctactgggtagcaaaggaccgatcttcacctcgcatgaccgttgaatgtggcgcaattctagtacttgaatggcctggccaagcttatagaacaatcgagacgaattgaacaccactatcaaaatccaaattatgcggttttcaatcatacatgtggctttatggctggccatggcggccggttcatttacagctaacgcttacggagctgttcgatgcgtcatgtatctcacagggcaacacgtggtagtcccttcagaacctcatctcgtggattccataacccatttgatactggcttttatgcgctctgatgtcttcaatgtggacaaaacgcctgctgaattcccgctcttcgcatcagttgctgagacgcgcgagaagttcaacgcaaataccaagatcatggtcgcaatcggcggttggggagacgcaggatttgaagaagctgcgcgtgacgattcatcgagaaagcggtgggctggccacgtcaaggccatggttgaccagacaggagccgatggcattgacattgactgggaatatccgggagggaaccgtgacgattataaactcattccgaactctcagcgggaatgggagattcaggcattcgtgctccttcttcaagaattgcgttccgccttgggacaaacgaaactgctctcaattgcggtgccagcgctggaacgagatttgatggccttcacaaactcgactattccgtccattgtggatcaagttgactttatcagtgtaatgacttacgacatgatgaaccgacgcgacaacgttgtcaagcaccacagcggcgtggctgactcctgggaagcaatgaagcgatatatagatcgcggcgtccctccgcacaagctgaactttggacttggttactatgccaaatggttcatgactgaacaatgcgatgtacagcacccattaggatgccgcactcaactgctagaagaccccgccaatggagccgatcttggcaagactgcagcttttagctggcacgacgaggttcccgcagaaatggccaattcctttggcaaagcccatgctcatggccgctactatgaagatggaagctacggctattgggacgatgaagagaagagatggtggtcctacgacacgcctctcaccatcaaagccaaagtgcctcggcttctcggcgagctgcagctgggcggggtgtttgcctgggggctgggcgaggacgctccgcagtttctgcacctgaaggcgactgttgacggcattcgggttctgcgcggagatgacgatgctgtgaaggatgagctgtaacaagcagcggcgtgtgtacgtctggagtttggcgacatcattgaaggcgtgggatgcaattcaagccattgatactgcagataagttgaagcagcaacagccaaaactctgcacgtggcccgagacttccgtcttatagcagggaacagacaggatggtgctgacggaatatgcagagctcgagctcaaccggtaggctagcttgaaaaccccatgagctagcagttaaccttgcagttaagggtaaccagagcttaaacacgtctgatttggtgcaatgaatacaggctgcgaaaaaccaaacgtaacacc

SEQ ID NO: 9 - RNA, 30 nt, Artificial Sequence
  Synthetic nucleotide sequence
ggaaucucucucacgaacugacguaaucuu

SEQ ID NO: 10 - protein, 511 aa, Artificial Sequence
  Synthetic peptide sequence
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 11 - DNA, 1536 bp, Artificial Sequence
  Synthetic nucleotide sequence
atgaagtgcctgctgtacctggccttcctgttcatcggcgtgaactgcaagtttaccatcgtgttccctcacaaccagaaaggcaactggaagaacgtgcccagcaactaccactactgccccagcagcagcgacctgaactggcacaatgacctgatcggcacagccctgcaagtgaagatgcccaagagccacaaggccatccaggccgatggatggatgtgccacgccagcaaatgggtcaccacctgtgacttccgttggtacggccccaagtacatcacccacagcatccgcagcttcacccctagcgtggaacagtgcaaagagagcatcgagcagaccaagcaaggcacctggctgaaccctggattcccacctcagagctgtggctacgccactgtgacagatgccgaggccgtgatcgtgcaagtgacacctcatcacgtgctggtggacgagtacacaggcgagtgggtcgacagccagttcatcaacggcaagtgctccaactacatctgccccaccgtgcacaacagcaccacctggcacagcgactacaaagtgaagggcctgtgcgacagcaacctgatcagcatggacatcacctttttcagcgaggacggcgagctgagcagcctgggaaaagaaggcaccggcttcagaagcaactacttcgcctatgagacaggcggcaaggcctgcaagatgcagtattgcaagcactggggcgtcagactgcctagcggagtttggttcgagatggccgacaaggacctgtttgccgccgctagattccctgagtgtcctgagggcagcagcatctctgcccctagccagacatctgtggacgtgtccctgatccaggacgtggaacggatcctggactacagcctgtgccaagagacatggtccaagatcagagccggcctgcctatcagccctgtggacctgtcttatctggcccctaagaaccctggcaccggacctgccttcaccatcatcaatggcaccctgaagtactttgagacacggtacatccgcgtggacattgccgctcctatcctgagcagaatggtcggaatgatcagcggcaccaccaccgagagggaactgtgggatgattgggccccttacgaggatgtggaaatcggccctaacggcgtgctgagaacaagcagcggctacaagttcccactgtacatgatcggccacggcatgctggacagcgatctgcacctgtctagcaaggcccaggtgttcgagcaccctcacattcaggatgccgccagccagctgcctgatgacgagagcctgttctttggcgataccggcctgagcaagaaccccatcgaactggtggaaggctggttcagcagctggaagtcctctatcgccagcttcttcttcatcatcgggctgattatcggcctgttcctggtgctgagagtgggcatccacctgtgcatcaagctgaagcacaccaagaagcggcagatctacaccgacatcgagatgaaccggctgggcaagtga

SEQ ID NO: 12 - protein, 623 aa, Artificial Sequence
  Synthetic peptide sequence
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLAAVVLLCLVIFLICTAKRMRVKAYRVDKSPYNQSMYYAGLPVDDFEDSESTDTEEEFGNAIGGSHGGSSYTVYIDKTR

SEQ ID NO: 13 - DNA, 1872 bp, Artificial Sequence
  Synthetic nucleotide sequence
atggggacagttaataaacctgtggtgggggtattgatggggttcggaattatcacgggaacgttgcgtataacgaatccggtcagagcatccgtcttgcgatacgatgattttcacatcgatgaagacaaactggatacaaactccgtatatgagccttactaccattcagatcatgcggagtcttcatgggtaaatcggggagagtcttcgcgaaaagcgtacgatcataactcaccttatatatggccacgtaatgattatgatggatttttagagaacgcacacgaacaccatggggtgtataatcagggccgtggtatcgatagcggggaacggttaatgcaacccacacaaatgtctgcacaggaggatcttggggacgatacgggcatccacgttatccctacgttaaacggcgatgacagacataaaattgtaaatgtggaccaacgtcaatacggtgacgtgtttaaaggagatcttaatccaaaaccccaaggccaaagactcattgaggtgtcagtggaagaaaatcacccgtttactttacgcgcaccgattcagcggatttatggagtccggtacaccgagacttggagctttttgccgtcattaacctgtacgggagacgcagcgcccgccatccagcatatatgcttaaaacatacaacatgctttcaagacgtggtggtggatgtggattgcgcggaaaatactaaagaggatcagttggccgaaatcagttaccgttttcaaggtaagaaggaagcggaccaaccgtggattgttgtaaacacgagcacactgtttgatgaactcgaattagacccccccgagattgaaccgggtgtcttgaaagtacttcggacagaaaaacaatacttgggtgtgtacatttggaacatgcgcggctccgatggtacgtctacctacgccacgtttttggtcacctggaaaggggatgaaaaaacaagaaaccctacgcccgcagtaactcctcaaccaagaggggctgagtttcatatgtggaattaccactcgcatgtattttcagttggtgatacgtttagcttggcaatgcatcttcagtataagatacatgaagcgccatttgatttgctgttagagtggttgtatgtccccatcgatcctacatgtcaaccaatgcggttatattctacgtgtttgtatcatcccaacgcaccccaatgcctctctcatatgaattccggttgtacatttacctcgccacatttagcccagcgtgttgcaagcacagtgtatcaaaattgtgaacatgcagataactacaccgcatattgtctgggaatatctcatatggagcctagctttggtctaatcttacacgacgggggcaccacgttaaagtttgtagatacacccgagagtttgtcgggattatacgtttttgtggtgtattttaacgggcatgttgaagccgtagcatacactgttgtatccacagtagatcattttgtaaacgcaattgaagagcgtggatttccgccaacggccggtcagccaccggcgactactaaacccaaggaaattacccccgtaaaccccggaacgtcaccacttatacgatatgccgcatggaccggagggcttgcagcagtagtacttttatgtctcgtaatatttttaatctgtacggctaaacgaatgagggttaaagcctatagggtagacaagtccccgtataaccaaagcatgtattacgctggccttccagtggacgatttcgaggactcggaatctacggatacggaagaagagtttggtaacgcgattggagggagtcacgggggttcgagttacacggtgtatatagataagacccggtga

SEQ ID NO: 14 - RNA, 1872 nt, Artificial Sequence
  Synthetic nucleotide sequence
auggggacaguuaauaaaccugugguggggguauugaugggguucggaauuaucacgggaacguugcguauaacgaauccggucagagcauccgucuugcgauacgaugauuuucacaucgaugaagacaaacuggauacaaacuccguauaugagccuuacuaccauucagaucaugcggagucuucauggguaaaucggggagagucuucgcgaaaagcguacgaucauaacucaccuuauauauggccacguaaugauuaugauggauuuuuagagaacgcacacgaacaccaugggguguauaaucagggccgugguaucgauagcggggaacgguuaaugcaacccacacaaaugucugcacaggaggaucuuggggacgauacgggcauccacguuaucccuacguuaaacggcgaugacagacauaaaauuguaaauguggaccaacgucaauacggugacguguuuaaaggagaucuuaauccaaaaccccaaggccaaagacucauugaggugucaguggaagaaaaucacccguuuacuuuacgcgcaccgauucagcggauuuauggaguccgguacaccgagacuuggagcuuuuugccgucauuaaccuguacgggagacgcagcgcccgccauccagcauauaugcuuaaaacauacaacaugcuuucaagacguggugguggauguggauugcgcggaaaauacuaaagaggaucaguuggccgaaaucaguuaccguuuucaagguaagaaggaagcggaccaaccguggauuguuguaaacacgagcacacuguuugaugaacucgaauuagacccccccgagauugaaccgggugucuugaaaguacuucggacagaaaaacaauacuuggguguguacauuuggaacaugcgcggcuccgaugguacgucuaccuacgccacguuuuuggucaccuggaaaggggaugaaaaaacaagaaacccuacgcccgcaguaacuccucaaccaagaggggcugaguuucauauguggaauuaccacucgcauguauuuucaguuggugauacguuuagcuuggcaaugcaucuucaguauaagauacaugaagcgccauuugauuugcuguuagagugguuguauguccccaucgauccuacaugucaaccaaugcgguuauauucuacguguuuguaucaucccaacgcaccccaaugccucucucauaugaauuccgguuguacauuuaccucgccacauuuagcccagcguguugcaagcacaguguaucaaaauugugaacaugcagauaacuacaccgcauauugucugggaauaucucauauggagccuagcuuuggucuaaucuuacacgacgggggcaccacguuaaaguuuguagauacacccgagaguuugucgggauuauacguuuuugugguguauuuuaacgggcauguugaagccguagcauacacuguuguauccacaguagaucauuuuguaaacgcaauugaagagcguggauuuccgccaacggccggucagccaccggcgacuacuaaacccaaggaaauuacccccguaaaccccggaacgucaccacuuauacgauaugccgcauggaccggagggcuugcagcaguaguacuuuuaugucucguaauauuuuuaaucuguacggcuaaacgaaugaggguuaaagccuauaggguagacaaguccccguauaaccaaagcauguauuacgcuggccuuccaguggacgauuucgaggacucggaaucuacggauacggaagaagaguuugguaacgcgauuggagggagucacggggguucgaguuacacgguguauauagauaagacccgguga

SEQ ID NO: 15 - protein, 907 aa, Artificial Sequence
  Synthetic peptide sequence
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIIIYLIYTRQRRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEEENV

SEQ ID NO: 16 - DNA, 2724 bp, Artificial Sequence
  Synthetic nucleotide sequence
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcattctcgatccggttcagtctctcaacgcgtaacttcttcccaaacggtcagccatggtgttaacgagaccatctacaacactaccctcaagtacggagatgtggtgggggtcaacaccaccaagtacccctatcgcgtgtgttctatggcacagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacgtccacaccacttatctgctgggcagcaacacggaatacgtggcgcctcctatgtgggagattcatcatatcaacagtcacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaagtatccctatcattttttcgcaacttccacgggtgatgtggttgacatttctcctttctacaacggaactaatcgcaatgccagctattttggagaaaacgccgacaagtttttcatttttccgaactacactatcgtctccgactttggaagaccgaattctgcgttagagacccacaggttggtggcttttcttgaacgtgcggactcagtgatctcctgggatatacaggacgagaagaatgttacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgtgtacgtgatgaggccataaataagttacagcagattttcaatacttcatacaatcaaacatatgaaaaatatggaaacgtgtccgtctttgaaaccactggtggtttggtggtgttctggcaaggtatcaagcaaaaatctctggtggaactcgaacgtttggccaaccgctccagtctgaatcttactcataatagaaccaaaagaagtacagatggcaacaatgcaactcatttatccaacatggagtcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaacttagcaagatcaacccgtcagctattctctcggccatctacaacaaaccgattgccgcgcgtttcatgggcgatgtcctgggtctggccagctgcgtgaccattaaccaaaccagcgtcaaggtgctgcgtgatatgaatgtgaaggaatcgccaggacgctgctactcacgaccagtggtcatctttaatttcgccaacagctcgtacgtgcagtacggtcaactgggcgaggataacgaaatcctgttgggcaaccaccgcactgaggaatgtcagcttcccagcctcaagatcttcatcgccggcaactcggcctacgagtacgtggactacctcttcaaacgcatgattgacctcagcagcatctccaccgtcgacagcatgatcgccctagacatcgacccgctggaaaacaccgacttcagggtactggaactttactcgcagaaagaattgcgttccagcaacgtttttgatctcgaggagatcatgcgcgagttcaattcgtataagcagcgggtaaagtacgtggaggacaaggtagtcgacccgctgccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagccttcaccatcatcctcgtggccatagccgtcgtcattatcatttatttgatctatactcgacagcggcgtctctgcatgcagccgctgcagaacctctttccctatctggtgtccgccgacgggaccaccgtgacgtcgggcaacaccaaagacacgtcgttacaggctccgccttcctacgaggaaagtgtctataattctggtcgcaaaggaccgggaccaccgtcgtctgatgcatccacggcggctccgccttacaccaacgagcaggcttaccagatgcttctggccctggtccgtctggacgcagagcagcgagcgcagcagaacggtacagattctttggacggacagactggcacgcaggacaagggacagaagcccaacctgctagaccgactgcgacaccgcaaaaacggctaccgacacttgaaagactccgacgaagaagagaacgtctga

SEQ ID NO: 17 - RNA, 2724 nt, Artificial Sequence
  Synthetic nucleotide sequence
auggaauccaggaucuggugccugguagucugcguuaacuuguguaucgucugucugggugcugcgguuuccucaucuucuacucguggaacuucugcuacucacagucaccauuccucucauacgacgucugcugcucauucucgauccgguucagucucucaacgcguaacuucuucccaaacggucagccaugguguuaacgagaccaucuacaacacuacccucaaguacggagauguggugggggucaacaccaccaaguaccccuaucgcguguguucuauggcacaggguacggaucuuauucgcuuugaacguaauaucgucugcaccucgaugaagcccaucaaugaagaccuggacgagggcaucaugguggucuacaaacgcaacaucgucgcgcacaccuuuaagguacgagucuaccagaagguuuugacguuucgucguagcuacgcuuacguccacaccacuuaucugcugggcagcaacacggaauacguggcgccuccuaugugggagauucaucauaucaacagucacagucagugcuacaguuccuacagccgcguuauagcaggcacgguuuucguggcuuaucauagggacagcuaugaaaacaaaaccaugcaauuaaugcccgacgauuauuccaacacccacaguacccguuacgugacggucaaggaucaauggcacagccgcggcagcaccuggcucuaucgugagaccuguaaucugaauuguauggugaccaucacuacugcgcgcuccaaguaucccuaucauuuuuucgcaacuuccacgggugaugugguugacauuucuccuuucuacaacggaacuaaucgcaaugccagcuauuuuggagaaaacgccgacaaguuuuucauuuuuccgaacuacacuaucgucuccgacuuuggaagaccgaauucugcguuagagacccacagguugguggcuuuucuugaacgugcggacucagugaucuccugggauauacaggacgagaagaauguuacuugucaacucacuuucugggaagccucggaacgcaccauucguuccgaagccgaggacucguaucacuuuucuucugccaaaaugaccgccacuuucuuaucuaagaagcaagaggugaacauguccgacucugcgcuggacuguguacgugaugaggccauaaauaaguuacagcagauuuucaauacuucauacaaucaaacauaugaaaaauauggaaacguguccgucuuugaaaccacuggugguuuggugguguucuggcaagguaucaagcaaaaaucucugguggaacucgaacguuuggccaaccgcuccagucugaaucuuacucauaauagaaccaaaagaaguacagauggcaacaaugcaacucauuuauccaacauggagucggugcacaaucuggucuacgcccagcugcaguucaccuaugacacguugcgcgguuacaucaaccgggcgcuggcgcaaaucgcagaagccugguguguggaucaacggcgcacccuagaggucuucaaggaacuuagcaagaucaacccgucagcuauucucucggccaucuacaacaaaccgauugccgcgcguuucaugggcgauguccugggucuggccagcugcgugaccauuaaccaaaccagcgucaaggugcugcgugauaugaaugugaaggaaucgccaggacgcugcuacucacgaccaguggucaucuuuaauuucgccaacagcucguacgugcaguacggucaacugggcgaggauaacgaaauccuguugggcaaccaccgcacugaggaaugucagcuucccagccucaagaucuucaucgccggcaacucggccuacgaguacguggacuaccucuucaaacgcaugauugaccucagcagcaucuccaccgucgacagcaugaucgcccuagacaucgacccgcuggaaaacaccgacuucaggguacuggaacuuuacucgcagaaagaauugcguuccagcaacguuuuugaucucgaggagaucaugcgcgaguucaauucguauaagcagcggguaaaguacguggaggacaagguagucgacccgcugccgcccuaccucaagggucuggacgaccucaugagcggccugggcgccgcgggaaaggccguuggcguagccauuggggccguggguggcgcgguggccuccguggucgaaggcguugccaccuuccucaaaaaccccuucggagccuucaccaucauccucguggccauagccgucgucauuaucauuuauuugaucuauacucgacagcggcgucucugcaugcagccgcugcagaaccucuuucccuaucugguguccgccgacgggaccaccgugacgucgggcaacaccaaagacacgucguuacaggcuccgccuuccuacgaggaaagugucuauaauucuggucgcaaaggaccgggaccaccgucgucugaugcauccacggcggcuccgccuuacaccaacgagcaggcuuaccagaugcuucuggcccugguccgucuggacgcagagcagcgagcgcagcagaacgguacagauucuuuggacggacagacuggcacgcaggacaagggacagaagcccaaccugcuagaccgacugcgacaccgcaaaaacggcuaccgacacuugaaagacuccgacgaagaagagaacgucuga

SEQ ID NO: 18 - protein, 574 aa, Artificial Sequence
  Synthetic peptide sequence
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 19 - DNA, 1724 bp, Artificial Sequence
  Synthetic nucleotide sequence
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaatagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 20 - RNA, 1724 nt, Artificial Sequence
  Synthetic peptide sequence
auggaguugcuaauccucaaagcaaaugcaauuaccacaauccucacugcagucacauuuuguuuugcuucuggucaaaacaucacugaagaauuuuaucaaucaacaugcagugcaguuagcaaaggcuaucuuagugcucugagaacugguugguauaccaguguuauaacuauagaauuaaguaauaucaaggaaaauaaguguaauggaacagaugcuaagguaaaauugauaaaacaagaauuagauaaauauaaaaaugcuguaacagaauugcaguugcucaugcaaagcacaccaccaacaaacaaucgagccagaagagaacuaccaagguuuaugaauuauacacucaacaaugccaaaaaaaccaauguaacauuaagcaagaaaaggaaaagaagauuucuugguuuuuuguuagguguuggaucugcaaucgccaguggcguugcuguaucuaagguccugcaccuagaaggggaagugaacaagaucaaaagugcucuacuauccacaaacaaggcuguagucagcuuaucaaauggaguuagugucuuaaccagcaaaguguuagaccucaaaaacuauauagauaaacaauuguuaccuauugugaacaagcaaagcugcagcauaucaaauauagaaacugugauagaguuccaacaaaagaacaacagacuacuagagauuaccagggaauuuaguguuaaugcagguguaacuacaccuguaagcacuuacauguuaacuaauagugaauuauugucauuaaucaaugauaugccuauaacaaaugaucagaaaaaguuaauguccaacaauguucaaauaguuagacagcaaaguuacucuaucauguccauaauaaaagaggaagucuuagcauauguaguacaauuaccacuauaugguguuauagauacacccuguuggaaacuacacacauccccucuauguacaaccaacacaaaagaaggguccaacaucuguuuaacaagaacugacagaggaugguacugugacaaugcaggaucaguaucuuucuucccacaagcugaaacauguaaaguucaaucaaaucgaguauuuugugacacaaugaacaguuuaacauuaccaagugaaauaaaucucugcaauguugacauauucaaccccaaauaugauuguaaaauuaugacuucaaaaacagauguaagcagcuccguuaucacaucucuaggagccauugugucaugcuauggcaaaacuaaauguacagcauccaauaaaaaucguggaaucauaaagacauuuucuaacgggugcgauuauguaucaaauaaagggauggacacugugucuguagguaacacauuauauuauguaaauagcaagaagguaaaagucucuauguaaaaggugaaccaauaauaaauuucuaugacccauuaguauuccccucugaugaauuugaugcaucaauaucucaagucaacgagaagauuaaccagagccuagcauuuauucguaaauccgaugaauuauuacauaauguaaaugcugguaaauccaccacaaauaucaugauaacuacuauaauuauagugauuauaguaauauuguuaucauuaauugcuguuggacugcucuuauacuguaaggccagaagcacaccagucacacuaagcaaagaucaacugagugguauaaauaauauugcauuuaguaacuaa

SEQ ID NO: 21 - protein, 574 aa, Artificial Sequence
  Synthetic peptide sequence
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 22 - DNA, 1725 bp, Artificial Sequence
  Synthetic peptide sequence
atggagttgctgatccacaggtcaagtgcaatcttcctaactcttgctgttaatgcattgtacctcacctcaagtcagaacataactgaggagttttaccaatcgacatgtagtgcagttagcagaggttattttagtgctttaagaacaggttggtataccagtgtcataacaatagaattaagtaatataaaagaaaccaaatgcaatggaactgacactaaagtaaaacttataaaacaagaattagataagtataagaatgcagtaacagaattacagctacttatgcaaaacacgccagctgccaacaaccgggccagaagagaagcaccacagtacatgaactacacaatcaataccacaaaaaacctaaatgtatcaataagcaagaaaaggaaacgaagatttctgggcttcttgttaggtgtaggatctgcaatagcaagtggtatagctgtatccaaagttttacaccttgaaggagaagtgaacaaaatcaaaaatgctttgttgtctacaaacaaagctgtagtcagtctatcaaatggggtcagtgttttaaccagcaaagtgttagatctcaagaattacataaataaccgattattacccatagtaaatcaacagagctgtcgcatctccaacattgaaacagttatagaattccagcagatgaatagcagattgttggaaatcaccagagaatttagtgttaatgcaggtgtaacaacacctttaagcacttacatgttaacaaacagtgagttactatcattgatcaatgatatgcctataacaaatgaccagaaaaaattaatgtcaagcaatgttcagatagtaaggcaacaaagttattctatcatgtctataataaaggaagaagtccttgcatatgttgtacagctacctatctatggtgtaatagatacaccttgctggaaattacacacatcacctctatgcaccaccaacatcaaagaaggatcaaatatttgtttaacaaggactgatagaggatggtattgtgataatgcaggatcagtatccttcttcccacaggctgatacttgcaaagtacagtccaatcgagtattttgtgacactatgaacagtttaacattaccaagtgaagtcagcctttgtaacactgacatattcaattccaagtatgactgcaaaattatgacatcaaaaacagacataagcagctcagtaattacttctcttggagctatagtgtcatgctatggaaaaactaaatgcactgcatccaataaaaatcgtgggattataaagacattttctaatggttgtgactatgtgtcaaacaaaggagtagatactgtgtcagtgggcaacactttatactatgtaaacaagctggaaggcaaaaacctttatgtaaaaggggaacctataataaattactatgatcctctagtgtttccttctgatgagtttgatgcatcaatatctcaagtcaatgaaaaaatcaatcaaagtttagcttttattcgtagatctgatgaattactacataatgtaaatactggcaaatctactacaaatattatgataactacaattattatagtaatcattgtagtattgttatcattaatagctattggtttactgttgtattgcaaagccaaaaacacaccagttacactaagcaaagaccaactaagtggaatcaataatattgcattcagcaaatag

SEQ ID NO: 23 - RNA, 1725 nt, Artificial Sequence
  Synthetic nucleotide sequence
auggaguugcugauccacaggucaagugcaaucuuccuaacucuugcuguuaaugcauuguaccucaccucaagucagaacauaacugaggaguuuuaccaaucgacauguagugcaguuagcagagguuauuuuagugcuuuaagaacagguugguauaccagugucauaacaauagaauuaaguaauauaaaagaaaccaaaugcaauggaacugacacuaaaguaaaacuuauaaaacaagaauuagauaaguauaagaaugcaguaacagaauuacagcuacuuaugcaaaacacgccagcugccaacaaccgggccagaagagaagcaccacaguacaugaacuacacaaucaauaccacaaaaaaccuaaauguaucaauaagcaagaaaaggaaacgaagauuucugggcuucuuguuagguguaggaucugcaauagcaagugguauagcuguauccaaaguuuuacaccuugaaggagaagugaacaaaaucaaaaaugcuuuguugucuacaaacaaagcuguagucagucuaucaaauggggucaguguuuuaaccagcaaaguguuagaucucaagaauuacauaaauaaccgauuauuacccauaguaaaucaacagagcugucgcaucuccaacauugaaacaguuauagaauuccagcagaugaauagcagauuguuggaaaucaccagagaauuuaguguuaaugcagguguaacaacaccuuuaagcacuuacauguuaacaaacagugaguuacuaucauugaucaaugauaugccuauaacaaaugaccagaaaaaauuaaugucaagcaauguucagauaguaaggcaacaaaguuauucuaucaugucuauaauaaaggaagaaguccuugcauauguuguacagcuaccuaucuaugguguaauagauacaccuugcuggaaauuacacacaucaccucuaugcaccaccaacaucaaagaaggaucaaauauuuguuuaacaaggacugauagaggaugguauugugauaaugcaggaucaguauccuucuucccacaggcugauacuugcaaaguacaguccaaucgaguauuuugugacacuaugaacaguuuaacauuaccaagugaagucagccuuuguaacacugacauauucaauuccaaguaugacugcaaaauuaugacaucaaaaacagacauaagcagcucaguaauuacuucucuuggagcuauagugucaugcuauggaaaaacuaaaugcacugcauccaauaaaaaucgugggauuauaaagacauuuucuaaugguugugacuaugugucaaacaaaggaguagauacugugucagugggcaacacuuuauacuauguaaacaagcuggaaggcaaaaaccuuuauguaaaaggggaaccuauaauaaauuacuaugauccucuaguguuuccuucugaugaguuugaugcaucaauaucucaagucaaugaaaaaaucaaucaaaguuuagcuuuuauucguagaucugaugaauuacuacauaauguaaauacuggcaaaucuacuacaaauauuaugauaacuacaauuauuauaguaaucauuguaguauuguuaucauuaauagcuauugguuuacuguuguauugcaaagccaaaaacacaccaguuacacuaagcaaagaccaacuaaguggaaucaauaauauugcauucagcaaauag

SEQ ID NO: 24 - protein, 574 aa, Artificial Sequence
  Synthetic peptide sequence
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPAANSRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLALIAVGLLLYCKARSTPVTLSKDQLSGINNIAFSN

SEQ ID NO: 25 - DNA, 1725 bp, Artificial Sequence
  Synthetic nucleotide sequence
atggagttgccaatcctcaaaacaaatgctattaccacaatccttgctgcagtcacactctgcttcgcttccagtcaaaacatcactgaagaattttatcaatcaacatgcagtgcagttagcaaaggctatcttagtgctctaagaactggttggtatactagtgttataactatagaattaagtaatatcaaggaaaataagtgtaatggtacagatgctaaggtaaaattaataaaacaagaattagataaatataaaaatgctgtaacagaattgcagttgctcatgcaaagcacaccagcagccaacagtcgagccagaagagaactgccaagatttatgaattatacactcaacaataccaaaaacaccaatgtaacattaagtaagaaaaggaaaagaagatttcttggatttttgttaggtgttggatctgcaatcgccagtggcattgccgtatccaaggtcctgcacctagaaggggaagtgaacaaaatcaaaagtgctctactatccacaaacaaggctgtagtcagcttatctaatggagtcagtgtcttaaccagcaaggtgttagacctcaaaaactatatagataaacagttgttacctattgttaacaagcaaagctgcagcatatcaaacattgaaactgtgatagagttccaacaaaagaacaacagactactagagattaccagagaatttagtgttaatgcaggtgtaactacacctgtaagcacttatatgttaactaatagtgagttattatcattaatcaatgatatgcctataacaaatgatcagaaaaagttaatgtccagcaatgttcaaatagttagacagcaaagttactctatcatgtcaataataaaagaggaagtcttagcatatgtagtacaattaccactatatggtgtaatagatactccttgttggaaactacacacatcccctctatgtacaaccaacacaaaggaaggatccaacatctgcttaacaagaaccgacagaggatggtactgtgacaatgcaggatcagtatcctttttcccacaagctgaaacatgtaaagttcaatcgaatcgggtattttgtgacacaatgaacagtttaacattaccaagtgaggtaaatctctgcaacattgacatattcaaccccaaatatgattgcaaaattatgacttcaaaaacagatgtaagcagctccgttatcacatctctaggagccattgtgtcatgctatggcaaaaccaaatgtacagcatccaataaaaatcgtgggatcataaagacattctctaacgggtgtgattatgtatcaaataagggggtggatactgtgtctgtaggtaatacattatattatgtaaataagcaagaaggcaaaagtctctatgtaaaaggtgaaccaataataaatttctatgatccattagtgttcccctctgatgaatttgatgcatcaatatctcaagtcaatgagaaaattaatcagagtctagcatttatccgtaaatcagatgaattattacataatgtaaatgctggtaaatccaccacaaatatcatgataactaccataattatagtaattatagtaatattgttagcattaattgcagttggactgcttctatactgcaaggccagaagcacaccagtcacattaagtaaggatcaactgagtggtataaataatattgcatttagtaactga

SEQ ID NO: 26 - RNA, 1725 nt, Artificial Sequence
  Synthetic nucleotide sequence
auggaguugccaauccucaaaacaaaugcuauuaccacaauccuugcugcagucacacucugcuucgcuuccagucaaaacaucacugaagaauuuuaucaaucaacaugcagugcaguuagcaaaggcuaucuuagugcucuaagaacugguugguauacuaguguuauaacuauagaauuaaguaauaucaaggaaaauaaguguaaugguacagaugcuaagguaaaauuaauaaaacaagaauuagauaaauauaaaaaugcuguaacagaauugcaguugcucaugcaaagcacaccagcagccaacagucgagccagaagagaacugccaagauuuaugaauuauacacucaacaauaccaaaaacaccaauguaacauuaaguaagaaaaggaaaagaagauuucuuggauuuuuguuagguguuggaucugcaaucgccaguggcauugccguauccaagguccugcaccuagaaggggaagugaacaaaaucaaaagugcucuacuauccacaaacaaggcuguagucagcuuaucuaauggagucagugucuuaaccagcaagguguuagaccucaaaaacuauauagauaaacaguuguuaccuauuguuaacaagcaaagcugcagcauaucaaacauugaaacugugauagaguuccaacaaaagaacaacagacuacuagagauuaccagagaauuuaguguuaaugcagguguaacuacaccuguaagcacuuauauguuaacuaauagugaguuauuaucauuaaucaaugauaugccuauaacaaaugaucagaaaaaguuaauguccagcaauguucaaauaguuagacagcaaaguuacucuaucaugucaauaauaaaagaggaagucuuagcauauguaguacaauuaccacuauaugguguaauagauacuccuuguuggaaacuacacacauccccucuauguacaaccaacacaaaggaaggauccaacaucugcuuaacaagaaccgacagaggaugguacugugacaaugcaggaucaguauccuuuuucccacaagcugaaacauguaaaguucaaucgaaucggguauuuugugacacaaugaacaguuuaacauuaccaagugagguaaaucucugcaacauugacauauucaaccccaaauaugauugcaaaauuaugacuucaaaaacagauguaagcagcuccguuaucacaucucuaggagccauugugucaugcuauggcaaaaccaaauguacagcauccaauaaaaaucgugggaucauaaagacauucucuaacgggugugauuauguaucaaauaaggggguggauacugugucuguagguaauacauuauauuauguaaauaagcaagaaggcaaaagucucuauguaaaaggugaaccaauaauaaauuucuaugauccauuaguguuccccucugaugaauuugaugcaucaauaucucaagucaaugagaaaauuaaucagagucuagcauuuauccguaaaucagaugaauuauuacauaauguaaaugcugguaaauccaccacaaauaucaugauaacuaccauaauuauaguaauuauaguaauauuguuagcauuaauugcaguuggacugcuucuauacugcaaggccagaagcacaccagucacauuaaguaaggaucaacugagugguauaaauaauauugcauuuaguaacuga

SEQ ID NO: 27 - protein, 574 aa, Artificial Sequence
  Synthetic peptide sequence
MELLIHRSSAIFLTLAINALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 28 - DNA, 1725 bp, Artificial Sequence
  Synthetic nucleotide sequence
atggagttgctgatccatagatcaagtgcaatcttcctaactcttgctattaatgcattgtacctcacctcaagtcagaacataactgaggagttttaccaatcgacatgtagtgcagttagcagaggttactttagtgctttaagaacaggttggtataccagtgtcataacaatagaattaagtaatataaaagaaaccaaatgcaatggaactgacactaaagtaaaacttataaaacaagaattagataagtataagaatgcagtaacagaattacagttacttatgcaaaacacaccagctgccaacaaccgagccagaagagaagcaccacagtatatgaactacacaatcaataccactaaaaacctaaatgtatcaataagcaagaagaggaaacgaagatttctgggcttcttgttaggtgtaggatctgcaatagcaagtggcatagctgtatccaaagttctacaccttgaaggagaagtgaacaagatcaaaaatgctttgctgtctacaaacaaagctgtagtcagtctatcaaatggggtcagtgttttaaccagcaaagtgttagatctcaagaattatataaacaaccaattattacctatagtaaatcaacagagttgtcgcatctccaacattgaaacagttatagaattccagcagaagaacagcagattgttggaaatcaccagagaatttagtgtcaatgcaggtgtaacgacacctttaagcacttacatgttaacaaacagtgagttactatcattaatcaatgatatgcctataacaaatgatcagaagaaattaatgtcaagcaatgttcagatagtaaggcaacaaagttattctatcatgtctataataaaggaagaagtcctcgcatatgttgtacagctacctatctatggtgtaatagatacaccttgctggaaattacacacatcacctctgtgcaccaccaacatcaaagaaggatcaaatatttgtttaacaaggactgatagaggatggtactgtgataatgcaggatcagtatccttctttccacaggctgacacttgtaaagtacagtccaatcgagtattttgtgacactatgaacagtttgacattaccaagtgaagtcagcctttgtaacactgacatattcaattccaagtatgactgcaaaattatgacatcaaaaacagacataagcagctcagtaattacttctctaggagctatagtgtcatgctatggtaaaactaaatgcactgcatccaacaaaaatcgtggaattataaagacattttctaatggttgtgattatgtgtcaaacaaaggagtagatactgtatcagtaggcaacactttatactatgtcaacaagctggaaggcaaaaacctttatgtaaaaggggaacctataataaattactatgaccctctagtgtttccttctgatgagtttgatgcatcaatatctcaagtcaatgaaaaaattaatcaaagtttagcttttattcgtagatccgatgaattattacataatgtaaatactggaaaatctactacaaatattatgataactgcaattattatagtaatcattgtagtattgttatcattaatagctattggtttactgttgtattgcaaagccaaaaacacaccagttacactaagcaaagaccaactaagtggaatcaacaacattgcattcagcaaatag

SEQ ID NO: 29 - RNA, 1725 nt, Artificial Sequence
  Synthetic nucleotide sequence
auggaguugcugauccauagaucaagugcaaucuuccuaacucuugcuauuaaugcauuguaccucaccucaagucagaacauaacugaggaguuuuaccaaucgacauguagugcaguuagcagagguuacuuuagugcuuuaagaacagguugguauaccagugucauaacaauagaauuaaguaauauaaaagaaaccaaaugcaauggaacugacacuaaaguaaaacuuauaaaacaagaauuagauaaguauaagaaugcaguaacagaauuacaguuacuuaugcaaaacacaccagcugccaacaaccgagccagaagagaagcaccacaguauaugaacuacacaaucaauaccacuaaaaaccuaaauguaucaauaagcaagaagaggaaacgaagauuucugggcuucuuguuagguguaggaucugcaauagcaaguggcauagcuguauccaaaguucuacaccuugaaggagaagugaacaagaucaaaaaugcuuugcugucuacaaacaaagcuguagucagucuaucaaauggggucaguguuuuaaccagcaaaguguuagaucucaagaauuauauaaacaaccaauuauuaccuauaguaaaucaacagaguugucgcaucuccaacauugaaacaguuauagaauuccagcagaagaacagcagauuguuggaaaucaccagagaauuuagugucaaugcagguguaacgacaccuuuaagcacuuacauguuaacaaacagugaguuacuaucauuaaucaaugauaugccuauaacaaaugaucagaagaaauuaaugucaagcaauguucagauaguaaggcaacaaaguuauucuaucaugucuauaauaaaggaagaaguccucgcauauguuguacagcuaccuaucuaugguguaauagauacaccuugcuggaaauuacacacaucaccucugugcaccaccaacaucaaagaaggaucaaauauuuguuuaacaaggacugauagaggaugguacugugauaaugcaggaucaguauccuucuuuccacaggcugacacuuguaaaguacaguccaaucgaguauuuugugacacuaugaacaguuugacauuaccaagugaagucagccuuuguaacacugacauauucaauuccaaguaugacugcaaaauuaugacaucaaaaacagacauaagcagcucaguaauuacuucucuaggagcuauagugucaugcuaugguaaaacuaaaugcacugcauccaacaaaaaucguggaauuauaaagacauuuucuaaugguugugauuaugugucaaacaaaggaguagauacuguaucaguaggcaacacuuuauacuaugucaacaagcuggaaggcaaaaaccuuuauguaaaaggggaaccuauaauaaauuacuaugacccucuaguguuuccuucugaugaguuugaugcaucaauaucucaagucaaugaaaaaauuaaucaaaguuuagcuuuuauucguagauccgaugaauuauuacauaauguaaauacuggaaaaucuacuacaaauauuaugauaacugcaauuauuauaguaaucauuguaguauuguuaucauuaauagcuauugguuuacuguuguauugcaaagccaaaaacacaccaguuacacuaagcaaagaccaacuaaguggaaucaacaacauugcauucagcaaauag

SEQ ID NO: 30 - protein, 84 aa, Artificial Sequence
  Synthetic peptide sequence
QNITEEFYQSTCSAVSKGYLSALRTGWYTSVITIELSNIKENKCNGTDAKVKLIKQELDKYKNAVTELQLLMQSTPACNSRARR

SEQ ID NO: 31 - protein, 377 aa, Artificial Sequence
  Synthetic peptide sequence
FLGFLLGVGSACASGIAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSSNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELL

SEQ ID NO: 32 - protein, 84 aa, Artificial Sequence
  Synthetic peptide sequence
QNITEEFYQSTCSAVSRGYFSALRTGWYTSVITIELSNIKETKCNGTDTKVKLIKQELDKYKNAVTELQLLMQNTPACNNRARR

SEQ ID NO: 33 - protein, 377 aa, Artificial Sequence
  Synthetic peptide sequence
FLGFLLGVGSACASGIAVSKVLHLEGEVNKIKNALLSTNKAVVSLSNGVSVLTIKVLDLKNYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSSEFDASISQVNEKINQSLAFIRRSDELL

SEQ ID NO: 34 - protein, 568 aa, Artificial Sequence
  Synthetic peptide sequence
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSLAFIRKSDELLSAIGGYIPEAPRDGQAYVRKDGEWVLLSTFLGGLVPRGSHHHHHHGSWSHPQFEK

SEQ ID NO: 35 - DNA, 1707 bp, Artificial Sequence
  Synthetic nucleotide sequence
atggaacttctgatcctgaaagccaacgcgattaccactatcctgactgccgtcaccttctgcttcgcatcgggacagaacattaccgaggagttctaccagtccacctgttcggcggtgtccaagggttacctctcggccctgagaactggctggtacaccagcgtgattactatcgagctgagcaacatcaaggagaacaagtgcaatggaacggacgcgaaggtcaagctgattaagcaggaactcgataagtacaagaacgccgtgaccgagctccagctgctgatgcaatcgacccctgcctgtaacaacagagctcgccgggaactgccgcgcttcatgaattacaccctcaacaacgcgaagaaaaccaacgtgaccctgtccaagaagcgcaagcggaggttcctgggattcctgttgggcgtgggctccgcatgtgcatccggagtggccgtgtccaaagtgctgcatctggagggggaagtgaacaagatcaagtccgccctcctgtcaactaataaggcggtggtgtccctgagcaacggagtcagcgtgctgacaatcaaggtcctggacctcaagaactacatcgacaagcagctgttgcccatcgtcaacaagcagtcatgctcgattagcaatatcgaaaccgtgattgagttccagcagaagaacaacagactgctcgaaattacccgggagttttccgtgaacgccggagtgaccactcctgtgtccacctacatgcttacgaactccgaactgctcagcctcatcaacgatatgccgatcactaacgaccagaagaagttgatgagcaacaatgtgcagatcgtgcgccaacagtcctactcaatcatgtcaattatcaaggaggaagtgctcgcctatgtggtgcaattgcctctgtacggagtcatcgacacaccctgctggaagctgcacactagcccactctgtacgaccaacaccaaggaaggttccaacatctgcctgactaggaccgatcggggctggtattgcgataatgctgggtccgtgagcttcttcccgcaagccgagacttgcaaagtgcagtcaaaccgcgtgttctgtgacaccatgaacagcctgaccctgccatccgaagtcaacctctgcaacgtggacatctttaacccgaaatacgactgcaagattatgacctccaagaccgacgtcagcagctctgtcatcactagcctgggagctattgtgtcctgctacggaaagaccaaatgcactgcctcgaacaagaacagaggcatcatcaagaccttcagcaacggctgtgactacgtgtccaacaagggagtggacaccgtgtccgtcgggaacaccctgtactacgtgaacaagcaggaggggaagtcgctctacgtcaagggggaaccgattatcaatttctacgaccccctggtgttcccttcctccgagttcgatgcctccatatcccaagtcaacgagaagatcaaccagtctcttgccttcatccggaagtcggacgaactgctgtccgccatcggtggctatattccggaagcccccagggatggacaggcctacgtgcggaaggatggagaatgggtgcttttgtccaccttcctgggcggtctggtgccccgcggctcacaccatcatcaccaccacggttcgtggtcccaccctcaatttgagaagtga

SEQ ID NO: 36 - RNA, 1707 nt, Artificial Sequence
  Synthetic nucleotide sequence
auggaacuucugauccugaaagccaacgcgauuaccacuauccugacugccgucaccuucugcuucgcaucgggacagaacauuaccgaggaguucuaccaguccaccuguucggcgguguccaaggguuaccucucggcccugagaacuggcugguacaccagcgugauuacuaucgagcugagcaacaucaaggagaacaagugcaauggaacggacgcgaaggucaagcugauuaagcaggaacucgauaaguacaagaacgccgugaccgagcuccagcugcugaugcaaucgaccccugccuguaacaacagagcucgccgggaacugccgcgcuucaugaauuacacccucaacaacgcgaagaaaaccaacgugacccuguccaagaagcgcaagcggagguuccugggauuccuguugggcgugggcuccgcaugugcauccggaguggccguguccaaagugcugcaucuggagggggaagugaacaagaucaaguccgcccuccugucaacuaauaaggcgguggugucccugagcaacggagucagcgugcugacaaucaagguccuggaccucaagaacuacaucgacaagcagcuguugcccaucgucaacaagcagucaugcucgauuagcaauaucgaaaccgugauugaguuccagcagaagaacaacagacugcucgaaauuacccgggaguuuuccgugaacgccggagugaccacuccuguguccaccuacaugcuuacgaacuccgaacugcucagccucaucaacgauaugccgaucacuaacgaccagaagaaguugaugagcaacaaugugcagaucgugcgccaacaguccuacucaaucaugucaauuaucaaggaggaagugcucgccuauguggugcaauugccucuguacggagucaucgacacacccugcuggaagcugcacacuagcccacucuguacgaccaacaccaaggaagguuccaacaucugccugacuaggaccgaucggggcugguauugcgauaaugcuggguccgugagcuucuucccgcaagccgagacuugcaaagugcagucaaaccgcguguucugugacaccaugaacagccugacccugccauccgaagucaaccucugcaacguggacaucuuuaacccgaaauacgacugcaagauuaugaccuccaagaccgacgucagcagcucugucaucacuagccugggagcuauuguguccugcuacggaaagaccaaaugcacugccucgaacaagaacagaggcaucaucaagaccuucagcaacggcugugacuacguguccaacaagggaguggacaccguguccgucgggaacacccuguacuacgugaacaagcaggaggggaagucgcucuacgucaagggggaaccgauuaucaauuucuacgacccccugguguucccuuccuccgaguucgaugccuccauaucccaagucaacgagaagaucaaccagucucuugccuucauccggaagucggacgaacugcuguccgccaucgguggcuauauuccggaagcccccagggauggacaggccuacgugcggaaggauggagaaugggugcuuuuguccaccuuccugggcggucuggugccccgcggcucacaccaucaucaccaccacgguucguggucccacccucaauuugagaaguga

SEQ ID NO: 37 - DNA, 13476 bp, Artificial Sequence
  Synthetic nucleotide sequence
taatacgactcactatagataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcgcaccatgaagtgcctgctgtacctggccttcctgttcatcggcgtgaactgcaagtttaccatcgtgttccctcacaaccagaaaggcaactggaagaacgtgcccagcaactaccactactgccccagcagcagcgacctgaactggcacaatgacctgatcggcacagccctgcaagtgaagatgcccaagagccacaaggccatccaggccgatggatggatgtgccacgccagcaaatgggtcaccacctgtgacttccgttggtacggccccaagtacatcacccacagcatccgcagcttcacccctagcgtggaacagtgcaaagagagcatcgagcagaccaagcaaggcacctggctgaaccctggattcccacctcagagctgtggctacgccactgtgacagatgccgaggccgtgatcgtgcaagtgacacctcatcacgtgctggtggacgagtacacaggcgagtgggtcgacagccagttcatcaacggcaagtgctccaactacatctgccccaccgtgcacaacagcaccacctggcacagcgactacaaagtgaagggcctgtgcgacagcaacctgatcagcatggacatcacctttttcagcgaggacggcgagctgagcagcctgggaaaagaaggcaccggcttcagaagcaactacttcgcctatgagacaggcggcaaggcctgcaagatgcagtattgcaagcactggggcgtcagactgcctagcggagtttggttcgagatggccgacaaggacctgtttgccgccgctagattccctgagtgtcctgagggcagcagcatctctgcccctagccagacatctgtggacgtgtccctgatccaggacgtggaacggatcctggactacagcctgtgccaagagacatggtccaagatcagagccggcctgcctatcagccctgtggacctgtcttatctggcccctaagaaccctggcaccggacctgccttcaccatcatcaatggcaccctgaagtactttgagacacggtacatccgcgtggacattgccgctcctatcctgagcagaatggtcggaatgatcagcggcaccaccaccgagagggaactgtgggatgattgggccccttacgaggatgtggaaatcggccctaacggcgtgctgagaacaagcagcggctacaagttcccactgtacatgatcggccacggcatgctggacagcgatctgcacctgtctagcaaggcccaggtgttcgagcaccctcacattcaggatgccgccagccagctgcctgatgacgagagcctgttctttggcgataccggcctgagcaagaaccccatcgaactggtggaaggctggttcagcagctggaagtcctctatcgccagcttcttcttcatcatcgggctgattatcggcctgttcctggtgctgagagtgggcatccacctgtgcatcaagctgaagcacaccaagaagcggcagatctacaccgacatcgagatgaaccggctgggcaagtgatgaatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgccgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgacgttaat